(19) United States
(12) Patent Application Publication
(10) Pub. No.: US 2026/0216288 A1
(43) Pub. Date: Jul. 30, 2026

(54) COMBINATION THERAPY WITH CD13-TARGETED CHIMERIC PROTEINS OR CHIMERIC PROTEIN COMPLEXES

(71) Applicants: Orionis Biosciences, Inc., Waltham, MA (US); Orionis Biosciences BV, Zwijnaarde (BE); VIB VZW, Gent (BE); Universiteit Gent, Gent (BE)

(72) Inventors: Nikolai KLEY, Waltham, MA (US); Jan TAVERNIER, Zwijnaarde (BE); Leander HUYGHE, Beernem (BE)

(21) Appl. No.: 19/444,278

(22) Filed: Jan. 9, 2026

Related U.S. Application Data
(63) Continuation of application No. 17/620,329, filed on Jun. 18, 2020, now Pat. No. 12,544,426.
(60) Provisional application No. 62/863,447, filed on Jun. 19, 2019.

(51) Int. Cl. A61K 38/17; A61K 9/00; A61K 9/1271; A61K 31/704; A61K 38/19; A61K 38/21; A61K 39/00; A61P 35/00

(52) U.S. Cl. CPC A61K 38/1774; A61K 9/0019; A61K 9/1271; A61K 31/704; A61K 38/191; A61K 38/217; A61P 35/00; A61K 2039/505

(57) ABSTRACT

The present invention relates, in part, to chimeric protein or chimeric protein complex comprising a CD13 targeting moiety and a signaling agent (e.g., without limitation TNF or IFN-γ) and methods of treatment using such compositions.

### RELATED APPLICATIONS

This application is a continuation application of U.S. application Ser. No. 17/620,329, filed Dec. 17, 2021 (now U.S. Pat. No. 12,544,426), which is a National Stage Entry of PCT/US20/38373, filed Jun. 18, 2020, which claims the benefit of U.S. Provisional Patent Application No. 62/863,447, filed Jun. 19, 2019, the entire disclosure of which is hereby incorporated by reference in its entirety.

### FIELD

The present invention relates, in part, to CD13-targeted chimeric proteins or chimeric protein complexes and their use as therapeutic agents in combination therapy.

### DESCRIPTION OF THE SEQUENCE LISTING

The contents of the XML Sequence Listing submitted electronically herewith are incorporated herein by reference in their entirety: a XML copy of the Sequence Listing (filename: ORN-047C1_114384-5047.xml; date recorded: Apr. 6, 2026; file size: 493,709 bytes).

### BACKGROUND

Cancer is a global health challenge that causes nearly 7 million deaths each year worldwide and which has, to date, proven largely untreatable despite major advances in medicine. Frustratingly, cancers appear to develop strategies to evade immune detection and destruction thereby sidestepping the body's main protection against the disease. Increased expression of various hydrolytic enzymes like peptidases, esterases, and proteases has been described in several types of human malignancies, especially those characterized by fast-growing and aggressive phenotypes. One of the peptidases involved in cancer is aminopeptidase N (also known as CD13), a Zn²⁺ dependent membrane-bound ectopeptidase that degrades preferentially proteins and peptides with a N-terminal neutral amino acid.

CD13 is highly expressed in multiple human cancers. Its expression is induced and particularly pronounced in endothelial cells of the tumor neovasculature compared to normal vasculature. It can also be expressed on some tumor cells, e.g. from epithelial origin. Multiple regulatory functions have been ascribed to CD13, including regulation of endothelial cell morphology, formation of neovascular blood vessels (neoangiogenesis), cell differentiation, proliferation and motility. Therefore, CD13 is both a marker and a functional regulator of the tumor microenvironment. There remains a need for novel therapeutic agents that can effectively target cancers.

By virtue of its function and, in particular, its disease-associated expression profiles, CD13 represents an intriguing target for various strategies aimed at inhibiting cancer growth and cancer immunoregulation. We describe novel strategies and therapeutic agents targeting CD13 to modulate endothelial cell functions and various processes associated with tumor neovasculature and cancer growth.

Accordingly, there remains a need for novel therapeutic agents that can effectively target cancers, including CD13-driven cancers.

### SUMMARY

In various aspects, the present technology relates to therapeutic uses of CD13-targeted chimeric proteins or chimeric protein complexes having at least one targeting moiety that specifically binds to CD13 and at least one signaling agent that is a tumor necrosis factor (TNF). In various embodiments, the TNF signaling agent may be modified to attenuate activity. In some embodiments, the present CD13-targeted chimeric proteins or chimeric protein complexes may directly or indirectly recruit an immune cell to a site of action (such as, by way of non-limiting example, the tumor microenvironment).

In some aspects, the present technology relates to therapeutic uses of CD13-targeted chimeric proteins or chimeric protein complexes having at least one targeting moiety that specifically binds to CD13 and at least one signaling agent that is an interferon (IFN) or a modified form thereof. In various embodiments, the IFN signaling agent may be modified to attenuate activity. In one embodiment, the interferon is IFN-γ or a modified form thereof.

In various aspects, the present technology relates to the co-administration of the CD13-targeted chimeric proteins or chimeric protein complexes with at least one other therapeutic agent. In some embodiments, the other therapeutic agent is a CD8-targeted chimeric protein or chimeric protein complex having at least one targeting moiety that specifically binds to CD8 and at least one signaling agent that is an interferon (IFN) or a modified form thereof. In various embodiments, the IFN signaling agent may be modified to attenuate activity. In some embodiments, the other therapeutic agent is another CD13-targeted chimeric protein or chimeric protein complex having at least one targeting moiety that specifically binds to CD13 and at least one signaling agent that is an interferon (IFN) or a modified form thereof. In various embodiments, the IFN signaling agent may be modified to attenuate activity. In one embodiment, the interferon is IFN-γ or a modified form thereof.

In some embodiments, the at least one other therapeutic agent is one or more agents selected from a phosphoinositide-3-kinase 9 (PI3K) inhibitor, anthracycline, and SMAC mimetic. In some embodiments, the anthracycline is a liposomal anthracycline.

In various embodiments, the co-administration of CD13-targeted chimeric proteins or chimeric protein complexes with at least one other therapeutic agent is useful in the treatment of various diseases or disorders such as cancer, immune disorders, and other diseases and disorders.

In some embodiments, the present invention relates to chimeric protein complexes where the chimeric protein complex includes one or more signaling agents, one or more targeting agents, and one or more fragment crystallizable domains (Fc domains). These Fc-based chimeric protein complexes of the present invention are highly target selective, enable conditional and/or regulated modulation of receptor signaling, and are highly active and/or long-acting active and/or long-acting while eliciting minimal side effects.

### BRIEF DESCRIPTION OF DRAWINGS

FIG. 1 shows immunohistochemical analysis of B16BI6 tumor after p.l. treatment with 5 μg wild-type sc mTNF or 50 μg of the AcTakine mCD13 VHH-sc mTNF Y114L. The upper panel was taken 4 hours after treatment and stained for mouse ICAM-1. The lower panel was taken 24 hours after treatment and shows a specific fluorescence in blood clots. Sections were made transverse and pictures were taken close to the skin, near the site of injection.

FIG. 2A is a graph showing the change in B16BI6 tumor size in B16 mice treated with PBS, CD8-targeted chimeric protein (CD8-Actaferon) alone, Bcll10 targeted mutated murine TNF (Y114L) alone, or CD8-targeted chimeric protein (CD8-Actaferon) and Bcll10 targeted mutated murine TNF (Y114L). FIG. 2B is a graph showing the change in B16BI6 tumor size in B16 mice treated with PBS, CD8-targeted chimeric protein (CD8-Actaferon) alone, CD13 targeted mutated murine TNF (Y114L) alone, or CD8-targeted chimeric protein (CD8-Actaferon) and CD13 targeted mutated murine TNF (Y114L). FIG. 2C shows B16BI6 tumor growth and body weight change after daily p.l. injection of 30 μg mCD8-AFN, 50 μg mCD13-AFR or combinations thereof. Tumor growth is shown as mean TSI+SEM (n=6 per group). The line under the graph represents the treatment period. FIG. 2D shows TSI of individual tumors of the indicated treatment groups at day 19 after tumor inoculation. Error bars are SEM. ***, p<0.001 by one-way ANOVA with Bonferroni's multiple comparison test.

FIG. 3A is a graph showing the change in B16BI6 tumor size in B16 mice treated with PBS or mTNF. FIG. 3B is a graph showing the change in B16BI6 tumor size in B16 mice treated with PBS, CD13 targeted mutated murine TNF (Y114L) alone, Wortmannin alone, or CD13 targeted mutated murine TNF (Y114L) and Wortmannin.

FIG. 4A-B are graphs showing the change in B16BI6 tumor size (FIG. 4A) and change in weight (FIG. 4B) in B16 mice treated with PBS, CD13 targeted mutated murine TNF (Y114L) alone, PEGylated liposomal doxorubicin alone, or CD13 targeted mutated murine TNF (Y114L) and PEGylated liposomal doxorubicin.

FIGS. 5A-B are graphs showing the change in B16BI6 tumor size (FIG. 5A) and change in weight (FIG. 5B) in B16 mice treated with PBS, CD13 targeted mutated murine TNF (Y114L) alone, birinapant alone, or CD13 targeted mutated murine TNF (Y114L) and birinapant.

FIGS. 6A-B are exemplary, non-limiting embodiments of CD13-targeted chimeric proteins (hTNF=human tumor necrosis factor).

FIGS. 7A-E show that sc mTNF Y86F is non-toxic and mCD13-AFR is active in mice. FIG. 7A: Toxicity of wt sc mTNF and mutant Y86F after injection (1) in naïve C56BL/6 mice. First injection was i.v., other injections were i.p. Mean rectal body temperature±SEM and cumulative survival rates are shown (n=4 per group). For continuity of the temperature graphs, dead mice were included with a temperature of 20° C. FIG. 7B: Plasma IL-6 concentrations measured via ELISA. Blood samples were taken 6 hours after i.v. injection of wt sc mTNF or mutant Y86F. ns, non-significant by one-way ANOVA with Bonferroni's multiple comparison test. FIG. 7C: Immunohistochemical staining for PECAM-1 (red), ICAM-1 (green) and DNA (blue) of B16BI6 tumors after p.l. treatment with 7 μg wt mTNF or 50 μg of mCD13-AFR. Scale bar is 50 μm. FIG. 7D: Expression of ICAM-1, E-selectin and VEGF-R2 in B16BI6 tumor 6 h after treatment, by qPCR analysis of whole tumor RNA. Error bars are SEM. ns, non-significant; *, p<0.05; **, p<0.01; ***, p<0.001 by one-way ANOVA with Bonferroni's multiple comparison test. FIG. 7E: Tumor growth and body weight change after daily p.l. injection of 50 μg mCD13-AFR in the B16BI6 melanoma model. The line under the graph represents the treatment period. Tumor growth is shown as mean TSI, error bars are SEM (n=5 per group). ***, p<0.001 by two-way ANOVA with Bonferroni's multiple comparison test on day 21.

FIG. 8A-E show that IFN-γ sensitizes tumor endothelial cells for TNF. FIGS. 8A and 8B: B16BI6 tumor growth after daily p.l. treatment with 7 μg mTNF, 30 μg hTNF alone or in combination with 10000 IU mIFN-γ in wt C57BL/6 mice (a) or homozygous Flk1 dnIFN-γR transgenic mice. The line under the graph represents the treatment period. Tumor growth is depicted as mean TSI, error bars are SEM (n=5 per group). FIG. 8C: IL-8 concentration in HUVEC supernatant after 24 h stimulation with the indicated concentration of sc hTNF mutant Y87F (Y87F), hCD13-AFR or wt hTNF, alone or in combination with 200 ng/ml hIFN-γ, measured via ELISA. Error bars are SEM. ns, non-significant; **, p<0.01; ***, p<0.001 by one-way ANOVA with Bonferroni's multiple comparison test. FIG. 8D: Schematic representation of AFN-II, consisting of a VHH fused via a 20xGGS linker to an engineered IFN-γ (e-IFN-γ) variant and N-terminal affinity tag. FIG. 8E: Viability of B16BI6 parental or mCD20-expressing cells after 72 h stimulation with wt mIFN-γ or del8 mutant fused to a Bcll10 or mCD20 VHH, in the presence of 60 ng/ml mTNF. Cell viability was measured via an ATP luminescence assay. Each point is the mean of three replicates and error bars are SEM.

FIGS. 9A-B show the synergistic antitumor effect of TNF and IFN-γ depends on expression of IFN-γR on host cells but not on tumor cells. FIGS. 9A and 9B: TSI of B16-dnIFN-γR tumor in wt C57BL/6 mice (a) or B16BI6 parental tumor in IFN-γR^{-/-} mice (b). In wt mice, the growth rate of a B16-dnIFN-γR tumor was similar to B16BI6 parental tumor. Mice were treated daily via p.l. injection of 7 μg mTNF, 30 μg hTNF, or a combination of 30 μg hTNF and 10000 IU mIFN-γ. Error bars are SEM (n=7 per group). The line under the graph represents the treatment period.

FIG. 10A-B show endothelial expression of the dnIFN-γR transgene in Flk1 dnIFN-γR^{tg/tg} mice. FIG. 10A: Cryosections of embryos on day E10.5 were stained with anti-c-Myc Ab, followed by chromogenic detection with Alkaline Phosphatase conjugated anti-mlgG Ab and BCIP/NBT substrate. Nuclei were stained with haematoxylin. Expression was visible in endothelial cells of large veins (v), sinus venosus (sv) and aorta (ao). FIG. 10B: Cryosections of B16BI6 tumors inoculated in adult transgenic mice were stained with biotinylated anti-c-Myc Ab and visualised using streptavidin-FITC. Nuclei were stained with DAPI. Tumor endothelial cells showed positive staining. Scale bars are 50 μm.

FIG. 11 shows the bioactivity of mIFN-γ del8 mutant in the EMCV cytopathic effect assay in L929 cells. L929 cells were stimulated for 24 h with wt mIFN-γ or del8 mutant before virus (~99% CPE) was added. One day later, cell viability was measured via an ATP luminescence assay.

FIGS. 12A-C show that CD13-AFN-II synergizes with CD13-AFR for tumor destruction. FIGS. 12A and 12B: Tumor growth, body weight change and rectal body temperature after daily p.l. treatment with mCD13-AFR, mCD13-AFN-II or combination thereof, in the B16BI6 model in C57BL/6 mice (a) or RL model in NSG mice (b). Tumor growth is shown as mean TSI, error bars are SEM (n=5 per group) (upper panel), or as TSI of individual mice (lower panel). The line under the graph represents the treatment period. FIG. 12C: Immunohistochemical staining for PECAM-1 (red), cleaved Caspase-3 (green) and DNA (blue) of B16BI6 tumors after p.l. treatment with 50 μg mCD13-AFR and 23.8 μg mCD13-AFN-II. Scale bar is 100 μm.

FIGS. 13A-F, 14A-H, 15A-H, 16A-D, 17A-F, 18A-J, 19A-D, 20A-F, 21A-J, 22A-F, 23A-L, 24A-L, 25A-F, 26A-L, 27A-L, 28A-J, 29A-J, 30A-F, and 31A-F show various non-limiting illustrative schematics of the Fc-based chimeric protein complexes of the present invention. In embodiments, each schematic is a composition of the present invention. Where applicable in the figures, “TM” refers to a “targeting moiety” as described herein, “SA” refers to a “signaling agent” as described herein, [symbol] is an optional “linker” as described herein, the two long parallel rectangles are human Fc domains, e.g. from IgG1, from IgG2, or from IgG4, as described herein and optionally with effector knock-out and/or stabilization mutations as also described herein, and the two long parallel rectangles with one having a protrusion and the other having an indentation are human Fc domains, e.g. from IgG1, from IgG2, or from IgG4 as described herein, with knob-in-hole and/or ionic pair (a/k/a charged pairs, ionic bond, or charged residue pair) mutations as described herein and optionally with effector knock-out and/or stabilization mutations as also described herein.

FIGS. 13A-F show illustrative homodimeric 2-chain complexes. These figures show illustrative configurations for the homodimeric 2-chain complexes.

FIGS. 14A-H show illustrative homodimeric 2-chain complexes with two targeting moieties (TM) (as described herein, more targeting moieties may be present in some embodiments). In embodiments, the position of TM1 and TM2 are interchangeable. In embodiments, the constructs shown in the box (i.e., FIGS. 14G and 14H) have signaling agent (SA) between TM1 and TM2 or between TM1 and Fc.

FIGS. 15A-H show illustrative homodimeric 2-chain complexes with two signaling agents (as described herein, more signaling agents may be present in some embodiments). In embodiments, the position of SA1 and SA2 are interchangeable. In embodiments, the constructs shown in the box (i.e., FIGS. 15G and 15H) have TM between SA1 and SA2 or TM at N- or C-terminus).

FIGS. 16A-D show illustrative heterodimeric 2-chain complexes with split TM and SA chains, namely the TM on the knob chain of the Fc and the SA on hole chain of the Fc.

FIGS. 17A-F show illustrative heterodimeric 2-chain complexes with split TM and SA chains, namely with both TMs on the knob chain of the Fc and with SA on hole chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 18A-J show illustrative heterodimeric 2-chain complexes with split TM and SA chains, namely with TM on the knob chain of the Fc and with a SA on the hole chain of the Fc, with two signaling agents (as described herein, more signaling agents may be present in some embodiments). In these orientations and/or configurations, one SA is on the knob chain and one SA is on the hole chain. In embodiments, the position of SA1 and SA2 are interchangeable.

FIGS. 19A-D show illustrative heterodimeric 2-chain complexes with split TM and SA chains, namely the SA on the knob chain of the Fc and the TM on hole chain of the Fc.

FIGS. 20A-F show illustrative heterodimeric 2-chain complexes with split TM and SA chains, namely with SA on the knob chain of the Fc and both TMs on hole chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 21A-J show illustrative heterodimeric 2-chain complexes with split TM and SA chains, namely with SA on the knob chain of the Fc and TM on hole chain of the Fc, with two signaling agents (as described herein, more signaling agents may be present in some embodiments). In these orientations and/or configurations, one SA is on the knob chain and one SA is on the hole chain. In embodiments, the position of SA1 and SA2 are interchangeable.

FIGS. 22A-F show illustrative heterodimeric 2-chain complexes with TM and SA on the same chain, namely the SA and TM both on the knob chain of the Fc.

FIGS. 23A-L show illustrative heterodimeric 2-chain complexes with a TM and a SA on the same chain, namely with SA and with TM both on the knob chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 24A-L show illustrative heterodimeric 2-chain complexes with a TM and a SA on the same chain, namely with SA and with TM both on the knob chain of the Fc, with two signaling agents (as described herein, more signaling agents may be present in some embodiments). In embodiments, the position of SA1 and SA2 are interchangeable.

FIGS. 25A-F show illustrative heterodimeric 2-chain complexes with TM and SA on the same chain, namely the SA and TM both on the hole chain of the Fc.

FIGS. 26A-L show illustrative heterodimeric 2-chain complexes with a TM and a SA on the same chain, namely with SA and with TM both on the hole chain of the Fc, with two targeting moieties (as described herein, more targeting moieties are present in some embodiments). In embodiments, the position of TM1 and TM2 are interchangeable. In embodiments, TM1 and TM2 can be identical.

FIGS. 27A-L show illustrative heterodimeric 2-chain complexes with a TM and a SA on the same chain, namely with SA and with TM both on the hole chain of the Fc, with two signaling agents (as described herein, more signaling agents may be present in some embodiments). In embodiments, the position of SA1 and SA2 are interchangeable.

FIGS. 28A-J show illustrative heterodimeric 2-chain complexes with two targeting moieties (as described herein, more targeting moieties may be present in some embodiments) and with SA on knob Fc and TM on each chain. In embodiments, TM1 and TM2 can be identical.

FIGS. 29A-J show illustrative heterodimeric 2-chain complexes with two targeting moieties (as described herein, more targeting moieties may be present in some embodiments) and with SA on hole Fc and TM on each chain. In embodiments, TM1 and TM2 can be identical.

FIGS. 30A-F show illustrative heterodimeric 2-chain complexes with two signaling agents (as described herein, more signaling agents may be present in some embodiments) and with split SA and TM chains: SA on knob and TM on hole Fc.

FIGS. 31A-F show illustrative heterodimeric 2-chain complexes with two signaling agents (as described herein, more signaling agents may be present in some embodiments) and with split SA and TM chains: TM on knob and SA on hole Fc.

### DETAILED DESCRIPTION

The present technology is based, in part, on the discovery of the use of CD13-targeted chimeric proteins or chimeric protein complexes in combination with at least one other therapeutic agent in the treatment of diseases and disorders. In some embodiments, the CD13-targeted chimeric proteins or chimeric protein complexes include at least one CD13 targeting moiety and at least one tumor necrosis factor (TNF) signaling agent. In various embodiments, the TNF signaling agent may be modified to have attenuated activity.

In some embodiments, these CD13-targeted chimeric proteins or chimeric protein complexes may bind and directly or indirectly recruit immune cells to sites in need of therapeutic action (e.g. a tumor or tumor microenvironment or tumor vasculature). In some embodiments, these CD13-targeted chimeric proteins or chimeric protein complexes bind to, but do not functionally modulate CD13. In some embodiments, the CD13-targeted chimeric proteins or chimeric protein complexes enhance tumor antigen presentation for elicitation of effective antitumor immune response. In some embodiments, a) the cancer or tumor cells overexpress a CD13 protein or b) endothelial cells of tumor neovasculature overexpress a CD13 protein. In some embodiments, the CD13 protein is overexpressed by neovascular endothelial cells in disease indications associated with increased angiogenesis.

In some embodiments, the chimeric proteins or chimeric protein complexes or their combination with therapeutic agents described herein have a cytotoxic, cell modulatory, or otherwise anti-cellular effect against the tumor vasculature, e.g., they suppress the growth or cell division of vascular endothelial cells of the tumor vasculature, shrink or destroy the developed vasculature around an established tumor, or activate tumor neovasculature endothelial cells and/or endothelial cells associated with neoangiogenesis. The agents described herein can lead to a tumor-localized vascular collapse, depriving the tumor cells, particularly those tumor cells distal of the vasculature, of oxygen and nutrients, ultimately leading to cell death and tumor necrosis. The agents described herein can activate the tumor endothelium (or neoangiogenic endothelium in general) to recruit immune cells and promote immune cell infiltration.

In some embodiments, the chimeric proteins or chimeric protein complexes or their combination with therapeutic agents described herein cause activation of tumor vasculature, e.g. evidenced by expression of leukocyte adhesion markers. In some embodiments, the chimeric proteins or chimeric protein complexes or their combination with therapeutic agents described herein cause disruption of tumor vasculature or tumor necrosis. In some embodiments, the chimeric proteins or chimeric protein complexes or their combination with therapeutic agents described herein cause activation of tumor vasculature, e.g. evidenced by expression of leukocyte adhesion markers and disruption of tumor vasculature or tumor necrosis.

In some embodiments, the chimeric proteins or chimeric protein complexes or their combination with therapeutic agents described herein cause activation of tumor vasculature. In some embodiments, the chimeric proteins or chimeric protein complexes or their combination with therapeutic agents described herein cause activation of tumor vasculature, which is non-injurious. In some embodiments, the chimeric proteins or chimeric protein complexes or their combination with therapeutic agents described herein allow for infiltration of T cells.

In some embodiments, the methods and compositions of the invention are applicable to the treatment or diagnosis of any tumor mass having a vascular endothelial component. Typical vascularized tumors are the solid tumors, particularly carcinomas, which require a vascular component for the provision of oxygen and nutrients. Exemplary solid tumors to which the present invention is directed include but are not limited to carcinomas of the lung, breast, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, neuroblastomas, and the like. In embodiments, the methods and compositions of the invention are applicable to the treatment or diagnosis of a cancer selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments, the methods and compositions of the invention are applicable to the treatment or diagnosis of leukemia or lymphoma. Illustrative leukemias or lymphomas include, but are not limited to, a leukemia or lymphoma selected from B cell lymphoma, non-Hodgkin's lymphoma (NHL) including low grade and intermediate grade non-Hodgkin's lymphomas (NHLs), relapsed Hodgkin's disease, resistant Hodgkin's disease high grade, lymphocyte predominant subtype of Hodgkin's lymphoma, precursor B cell lymphoblastic leukemia/lymphoma, mature B cell neoplasm, B cell chronic lymphocytic leukemia (CLL), small lymphocytic lymphoma (SLL), B cell prolymphocytic leukemia, lymphoplasmacytic lymphoma, mantle cell lymphoma (MCL), follicular lymphoma (FL) including low-grade, intermediate-grade and high-grade FL, cutaneous follicle center lymphoma, marginal zone B cell lymphoma, MALT type marginal zone B cell lymphoma, nodal marginal zone B cell lymphoma, splenic type marginal zone B cell lymphoma, hairy cell leukemia, diffuse large B cell lymphoma, Burkitt's lymphoma, plasmacytoma, plasma cell myeloma, post-transplant lymphoproliferative disorder, Waldenstrom's macroglobulinemia, multiple myeloma, and anaplastic large-cell lymphoma (ALCL). In embodiments, the cancer is a hematologic malignancy, optionally selected from multiple myeloma and 5q-deletion-associated myelodysplastic syndrome (del(5q) MDS). In some embodiments, the cancer is multiple myeloma.

In some embodiments, the methods and composition of the invention are applicable to the treatment or diagnosis of brain metastatic lesions. In some embodiments, the compositions and methods disclosed herein are capable of targeting blood brain barrier at sites of brain metastatic lesions. In some embodiments, the compositions and methods disclosed herein are capable of breaking down the blood brain barrier and/or promote immune cell infiltration. In some embodiments, the chimeric proteins or chimeric protein complexes (whether alone or in combination with therapeutic agents described herein) targeted to CD13 enable selective activation of the tumor neovasculature without detectable toxicity in vivo. In some embodiments, the agents described herein cause upregulation of adhesion markers and support enhanced T cell infiltration leading to elimination of solid tumors. In some embodiments, the chimeric proteins or chimeric protein complexes in combination with the therapeutic agents lead to selective, non-injurious activation of tumor vasculature such that circulating immune cells are attracted to the tumor vasculature.

### CD13 Targeting Moieties

In various embodiments, the present CD13-targeted chimeric proteins or chimeric protein complexes comprise a CD13 targeting moiety. In some embodiments, the CD13-targeted chimeric proteins or chimeric protein complexes include a CD13 targeting moiety and a TNF or a modified form thereof. In some embodiments, the CD13-targeted chimeric proteins or chimeric protein complexes include a CD13 targeting moiety and IFN or a modified form thereof. In some embodiments, the present invention relates to a combination of two or more CD13-targeted chimeric proteins or chimeric protein complexes. For example, in some embodiments, the present invention is related to a combination of a first CD13-targeting chimeric protein or chimeric protein complex which includes a CD13 targeting moiety and a TNF or a modified form thereof and a second CD-13 targeting chimeric protein or chimeric protein complex, which includes a CD13 targeting moiety and a IFN or a modified form thereof. In some embodiments, the CD13 targeting moiety is a protein-based agent capable of specific binding to CD13. In various embodiments, the present CD13 targeting moiety is a protein-based agent capable of specific binding to CD13 without functional modulation (e.g., partial or full neutralization) of CD13. CD13 (also known as aminopeptidase N (APN) is a Zn²⁺ dependent membrane-bound ectopeptidase that degrades, preferentially, proteins and peptides with a N-terminal neutral amino acid. CD13 has been associated with the growth of different human cancers.

In various embodiments, the CD13 targeting moiety of the technology comprises an antigen recognition domain that recognizes an epitope present on CD13. In an embodiment, the antigen-recognition domain recognizes one or more linear epitopes present on CD13. As used herein, a linear epitope refers to any continuous sequence of amino acids present on CD13. In another embodiment, the antigen-recognition domain recognizes one or more conformational epitopes present on CD13. As used herein, a conformation epitope refers to one or more sections of amino acids (which may be discontinuous) which form a three-dimensional surface with features and/or shapes and/or tertiary structures capable of being recognized by an antigen recognition domain.

In various embodiments, the CD13 targeting moiety of the present invention may bind to the full-length and/or mature forms and/or isoforms and/or splice variants and/or fragments and/or any other naturally occurring or synthetic analogs, variants, or mutants of human CD13. In various embodiments, the CD13 targeting moiety of the invention may bind to any forms of the human CD13, including monomeric, dimeric, heterodimeric, multimeric and associated forms. In an embodiment, the CD13 targeting moiety binds to the monomeric form of CD13. In another embodiment, the CD13 targeting moiety binds to a dimeric form of CD13. In a further embodiment, the CD13 targeting moiety binds to glycosylated form of CD13, which may be either monomeric or dimeric.

In an embodiment, the present CD13 targeting moiety comprises a targeting moiety with an antigen recognition domain that recognizes one or more epitopes present on human CD13. In an embodiment, the human CD13 comprises the amino acid sequence of:

(SEQ ID NO: 1)
MAKGFYISKSLGILGILLGVAAVCTIIALSVVYSQEKNKNANSSPVAST
TPSASATTNPASATTLDQSKAWNRYRLPNTLKPDSYRVTLRPYLTPNDR
GLYVFKGSSTVRFTCKEATDVIIIHSKKLNYTLSQGHRVVLRGVGGSQP
PDIDKTELVEPTEYLVVHLKGSLVKDSQYEMDSEFEGELADDLAGFYRS
EYMEGNVRKVVATTQMQAADARKSFPCFDEPAMKAEFNITLIHPKDLTA
LSNMLPKGPSTPLPEDPNWNVTEFHTTPKMSTYLLAFIVSEFDYVEKQA
SNGVLIRIWARPSAIAAGHGDYALNVTGPILNFFAGHYDTPYPLPKSDQ
IGLPDFNAGAMENWGLVTYRENSLLFDPLSSSSSNKERVVTVIAHELAH
QWFGNLVTIEWWNDLWLNEGFASYVEYLGADYAEPTWNLKDLMVLNDVY
RVMAVDALASSHPLSTPASEINTPAQISELFDAISYSKGASVLRMLSSF
LSEDVFKQGLASYLHTFAYQNTIYLNLWDHLQEAVNNRSIQLPTTVRDI
MNRWTLQMGFPVITVDTSTGTLSQEHFLLDPDSNVTRPSEFNYVWIVPI
TSIRDGRQQQDYWLIDVRAQNDLFSTSGNEWVLLNLNVTGYYRVNYDEE
NWRKIQTQLQRDHSAIPVINRAQIINDAFNLASAHKVPVTLALNNTLFL
IEERQYMPWEAALSSLSYFKLMFDRSEVYGPMKNYLKKQVTPLFIHFRN
NTNNWREIPENLMDQYSEVNAISTACSNGVPECEEMVSGLFKQWMENPN
NNPIHPNLRSTVYCNAIAQGGEEEWDFAWEQFRNATLVNEADKLRAALA
CSKELWILNRYLSYTLNPDLIRKQDATSTIISITNNVIGQGLVWDFVQS
NWKKLFNDYGGGSFSFSNLIQAVTRRFSTEYELQQLEQFKKDNEETGFG
SGTRALEQALEKTKANIKWVKENKEVVLQWFTENSK.

In some embodiments, the CD13 targeting moiety is a protein-based agent capable of specific binding, such as an antibody or derivatives thereof. In an embodiment, the CD13 targeting moiety comprises an antibody. In various embodiments, the antibody is a full-length multimeric protein that includes two heavy chains and two light chains. Each heavy chain includes one variable region (e.g., V_{H}) and at least three constant regions (e.g., CH₁, CH₂ and CH₃), and each light chain includes one variable region (V_{L}) and one constant region (C_{L}). The variable regions determine the specificity of the antibody. Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity. In some embodiments, the antibody is a chimeric antibody. In some embodiments, the antibody is a humanized antibody. In some embodiments, the CD13 targeting moiety comprises antibody derivatives or formats. In some embodiments, the CD13 targeting moiety of the present CD-13-based chimeric protein or chimeric protein complex is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; a Microbody; a peptide aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; affimers, an alphabody, a bicyclic peptide, a DuoBody, a Fv, a Fab, a Fab′, a F(ab′)2, a peptide mimetic molecule, or a synthetic molecule, as described in U.S. Pat. Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-Jun; 3 (3): 310-317.

In one embodiment, the CD13 targeting moiety comprises a single-domain antibody, such as VHH from, for example, an organism that produces VHH antibody such as a camelid, a shark, or a designed VHH. VHHs are antibody-derived therapeutic proteins that contain the unique structural and functional properties of naturally occurring heavy-chain antibodies. VHH technology is based on fully functional antibodies from camelids that lack light chains. These heavy-chain antibodies contain a single variable domain (VHH) and two constant domains (CH2 and CH3).

In an embodiment, the CD13 targeting moiety comprises a VHH. In some embodiments, the VHH is a humanized VHH or camelized VHH.

In some embodiments, the VHH comprises a fully human VH domain, e.g. a HUMABODY (Crescendo Biologics, Cambridge, UK). In some embodiments, fully human VH domain, e.g. a HUMABODY is monovalent, bivalent, or trivalent. In some embodiments, the fully human VH domain, e.g. a HUMABODY is mono- or multi-specific such as monospecific, bispecific, or trispecific. Illustrative fully human VH domains, e.g. HUMABODIES are described in, for example, WO 2016/113555 and WO2016/113557, the entire disclosure of which is incorporated by reference. In various embodiments, the CD13 targeting moiety's target (e.g. antigen, receptor) is found on one or more immune cells, which can include, without limitation, T cells, cytotoxic T lymphocytes, T helper cells, natural killer (NK) cells, natural killer T (NKT) cells, anti-tumor macrophages (e.g. M1 macrophages), B cells, dendritic cells, or subsets thereof. In some embodiments, the recognition domains specifically bind to a target (e.g. antigen, receptor) of interest and effectively, directly or indirectly, recruit one of more immune cells. In some embodiments, the target (e.g. antigen, receptor) of interest can be found on one or more endothelial cells of tumor neovasculature and/or tumor cells. In some embodiments, the target (e.g. antigen, receptor) of interest can be found on tumor vasculature, e.g., on epithelial cells of the tumor vasculature. In some embodiments, the disclosed chimeric proteins or chimeric protein complexes may directly or indirectly recruit an immune cell, e.g., in some embodiments, to a therapeutic site (e.g. a locus with one or more disease cell or cell to be modulated for a therapeutic effect). In some embodiments, the present CD13-targeted chimeric proteins or chimeric protein complexes may directly or indirectly recruit an immune cell, e.g. an immune cell that can kill and/or suppress a tumor cell, to a site of action (such as, by way of non-limiting example, the tumor microenvironment).

In various embodiments, the CD13 targeting moieties can directly or indirectly recruit cells, such as disease cells and/or effector cells. In some embodiments, the present CD13-targeted chimeric proteins or chimeric protein complexes are capable of, or find use in methods involving, shifting the balance of immune cells in favor of immune attack of a tumor. For instance, the present CD13-targeted chimeric proteins or chimeric protein complexes can shift the ratio of immune cells at a site of clinical importance in favor of cells that can kill and/or suppress a tumor (e.g. T cells, cytotoxic T lymphocytes, T helper cells, natural killer (NK) cells, natural killer T (NKT) cells, anti-tumor macrophages (e.g. M1 macrophages), B cells, dendritic cells, or subsets thereof) and in opposition to cells that protect tumors (e.g. myeloid-derived suppressor cells (MDSCs), regulatory T cells (Tregs); tumor associated neutrophils (TANs), M2 macrophages, tumor associated macrophages (TAMs), or subsets thereof). In some embodiments, the present CD13-targeted chimeric protein or chimeric protein complex is capable of increasing a ratio of effector T cells to regulatory T cells.

### TNF Signaling Agents

In various embodiments, the present CD13-targeted chimeric proteins or chimeric protein complexes comprise a tumor necrosis factor (TNF) signaling agent. In some embodiments, the TNF signaling agent comprises a modified TNF as a signaling agent. In some embodiments, the modified TNF is a modified wild type TNF. In some embodiments, the modified TNF signaling agent is modified to have attenuated activity.

The TNF superfamily consists of pro-inflammatory cytokines with crucial functions in the immune system by regulating cell death, proliferation and differentiation. In addition, members of the family were described to exert functions on bone metabolism, the nervous system, on neo-vasculature and carcinogenesis. Although most TNF superfamily ligands are synthesized as membrane-bound proteins, soluble forms can be generated by proteolytic cleavage. All of them bind to one or more molecules from the TNF receptor superfamily through their C-terminal TNF homology domain, which exhibits-20-30% sequence homology between family members.

Twenty nine (29) TNF superfamily receptors have been identified in humans. These are primarily type I (extracellular N terminus, intracellular C terminus) transmembrane glycoproteins with a cystein-rich motif in the ligand-binding extracellular domain. However, there are some exceptions like TRAIL-R3 that is attached to the membrane by a covalently linked C-terminal glycolipid. Soluble receptors can be generated by proteolytic cleavage (e.g. TNF-R1 and TNF-R2) or by alternative splicing of the exon encoding the transmembrane domain. The receptors of this superfamily can be divided in 3 groups based on their signaling properties: receptors with a cytoplasmic death domain that induce apoptosis; receptors with a TRAF-interacting motif that induce several signaling pathways such as NF-KB, JNK, p38, ERK and PI3K; and the decoy receptors that lack intracellular signaling domains. TNF induces apoptosis through interaction with TNF-R1 (p55), while binding to TNF-R2 (p75, primarily expressed on immune cells) promotes proliferation. TRAIL signaling is more complex as it can bind to two death receptors (TRAIL-R1 (DR4) and TRAIL-R2 (DR5), to two decoy receptors (TRAIL-R3 (DCR1) and TRAIL-R4 (DCR2) and to the soluble osteoprotegerin (OPG). Binding to one of the latter three receptors inhibits TRAIL-mediated apoptosis as it tethers TRAIL away from the death receptors (Gaur and Aggarwal, 2003; Hehlgans and Pfeffer, 2005; Huang and Sheikh, 2007).

The death-inducing TNF superfamily members TNF, CD95L (FasL) and TRAIL are potential therapeutics for cancers that express their respective receptor TNF-R1, CD95, TRAIL-R1 and TRAIL-R2. In fact, TNF was originally discovered more than 25 years ago as a factor with extraordinary antitumor activity, by causing hemorrhagic necrosis of certain tumors in vivo. Later it became clear that the selective damage attributed by TNF to tumor neovasculature also defines its anti-tumor potential (Lejeune et al., 2006; van Horssen et al., 2006). Unfortunately, systemic use of TNF in cancer treatment is still hampered by its shock-inducing properties. It is currently only clinically used in the setting of isolated limb perfusion in combination with chemotherapy to treat soft tissue sarcomas and in-transit melanoma (Roberts et al., 2011). Also, CD95L is toxic when administered systemically as it causes lethal hepatotoxicity due to massive hepatocyte apoptosis (Galle et al., 1995). TRAIL, however, has been shown to induce apoptosis in cancer cells with little or no cytotoxicity against non-transformed cells, and clinical trials in various advanced cancers report stable disease in many cases. Still, to obtain sufficient overall therapeutic activity combined treatment is required, which implies possible side effects due to sensitization of normal cells to TRAIL-induced apoptosis (Ashkenazi and Herbst, 2008; Falschlehner et al., 2009). Different approaches have been undertaken to minimize the toxicity upon systemic administration of death-inducing TNF superfamily members, such as mutant TNF with lower toxicity and higher efficiency (Li et al., 2012), delivery of TNF or TRAIL, normally as a single chain construct, by tumor-specific moieties (de Bruyn et al., 2013; Gregorc et al., 2009; Liu et al., 2006; Siegemund et al., 2012; Wang et al., 2006), chimeric soluble CD95L (Daburon et al., 2013) or agonistic TRAIL-R1-, TRAIL-R2 or CD95-specific antibodies (Johnstone et al., 2008; Ogasawara et al., 1993; Fox et al., 2010). Some of them can increase the therapeutic index but never to such an extent that it dramatically improves clinical outcome.

In some embodiments, the TNF signaling agent is TNF-α. TNF-α is a pleiotropic cytokine with many diverse functions, including regulation of cell growth, differentiation, apoptosis, tumorigenesis, viral replication, autoimmunity, immune cell functions and trafficking, inflammation, and septic shock. It binds to two distinct membrane receptors on target cells: TNFR1 (p55) and TNFR2 (p75). TNFR1 exhibits a very broad expression pattern whereas TNFR2 is expressed preferentially on certain populations of lymphocytes, Tregs, endothelial cells, certain neurons, microglia, cardiac myocytes and mesenchymal stem cells. Very distinct biological pathways are activated in response to receptor activation, although there is also some overlap. As a general rule, without wishing to be bound by theory, TNFR1 signaling is associated with induction of apoptosis (cell death) and TNFR2 signaling is associated with activation of cell survival signals (e.g. activation of NFkB pathway). Administration of TNF is systemically toxic, and this is largely due to TNFR1 engagement. However, it should be noted that activation of TNFR2 is also associated with a broad range of activities and, as with TNFR1, in the context of developing TNF-α based therapeutics, control over TNF-α targeting and activity is important.

In some embodiments, the TNF-α signaling agent is a modified TNF-α signaling agent. In some embodiments, the modified TNF-α signaling agent has reduced affinity and/or activity for TNFR1 and/or TNFR2. In some embodiments, the modified TNF-α signaling agent has substantially reduced or ablated affinity and/or activity for TNFR1 and/or TNFR2. TNFR1 is expressed in most tissues, and is involved in cell death signaling while, by contrast, TNFR2 is involved in cell survival signaling. Accordingly, in embodiments directed to methods of treating cancer, the modified TNF-α signaling agent has reduced affinity and/or activity for TNFR1 and/or substantially reduced or ablated affinity and/or activity for TNFR2. In these embodiments, the CD13-targeted chimeric proteins or chimeric protein complexes may be targeted to a cell for which apoptosis is desired, e.g. a tumor cell or a tumor vasculature endothelial cell. In embodiments directed to methods of promoting cell survival, for example, in neurogenesis for the treatment of neurodegenerative disorders, the modified TNF-α signaling agent has reduced affinity and/or activity for TNFR2 and/or substantially reduced or ablated affinity and/or activity for TNFR1. Stated another way, the present CD13-targeted chimeric proteins or chimeric protein complexes, in some embodiments, comprise modified TNF-α agent that allows of favoring either death or survival signals.

In some embodiments, the modified TNF-α signaling agent has reduced affinity and/or activity for TNFR1 and/or substantially reduced or ablated affinity and/or activity for TNFR2. Such a CD13-targeted chimeric protein or chimeric protein complex, in some embodiments, is a more potent inducer of apoptosis as compared to a wild type TNF and/or a chimera bearing only mutation(s) causing reduced affinity and/or activity for TNFR1. Such a CD13-targeted chimeric protein or chimeric protein complex, in some embodiments, finds use in inducing tumor cell death or a tumor vasculature endothelial cell death (e.g. in the treatment of cancers). Also, in some embodiments, these CD13-targeted chimeric proteins or chimeric protein complexes avoid or reduce activation of Treg cells via TNFR2, for example, thus further supporting TNFR1-mediated antitumor activity in vivo.

In some embodiments, the modified TNF-α signaling agent has reduced affinity and/or activity for TNFR2 and/or substantially reduced or ablated affinity and/or activity for TNFR1. Such a CD13-targeted chimeric protein or chimeric protein complex, in some embodiments, is a more potent activator of cell survival in some cell types, which may be a specific therapeutic objective in various disease settings, including without limitation, stimulation of neurogenesis. In addition, such a TNFR2-favoring chimeras also are useful in the treatment of autoimmune diseases (e.g. Crohn's, diabetes, MS, colitis, Sjogren's syndrome, multiple sclerosis, ankylosing spondylitis, and rheumatoid arthritis). In some embodiments, the CD13-targeted chimeric protein or chimeric protein complex is targeted to auto-reactive T cells. In some embodiments, the CD13-targeted chimeric protein or chimeric protein complex promotes Treg cell activation and indirect suppression of cytotoxic T cells.

In some embodiments, the CD13-targeted chimeric protein or chimeric protein complex causes the death of auto-reactive T cells, e.g. by activation of TNFR2 and/or avoidance TNFR1 (e.g. a modified TNF-α signaling agent having reduced affinity and/or activity for TNFR2 and/or substantially reduced or ablated affinity and/or activity for TNFR1). Without wishing to be bound by theory these auto-reactive T cells, have their apoptosis/survival signals altered e.g. by NFkB pathway activity/signaling alterations. In some embodiments, the CD13-targeted chimeric protein or chimeric protein complex causes the death of autoreactive T cells having lesions or modifications in the NFkB pathway, which underlie an imbalance of their cell death (apoptosis)/survival signaling properties and, optionally, altered susceptibility to certain death-inducing signals (e.g., TNFR2 activation).

In some embodiments, a TNFR-2 targeted TNF-α signaling agent has additional therapeutic applications in diseases, including autoimmune disease, various heart disease, de-myelinating and neurodegenerative disorders, and infectious disease, among others.

In an embodiment, the wild type TNF-α has the amino acid sequence of:

(SEQ ID NO: 2)
VRSSSRTPSDKPVAHVVANPQAEGQLQWLNRRANALLANGVELRDNQLV
VSEGLYLIYSQVLFKGQGCPSTHVLLTHTISRIAVSYQTKVNLLSAIKS
PCQRETPEGAEAKPWYEPIYLGGVFQLEKGDRLSAEINRPDYLDFAESG
QVYFGIIAL.

In such embodiments, the modified TNF-α agent has mutations at one or more amino acid positions 29, 31, 32, 84, 85, 86, 87, 88, 89, 145, 146 and 147 which produces a modified TNF-α with reduced receptor binding affinity. See, for example, U.S. Pat. No. 7,993,636, the entire contents of which are hereby incorporated by reference. In some embodiments, the modified human TNF-α signaling agent has mutations at one or more amino acid positions R32, N34, Q67, H73, L75, T77, S86, Y87, V91, 197, T105, P106, A109, P113, Y115, E127, N137, D143, A145, and E146 as described, for example, in WO/2015/007903, the entire contents of which is hereby incorporated by reference (numbering according to the human TNF sequence, Genbank accession number BAG70306, version BAG70306.1 GI: 197692685). In some embodiments, the modified human TNF-α moiety has substitution mutations selected from L29S, R32G, R32W, N34G, Q67G, H73G, L75G, L75A, L75S, T77A, S86G, S86T, Y87Q, Y87L, Y87A, Y87F, Y87H, V91G, V91A, 197A, 197Q, 197S, T105G, P106G, A109Y, P113G, Y115G, Y115A, E127G, N137G, D143N, A145G, A145R, A145T, E146D, E146K, and S147D. In some embodiments, the human TNF-α signaling agent has a mutation selected from Y87Q, Y87L, Y87A, Y87F, and Y87H. In another embodiment, the human TNF-α signaling agent has a mutation selected from 197A, 197Q, and 197S. In a further embodiment, the human TNF-α signaling agent has a mutation selected from Y115A and Y115G. In some embodiments, the human TNF-α signaling agent has an E146K mutation. In some embodiments, the human TNF-α signaling agent has an Y87H and an E146K mutation. In some embodiments, the human TNF-α signaling agent has an Y87H and an A145R mutation. In some embodiments, the human TNF-α signaling agent has a R32W and a S86T mutation. In some embodiments, the human TNF-α signaling agent has a R32W and an E146K mutation. In some embodiments, the human TNF-α signaling agent has a L29S and a R32W mutation. In some embodiments, the human TNF-α signaling agent has a D143N and an A145R mutation. In some embodiments, the human TNF-α signaling agent has a D143N and an A145R mutation. In some embodiments, the human TNF-α signaling agent has an A145T, an E146D, and a S147D mutation. In some embodiments, the human TNF-signaling agent has an A145T and a S147D mutation.

In some embodiments, the modified TNF-α signaling agent has one or more mutations selected from N39Y, S147Y, and Y87H, as described in WO2008/124086, the entire contents of which is hereby incorporated by reference. In some embodiments, the modified human TNF-α signaling agent has mutations that provide receptor selectivity as described in PCT/IB2016/001668, the entire contents of which are hereby incorporated by reference. In some embodiments, the mutations to TNF-α are TNF-R1 selective. In some embodiments, the mutations to TNF-α which are TNF-R1 selective are at one or more of positions R32, S86, and E146. In some embodiments, the mutations to TNF-α which are TNF-R1 selective are one or more of R32W, S86T, and E146K. In some embodiments, the mutations to TNF-α which are TNF-R1 selective are one or more of R32W, R32W/S86T, R32W/E146K and E146K. In some embodiments, the mutations to TNF-α are TNF-R2 selective. In some embodiments, the mutations to TNF-α which are TNF-R2 selective are at one or more of positions A145, E146, and S147. In some embodiments, the mutations to TNF-α which are TNF-R2 selective are one or more of A145T, A145R, E146D, and S147D. In some embodiments, the mutations to TNF-α which are TNF-R2 selective are one or more of A145R, A145T/S147D, and A145T/E146D/S147D.

In some embodiments, the TNF signaling agent is TNF-β. TNF-β can form a homotrimer or a heterotrimer with LT-β (LT-α1β2). In some embodiments, the TNF-β signaling agent is a modified TNF-β signaling agent. In some embodiments, the modified TNF-β signaling agent has substantially reduced or ablated affinity and/or activity for TNFR1 and/or TNFR2 and/or herpes virus entry mediator (HEVM) and/or LT-BR.

In an embodiment, the wild type TNF-β has the amino acid sequence of:

(SEQ ID NO: 3)
LPGVGLTPSAAQTARQHPKMHLAHSNLKPAAHLIGDPSKQNSLLWRANT
DRAFLQDGFSLSNNSLLVPTSGIYFVYSQVVFSGKAYSPKATSSPLYLA
HEVQLFSSQYPFHVPLLSSQKMVYPGLQEPWLHSMYHGAAFQLTQGDQL
STHTDGIPHLVLSPSTVFFGAFAL.

In such embodiments, the modified TNF-β signaling agent may comprise mutations at one or more amino acids at positions 106-113, which produce a modified TNF-β signaling agent with reduced receptor binding affinity to TNFR2. In an embodiment, the modified TNF-β signaling agent has one or more substitution mutations at amino acid positions 106-113. In illustrative embodiments, the substitution mutations are selected from Q107E, Q107D, S106E, S106D, Q107R, Q107N, Q107E/S106E, Q107E/S106D, Q107D/S106E, and Q107D/S106D. In another embodiment, the modified TNF-β signaling agent has an insertion of about 1 to about 3 amino acids at positions 106-113.

In some embodiments, the modified TNF signaling agent is a TNF family member (e.g. TNF-alpha, TNF-beta) which can be a single chain trimeric version as described in WO 2015/007903 and PCT/IB2016/001668, the entire contents of which are incorporated by reference.

In some embodiments, the modified TNF signaling agent is a TNF family member (e.g. TNF-alpha, TNF-beta) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at TNFR1. In these embodiments, the modified TNF signaling agent is a TNF family member (e.g. TNF-alpha, TNF-beta) which also, optionally, has substantially reduced or ablated affinity and/or activity for TNFR2. In some embodiments, the modified TNF signaling agent is a TNF family member (e.g. TNF-alpha, TNF-beta) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at TNFR2. In these embodiments, the modified TNF signaling agent is a TNF family member (e.g. TNF-alpha, TNF-beta) which also, optionally, has substantially reduced or ablated affinity and/or activity for TNFR1. The constructs of such embodiments find use in, for example, methods of dampening TNF response in a cell specific manner. In some embodiments, the antagonistic TNF family member (e.g. TNF-alpha, TNF-beta) is a single chain trimeric version as described in WO 2015/007903.

In some embodiments, the TNF signaling agent is TNF-related apoptosis-inducing ligand (TRAIL). In some embodiments, the TRAIL is a modified TRAIL agent. In some embodiments, the modified TRAIL agent has reduced affinity and/or activity for DR4 (TRAIL-RI) and/or DR5 (TRAIL-RII) and/or DcR1 and/or DcR2. In some embodiments, the modified TRAIL agent has substantially reduced or ablated affinity and/or activity for DR4 (TRAIL-RI) and/or DR5 (TRAIL-RII) and/or DcR1 and/or DcR2.

In an embodiment, the wild type TRAIL has the amino acid sequence of:

(SEQ ID NO: 4)
MAMMEVQGGPSLGQTCVLIVIFTVLLQSLCVAVTYVYFTNELKQMQDKY
SKSGIACFLKEDDSYWDPNDEESMNSPCWQVKWQLRQLVRKMILRTSEE
TISTVQEKQQNISPLVRERGPQRVAAHITGTRGRSNTLSSPNSKNEKAL
GRKINSWESSRSGHSFLSNLHLRNGELVIHEKGFYYIYSQTYFRFQEEI
KENTKNDKQMVQYIYKYTSYPDPILLMKSARNSCWSKDAEYGLYSIYQG
GIFELKENDRIFVSVTNEHLIDMDHEASFFGAFLVG.

In such embodiments, the modified TRAIL agent may comprise a mutation at amino acid positions T127-R132, E144-R149, E155-H161, Y189-Y209, T214-1220, K224-A226, W231, E236-L239, E249-K251, T261-H264 and H270-E271 (Numbering based on the human sequence, Genbank accession number NP _003801, version 10 NP 003801.1, GI: 4507593; see above).

In some embodiments, the modified TRAIL agent may comprise one or more mutations that substantially reduce its affinity and/or activity for TRAIL-R1. In such embodiments, the modified TRAIL agent may specifically bind to TRIL-R2. Illustrative mutations include mutations at one or more amino acid positions Y189, R191, Q193, H264, 1266, and D267. For example, the mutations may be one or more of Y189Q, R191K, Q193R, H264R, 1266L and D267Q. In an embodiment, the modified TRAIL agent comprises the mutations Y189Q, R191K, Q193R, H264R, 1266L and D267Q.

In some embodiments, the modified TRAIL agent may comprise one or more mutations that substantially reduce its affinity and/or activity for TRAIL-R2. In such embodiments, the modified TRAIL agent may specifically bind to TRIL-R1. Illustrative mutations include mutations at one or more amino acid positions G131, R149, S159, N199, K201, and S215. For example, the mutations may be one or more of G131R, R1491, S159R, N199R, K201H, and S215D. In an embodiment, the modified TRAIL agent comprises the mutations G131R, R1491, S159R, N199R, K201H, and S215D. Additional TRAIL mutations are described in, for example, Trebing et al., (2014) Cell Death and Disease, 5: e1035, the entire disclosure of which is hereby incorporated by reference.

### IFN Signaling Agents

In some aspects, the present technology relates to therapeutic uses of CD13-targeted chimeric proteins or chimeric protein complexes having at least one targeting moiety that specifically binds to CD13 and at least one signaling agent that is an interferon (IFN) or a modified form thereof. In various embodiments, the IFN signaling agent may be modified to attenuate activity. In one embodiment, the interferon is IFN-γ or a modified form thereof. In some embodiments, the present invention includes the use of CD13 targeted chimeric proteins or chimeric protein complexes having at least one signaling moiety that is an IFN or a modified form thereof as the sole therapeutic agent. In other embodiments, the present invention includes the use of CD13 targeted chimeric protein or chimeric protein complexes having at least one signaling moiety that is an IFN or a modified form thereof for use in combination therapy as described herein. Various embodiments of the IFN or a modified form thereof that may be used in the present invention are described below. In one embodiment, the present invention includes the use of CD13 targeted chimeric protein or chimeric protein complexes having at least one IFN-γ signaling moiety or a modified form thereof for use in combination therapy. In another embodiment, the present invention includes the use of CD13 targeted chimeric protein or chimeric protein complexes having at least one IFN-γ signaling moiety or a modified form thereof for use as a sole therapeutic agent.

### CD13-Targeted Chimeric Proteins

### Linkers

In some embodiments, the present CD13-targeted chimeric protein or chimeric protein complex optionally comprises one or more linkers. In some embodiments, the present CD13-targeted chimeric protein or chimeric protein complex comprises a linker connecting the CD13 targeting moiety and the TNF signaling agent (e.g., modified TNF signaling agent). In some embodiments, the present CD13-targeted chimeric protein or chimeric protein complex comprises a linker connecting the CD13 targeting moiety and the Interferon signaling agent (e.g., modified IFN signaling agent). In some embodiments, the present CD13-targeted chimeric protein or chimeric protein complex comprises a linker within the TNF signaling agent (e.g., modified TNF signaling agent). In some embodiments, the present CD13-targeted chimeric protein or chimeric protein complex comprises a linker within the IFN signaling agent (e.g., modified IFN signaling agent). In some embodiments, the linker may be utilized to link various functional groups, residues, or moieties as described herein to the chimeric protein or chimeric protein complex. In some embodiments, the linker is a single amino acid or a plurality of amino acids that does not affect or reduce the stability, orientation, binding, neutralization, and/or clearance characteristics of the binding regions and the binding protein. In various embodiments, the linker is selected from a peptide, a protein, a sugar, or a nucleic acid.

In some embodiments, vectors encoding the present CD13-targeted chimeric proteins or chimeric protein complexes linked as a single nucleotide sequence to any of the linkers described herein are provided and may be used to prepare such chimeric proteins or chimeric protein complexes.

In some embodiments, the linker length allows for efficient binding of a CD13 targeting moiety and the TNF signaling agent (e.g., modified TNF signaling agent) to their receptors. For instance, in some embodiments, the linker length allows for efficient binding of one of the CD13 targeting moieties and the TNF signaling agent or the IFN signaling agent to receptors on the same cell.

In some embodiments, the linker length is at least equal to the minimum distance between the binding sites of one of the CD13 targeting moieties and the TNF signaling agent or the IFN signaling agent to receptors on the same cell. In some embodiments the linker length is at least twice, or three times, or four times, or five times, or ten times, or twenty times, or 25 times, or 50 times, or one hundred times, or more the minimum distance between the binding sites of one of the CD13 targeting moieties and the TNF signaling agent or the IFN signaling agent to receptors on the same cell.

As described herein, the linker length allows for efficient binding of one of the CD13 targeting moieties and the TNF signaling agent or the IFN signaling agent to receptors on the same cell, the binding being sequential, e.g. CD13 targeting moiety/receptor binding preceding TNF signaling agent/receptor binding or CD13 targeting moiety/receptor binding preceding IFN signaling agent/receptor binding.

In some embodiments, there are two linkers in a single chimera, each connecting the TNF signaling agent or the IFN signaling agent to a CD13 targeting moiety. In various embodiments, the linkers have lengths that allow for the formation of a site that has a disease cell and an effector cell without steric hindrance that would prevent modulation of the either cell.

The invention contemplates the use of a variety of linker sequences. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et al., (2000), Protein Eng. 13(5):309-312, the entire contents of which are hereby incorporated by reference. In various embodiments, the linker may be functional. For example, without limitation, the linker may function to improve the folding and/or stability, improve the expression, improve the pharmacokinetics, and/or improve the bioactivity of the present chimeric protein or chimeric protein complex.

In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is a polypeptide. In some embodiments, the linker is greater than about 100 amino acids long. For example, the linker may be greater than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid.

In some embodiments directed to CD13 targeted chimeric proteins or chimeric protein complexes having two or more CD13 targeting moieties, a linker connects the two CD13 targeting moieties to each other and this linker has a short length and a linker connects a CD13 targeting moiety and a TNF signaling agent this linker is longer than the linker connecting the two CD13 targeting moieties. For example, the difference in amino acid length between the linker connecting the two targeting moieties and the linker connecting a targeting moiety and a signaling agent may be about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids.

In various embodiments, the linker is substantially comprised of glycine and serine residues (e.g. about 30%, or about 40%, or about 50%, or about 60%, or about 70%, or about 80%, or about 90%, or about 95%, or about 97% glycines and serines). For example, in some embodiments, the linker is (Gly₄Ser)ₙ, where n is from about 1 to about 8, e.g. 1, 2, 3, 4, 5, 6, 7, or 8 (SEQ ID NOs: 5-12, respectively). In an embodiment, the linker sequence is GGSGGSGGGGSGGGGS (SEQ ID NO: 13). Additional illustrative linkers include, but are not limited to, linkers having the sequence LE, GGGGS (SEQ ID NO: 5), (GGGGS)ₙ (n=1-4) (SEQ ID NOs: 5-8), (Gly)₈ (SEQ ID NO: 14), (Gly)₆ (SEQ ID NO: 15), (EAAAK)ₙ (n=1-3) (SEQ ID NOs: 16-18), A(EAAAK)ₙA (n=2-5) (SEQ ID NOs: 19-22), AEAAAKEAAAKA (SEQ ID NO: 23), A(EAAAK)₄ALEA(EAAAK)₄A (SEQ ID NO: 24), PAPAP (SEQ ID NO: 25), KESGSVSSEQLAQFRSLD (SEQ ID NO: 26), EGKSSGSGSESKST (SEQ ID NO: 27), GSAGSAAGSGEF (SEQ ID NO: 28), and (XP)ₙ, with X designating any amino acid, e.g., Ala, Lys, or Glu. In various embodiments, the linker is (GGS)ₙ (n=1-20) (SEQ ID NOs: 29-48). In some embodiments, the linker is G. In some embodiments, the linker is AAA. In some embodiments, the linker is (GGGGS) n (n=5-20) (SEQ ID NOs: 9-12 and 49-60).

In some embodiments, the linker is one or more of GGGSE (SEQ ID NO: 61), GSESG (SEQ ID NO: 62), GSEGS (SEQ ID NO: 63), GEGGSGEGSSGEGSSSEGGGSEGGGSEGGGSEGGS (SEQ ID NO: 64), and a linker of randomly placed G, S, and E every 4 amino acid intervals.

In some embodiments, the linker is a synthetic linker such as PEG.

In various embodiments, the linker may be functional. For example, without limitation, the linker may function to improve the folding and/or stability, improve the expression, improve the pharmacokinetics, and/or improve the bioactivity of the present chimeric protein or chimeric protein complex. In another example, the linker may function to target the chimeric protein or chimeric protein complex to a particular cell type or location.

### Multiple TNF Signaling Agents

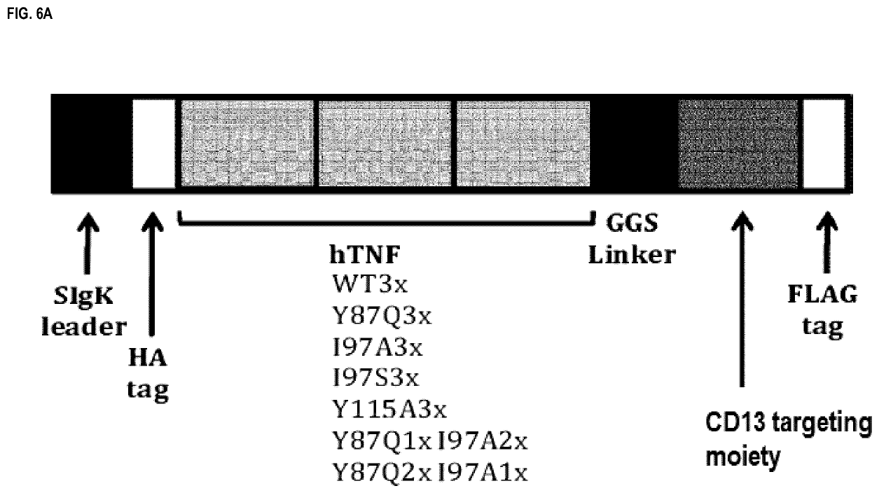

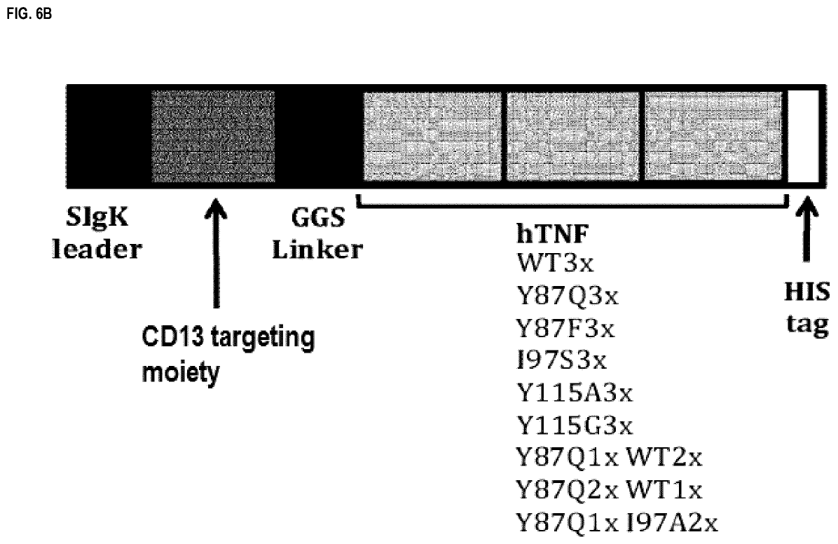

In some embodiments, the CD13-targeted chimeric protein or chimeric protein complex comprises 2 or more TNF signaling agents. In some embodiments, the CD13-targeted chimeric protein or chimeric protein complex comprises at least one wild type TNF signaling agent and at least one modified TNF signaling agent disclosed above. FIGS. 6A-B shows exemplary, non-limiting embodiments of a CD13-targeted chimeric protein or chimeric protein complex having 3 TNF signaling agents.

In some embodiments, the CD13-targeted chimeric protein or chimeric protein complex comprises 2 or more modified TNF signaling agents disclosed above. In some embodiments, the CD13-targeted chimeric protein or chimeric protein complex comprises 3 modified TNF signaling agents disclosed above.

In some embodiments, the 2 or more modified TNF signaling agents are members of the same TNF family, e.g., all are modified TNF-α signaling agents. In some embodiments, the 2 or more modified TNF signaling agents are members of the same TNF family and have identical modifications, e.g., all are modified human TNF-α signaling agents having a Y87Q modification. In some embodiments, the 2 or more modified TNF signaling agents are members of the same TNF family and each have different modifications, e.g., two modified human TNF-α signaling agents, one having a Y87Q modification and the other having a 197A modification or one having a 197S modification and the other having a 197A modification. In an exemplary embodiment, the CD13-targeted chimeric protein or chimeric protein complex comprises 3 copies of the same modified TNF signaling agent having the same mutation.

In some embodiments, the 2 or more modified TNF signaling agents are members of different TNF families, e.g., one modified TNF-α signaling agent and one modified TNF-β signaling agent.

In some embodiments, the CD13-targeted chimeric protein or chimeric protein complex comprises 3 or more modified TNF signaling agents, wherein the modified TNF signaling agents are members of the same TNF family and at least two have the same modification, e.g., two modified human TNF-α signaling agents having a Y87Q modification and a third modified human TNF-α signaling agent having a 197A modification.

In some embodiments, the CD13-targeted chimeric protein or chimeric protein complex comprises 3 or more modified TNF signaling agents, wherein at least two modified TNF signaling agents are members of the same TNF family.

In some embodiments, the CD13-targeted chimeric protein or chimeric protein complex comprises 2 or more modified TNF signaling agents, wherein the modified TNF signaling agents are consecutive monomers within a single chain polypeptide (see, e.g., FIGS. 6A-B). In an exemplary embodiment, the CD13-targeted chimeric protein or chimeric protein complex comprises 3 copies of the same modified TNF signaling agent having the same mutation in a single polypeptide chain.

### Multiple IFN Signaling Agents

In some embodiments, the CD13-targeted chimeric protein or chimeric protein complex comprises 2 or more IFN signaling agents. In some embodiments, the CD13-targeted chimeric protein or chimeric protein complex comprises at least one wild type IFN signaling agent and at least one modified IFN signaling agent disclosed below. In some embodiments, the CD13-targeted chimeric protein or chimeric protein complex comprises 2 or more modified IFN signaling agents disclosed below. In some embodiments, the CD13-targeted chimeric protein or chimeric protein complex comprises 3 modified IFN signaling agents disclosed below. In some embodiments, the 2 or more modified IFN signaling agents are the same or different. In some embodiments, the 2 or more modified IFN signaling agents have identical modifications. In some embodiments, the 2 or more modified IFN signaling agents have different modifications. In some embodiments, the CD13-targeted chimeric protein or chimeric protein complex comprises 3 or more modified IFN signaling agents.

### Use of CD13-Targeted Chimeric Proteins in Combination Therapy

In some embodiments, the present technology relates to use of CD13-targeted chimeric proteins or chimeric protein complexes in combination therapy. In some embodiments, the present CD13-targeted chimeric protein or chimeric protein complex is co-administered with at least one additional therapeutic agent. In some embodiments, the additional therapeutic agent is a CD8-targeted chimeric protein or chimeric protein complex. In some embodiments, the additional therapeutic agent is a CD13-targeted chimeric protein or chimeric protein complex. In some embodiments, the additional therapeutic agent is a chimeric protein or chimeric protein complex where the targeting moiety is directed to CD13 and the signaling agent is an interferon or a modified form thereof. In some embodiments, the signaling agent is IFN-γ or a modified form thereof. In some embodiments, the additional therapeutic agent is one or more agents selected from a phosphoinositide-3-kinase 9 (PI3K) inhibitor, anthracycline, and SMAC mimetic. In some embodiments, the anthracycline is a liposomal anthracycline.

### CD8-Targeted or CD13-Targeted Chimeric Proteins

In some embodiments, the additional therapeutic agent is a CD8-targeted chimeric protein or chimeric protein complex having at least one targeting moiety that specifically binds to CD8 and at least one signaling agent that is an interferon (IFN) or a modified form thereof. In some embodiments, the additional therapeutic agent is a CD13-targeted chimeric protein or chimeric protein complex having at least one targeting moiety that specifically binds to CD13, as described herein, and at least one signaling agent that is an interferon (IFN) or a modified form thereof. In various embodiments, the IFN signaling agent is modified to have attenuate activity.

### CD8 Targeting Moieties

In some embodiments, the additional therapeutic agent—that is used in the combination therapy described herein—is a CD8-targeted chimeric protein or chimeric protein complex having at least one targeting moiety that specifically binds to CD8 and at least one signaling agent that is an interferon (IFN) or a modified form thereof. In various embodiments, the CD8-targeted chimeric protein or chimeric protein complex comprises a CD8 targeting moiety that is a protein-based agent capable of specific binding to CD8. In various embodiments, the CD8 targeting moiety is a protein-based agent capable of specific binding to CD8 without functionally modulating (e.g. partial or complete neutralization) CD8.

CD8 is a heterodimeric type I transmembrane glycoprotein, whose α and β chains are both comprised of an immunoglobulin (lg)-like extracellular domain connected by an extended O-glycosylated stalk to a single-pass transmembrane domain and a short cytoplasmic tail (Li et al., 2013). The cytoplasmic region of the CD8 α-chain contains two cysteine motifs that serve as a docking site for src tyrosine kinase p56lck (Lck). In contrast, this Lck binding domain appears to be absent from the CD8 β chain, suggesting that the β chain is not involved in downstream signaling (Artyomov et al., 2010). CD8 functions as a co-receptor for the T-cell receptor with its principle role being the recruitment of Lck to the TCR-pMHC complex following co-receptor binding to MHC (Turner et al., 1990, Veillette et al., 1988). The increase in the local concentration of this kinase activates a signaling cascade that recruits and activates ζ-chain-associated protein kinase 70 (ZAP-70), subsequently leading to the amplification of T-cell activation signals (Purbhoo et al., 2001, Laugel et al., 2007a).

In some embodiments, the CD8 targeting moiety comprises an antigen recognition domain that recognizes an epitope present on the CD8 α and/or β chains. In an embodiment, the antigen-recognition domain recognizes one or more linear epitopes on the CD8 α and/or β chains. In some embodiment, a linear epitope refers to any continuous sequence of amino acids present on the CD8 α and/or β chains. In another embodiment, the antigen-recognition domain recognizes one or more conformational epitopes present on the CD8 α and/or β chains. As used herein, a conformation epitope refers to one or more sections of amino acids (which may be discontinuous) which form a three-dimensional surface with features and/or shapes and/or tertiary structures capable of being recognized by an antigen recognition domain.

In various embodiments, the CD8 targeting moiety may bind to the full-length and/or mature forms and/or isoforms and/or splice variants and/or fragments and/or any other naturally occurring or synthetic analogs, variants, or mutants of human CD8 α and/or β chains. In various embodiments, the CD8 targeting moiety may bind to any forms of the human CD8 α and/or β chains, including monomeric, dimeric, heterodimeric, multimeric and associated forms. In an embodiment, the CD8 binding agent binds to the monomeric form of CD8 α chain or CD8 β chain. In another embodiment, the CD8 targeting moiety binds to a homodimeric form comprised of two CD8 α chains or two CD8 β chains. In a further embodiment, the CD8 binding agent binds to a heterodimeric form comprised of one CD8 α chain and one CD8 β chain.

In an embodiment, the CD8 targeting moiety comprises an antigen recognition domain that recognizes one or more epitopes present on the human CD8 α chain. In an embodiment, the human CD8 α chain comprises the amino acid sequence of:

### Isoform 1

(SEQ ID NO: 66)
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYV.

In an embodiment, the human CD8 α chain comprises the amino acid sequence of:

### Isoform 2

(SEQ ID NO: 67)
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAGNRRRVCKCPRPWKSGDKPSLSARY
V.

In an embodiment, the human CD8 α chain comprises the amino acid sequence of:

### Isoform 3

(SEQ ID NO: 68)
MRNQAPGRPKGATFPPRRPTGSRAPPLAPELRAKQRPGERVMALPVTAL
LLPLALLLHAARPSQFRVSPLDRTWNLGETVELKCQVLLSNPTSGCSWL
FQPRGAAASPTFLLYLSQNKPKAAEGLDTQRFSGKRLGDTFVLTLSDFR
RENEGYYFCSALSNSIMYFSHFVPVFLPAKPTTTPAPRPPTPAPTIASQ
PLSLRPEACRPAAGGAVHTRGLDFACDIYIWAPLAGTCGVLLLSLVITL
YCNHRNRRRVCKCPRPVVKSGDKPSLSARYV.

In an embodiment, the CD8 targeting moiety comprises an antigen recognition domain that recognizes one or more epitopes present on the human CD8 β chain. In an embodiment, the human CD8 β chain comprises the amino acid sequence of:

### Isoform 1

(SEQ ID NO: 69)
MRPRLWLLLAAQLTVLHGNSVLQQTPAYIKVQTNKMVMLSCEAKISLSN
MRIYWLRQRQAPSSDSHHEFLALWDSAKGTIHGEEVEQEKIAVFRDASR
FILNLTSVKPEDSGIYFCMIVGSPELTFGKGTQLSVVDFLPTTAQPTKK
STLKKRVCRLPRPETQKGPLCSPITLGLLVAGVLVLLVSLGVAIHLCCR
RRRARLRFMKQFYK.

In an embodiment, the human CD8 β chain comprises the amino acid sequence of:

### Isoform 2

(SEQ ID NO: 70)
MRPRLWLLLAAQLTVLHGNSVLQQTPAYIKVQTNKMVMLSCEAKISLSN
MRIYWLRQRQAPSSDSHHEFLALWDSAKGTIHGEEVEQEKIAVFRDASR
FILNLTSVKPEDSGIYFCMIVGSPELTFGKGTQLSVVDFLPTTAQPTKK
STLKKRVCRLPRPETQKGPLCSPITLGLLVAGVLVLLVSLGVAIHLCCR
RRRARLRFMKQLRLHPLEKCSRMDY.

In an embodiment, the human CD8 β chain comprises the amino acid sequence of:

### Isoform 3

(SEQ ID NO: 71)
MRPRLWLLLAAQLTVLHGNSVLQQTPAYIKVQTNKMVMLSCEAKISLSN
MRIYWLRQRQAPSSDSHHEFLALWDSAKGTIHGEEVEQEKIAVFRDASR
FILNLTSVKPEDSGIYFCMIVGSPELTFGKGTQLSVVDFLPTTAQPTKK
STLKKRVCRLPRPETQKGRRRRARLRFMKQPQGEGISGTFVPQCLHGYY
SNTTTSQKLLNPWILKT.

In an embodiment, the human CD8 β chain comprises the amino acid sequence of:

### Isoform 4

(SEQ ID NO: 72)
MRPRLWLLLAAQLTVLHGNSVLQQTPAYIKVQTNKMVMLSCEAKISLSN
MRIYWLRQRQAPSSDSHHEFLALWDSAKGTIHGEEVEQEKIAVFRDASR
FILNLTSVKPEDSGIYFCMIVGSPELTFGKGTQLSVVDFLPTTAQPTKK
STLKKRVCRLPRPETQKGPLCSPITLGLLVAGVLVLLVSLGVAIHLCCR
RRRARLRFMKQKFNIVCLKISGFTTCCCFQILQISREYGFGVLLQKDIG
Q.

In an embodiment, the human CD8 β chain comprises the amino acid sequence of:

### Isoform 5

(SEQ ID NO: 73)
MRPRLWLLLAAQLTVLHGNSVLQQTPAYIKVQTNKMVMLSCEAKISLSNM
RIYWLRQRQAPSSDSHHEFLALWDSAKGTIHGEEVEQEKIAVFRDASRFI
LNLTSVKPEDSGIYFCMIVGSPELTFGKGTQLSVVDFLPTTAQPTKKSTL
KKRVCRLPRPETQKGPLCSPITLGLLVAGVLVLLVSLGVAIHLCCRRRRA
RLRFMKQPQGEGISGTFVPQCLHGYYSNTTTSQKLLNPWILKT.

In an embodiment, the human CD8 β chain comprises the amino acid sequence of:

### Isoform 6

(SEQ ID NO: 74)
MRPRLWLLLAAQLTVLHGNSVLQQTPAYIKVQTNKMVMLSCEAKISLSNM
RIYWLRQRQAPSSDSHHEFLALWDSAKGTIHGEEVEQEKIAVFRDASRFI
LNLTSVKPEDSGIYFCMIVGSPELTFGKGTQLSVVDFLPTTAQPTKKSTL
KKRVCRLPRPETQKGRRRRARLRFMKQFYK.

In an embodiment, the human CD8 β chain comprises the amino acid sequence of:

### Isoform 7

(SEQ ID NO: 75)
MRPRLWLLLAAQLTVLHGNSVLQQTPAYIKVQTNKMVMLSCEAKISLSNM
RIYWLRQRQAPSSDSHHEFLALWDSAKGTIHGEEVEQEKIAVFRDASRFI
LNLTSVKPEDSGIYFCMIVGSPELTFGKGTQLSVVDFLPTTAQPTKKSTL
KKRVCRLPRPETQKDFTNKQRIGFWCPATKRHRSVMSTMWKNERRDTFNP
GEFNGC.

In an embodiment, the human CD8 β chain comprises the amino acid sequence of:

### Isoform 8

(SEQ ID NO: 76)
MRPRLWLLLAAQLTVLHGNSVLQQTPAYIKVQTNKMVMLSCEAKISLSNM
RIYWLRQRQAPSSDSHHEFLALWDSAKGTIHGEEVEQEKIAVFRDASRFI
LNLTSVKPEDSGIYFCMIVGSPELTFGKGTQLSVVDFLPTTAQPTKKSTL
KKRVCRLPRPETQKGLKGKVYQEPLSPNACMDTTAILQPHRSCLTHGS.

In some embodiments, the CD8 targeting moiety is capable of specific binding. In various embodiments, the CD8 targeting moiety comprises an antigen recognition domain such as an antibody or derivatives thereof. In an embodiment, the CD8 targeting moiety comprises an antibody. In various embodiments, the antibody is a full-length multimeric protein that includes two heavy chains and two light chains. Each heavy chain includes one variable region (e.g., V_{H}) and at least three constant regions (e.g., CH₁, CH₂ and CH₃), and each light chain includes one variable region (V_{L}) and one constant region (C_{L}). The variable regions determine the specificity of the antibody. Each variable region comprises three hypervariable regions also known as complementarity-determining regions (CDRs) flanked by four relatively conserved framework regions (FRs). The three CDRs, referred to as CDR1, CDR2, and CDR3, contribute to the antibody binding specificity. In some embodiments, the antibody is a chimeric antibody. In some embodiments, the antibody is a humanized antibody.

In some embodiments, the CD8 targeting moiety comprise an antibody derivative or format. In some embodiments, the CD8 targeting moiety comprises a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer; an alphabody; a bicyclic peptide; a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)₂, a peptide mimetic molecule, or a synthetic molecule, as described in U.S. Pat. Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-Jun; 3 (3): 310-317.

In some embodiments, the CD8 targeting moiety comprises a single-domain antibody, such as a VHH. The VHH may be derived from, for example, an organism that produces VHH antibody such as a camelid, a shark, or the VHH may be a designed VHH. VHHs are antibody-derived therapeutic proteins that contain the unique structural and functional properties of naturally occurring heavy-chain antibodies. VHH technology is based on fully functional antibodies from camelids that lack light chains. These heavy-chain antibodies contain a single variable domain (VHH) and two constant domains (CH2 and CH3).

In an embodiment, the CD8 targeting moiety comprises a VHH. In some embodiments, the VHH is a humanized VHH or camelized VHH.

In some embodiments, the VHH comprises a fully human V_{H} domain, e.g. a HUMABODY (Crescendo Biologics, Cambridge, UK). In some embodiments, fully human V_{H} domain, e.g. a HUMABODY is monovalent, bivalent, or trivalent. In some embodiments, the fully human V_{H} domain, e.g. a HUMABODY is mono- or multi-specific such as monospecific, bispecific, or trispecific. Illustrative fully human V_{H} domains, e.g. a HUMABODIES are described in, for example, WO2016/113555 and WO2016/113557, the entire disclosure of which is incorporated by reference.

In some embodiments, the CD8 targeting moiety comprises a VHH comprising a single amino acid chain having four “framework regions” or FRs and three “complementary determining regions” or CDRs. As used herein, “framework region” or “FR” refers to a region in the variable domain, which is located between the CDRs. As used herein, “complementary determining region” or “CDR” refers to variable regions in VHHs that contains the amino acid sequences capable of specifically binding to antigenic targets.

In various embodiments, the CD8 targeting moiety comprises a VHH having a variable domain comprising at least one CDR1, CDR2, and/or CDR3 sequences.

In some embodiments, the CD8 CDR1 sequence is selected from:

(SEQ ID NO: 77)
GFTFDDYAMS;
(SEQ ID NO: 78)
GFTFDDYAIG;
(SEQ ID NO: 79)
GRSFSSYTLA;
(SEQ ID NO: 80)
GRTFSSYTMG;
(SEQ ID NO: 81)
GRTFSSYIMG;
(SEQ ID NO: 82)
GRTFSSYTMG;
(SEQ ID NO: 83)
GRTSGRTFSSYTMG;
(SEQ ID NO: 84)
GRTFSSYAMG;
(SEQ ID NO: 85)
GLTFSNYIMG;
(SEQ ID NO: 86)
GRTFSSYTMG;
(SEQ ID NO: 87)
GRTFSSDTMG;
(SEQ ID NO: 88)
GLTFSNYIMG;
(SEQ ID NO: 89)
GFTLDYYGIG;
(SEQ ID NO: 90)
GHTFSSYTMG;
(SEQ ID NO: 91)
GRTFSSYVIG;
(SEQ ID NO: 92)
GFAFDGYAIG;
(SEQ ID NO: 93)
GFAFGFFDMT;
(SEQ ID NO: 94)
GRTFSNYVIG;
(SEQ ID NO: 95)
GSIFSINVMG;
(SEQ ID NO: 96)
GRTFSNYNVG;
(SEQ ID NO: 97)
GHTFSSYTMG;
(SEQ ID NO: 98)
GRTFSTYPVG;
(SEQ ID NO: 99)
GRTFSNYAMG;
(SEQ ID NO: 100)
GRTFSDYRMG;
(SEQ ID NO: 101)
GLTFSNYIMA;
(SEQ ID NO: 102)
GRTFSNSVMG;
(SEQ ID NO: 103)
GRTFSSYIIG;
(SEQ ID NO: 104)
GRTFSSYVMG;
(SEQ ID NO: 105)
GGTFSNYVMG;
(SEQ ID NO: 106)
GRTFSNYGIG;
(SEQ ID NO: 107)
GFTFDDYAIA;
(SEQ ID NO: 108)
GRTFSSYTVA;
(SEQ ID NO: 109)
GFPFDDYAIA;
(SEQ ID NO: 110)
GRTFSSYVMG;
(SEQ ID NO: 111)
GRTLSSNPMA;
(SEQ ID NO: 112)
GFTFDNYAIG;
(SEQ ID NO: 113)
GRAFSSYFMG;
(SEQ ID NO: 114)
TPTFSSYNMG;
(SEQ ID NO: 115)
GFTFDDYAIA;
(SEQ ID NO: 116)
GGTFSGYIMG;
(SEQ ID NO: 117)
GRSFSSYTIA;
(SEQ ID NO: 118)
GFSSDDYTIG;
(SEQ ID NO: 119)
GFTFDDYTIG;
(SEQ ID NO: 120)
GFSSDDYTIG;
(SEQ ID NO: 121)
GFTFDQYTIA;
(SEQ ID NO: 122)
GRTFSSYAMA;
(SEQ ID NO: 123)
GFAFDGYAIG;
(SEQ ID NO: 124)
GFSSDDYTIA;
(SEQ ID NO: 125)
GFSSDDYTIG;
(SEQ ID NO: 126)
GFTFDDYTIG;
(SEQ ID NO: 127)
GFSSDDYTIG;
(SEQ ID NO: 128)
GFSSDDYTIG;
(SEQ ID NO: 129)
GFSFDDYAIA;
(SEQ ID NO: 130)
GFSSDDYTIG;
(SEQ ID NO: 131)
GFTGNDLAIG;
(SEQ ID NO: 132)
GFSSDDYTIA;
(SEQ ID NO: 133)
EGTLSSYGIG;
(SEQ ID NO: 134)
GFSSDDYTIA;
(SEQ ID NO: 135)
GFTFDDYAIA;
(SEQ ID NO: 136)
GLSSDDYTIG;
(SEQ ID NO: 137)
GLSSDDYTIG;
(SEQ ID NO: 138)
GFSSDDYTIG;
(SEQ ID NO: 139)
GFSFDDYTIG;
(SEQ ID NO: 140)
GFTFDDYAIA;
(SEQ ID NO: 141)
GFTFDDYAIG;
(SEQ ID NO: 142)
GFTFGDYTIG;
(SEQ ID NO: 143)
EGTFSSYGIG;
(SEQ ID NO: 144)
GFSSDDYTIG;
(SEQ ID NO: 145)
GVSIGDYNIG;
(SEQ ID NO: 146)
GFTFDDYTIA;
or
(SEQ ID NO: 147)
GFTFDDYTIA.

In some embodiments, the CD8 CDR2 sequence is selected from:

(SEQ ID NO: 148)
TINWNGGSAEYAEPVKG;
(SEQ ID NO: 149)
CIRVSDGSTYYADPVKG;
(SEQ ID NO: 150)
ASITWGGGNTY;
(SEQ ID NO: 151)
AATVWTGAGTV;
(SEQ ID NO: 152)
AAIGWSADITV;
(SEQ ID NO: 153)
AFIDWSGGGTY;
(SEQ ID NO: 154)
ATITWGGGSTY;
(SEQ ID NO: 155)
AAISWSGGPTV;
(SEQ ID NO: 156)
AAITWGGGSTV;
(SEQ ID NO: 157)
AAITWSGVSTV;
(SEQ ID NO: 158)
GAIMWSGAFTH;
(SEQ ID NO: 159)
AAITWGGGSTV;
(SEQ ID NO: 160)
SCISSSDRNTY;
(SEQ ID NO: 161)
AFIDWSGGGTY;
(SEQ ID NO: 162)
AVITWSGDSTY;
(SEQ ID NO: 163)
ACISSKDGSTY;
(SEQ ID NO: 164)
SGINSIGGSTT;
(SEQ ID NO: 165)
AVVTWSGDSTY;
(SEQ ID NO: 166)
AKITNFGITS;
(SEQ ID NO: 167)
SFISWISDITY;
(SEQ ID NO: 168)
AFIDWSGGGTY;
(SEQ ID NO: 169)
AVILWSGVSTY;
(SEQ ID NO: 170)
AAIVWSGGSTY;
(SEQ ID NO: 171)
AAISSSGYHTY;
(SEQ ID NO: 172)
SCISSPDGSTY;
(SEQ ID NO: 173)
AAVLWSGVSTA;
(SEQ ID NO: 174)
VAITWDGSATT;
(SEQ ID NO: 175)
AAIGWNGGITY;
(SEQ ID NO: 176)
GFITWSGASTY;
(SEQ ID NO: 177)
AGINWSGESAD;
(SEQ ID NO: 178)
SCIERSDGSTY;
(SEQ ID NO: 179)
SCISNTDSSTY;
(SEQ ID NO: 180)
SCISNTDSSTY;
(SEQ ID NO: 181)
AQISWSAGSIY;
(SEQ ID NO: 182)
AGMSWNPGPAV;
(SEQ ID NO: 183)
SCISRSDGSTY;
(SEQ ID NO: 184)
ANIGWTGDMTY;
(SEQ ID NO: 185)
AAIIWSGSMTY;
(SEQ ID NO: 186)
SCISNTDSSTY;
(SEQ ID NO: 187)
AANTWSGGPTY;
(SEQ ID NO: 188)
SCISSDGSTG;
(SEQ ID NO: 189)
SCYSSSDGSTG;
(SEQ ID NO: 190)
SCISSDGSTG;
(SEQ ID NO: 191)
GCIKSSDGTTG;
(SEQ ID NO: 192)
SCISNTDSSTY;
(SEQ ID NO: 193)
AAIAWSAGSTY;
(SEQ ID NO: 194)
SCISSKEGSTY;
(SEQ ID NO: 195)
SCISSSDGSTG;
(SEQ ID NO: 196)
SCYSSRDGTTG;
(SEQ ID NO: 197)
SCISSDGSTG;
(SEQ ID NO: 198)
SCYSSSDGSTG;
(SEQ ID NO: 199)
SCFSSSDGSTG;
(SEQ ID NO: 200)
SCISNTDSSTF;
(SEQ ID NO: 201)
SCYSSSDGSTG;
(SEQ ID NO: 202)
SCISNTDSSTY;
(SEQ ID NO: 203)
SCISSSDGSTG;
(SEQ ID NO: 204)
GGINWSGDSTD;
(SEQ ID NO: 205)
SCFSSSDGSAG;
(SEQ ID NO: 206)
SCISNTDSSTY;
(SEQ ID NO: 207)
SCFSTRDGNAG;
(SEQ ID NO: 208)
SCFSSRDGSTG;
(SEQ ID NO: 209)
SCFSSRDGSTG;
(SEQ ID NO: 210)
SCISSDGSTG;
(SEQ ID NO: 211)
SCISNTDSSTY;
(SEQ ID NO: 212)
SCISSPDGSTY;
(SEQ ID NO: 213)
SCYSSSDGNTG;
(SEQ ID NO: 214)
GGINWSGDSTD;
(SEQ ID NO: 215)
SCFSSSDGSTG;
(SEQ ID NO: 216)
SCISSGDGTTY;
(SEQ ID NO: 217)
SCISSDGSTG;
or
(SEQ ID NO: 218)
SCISSDGSTG.

In some embodiments, the CD8 CDR3 sequence is selected from:

(SEQ ID NO: 219)
KDADLVWYNLS;
(SEQ ID NO: 220)
KDADLVWYNLR;
(SEQ ID NO: 221)
AGSLYTCVQSIVVVPARPYYDMDY;
(SEQ ID NO: 222)
AKGLRNSDWDLRRGYEYDY;
(SEQ ID NO: 223)
ADQASVPPPYGSERYDIASPSEYDY;
(SEQ ID NO: 224)
ANSRAYYSSSYDLGRLASYDY;
(SEQ ID NO: 225)
AAQRLGSVTDYTKYDY;
(SEQ ID NO: 226)
ASVKVVAGSGIDISGSRNYDY;
(SEQ ID NO: 227)
AKRLDYSATDKGVDLSDEYDY;
(SEQ ID NO: 228)
AAGGSGRLRDLKVGQNYDY;
(SEQ ID NO: 229)
ADSPPRTYSSGSVNLEDGSEYDY;
(SEQ ID NO: 230)
VIPGRGSALPIDVGKSDEYEY;
(SEQ ID NO: 231)
AAGASGRLRDLKVGQNYDY;
(SEQ ID NO: 232)
ADGNVWSPPICGSAGPPPGGMDY;
(SEQ ID NO: 233)
AAQRLGSVTDYTKYDY;
(SEQ ID NO: 234)
AIPPRAYSGGSYSLKDQSKYEY;
(SEQ ID NO: 235)
ADGNVWSPPICSSAGPPPGGMDY;
(SEQ ID NO: 236)
KSRSSYSNN;
(SEQ ID NO: 237)
AMPPRAYTGRSVSLKDQSKYEY;
(SEQ ID NO: 238)
LDTTGWGPPPYQY;
(SEQ ID NO: 239)
AHPPDPSRGGEWRLQTPSEYDY;
(SEQ ID NO: 240)
AAQRLGSVTDYTKYDY;
(SEQ ID NO: 241)
VPRSHFTTAQDMGQDMGAPSWYEY;
(SEQ ID NO: 242)
AVLIRYYSGGYQGLSDANEYDY;
(SEQ ID NO: 243)
VVKYLSGSYSYAGQYNF;
(SEQ ID NO: 244)
ADFNVWSPPICGSVGPPPGGMDY;
(SEQ ID NO: 245)
AHESTYYSGTYYLTDPRRYVY;
(SEQ ID NO: 246)
AVPARGLTMDLENSDIYDH;
(SEQ ID NO: 247)
AATLQVTGSYYLDLSTVDIYDN;
(SEQ ID NO: 248)
ATLFRSNGPKDLSSGYEYDY;
(SEQ ID NO: 249)
AGESGVWVGGLDY;
(SEQ ID NO: 250)
VGSANSGEFRFGWVLKPDLYNY;
(SEQ ID NO: 251)
ADGNVWSPPICGSAGPPPGGMDY;
(SEQ ID NO: 252)
ADGNVWSPPICGSAGPPPGGMDY;
(SEQ ID NO: 253)
ERGYAYCSDDGCQRTQDYDY;
(SEQ ID NO: 254)
GAARAWWSGSYDYTRMNNYDY;
(SEQ ID NO: 255)
AETSADSGEFRFGWVLKPSLYDY;
(SEQ ID NO: 256)
AAGSAYSGSYWNITMAANYDY;
(SEQ ID NO: 257)
AQRIFGAQPMDLSGDYEY;
(SEQ ID NO: 258)
ADGNVWSPPICGSAGPPPGGMDY;
(SEQ ID NO: 259)
ARDYRGIKDLDLKGDYDY;
(SEQ ID NO: 260)
ADFNVWSPPICGSIWYGPPPRGMDY;
(SEQ ID NO: 261)
ADSNVWSPPICGSRWYGPPPGGMAY;
(SEQ ID NO: 262)
ADFNVWSPPICGSNWYGPPPGGMDY;
(SEQ ID NO: 263)
ADFNVWSPPICGSIWYGPPPGGMDY;
(SEQ ID NO: 264)
ADGNVWSPPICGSAGPPPGGMDY;
(SEQ ID NO: 265)
ARIITVATMRLDSDYDY;
(SEQ ID NO: 266)
ADGNVWSPPICGSAGPPPGGMDY;
(SEQ ID NO: 267)
ADSNVWSPPICGRTWYGPPPGGMDY;
(SEQ ID NO: 268)
ADFNVWSPPICGSIWYGPPPGGMAY;
(SEQ ID NO: 269)
ADFNVWSPPICGSNWYGPPPGGMDY;
(SEQ ID NO: 270)
ADFNVWSPPICGSSWYGPPPGGMDY;
(SEQ ID NO: 271)
ADFNVWSPPICGSRWYGPPPGGMEY;
(SEQ ID NO: 272)
ADGNVWSPPICGSAGPPPGGMDY;
(SEQ ID NO: 273)
ADFNVWSPPICGSRWYGPPPGGMAY;
(SEQ ID NO: 274)
ADGNVWSPPICGSAGPPPGGMDY;
(SEQ ID NO: 275)
ADSNVWSPPICGKTWYGPPPGGMDY;
(SEQ ID NO: 276)
AGESGVWVGGLDY;
(SEQ ID NO: 277)
ADSNVWSPPICGSTWYGPPPGGMAY;
(SEQ ID NO: 278)
ADGNVWSPPICGSAGPPPGGMDY;
(SEQ ID NO: 279)
ADFNVWSPPICGSRWYGPPPGGMDY;
(SEQ ID NO: 280)
ADFNVWSPPICGSRWYGPPPGGMDY;
(SEQ ID NO: 281)
ADFNVWSPPICGSRWYGPPPGGMDY;
(SEQ ID NO: 282)
ADFNVWSPPICGSIWYGPPPGGMDY;
(SEQ ID NO: 283)
ADGNVWSPPICGSAGPPPGGMDY;
(SEQ ID NO: 284)
ADFNVWSPPICGSVGPPPGGMDY;
(SEQ ID NO: 285)
ADFNVWSPPICGSSWYGPPPGGMAY;
(SEQ ID NO: 286)
AGESGVWVGGLDY;
(SEQ ID NO: 287)
ADFNVWSPPICGSSWYGPPPGGMEY;
(SEQ ID NO: 288)
ADGNVWSPPICGSAGPPPGGMDY;
(SEQ ID NO: 289)
ADFNVWSPPICSSNWYGPPPRGMDY;
or
(SEQ ID NO: 290)
ADFNVWSPPICGSIWYGPPPRGMDY.

In various embodiments, the CD8 targeting moiety comprises SEQ ID NO: 77, SEQ ID NO: 148, and SEQ ID NO: 219.

In various embodiments, the CD8 targeting moiety comprises SEQ ID NO: 77, SEQ ID NO: 148, and SEQ ID NO: 220.

In various embodiments, the CD8 targeting moiety comprises SEQ ID NO: 77, SEQ ID NO: 148, and SEQ ID NO: 221.

In various embodiments, the CD8 targeting moiety comprises SEQ ID NO: 77, SEQ ID NO: 149, and SEQ ID NO: 219.

In various embodiments, the CD8 targeting moiety comprises SEQ ID NO: 77, SEQ ID NO: 149, and SEQ ID NO: 220.

In various embodiments, the CD8 targeting moiety comprises SEQ ID NO: 77, SEQ ID NO: 149, and SEQ ID NO: 221.

In various embodiments, the CD8 targeting moiety comprises SEQ ID NO: 78, SEQ ID NO: 148, and SEQ ID NO: 219.

In various embodiments, the CD8 targeting moiety comprises SEQ ID NO: 78, SEQ ID NO: 148, and SEQ ID NO: 220.

In various embodiments, the CD8 targeting moiety comprises SEQ ID NO: 78, SEQ ID NO: 148, and SEQ ID NO: 221.

In various embodiments, the CD8 targeting moiety comprises SEQ ID NO: 78, SEQ ID NO: 149, and SEQ ID NO: 219.

In various embodiments, the CD8 targeting moiety comprises SEQ ID NO: 78, SEQ ID NO: 149, and SEQ ID NO: 220.

In various embodiments, the CD8 targeting moiety comprises SEQ ID NO: 78, SEQ ID NO: 149, and SEQ ID NO: 221.

By way of example, in some embodiments, the CD8 targeting moiety comprises an amino acid sequence selected from the following sequences:

R3HCD27
(SEQ ID NO: 291)
QVQLQESGGGSVQPGGSLRLSCAASGFTFDDYAMSWVRQVPGKGLEWV
STINWNGGSAEYAEPVKGRFTISRDNAKNTVYLQMNSLKLEDTAVYYCAK
DADLVWYNLSTGQGTQVTVSSAAAYPYDVPDYGS;
R3HCD129
(SEQ ID NO: 292)
QVQLQESGGGLVQPGGSLRLSCAASGFTFDDYAMSWVRQVPGKGLEWV
STINWNGGSAEYAEPVKGRFTISRDNAKNTVYLQMNSLKLEDTAVYYCAK
DADLVWYNLRTGQGTQVTVSSAAAYPYDVPDYGS;
or
R2HCD26
(SEQ ID NO: 293)
QVQLQESGGGLVQAGGSLRLSCAASGFTFDDYAIGWFRQAPGKEREGVS
CIRVSDGSTYYADPVKGRFTISSDNAKNTVYLQMNSLKPEDAAVYYCAAG
SLYTCVQSIVVVPARPYYDMDYWGKGTQVTVSSAAAYPYDVPDYGS.

In various embodiments, the CD8 targeting moiety comprises an amino acid sequence selected from the following sequences:

1CDA 7
(SEQ ID NO: 294)
QVQLQESGGGLVQAGGSLRLSCAASGRSFSSYTLAWFRQAPGKEREFVASITWGGGNTYYPDSVKGRFTISRD
DAKNTVYLQMNSLKPEDTAVYYCAAKGLRNSDWDLRRGYEYDYWGQGTQVTVSSAAAYPYDVPDYGSHHHHH
H;
1CDA 12
(SEQ ID NO: 295)
QVQLQESGGGLVQDGGSLRLSCAFSGRTFSSYTMGWFRQGPGKEREFVAATVWTGAGTVYADSVKGRFTISR
DNAKNTVYLQMNSLRPEDTAVYYCAADQASVPPPYGSERYDIASPSEYDYWGQGTQVTVSSAAAYPYDVPDYG
SHHHHHH;
1CDA 14
(SEQ ID NO: 296)
QVQLQESGGGLVQAGASLRLSCAASGRTFSSYIMGWFRQAPGKEREFVAAIGWSADITVYADSVKGRFTISRDN
AENMVYLQMNSLNPEDTAVYYCAANSRAYYSSSYDLGRLASYDYWGQGTQVTVSSAAAYPYDVPDYGSHHHH
HH;
1CDA 15
(SEQ ID NO: 297)
QVQLQESGGGLVQAGGSLRLSCAASGRTFSSYTMGWFRQAPGKEREFVAFIDWSGGGTYYDDSVKGRFTISR
DNAENTVYLQMNNLEPEDTAVYYCAAAQRLGSVTDYTKYDYWGQGTQVTVSSAAAYPYDVPDYGSHHHHHH;
1CDA 17
(SEQ ID NO: 298)
QVQLQESGGGLVQAGGSLRLSCAASGRTSGRTFSSYTMGWFRQAPGKEREFVATITWGGGSTYYADSVKGRF
TISRDNANNTVYLQMNSLKPEDTAVYYCAASVKWAGSGIDISGSRNYDYWGQGTQVTVSSAAAYPYDVPDYGS
HHHHHH;
1CDA 18
(SEQ ID NO: 299)
QVQLQESGGGLVQPGGSLRLSCLASGRTFSSYAMGWFRQAPGKEREFVAAISWSGGPTVYADHVKGRFTISRD
NAKNTVYLQVNSLKPEDTADYYCAAKRLDYSATDKGVDLSDEYDYWGQGTQVTVSSAAAYPYDVPDYGSHHHH
HH;
1CDA 19
(SEQ ID NO: 300)
QVQLQESGGGLVQAGDSLRLSCAASGLTFSNYIMGWFRQAPGKEREFVAAITWGGGSTVYADSVEGRFTISRD
GTKNTVSLQMNSLLPEDTAVYYCAAAGGSGRLRDLKVGQNYDYWGQGTQVTVSSAAAYPYDVPDYGSHHHHH
H;
1CDA 24
(SEQ ID NO: 301)
QVQLQESGGGLVQAGGSLRLSCAASGRTFSSYTMGWFRQAPGREREFVAAITWSGVSTVYTDSVKGRFTVSR
DNAKNTVYLQMNSLKPEDTAVYYCAADSPPRTYSSGSVNLEDGSEYDYWGQGTQVTVSSAAAYPYDVPDYGS
HHHHHH;
1CDA 26
(SEQ ID NO: 302)
QVQLQESGGGLVQAGGSLRLSCAASGRTFSSDTMGWFRQAPGKEREFVGAIMWSGAFTHYADSVKGRFTISR
DNAKNTVYLQMNALKPEDTAVYYCAVIPGRGSALPIDVGKSDEYEYWGQGTQVTVSSAAAYPYDVPDYGSHHH
HHH;
1CDA 28
(SEQ ID NO: 303)
QVQLQESGGGLVQAGDSLRLSCAASGLTFSNYIMGWFRQAPGKEREFVAAITWGGGSTVYADSVEGRFTISRD
GTKNTVSLQMNSLQPEDTAVYYCAAAGASGRLRDLKVGQNYDYWGQGTQVTVSSAAAYPYDVPDYGSHHHHH
H;
1CDA 37
(SEQ ID NO: 304)
QVQLQESGGGLVQAGGSLRLSCAGSGFTLDYYGIGWFRQAPGKEREGVSCISSSDRNTYYADSVKGRFTISGD
NAKNTVYLQMNNLKPEDTAVYYCAADGNVWSPPICGSAGPPPGGMDYWGKGTQVTVSSAAAYPYDVPDYGSH
HHHHH;
1CDA 43
(SEQ ID NO: 305)
QVQLQESGGGLVQAGGSLRLSCVASGHTFSSYTMGWFRQAPGKEREFVAFIDWSGGGTYYANSVKGRFTISR
DNAENTVYLQMNNLKPEDTAVYYCAAAQRLGSVTDYTKYDYWGQGTQVTVSSAAAYPYDVPDYGSHHHHHH;
1CDA 45
(SEQ ID NO: 306)
QVQLQESGGGLVQAGGSLRLSCAASGRTFSSYVIGWFRQAPGKEREFVAVITWSGDSTYSSDSLKGRFTISRDN
AKNTVYLQMNALNPEDTAVYYCAAIPPRAYSGGSYSLKDQSKYEYWGQGTQVTVSSAAAYPYDVPDYGSHHHH
HH;
1CDA 47
(SEQ ID NO: 307)
QVQLQESGGGLVQAEGSLKLSCISGFAFDGYAIGWFRQAPGKEREGVACISSKDGSTYYADSVKGRFTMSVDK
TKNTVYLQMSSLKPEDTAVYYCAADGNVWSPPICSSAGPPPGGMDYWGKGTQVTVSSAAAYPYDVPDYGSHH
HHHH;
1CDA 48
(SEQ ID NO: 308)
QVQLQESGGGLVQPGGSLTLSCAASGFAFGFFDMTWVRQAPGKGLEWVSGINSIGGSTTYADSVKGRFTISRD
NAKNELYLQMNSLKPDDTAVYYCAKSRSSYSNNWRPPGQGTQVTVSSAAAYPYDVPDYGSHHHHHH;
1CDA 58
(SEQ ID NO: 309)
QVQLQESGGGLVQARGSLTLSCAASGRTFSNYVIGWFRQAPGEEREFVAVVTWSGDSTYSSDSLKGRFTISRD
NAKNTVYLQMNNLNPEDTAVYYCAAMPPRAYTGRSVSLKDQSKYEYWGQGTQVTVSSAAAYPYDVPDYGSHH
HHHH;
1CDA 65
(SEQ ID NO: 310)
QVQLQESGGGLVQPGGSLRLSCAASGSIFSINVMGWYRQTPGKERELVAKITNFGITSYADSAQGRFTISRGNAK
NTVYLQMNSLKPEDTAVYYCNLDTTGWGPPPYQYWGQGTQVTVSSAAAYPYDVPDYGSHHHHHH;
1CDA 68
(SEQ ID NO: 311)
QVQLQESGGGLVQAGASLRLSCAASGRTFSNYNVGWFRQAPGKEREFVSFISWISDITYYSDSVKGRFIISRDNA
KNMVYLQMNSLKPEDTAVYYCAAHPPDPSRGGEWRLQTPSEYDYWGQGTQVTVSSAAAYPYDVPDYGSHHH
HHH;
1CDA 73
(SEQ ID NO: 312)
QVQLQESGGGLVQAGGSLRLSCAASGHTFSSYTMGWFRQAPGKEREFVAFIDWSGGGTYYADSVKGRFTISR
DNAENTVYLQMNNLKPEDTAVYYCAAAQRLGSVTDYTKYDYWGQGTQVTVSSAAAYPYDVPDYGSHHHHHH;
1CDA 75
(SEQ ID NO: 313)
QVQLQESGGGLVQAGGSLRLSCAASGRTFSTYPVGWFRQAPGKEREFVAVILWSGVSTYYADSVKGRFTISRD
NAQNTVYLQMDSLKPEDTAVYYCAVPRSHFTTAQDMGQDMGAPSWYEYWGQGTQVTVSSAAAYPYDVPDYG
SHHHHHH;
1CDA 86
(SEQ ID NO: 314)
QVQLQESGGGLVQAGGSLRLSCAASGRTFSNYAMGWFRQAPGKEREFVAAIVWSGGSTYYADSVKGRFTISR
DNAKNTVYLQMNSLKPEDTAVYYCAAVLIRYYSGGYQGLSDANEYDYWGQGTQVTVSSAAAYPYDVPDYGSHH
HHHH;
1CDA 87
(SEQ ID NO: 315)
QVQLQESGGGLVQAGASLRLSCSASGRTFSDYRMGWFRQAPGKEREWVAAISSSGYHTYYADSVKGRFTISR
DNAKNTGYLQMSSLKPEDTAVYYCAVVKYLSGSYSYAGQYNFWGQGTQVTVSSAAAYPYDVPDYGSHHHHHH;
1CDA 88
(SEQ ID NO: 316)
QVQLQESGGGLVQAGDSLKLSCAASGLTFSNYIMAWFRQAPGKEREGVSCISSPDGSTYYADSVKGRFTISSDN
AKNTVYLQMNSLKPEDTAVYYCAADFNVWSPPICGSVGPPPGGMDYWGKGTQVTVSSAAAYPYDVPDYGSHH
HHHH;
1CDA 89
(SEQ ID NO: 317)
QVQLQESGGGLVQAGGSLRLSCAASGRTFSNSVMGWFRQPPGKEREFVAAVLWSGVSTAYADSVKGRFTISR
DNAKNTVYLQMNNLKPDDTAVYYCAAHESTYYSGTYYLTDPRRYVYWGQGTQVTVSSAAAYPYDVPDYGSHH
HHHH;
1CDA 92
(SEQ ID NO: 318)
QVQLQESGGGLVQAGGSLRLSCVGDGRTFSSYIIGWFRQAPGNEREFVVAITWDGSATTYADSVKGRFTVSRD
SAKNTAYLQMNSLKPEDTAVYYCAAVPARGLTMDLENSDIYDHWGRGTQVTVSSAAAYPYDVPDYGSHHHHHH;
1CDA 93
(SEQ ID NO: 319)
QVQLQESGGGLVQAGGSLRLSCAASGRTFSSYVMGWFRQALGKEREFVAAIGWNGGITYYADSVKGRFAISRD
NAKNTVYLQMNSLKPEDTAVYYCAAATLQVTGSYYLDLSTVDIYDNWGQGTQVTVSSAAAYPYDVPDYGSHHH
HHH;
2CDA 1
(SEQ ID NO: 320)
QVQLQESGGGLVQAGGSLRLSCAASGGTFSNYVMGWFRQAPGKEREFVGFITWSGASTYYADSVKGRFTISR
DNAENTVYLQMNSLKPEDTAVYYCAATLFRSNGPKDLSSGYEYDYWGQGTQVTVSSAAAYPYDVPDYGSHHH
HHH;
2CDA 5
(SEQ ID NO: 321)
QVQLQESGGGLVQAGDSLRLTCTASGRTFSNYGIGWFRQAPGKEREFVAGINWSGESADYADSVKGRFTISRD
NAKNTVYLQMNSLKPEDTAVYYCAAGESGVWVGGLDYWXQGTQVTVSSAAAYPYDVPDYGSHHHHHH;
2CDA 22
(SEQ ID NO: 322)
QVQLQESGGGLVQAGGSLRLSCAASGFTFDDYAIAWFRQAPGKEREGVSCIERSDGSTYYADSVKGRFTISSDN
AKNTVYLQMNSLKPEDTAVYYCAVGSANSGEFRFGWVLKPDLYNYWGQGTQVTVSSAAAYPYDVPDYGSHHH
HHH;
2CDA 28
(SEQ ID NO: 323)
QVQLQESGGGLVQAGGSLRLSCTASGRTFSSYTVAWFRQSPGKEREGISCISNTDSSTYYADSVKGRFTISSDN
AKSTVHLQMSSLKPEDTAVYYCAADGNVWSPPICGSAGPPPGGMDYWGKGTQVTVSSAAAYPYDVPDYGSHH
HHHH;
2CDA 62
(SEQ ID NO: 324)
QVQLQESGGGLVQPGGSLRLSCATFGFPFDDYAIAWFRQAPGKEREGVSCISNTDSSTYYADSVKGRFTISSDN
AKNTVHLQMSSLKPEDTAVYYCAADGNVWSPPICGSAGPPPGGMDYWGKGTQVTVSSAAAYPYDVPDYGSHH
HHHH;
2CDA 68
(SEQ ID NO: 325)
QVQLQESGGGLVQAGGSLRLSCAASGRTFSSYVMGWFRQAPGKEREFVAQISWSAGSIYYADSVKGRFTISND
NAKRTVYLQMNSLKPEDTAVYYCAERGYAYCSDDGCQRTQDYDYWGQGTQVTVSSAAAYPYDVPDYGSHHH
HHH;
2CDA 73
(SEQ ID NO: 326)
QVQLQESGGGLVQAGGSLRLSCAASGRTLSSNPMAWFRQAAGKEREFVAGMSWNPGPAVYADSVKGRFTISR
DSAENTVYLQMNSLKPEDTAVYYCAGAARAWWSGSYDYTRMNNYDYWGPGTQVTVSSAAAYPYDVPDYGSH
HHHHH;
2CDA 74
(SEQ ID NO: 327)
QVQLQESGGGLVQAGGSLRLSCAVSGFTFDNYAIGWFRQAPGKEREGVSCISRSDGSTYYADSVRGRFTISSD
NAKNTVYLQMNSLKPEDTAVYYCAAETSADSGEFRFGWVLKPSLYDYWGQGTQVTVSSAAAYPYDVPDYGSH
HHHHH;
2CDA 75
(SEQ ID NO: 328)
QVQLQESGGGLVQAGGSLRLSCAASGRAFSSYFMGWFRQTPGKEREFVANIGWTGDMTYYADSVKGRFTISR
DNAKNTVYLQMNSLKPEDTAVYYCAAAGSAYSGSYWNITMAANYDYWGQGTQVTVSSAAAYPYDVPDYGSHH
HHHH;
2CDA 77
(SEQ ID NO: 329)
QVQLQESGGGLVQAGGSLRLSCAASTPTFSSYNMGWFRQAPGKEREFVAAIIWSGSMTYYADSMKGRFTVSID
NAKNTVYLQMNSLKPEDTAVYYCAAQRIFGAQPMDLSGDYEYWGQGTQVTVSSAAAYPYDVPDYGSHHHHHH;
2CDA 81
(SEQ ID NO: 330)
QVQLQESGGGLVQAGGSLRLSCATFGFTFDDYAIAWFRQAPGKEREGISCISNTDSSTYYADSVKGRFTISSDSA
KNTVHLQMSSLKPEDTAVYYCAADGNVWSPPICGSAGPPPGGMDYWGKGTQVTVSSAAAYPYDVPDYGSHHH
HHH;
2CDA 87
(SEQ ID NO: 331)
QVQLQESGGGLVQAGGSLRLSCKASGGTFSGYIMGWFRQAPGKEREFVAANTWSGGPTYYSDSVKGRFTISR
DNAKNTVYLQMNTLKPEDTAVYQCAARDYRGIKDLDLKGDYDYWGQGTQVTVSSAAAYPYDVPDYGSHHHHH
H;
2CDA 88
(SEQ ID NO: 332)
QVQLQESGGGLVQAGDSLKLSCATSGRSFSSYTIAWFRQAPGKEREGISCISSDGSTGYADSVRGRFTISSDNA
KNTVYLQMNSLKPEDTAVYYCAADFNVWSPPICGSIWYGPPPRGMDYWGKGTQVTVSSAAAYPYDVPDYGSH
HHHHH;
2CDA 89
(SEQ ID NO: 333)
QVQLQESGGGLVQAGGYLRLSCAASGFSSDDYTIGWFRQAPGKEREGISCYSSSDGSTGFADSVKGRFTISSD
NAKNTVYLQMNNLRPEDTAVYYCAADSNVWSPPICGSRWYGPPPGGMAYWGKGTQVTVSSAAAYPYDVPDY
GSHHHHHH;
2CDA 91
(SEQ ID NO: 334)
QVQLQESGGGLAQVGGSLRLSCTASGFTFDDYTIGWFRQAPGKEREGISCISSDGSTGYADSVKGRFTISSDNA
KNTVYLQMNSLKPEDTAVYYCAADFNVWSPPICGSNWYGPPPGGMDYWGKGTQVTVSSAAAYPYDVPDYGSH
HHHHH;
2CDA 92
(SEQ ID NO: 335)
QVQLQESGGGLVQAGGSLRLSCAASGFSSDDYTIGWFRQAPGKEREGIGCIKSSDGTTGYADSVKGRFTISSDN
AKNTVYLQMNSLKPEDTAVYYCAADFNVWSPPICGSIWYGPPPGGMDYWGKGTQVTVSSAAAYPYDVPDYGS
HHHHHH;
2CDA 93
(SEQ ID NO: 336)
QVQLQESGGGLAQAGGSLRLSCAASGFTFDQYTIAWFRQAPGKEREGVSCISNTDSSTYYADSVKGRFTISSDN
AKNTVYLQMSSLKPEDTAVYYCAADGNVWSPPICGSAGPPPGGMDYWGKGTQVTVSSAAAYPYDVPDYGSHH
HHHH;
2CDA 94
(SEQ ID NO: 337)
QVQLQESGGGLVQAGGSLRLSCAASGRTFSSYAMAWFRQAPGKEREFVAAIAWSAGSTYYADSVKGRFAISRD
NAENTVYLQMNSLKPEDTAVYYCAARIITVATMRLDSDYDYWGQGTQVTVSSAAAYPYDVPDYGSHHHHHH;
2CDA 95
(SEQ ID NO: 338)
QVQLQESGGGLVQAGGSLRLSCAASGFAFDGYAIGWFRQAPGKEREGVSCISSKEGSTYYADSVKGRFTISSD
NAKNTVYLQMSSLKPEDTAVYYCAADGNVWSPPICGSAGPPPGGMDYWGKGTQVTVSSAAAYPYDVPDYGSH
HHHHH;
3CDA 3
(SEQ ID NO: 339)
QVQLQESGGGLVQAGGSLRLSCAASGFSSDDYTIAWFRRAPGKEREGISCISSSDGSTGYADSVKGRFTITSDS
AKNTVYLQMNSLKPEDTAVYYCAADSNVWSPPICGRTWYGPPPGGMDYWGKGTQVTVSSAAAYPYDVPDYGS
HHHHHH;
3CDA 8
(SEQ ID NO: 340)
QVQLQESGGGLVQPGGSLRLSCAASGFSSDDYTIGWFRQAPGKEREGISCYSSRDGTTGYADSVKGRFTISSD
NAKNTVYLQMNSLKPEDTAVYYCAADFNVWSPPICGSIWYGPPPGGMAYWGQGTQVTVSSAAAYPYDVPDYG
SHHHHHH;
3CDA 11
(SEQ ID NO: 341)
QVQLQESGGGLVQAGGSLRLSCAASGFTFDDYTIGWFRQAPGKEREGISCISSDGSTGYADSVKGRFTISSDNA
KNTVYLQMNSLKPEDTAVYYCAADFNVWSPPICGSNWYGPPPGGMDYWGKGTQVTVSSAAAYPYDVPDYGSH
HHHHH;
3CDA 18
(SEQ ID NO: 342)
QVQLQESGGGLVQAGGSLRLSCAASGFSSDDYTIGWFRQAPGKEREGISCYSSSDGSTGYADSVKGRFTISSD
NAKNTVYLQMNSLKPEDTAVYYCAADFNVWSPPICGSSWYGPPPGGMDYWGKGTQVTVSSAAAYPYDVPDYG
SHHHHHH;
3CDA 19
(SEQ ID NO: 343)
QVQLQESGGGLVQAGGSLRLSCAASGFSSDDYTIGWFRQAPGKEREGISCFSSSDGSTGFADSVKGRFTISSD
NATNTVYLEMNSLKPEDTAVYYCAADFNVWSPPICGSRWYGPPPGGMEYWGKGTQVTVSSAAAYPYDVPDYG
SHHHHHH;
3CDA 21
(SEQ ID NO: 344)
QVQLQESGGGLVQAGGSLRLSCATFGFSFDDYAIAWFRQAPGKEREGISCISNTDSSTFYADSVKGRFTISSDNA
KNTVHLQMSSLKPEDTAVYYCAADGNVWSPPICGSAGPPPGGMDYWGKGTQVTVSSAAAYPYDVPDYGSHHH
HHH;
3CDA 24
(SEQ ID NO: 345)
QVQLQESGGGLVQAGGSLRLSCAASGFSSDDYTIGWFRQAPGKEREGISCYSSSDGSTGFADSVKGRFTISSD
NAKNTVYLQMNSLRPEDTAVYYCAADFNVWSPPICGSRWYGPPPGGMAYWGKGTQVTVSSAAAYPYDVPDYG
SHHHHHH;
3CDA 28
(SEQ ID NO: 346)
QVQLQESGGGLVQVGGSLRLSCTISGFTGNDLAIGWFRQAPGKDQREGISCISNTDSSTYYADSVKGRFTISSDN
AKNTVHLQMSSLKPEDTAVYYCAADGNVWSPPICGSAGPPPGGMDYWGKGTQVTVSSAAAYPYDVPDYGSHH
HHHH;
3CDA 29
(SEQ ID NO: 347)
QVQLQESGGGLVQAGGSLRLSCAASGFSSDDYTIAWFRRAPGKEREGISCISSSDGSTGYADSVKGRFTISSDN
AKNTVYLQMTSLKPEDTAVYYCAADSNVWSPPICGKTWYGPPPGGMDYWGKGTQVTVSSAAAYPYDVPDYGS
HHHHHH;
3CDA 31
(SEQ ID NO: 348)
QVQLQESGGGLVQAGDSLRLSCAGSEGTLSSYGIGWFRQAPGKEREFVGGINWSGDSTDYADSVKGRFTISRD
SAKNTVYLQMNSLKPEDTAVYYCAAGESGVWVGGLDYWGQGTQVTVSSAAAYPYDVPDYGSHHHHHH;
3CDA 32
(SEQ ID NO: 349)
QVQLQESGGGLVQAGGSLRLSCAASGFSSDDYTIAWFRRAPGKEREGISCFSSSDGSAGYADSVKGRFTVSSD
NAKNTVYLQMNSLKPEDTAVYYCAADSNVWSPPICGSTWYGPPPGGMAYWGKGTQVTVSSAAAYPYDVPDYG
SHHHHHH;
3CDA 33
(SEQ ID NO: 350)
QVQLQESGGGLVQAGGSLRLSCATSGFTFDDYAIAWFRQAPGKEREGVSCISNTDSSTYYADSVKGRFTISSDN
AKNTVYLQMSSLKPEDTAVYYCAADGNVWSPPICGSAGPPPGGMDYWGKGTQVTVSSAAAYPYDVPDYGSHH
HHHH;
3CDA 37
(SEQ ID NO: 351)
QVQLQESGGGLVQAGGSLRLSCEVSGLSSDDYTIGWFRQAPGKEREGFSCFSTRDGNAGYADSVKGRFTISSD
NAKNTVYLQMNNLKPEDTAVYYCAADFNVWSPPICGSRWYGPPPGGMDYWGKGTQVTVSSAAAYPYDVPDYG
SHHHHHH;
3CDA 40
(SEQ ID NO: 352)
QVQLQESGGGLVQAGGSLRLSCEVSGLSSDDYTIGWFRQAPGKKREGFSCFSSRDGSTGYADSVKGRFTISSD
NAKNTVYLQMNSLKPEDTAVYYCAADFNVWSPPICGSRWYGPPPGGMDYWGKGTQVTVSSAAAYPYDVPDYG
SHHHHHH;
3CDA 41
(SEQ ID NO: 353)
QVQLQESGGGLVQAGGSLRLSCAASGFSSDDYTIGWFRQAPGKEREGFSCFSSRDGSTGYADSVKGRFTISSD
NAKNTVYLQMNSLKPEDTAVYYCAADFNVWSPPICGSRWYGPPPGGMDYWGKGTQVTVSSAAAYPYDVPDYG
SHHHHHH;
3CDA 48
(SEQ ID NO: 354)
QVQLQESGGGLVQAGGSLRLSCAASGFSFDDYTIGWFRQVPGKEREGISCISSDGSTGYADSVKGRFTISSDNA
KNTVYLQINSLKPEDTAVYYCAADFNVWSPPICGSIWYGPPPGGMDYWGKGTQVTVSSAAAYPYDVPDYGSHH
HHHH;
3CDA 57
(SEQ ID NO: 355)
QVQLQESGGGLVQAGGSLRLSCATFGFTFDDYAIAWFRQAPGKEREGISCISNTDSSTYYADSVKGRFTISSDNA
KNTVHLQMSSLKPEDTAVYYCAADGNVWSPPICGSAGPPPGGMDYWGKGTQVTVSSAAAYPYDVPDYGSHHH
HHH;
3CDA 65
(SEQ ID NO: 356)
QVQLQESGGGLVQAGGSLXLSCAASGFTFDDYAIGWFRQAPGKEREGVSCISSPDGSTYYADSVKGRFTISSDN
AKNTVYLQMNSLKPEDTAVYYCAADFNVWSPPICGSVGPPPGGMDYWGKGTQVTVSSAAAYPYDVPDYGSHH
HHHH;
3CDA 70
(SEQ ID NO: 357)
QVQLQESGGGLVQAGASLRLSCKASGFTFGDYTIGWFRQAPGKEREGISCYSSSDGNTGYADSVKGRFTISSD
NAKNTVYLQMNSLRPEDTAVYYCAADFNVWSPPICGSSWYGPPPGGMAYWGKGTQVTVSSAAAYPYDVPDYG
SHHHHHH;
3CDA 73
(SEQ ID NO: 358)
QVQLQESGGGLVQAGDSLRLSCAGSEGTFSSYGIGWFRQAPGKEREFVGGINWSGDSTDYADSVKGRFTISRD
NAKNTVYLQMNSLKPEDTAVYYCAAGESGVWVGGLDYWGQGTQVTVSSAAAYPYDVPDYGSHHHHHH;
3CDA 83
(SEQ ID NO: 359)
QVQLQESGGGLVQAGGSLRLSCAASGFSSDDYTIGWFRQAPGKEREGISCFSSSDGSTGFADSVKGRFTISSD
NATNTVYLQMNSLKPEDTAVYYCAADFNVWSPPICGSSWYGPPPGGMEYWGKGTQVTVSSAAAYPYDVPDYG
SHHHHHH;
3CDA 86
(SEQ ID NO: 360)
QVQLQESGGGLVQAGDSLRLSCTASGVSIGDYNIGWFRQAPGKEREGVSCISSGDGTTYYTDSVKGRFTISTDN
AKNTVYLQMNSLKPEDTAVYYCAADGNVWSPPICGSAGPPPGGMDYWGKGTQVTVSSAAAYPYDVPDYGSHH
HHHH;
3CDA 88
(SEQ ID NO: 361)
QVQLQESGGGLVQAGGSLRLSCAASGFTFDDYTIAWFRQAPGGKEREGISCISSDGSTGYADSVKGRFTISSDN
AKNMVYLQMNSLKPEDTALYYCAADFNVWSPPICSSNWYGPPPRGMDYWGKGTQVTVSSAAAYPYDVPDYGS
HHHHHH;
or
3CDA 90
(SEQ ID NO: 362)
QVQLQESGGGLVQAGGSLRLSCAASGFTFDDYTIAWFRQAPGKEREGISCISSDGSTGYADSVRGRFTISSDNA
KNTVYLQMNSLKPEDTAVYYCAADFNVWSPPICGSIWYGPPPRGMDYWGKGTQVTVSSAAAYPYDVPDYGSH
HHHHH.

In some embodiments, the CD8 targeting moiety comprises an amino acid sequence selected from SEQ ID NOs: 294-362 (provided above) without the terminal histidine tag sequence (i.e., HHHHHH; SEQ ID NO: 363). In some embodiments, the CD8 targeting moiety comprises an amino acid sequence selected from SEQ ID NOs: 294-362 (provided above) without the HA tag (i.e., YPYDVPDYGS; SEQ ID NO: 364).

In some embodiments, the CD8 targeting moiety comprises an amino acid sequence selected from SEQ ID NOs: 294-362 (provided above) without the AAA linker (i.e., AAA).

In some embodiments, the CD8 targeting moiety comprises an amino acid sequence selected from SEQ ID NOs: 294-362 (provided above) without the AAA linker and HA tag.

In some embodiments, the CD8 targeting moiety comprises an amino acid sequence selected from SEQ ID NOs: 294-362 (provided above) without the AAA linker, HA tag, and terminal histidine tag sequence (i.e., AAAYPYDVPDYGSHHHHHH; SEQ ID NO: 365).

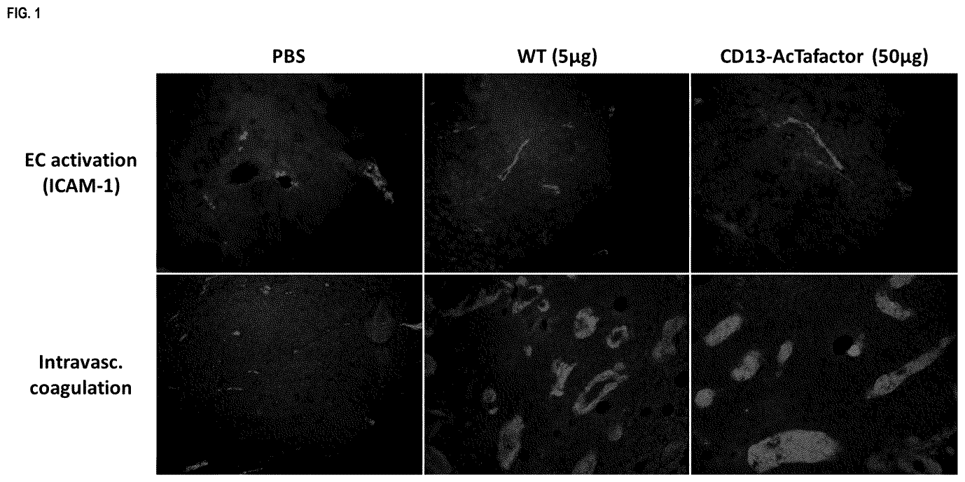

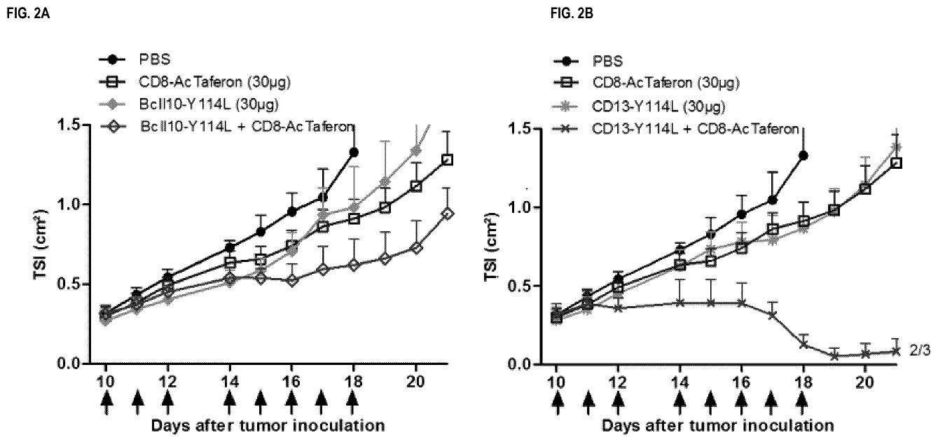

In some embodiments, the CD8 targeting moiety comprises an amino acid sequence described in US Patent Publication No. 2014/0271462, the entire contents of which are incorporated by reference. In various embodiments, the CD8 binding agent comprises an amino acid sequence described in Table 0.1, Table 0.2, Table 0.3, and/or FIGS. 1A-12I of US Patent Publication No. 2014/0271462, the entire contents of which are incorporated by reference. In various embodiments, the CD8 binding agent comprises a HCDR1 of a HCDR1 of SEQ ID NO: 366 or 367 and/or a HCDR2 of HCDR2 of SEQ ID NO: 366 or 367 and/or a HCDR3 of HCDR3 of SEQ ID NO: 366 or 367 and/or a LCDR1 of LCDR1 of SEQ ID NO: 368 and/or a LCDR2 of LCDR2 of SEQ ID NO: 368 and/or a LCDR3 of LCDR3 of SEQ ID NO: 368.

SEQ ID NO: 366:
Glu Val Gln Leu Val Glu Ser Gly Gly Gly Leu Val
Gln Pro Gly Gly Ser Leu Arg Leu Ser Cys Ala Ala
Ser Gly Phe Asn Ile Lys Asp Thr Tyr Ile His Trp
Val Arg Gln Ala Pro Gly Lys Gly Leu Glu Trp Val
Ala Arg Ile Asp Pro Ala Asn Asp Asn Thr Leu Tyr
Ala Ser Lys Phe Gln Gly Arg Ala Thr Ile Ser Ala
Asp Thr Ser Lys Asn Thr Ala Tyr Leu Gln Met Asn
Ser Leu Arg Ala Glu Asp Thr Ala Val Tyr Tyr Cys
Gly Arg Gly Tyr Gly Tyr Tyr Val Phe Asp His Trp
Gly Gln Gly Thr Leu Val Thr Val Ser Ser.
SEQ ID NO: 367:
Gln Val Gln Leu Val Gln Ser Gly Ala Glu Val Lys
Lys Pro Gly Ala Thr Val Lys Ile Ser Cys Lys Val
Ser Gly Phe Asn Ile Lys Asp Thr Tyr Ile His Trp
Val Gln Gln Ala Pro Gly Lys Gly Leu Glu Trp Met
Gly Arg Ile Asp Pro Ala Asn Asp Asn Thr Leu Tyr
Ala Ser Lys Phe Gln Gly Arg Val Thr Ile Thr Ala
Asp Thr Ser Thr Asp Thr Ala Tyr Met Glu Leu Ser
Ser Leu Arg Ser Glu Asp Thr Ala Val Tyr Tyr Cys
Ala Arg Gly Tyr Gly Tyr Tyr Val Phe Asp His Trp
Gly Gln Gly Thr Leu Val Thr Val Ser Ser.
SEQ ID NO: 368:
Asp Val Gln Ile Thr Gln Ser Pro Ser Ser Leu Ser
Ala Ser Val Gly Asp Arg Val Thr Ile Thr Cys Arg
Thr Ser Arg Ser Ile Ser Gln Tyr Leu Ala Trp Tyr
Gln Gln Lys Pro Gly Lys Val Pro Lys Leu Leu Ile
Tyr Ser Gly Ser Thr Leu Gln Ser Gly Val Pro Ser
Arg Phe Ser Gly Ser Gly Ser Gly Thr Asp Phe Thr
Leu Thr Ile Ser Ser Leu Gln Pro Glu Asp Val Ala
Thr Tyr Tyr Cys Gln Gln His Asn Glu Asn Pro Leu
Thr Phe Gly Gly Gly Thr Lys Val Glu Ile Lys.

In some embodiments, the present technology contemplates the use of any natural or synthetic analogs, mutants, variants, alleles, homologs and orthologs (herein collectively referred to as “analogs”) of the CD8 targeting moiety described herein. In some embodiments, the amino acid sequence of the CD8 targeting moiety further includes an amino acid analog, an amino acid derivative, or other non-classical amino acids.

In some embodiments, the CD8 targeting moiety comprises a targeting moiety comprising a sequence that is at least 60% identical to any one of the CD8 sequences disclosed herein. For example, the CD8 targeting moiety may comprise a targeting moiety comprising a sequence that is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any one of the CD8 sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity to any one of the CD8 sequences disclosed herein).

In various embodiments, the CD8 targeting moiety comprises an amino acid sequence having one or more amino acid mutations with respect to any one of the CD8 sequences disclosed herein. In various embodiments, the CD8 binding agent comprises a targeting moiety comprising an amino acid sequence having one, or two, or three, or four, or five, or six, or seen, or eight, or nine, or ten, or fifteen, or twenty amino acid mutations with respect to any one of the CD8 sequences disclosed herein. In some embodiments, the one or more amino acid mutations may be independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In various embodiments, the amino acid mutation may be in the CDRs of the targeting moiety (e.g., the CDR1, CDR2 or CDR3 regions). In another embodiment, amino acid alteration may be in the framework regions (FRs) of the targeting moiety (e.g., the FR1, FR2, FR3, or FR4 regions).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

In various embodiments, the mutations do not substantially reduce the CD8 targeting moiety's capability to specifically bind to CD8. In various embodiments, the mutations do not substantially reduce the CD8 targeting moiety's capability to specifically bind to CD8 without functionally modulating CD8.

In various embodiments, the binding affinity of the CD8 targeting moiety for the full-length and/or mature forms and/or isoforms and/or splice variants and/or fragments and/or any other naturally occurring or synthetic analogs, variants, or mutants (including monomeric, dimeric, heterodimeric, multimeric and/or associated forms) of human CD8 α and/or β chains may be described by the equilibrium dissociation constant (K_{D}). In various embodiments, the CD8 targeting moiety binds to the full-length and/or mature forms and/or isoforms and/or splice variants and/or fragments and/or any other naturally occurring or synthetic analogs, variants, or mutants (including monomeric, dimeric, heterodimeric, multimeric and/or associated forms) of human CD8 α and/or β chains with a K_{D} of less than about 1 μM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 50 nM, about 40 nM, about 30 nM, about 20 nM, about 10 nM, or about 5 nM, or about 1 nM.

In various embodiments, the CD8 targeting moiety binds but does not functionally modulate the antigen of interest, i.e., CD8. For instance, in various embodiments, the CD8 targeting moiety simply targets the antigen but does not substantially functionally modulate the antigen, e.g. it does not substantially inhibit, reduce or neutralize a biological effect that the antigen has. In various embodiments, the CD8 targeting moiety binds an epitope that is physically separate from an antigen site that is important for its biological activity (e.g. an antigen's active site).

Such non-functionally modulating (e.g. non-neutralizing) binding finds use in various embodiments of the present invention, including methods in which the CD8 targeting moiety is used to directly or indirectly recruit active immune cells to a site of need via an effector antigen. For example, in various embodiments, the CD8 targeting moiety may be used to directly or indirectly recruit cytotoxic T cells via CD8 to a tumor cell in a method of reducing or eliminating a tumor (e.g. the CD8 binding agent may comprise a targeting moiety having an anti-CD8 antigen recognition domain and a targeting moiety having a recognition domain (e.g. an antigen recognition domain) directed against a tumor antigen or receptor). In such embodiments, it is desirable to directly or indirectly recruit CD8-expressing cytotoxic T cells but not to neutralize the CD8 activity. In these embodiments, CD8 signaling is an important piece of the tumor reducing or eliminating effect.

### CD13 Targeting Moieties

In some embodiments, the additional therapeutic agent—that is used in the combination therapy described herein—is a CD13-targeted chimeric protein or chimeric protein complex having at least one targeting moiety that specifically binds to CD13 and at least one signaling agent that is an interferon (IFN) or a modified form thereof. Various CD13 targeting moieties are described above and may be used as a component of the additional therapeutic agent. For example, the additional therapeutic agent—that is used in combination therapy—is a CD13 targeted chimeric protein or chimeric protein complex, which includes a targeting moiety that specifically binds to CD13 and at least one signaling agent that is an interferon or a modified form thereof. In some embodiments, the interferon is IFN-γ. In some embodiments, the modified form of IFN is a modified form IFN-γ.

### Interferon Signaling Agent

In various embodiments, the CD8-targeted chimeric proteins or chimeric protein complexes or the CD13-targeted chimeric proteins or chimeric protein complexes comprise an interferon (IFN) signaling agent. In some embodiments, the IFN signaling agent comprises a modified IFN as a signaling agent.

In some embodiments, the IFN signaling agent is an interferon or a modified version of an interferon such as interferon types I, II, and III. Illustrative interferons, including for example, interferon-α-1, 2, 4, 5, 6, 7, 8, 10, 13, 14, 16, 17, and 21, interferon-β and interferon-γ, interferon κ, interferon ε, interferon τ, and interferon ω.

In some embodiments, the modified IFN signaling agent is interferon α. In such embodiments, the modified IFN-α agent has reduced affinity and/or activity for the IFN-α/B receptor (IFNAR), i.e., IFNAR1 and/or IFNAR2 chains. In some embodiments, the modified IFN-α agent has substantially reduced or ablated affinity and/or activity for the IFN-α/B receptor (IFNAR), i.e., IFNAR1 and/or IFNAR2 chains.

Mutant forms of interferon α are known to the person skilled in the art. In an illustrative embodiment, the modified signaling agent is the allelic form IFN-α2a having the amino acid sequence of:

IFN-α2a:
(SEQ ID NO: 369)
CDLPQTHSLGSRRTLMLLAQMRKISLFSCLKDRHDFGFPQEEFGNQFQK
AETIPVLHEMIQQIFNLFSTKDSSAAWDETLLDKFYTELYQQLNDLEAC
VIQGVGVTETPLMKEDSILAVRKYFQRITLYLKEKKYSPCAWEVVRAEI
MRSFSLSTNLQESLRSKE.

In an illustrative embodiment, the modified IFN signaling agent is the allelic form IFN-α2b having the amino acid sequence of (which differs from IFN-α2a at amino acid position 23):

IFN-α2b:
(SEQ ID NO: 370)
CDLPQTHSLGSRRTLMLLAQMRRISLFSCLKDRHDFGFPQEEFGNQFQK
AETIPVLHEMIQQIFNLFSTKDSSAAWDETLLDKFYTELYQQLNDLEAC
VIQGVGVTETPLMKEDSILAVRKYFQRITLYLKEKKYSPCAWEVVRAEI
MRSFSLSTNLQESLRSKE.

In some embodiments, said IFN-α2 mutant (IFN-α2a or IFN-α2b) is mutated at one or more amino acids at positions 144-154, such as amino acid positions 148, 149 and/or 153. In some embodiments, the IFN-α2 mutant comprises one or more mutations selected from L153A, R149A, and M148A. Such mutants are described, for example, in WO2013/107791 and Piehler et al., (2000) J. Biol. Chem, 275:40425-33, the entire contents of all of which are hereby incorporated by reference.

In some embodiments, the IFN-α2 mutants have reduced affinity and/or activity for IFNAR1. In some embodiments, the IFN-α2 mutant comprises one or more mutations selected from F64A, N65A, T69A, L80A, Y85A, and Y89A, as described in WO2010/030671, the entire contents of which is hereby incorporated by reference.

In some embodiments, the IFN-α2 mutant comprises one or more mutations selected from K133A, R144A, R149A, and L153A as described in WO2008/124086, the entire contents of which is hereby incorporated by reference.

In some embodiments, the IFN-α2 mutant comprises one or more mutations selected from R120E and R120E/K121E, as described in WO2015/007520 and WO2010/030671, the entire contents of which are hereby incorporated by reference. In such embodiments, said IFN-α2 mutant antagonizes wildtype IFN-α2 activity. In such embodiments, said mutant IFN-2 has reduced affinity and/or activity for IFNAR1 while affinity and/or activity of IFNR2 is retained.

In some embodiments, the human IFN-α2 mutant comprises (1) one or more mutations selected from R120E and R120E/K121E, which, without wishing to be bound by theory, create an antagonistic effect and (2) one or more mutations selected from K133A, R144A, R149A, and L153A, which, without wishing to be bound by theory, allow for an attenuated effect at, for example, IFNAR2. In an embodiment, the human IFN-α2 mutant comprises R120E and L153A.

In some embodiments, the human IFN-α2 mutant comprises one or more mutations selected from, L15A, A19W, R22A, R23A, L26A, F27A, L30A, L30V, K31A, D32A, R33K, R33A, R33Q, H34A, D35A, Q40A, D114R, L117A, R120A, R125A, K134A, R144A, A145G, A145M, M148A, R149A, S152A, L153A, and N156A as disclosed in WO 2013/059885, the entire disclosures of which are hereby incorporated by reference. In some embodiments, the human IFN-α2 mutant comprises the mutations H57Y, E58N, Q61S, and/or L30A as disclosed in WO 2013/059885. In some embodiments, the human IFN-α2 mutant comprises the mutations H57Y, E58N, Q61S, and/or R33A as disclosed in WO 2013/059885. In some embodiments, the human IFN-α2 mutant comprises the mutations H57Y, E58N, Q61S, and/or M148A as disclosed in WO 2013/059885. In some embodiments, the human IFN-α2 mutant comprises the mutations H57Y, E58N, Q61S, and/or L153A as disclosed in WO 2013/059885. In some embodiments, the human IFN-α2 mutant comprises the mutations N65A, L80A, Y85A, and/or Y89A as disclosed in WO 2013/059885. In some embodiments, the human IFN-α2 mutant comprises the mutations N65A, L80A, Y85A, Y89A, and/or D114A as disclosed in WO 2013/059885. In some embodiments, the human IFN-α2 mutant comprises one or more mutations selected from R144X₁, A145X₂, and R33A, wherein X₁ is selected from A, S, T, Y, L, and I, and wherein X₂ is selected from G, H, Y, K, and D.

In some embodiments, the human IFN-α2 mutant comprises one or more mutations selected from L15A, R22A, R23A, S25A, L26A, F27A, L30A, L30V, K31A, D32A, R33A, R33K, R33Q, H34A, Q40A, D113R, L116A, R119A, R119E, R124A, R124E, K130A, E131A, K132A, K133A, M147A, R148A, S 149A, L152A, N155A, (L30A, H57Y, E58N and Q61S), (M147A, H57Y, E58N and Q61S), (L152A, H57Y, E58N and Q61S), (R143A, H57Y, E58N and Q61S), (N65A, L80A, Y85A and Y89A) (N65A, L80A, Y85A, Y89A and D113A), (N65A, L80A, Y85A, Y89A and L116A), (N65A, L80A, Y85A, Y89A and RI 190A), (Y85A, Y89A and D113A), (D113A and RI119A), (L116A and R119A), (L116A, R119A and K120A), (R119A and K120A), (R119E and K120E), replacement of R at position 143 with A, D, E, G, H, I, K, L, N, Q, S, T, V or Y, replacement of A at position 144 with D, E, G, H, I, K, L, M, N, Q, S, T, V or Y, and deletion of residues L160 to E164.

In some embodiments, the human IFN-α2 mutant comprises a mutation which does not permit O-linked glycosylation at a position when, e.g., produced in mammalian cell culture. In some embodiments, the human IFN-α2 mutant comprises a mutation at T106. In some embodiments, T106 is substituted with A, C, D, E, F, G, H, I, K, L, M, N, P, Q, R, S, V, W, or Y.

In some embodiments, the human IFN-α2 mutant is a mutant of the IFN-α2-1b variant. Mutations in the IFN-2-1b variant are disclosed in WO 2015/168474, the entire disclosures of which are hereby incorporated by reference. By way of example, in some embodiments IFN-α2-1b comprises one or more of the following mutations: H58A, E59A, R145A, M149A, and R150A.

In some embodiments, the modified IFN signaling agent is interferon β. In such embodiments, the modified interferon β agent has reduced affinity and/or activity for the IFN-α/β receptor (IFNAR), i.e., IFNAR1 and/or IFNAR2 chains. In some embodiments, the modified interferon β agent has substantially reduced or ablated affinity and/or activity for the IFN-α/β receptor (IFNAR), i.e., IFNAR1 and/or IFNAR2 chains.

In an illustrative embodiment, the modified IFN signaling agent is IFN-β. In various embodiments, the IFN-β encompasses functional derivatives, analogs, precursors, isoforms, splice variants, or fragments of IFN-β. In various embodiments, the IFN-encompasses IFN-β derived from any species. In an embodiment, the chimeric protein or chimeric protein complex comprises a modified version of mouse IFN-β. In another embodiment, the chimeric protein or chimeric protein complex comprises a modified version of human IFN-β. Human IFN-β is a polypeptide with a molecular weight of about 22 kDa comprising 166 amino acid residues. The amino acid sequence of human IFN-β is:

(SEQ ID NO: 371)
MSYNLLGFLQRSSNFQCQKLLWQLNGRLEYCLKDRMNFDIPEEIKQLQQ
FQKEDAALTIYEMLQNIFAIFRQDSSSTGWNETIVENLLANVYHQINHL
KTVLEEKLEKEDFTRGKLMSSLHLKRYYGRILHYLKAKEYSHCAWTIVR
VEILRNFYFINRLTGYLRN.

In some embodiments, the human IFN-β is IFN-β-1a which is a glycosylated form of human IFN-β. In some embodiments, the human IFN-β is IFN-β-1b which is a non-glycosylated form of human IFN-β that has a Met-1 deletion and a Cys-17 to Ser mutation.

In various embodiments, the modified IFN-β has one or more mutations that reduce its binding to or its affinity for the IFNAR1 subunit of IFNAR. In one embodiment, the modified IFN-β has reduced affinity and/or activity at IFNAR1. In various embodiments, the modified IFN-β is human IFN-β and has one or more mutations at positions F67, R71, L88, Y92, 195, N96, K123, and R124. In some embodiments, the one or more mutations are substitutions selected from F67G, F67S, R71A, L88G, L88S, Y92G, Y92S, 195A, N96G, K123G, and R124G. In an embodiment, the modified IFN-β comprises the F67G mutation. In an embodiment, the modified IFN-comprises the K123G mutation. In an embodiment, the modified IFN-β comprises the F67G and R71A mutations. In an embodiment, the modified IFN-β comprises the L88G and Y92G mutations. In an embodiment, the modified IFN-β comprises the Y92G, 195A, and N96G mutations. In an embodiment, the modified IFN-β comprises the K123G and R124G mutations. In an embodiment, the modified IFN-β comprises the F67G, L88G, and Y92G mutations. In an embodiment, the modified IFN-β comprises the F67S, L88S, and Y92S mutations.

In some embodiments, the modified IFN-β has one or more mutations that reduce its binding to or its affinity for the IFNAR2 subunit of IFNAR. In one embodiment, the modified IFN-β has reduced affinity and/or activity at IFNAR2. In various embodiments, the modified IFN-β is human IFN-β and has one or more mutations at positions W22, R27, L32, R35, V148, L151, R152, and Y155. In some embodiments, the one or more mutations are substitutions selected from W22G, R27G, L32A, L32G, R35A, R35G, V148G, L151G, R152A, R152G, and Y155G. In an embodiment, the modified IFN-β comprises the W22G mutation. In an embodiment, the modified IFN-β comprises the L32A mutation. In an embodiment, the modified IFN-β comprises the L32G mutation. In an embodiment, the modified IFN-β comprises the R35A mutation. In an embodiment, the modified IFN-β comprises the R35G mutation. In an embodiment, the modified IFN-β comprises the V148G mutation. In an embodiment, the modified IFN-β comprises the R152A mutation. In an embodiment, the modified IFN-β comprises the R152G mutation. In an embodiment, the modified IFN-β comprises the Y155G mutation. In an embodiment, the modified IFN-β comprises the W22G and R27G mutations. In an embodiment, the modified IFN-β comprises the L32A and R35A mutation. In an embodiment, the modified IFN-β comprises the L151G and R152A mutations. In an embodiment, the modified IFN-β comprises the V148G and R152A mutations.

In some embodiments, the modified IFN-β has one or more of the following mutations: R35A, R35T, E42K, M621, G78S, A141Y, A142T, E149K, and R152H. In some embodiments, the modified IFN-β has one or more of the following mutations: R35A, R35T, E42K, M621, G78S, A141Y, A142T, E149K, and R152H in combination with C17S or C17A.

In some embodiments, the modified IFN-β has one or more of the following mutations: R35A, R35T, E42K, M621, G78S, A141Y, A142T, E149K, and R152H in combination with any of the other IFN-β mutations described herein. The crystal structure of human IFN-β is known and is described in Karpusas et al., (1998) PNAS, 94 (22): 11813-11818. Specifically, the structure of human IFN-β has been shown to include five α-helices (i.e., A, B, C, D, and E) and four loop regions that connect these helices (i.e., AB, BC, CD, and DE loops). In various embodiments, the modified IFN-β has one or more mutations in the A, B, C, D, E helices and/or the AB, BC, CD, and DE loops which reduce its binding affinity or activity at a therapeutic receptor such as IFNAR. Illustrative mutations are described in WO 2000/023114 and US20150011732, the entire contents of which are hereby incorporated by reference. In an illustrative embodiment, the modified IFN-β is human IFN-β comprising alanine substitutions at amino acid positions 15, 16, 18, 19, 22, and/or 23. In an illustrative embodiment, the modified IFN-β is human IFN-β comprising alanine substitutions at amino acid positions 28-30, 32, and 33. In an illustrative embodiment, the modified IFN-β is human IFN-β comprising alanine substitutions at amino acid positions 36, 37, 39, and 42. In an illustrative embodiment, the modified IFN-β is human IFN-β comprising alanine substitutions at amino acid positions 64 and 67 and a serine substitution at position 68. In an illustrative embodiment, the modified IFN-β is human IFN-β comprising alanine substitutions at amino acid positions 71-73. In an illustrative embodiment, the modified IFN-β is human IFN-β comprising alanine substitutions at amino acid positions 92, 96, 99, and 100. In an illustrative embodiment, the modified IFN-β is human IFN-β comprising alanine substitutions at amino acid positions 128, 130, 131, and 134. In an illustrative embodiment, the modified IFN-β is human IFN-β comprising alanine substitutions at amino acid positions 149, 153, 156, and 159. In some embodiments, the mutant IFNβ comprises SEQ ID NO: 371 and a mutation at W22, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at W22, the mutation being an aliphatic hydrophobic residue selected from glycine (G), alanine (A), leucine (L), isoleucine (I), methionine (M), and valine (V) and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at L32, the mutation being an aliphatic hydrophobic residue selected from glycine (G), alanine (A), isoleucine (I), methionine (M), and valine (V).

In some embodiments, the mutant IFNβ comprises SEQ ID NO: 371 and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at L32, the mutation being an aliphatic hydrophobic residue selected from glycine (G), alanine (A), isoleucine (I), methionine (M), and valine (V) and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at F67, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at F67, the mutation being an aliphatic hydrophobic residue selected from glycine (G), alanine (A), leucine (L), isoleucine (I), methionine (M), and valine (V) and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at L88, the mutation being an aliphatic hydrophobic residue selected from glycine (G), alanine (A), isoleucine (I), methionine (M), and valine (V).

In some embodiments, the mutant IFNβ comprises SEQ ID NO: 371 and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at F67, the mutation being an aliphatic hydrophobic residue selected from glycine (G), alanine (A), leucine (L), isoleucine (I), methionine (M), and valine (V) and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at 195, the mutation being an aliphatic hydrophobic residue selected from glycine (G), alanine (A), leucine (L), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at N96, the mutation being an aliphatic hydrophobic residue selected from glycine (G), alanine (A), leucine (L), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at Y92, the mutation being an aliphatic hydrophobic residue selected from glycine (G), alanine (A), leucine (L), isoleucine (I), methionine (M), and valine (V) and a mutation at 195, the mutation being an aliphatic hydrophobic residue selected from glycine (G), alanine (A), leucine (L), methionine (M), and valine (V) and a mutation at N96, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at K123, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at R124, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at K123, the mutation being an aliphatic hydrophobic residue selected from glycine (G), alanine (A), leucine (L), isoleucine (I), methionine (M), and valine (V) and a mutation at R124, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at L151, the mutation being an aliphatic hydrophobic residue selected from glycine (G), alanine (A), isoleucine (I), methionine (M), and valine (V).

In some embodiments, the mutant IFNβ comprises SEQ ID NO: 371 and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at L151, the mutation being an aliphatic hydrophobic residue selected from glycine (G), alanine (A), isoleucine (I), methionine (M), and valine (V) and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at V148, the mutation being an aliphatic hydrophobic residue selected from glycine (G), alanine (A), leucine (L), isoleucine (I), and methionine (M).

In some embodiments, the mutant IFNβ comprises SEQ ID NO: 371 and a mutation at V148, the mutation being an aliphatic hydrophobic residue selected from glycine (G), alanine (A), leucine (L), isoleucine (I), methionine (M), and valine (V) and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 371 and a mutation at Y155, the mutation being an aliphatic hydrophobic residue selected from glycine (G), alanine (A), leucine (L), isoleucine (I), methionine (M), and valine (V).

In an embodiment, the CD-8-targeted chimeric protein or chimeric protein complex comprising: (a) a modified IFN-β, having the amino acid sequence of SEQ ID NO: 371 and a mutation at position W22, wherein the mutation is an aliphatic hydrophobic residue and a CD8 targeting moiety disclosed herein. In various embodiments the mutation at position W22 is aliphatic hydrophobic residue is selected from G, A, L, I, M, and V. In various embodiments the mutation at position W22 is G.

Additional illustrative IFNβ mutants are provided in PCT/EP2017/061544, the entire disclosure of which is incorporated by reference herein.

In some embodiments, the modified IFN signaling agent is interferon γ. In such embodiments, the modified interferon γ agent has reduced affinity and/or activity for the interferon-gamma receptor (IFNGR), i.e., IFNGR1 and IFNGR2 chains. In some embodiments, the modified interferon γ agent has substantially reduced or ablated affinity and/or activity for the interferon-gamma receptor (IFNGR), i.e., IFNGR1 and/or IFNGR2 chains.

Among the various interferons, IFN-γ is the only member of the type II class of interferons. IFN-γ is produced predominantly by natural killer (NK) and natural killer T (NKT) cells as part of the innate immune response. IFN-γ is also produced by CD4 Th1 and CD8 cytotoxic T lymphocyte (CTL) effector T cells, macrophages, dendritic cells, and B cells. Activated IFN-γ forms a dimer which acts through a heterodimeric receptor (i.e., IFN-γ receptor or IFN-γR) composed of IFN-γ receptor 1 and IFN-γ receptor 2 subunits. IFN-γ receptor 1 is the major ligand-binding subunit, while IFN-γ receptor 2 is necessary for signal transduction and also increases the affinity of IFN-γ receptor 1 for its ligand. Binding of the IFN-γ dimer to the receptor activates the JAK-STAT signaling pathway to elicit various biological effects.

In various embodiments, the chimeric protein or chimeric protein complex of the invention comprises a modified version of IFN-γ as a signaling agent. In various embodiments, the IFN-γ encompasses functional derivatives, analogs, precursors, isoforms, splice variants, or fragments of IFN-γ. In various embodiments, the IFN-γ encompasses IFN-γ derived from any species. In an embodiment, the chimeric protein or chimeric protein complex comprises a modified version of mouse IFN-γ. In another embodiment, the chimeric protein or chimeric protein complex comprises a modified version of human IFN-γ.

Human IFN-γ is a polypeptide comprising 166 amino acid residues. In an embodiment, the human IFN-γ has the amino acid sequence of SEQ ID NO: 380, in which the signal peptide comprises the first 23 amino acids and is underlined.

(SEQ ID NO: 380)
MKYTSYILAFQLCIVLGSLGCYCQDPYVKEAENLKKYFNAGHSDVADNG
TLFLGILKNWKEESDRKIMQSQIVSFYFKLFKNFKDDQSIQKSVETIKE
DMNVKFFNSNKKKRDDFEKLTNYSVTDLNVQRKAIHELIQVMAELSPAA
KTGKRKRSQMLFRGRRASQ

As used herein, human IFN-γ may also refer to mature human IFN-γ without the N-terminal signal peptide. In this embodiment, the mature human IFN-γ comprises 143 amino acids and has the amino acid sequence of SEQ ID NO: 381.

(SEQ ID NO: 381)
QDPYVKEAENLKKYFNAGHSDVADNGTLFLGILKNWKEESDRKIMQSQI
VSFYFKLFKNFKDDQSIQKSVETIKEDMNVKFFNSNKKKRDDFEKLTNY
SVTDLNVQRKAIHELIQVMAELSPAAKTGKRKRSQMLFRGRRASQ

In some embodiments, the human IFN-γ is a glycosylated form of human IFN-γ. In some embodiments, the human IFN-γ is a non-glycosylated form of human IFN-γ.

In various embodiments, the IFN-γ is modified to have one or more mutations. In some embodiments, the mutations allow for the modified IFN-γ to have one or more of attenuated activity such as one or more of reduced binding affinity, reduced endogenous activity, and reduced specific bioactivity relative to unmutated, e.g., the wild type form of IFN-γ. For instance, the one or more of attenuated activity such as reduced binding affinity, reduced endogenous activity, and reduced specific bioactivity relative to unmutated, e.g., the wild type form of IFN-γ may be at a therapeutic receptor such as the IFN-γ receptor. Consequentially, in various embodiments, the mutations allow for the modified soluble agent to have reduced systemic toxicity, reduced side effects, and reduced off-target effects relative to unmutated, e.g., the wild type form of IFN-γ.

In various embodiments, the IFN-γ is modified to have a mutation that reduces its binding affinity and/or activity at a therapeutic receptor such as the IFN-γ receptor comprising the IFN-γ receptor 1 and IFN-γ receptor 2 subunits. In some embodiments, the activity provided by the wild type IFN-γ is agonism at the therapeutic receptor (e.g., activation of a cellular effect at a site of therapy). For example, the wild type IFN-γ may activate the therapeutic receptor. In such embodiments, the mutation results in the modified IFN-γ to have reduced activating activity at the therapeutic receptor.

In some embodiments, the reduced affinity and/or activity at the therapeutic receptor (e.g., IFN-γ receptor) is restorable by attachment with a targeting moiety. In other embodiments, the reduced affinity and/or activity at the therapeutic receptor is not substantially restorable by attachment with the targeting moiety. In various embodiments, the therapeutic chimeric proteins or chimeric protein complexes of the present invention reduce off-target effects because the IFN-γ has mutations that weaken binding affinity and/or activity at a therapeutic receptor. In various embodiments, this reduces side effects observed with, for example, the wild type IFN-γ. In various embodiments, the modified IFN-γ is substantially inactive en route to the site of therapeutic activity and has its effect substantially on specifically targeted cell types, which greatly reduces undesired side effects.

In various embodiments, the modified IFN-γ has one or more mutations that cause the IFN-γ to have attenuated or reduced affinity and/or activity, e.g., binding (e.g., K_{D}) and/or activation (measurable as, for example, KA and/or EC₅₀) for one or more therapeutic receptors (e.g., IFN-γ receptor). In various embodiments, the reduced affinity and/or activity at the therapeutic receptor allows for attenuation of activity and/or signaling from the therapeutic receptor.

In various embodiments, the modified IFN-γ has one or more mutations that reduce its binding to or its affinity for and/or biological activity for the IFN-γ receptor 1 subunit. In one embodiment, the modified IFN-γ has reduced affinity and/or activity at the IFN-γ receptor 1 subunit. In various embodiments, the modified IFN-γ is human IFN-γ that has one or more mutations at amino acid residues involved with binding to the IFN-γ receptor 1 subunit. In some embodiments, the modified IFN-γ is human IFN-γ that has one or more mutations at amino acids located at the interface with the IFN-γ receptor 1 subunit. In various embodiments, the one or more mutations are at amino acids selected from, but not limited to Q1, V5, E9, K12, H19, S20, V22, A23, D24, N25, G26, T27, L30, K108, H111, E112, 1114, Q115, A118, E119, and K125 (each with respect SEQ ID NO: 381, which is a wild type human IFN-γ and which lacks its N-terminal signal sequence). In some embodiments, the one or more mutations are substitutions selected from V5E, S20E, V22A, A23G, A23F, D24G, G26Q, H111A, H111D, 1114A, Q115A, and A118G (each with respect SEQ ID NO: 381). In embodiments, the one or more mutations are substitutions selected from V22A, A23G, D24G, H111A, H111D, 1114A, Q115A, and A118G. In an embodiment, the modified IFN-γ comprises the mutations A23G and D24G. In another embodiment, the modified IFN-γ comprises the mutations 1114A and A118G. In a further embodiment, the modified IFN-γ comprises the mutations V5E, S20E, A23F, and G26Q.

In various embodiments, the modified IFN-γ has one or more of the following mutations: deletion of residue A23, deletion of residue D24, an S201 substitution, an A23V substitution, a D21K substitution and a D24A substitution. In some embodiments, the modified IFN-γ has one or more mutations that reduce its binding to or its affinity and/or biological activity for the IFN-γ receptor 2 subunit.

In some embodiments, the modified IFN-γ has one or more mutations that reduce its binding to or its affinity and/or biological activity for both IFN-γ receptor 1 and IFN-γ receptor 2 subunits.

In some embodiments, the modified IFN-γ has one or more mutations that reduce its binding to or its affinity and/or biological activity for IFN-γ receptor 1 and one or more mutations that substantially reduce or ablate binding to or its affinity and/or biological activity for IFN-γ receptor 2. In some embodiments, chimeric proteins or chimeric protein complexes with such modified IFN-γ can provide target-selective IFN-γ receptor 1 activity (e.g., IFN-γ receptor 1 activity is restorable via targeting through the targeting moiety).

In some embodiments, the modified IFN-γ has one or more mutations that reduce its binding to or its affinity and/or biological activity for IFN-γ receptor 1 and one or more mutations that reduce its binding to or its affinity and/or biological activity for IFN-γ receptor 1. In some embodiments, chimeric proteins or chimeric protein complexes with such modified IFN-γ can provide target-selective IFN-γ receptor 1 and/or IFN-γ receptor 1 activity (e.g., IFN-γ receptor 1 and IFN-γ receptor 2 activities are restorable via targeting through the targeting moiety).

In various embodiments, the modified IFN-γ is truncated at the C-terminus. In some embodiments, the modified IFN-γ is mature IFN-γ comprising the amino acid sequence of SEQ ID NO: 381 with deletions of the C-terminal terminus. In such embodiments, the mature IFN-γ may comprise a C-terminal truncation of at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 amino acid residues. In an embodiment, the modified IFN-γ is mature IFN-γ comprising the amino acid sequence of SEQ ID NO: 381 with C-terminal deletions of 5 amino acids. In an embodiment, the modified IFN-γ is mature IFN-γ with C-terminal deletions of 7 amino acids. In an embodiment, the modified IFN-γ is mature IFN-γ comprising the amino acid sequence of SEQ ID NO: 381 with C-terminal deletions of 14 amino acids. In an embodiment, the modified IFN-γ is mature IFN-γ comprising the amino acid sequence of SEQ ID NO: 381 with C-terminal deletions of 15 amino acids. In an embodiment, the modified IFN-γ is mature IFN-γ comprising the amino acid sequence of SEQ ID NO: 381 with C-terminal deletions of 16 amino acids. Additional modified IFN-γ with C-terminal truncations that may be utilized in the present invention is described in Haelewyn et al., Biochem. J. (1997), 324:591-595 and Lundell et al., Protein Eng. (1991) 4:335-341, the entire contents are hereby incorporated by reference

In various embodiments, the modified IFN-γ is a single chain IFN-γ as described, for example, in Randal et al. (2001) Structure 9:155-163 and Randal et al. (1998) Protein Sci. 7:1057-1060, the entire contents are hereby incorporated by reference. In some embodiments, the single chain IFN-γ comprises a first IFN-γ chain linked at its C-terminus to the N-terminus of a second IFN-γ chain. In various embodiments, the first and second IFN-γ chains are linked by a linker, as described elsewhere herein.

In some embodiments, the first IFN-γ chain comprises a C-terminal truncation of at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 amino acid residues. In an embodiment, the first IFN-γ chain comprises a C-terminal truncation of about 24 amino acid residues. In some embodiments, the second IFN-γ chain comprises an N-terminal truncation of at least about 1, about 2, about 3, about 4, or about 5 amino acid residues. In an embodiment, the second IFN-γ chain comprises an N-terminal truncation of about 3 amino acid residues. In some embodiments, the second IFN-γ chain comprises a C-terminal truncation of at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 amino acid residues. In various embodiments, the first and/or second IFN-γ chains comprise one or more amino acid mutations at Q1, V5, E9, K12, H19, S20, V22, A23, D24, N25, G26, T27, L30, K108, H111, E112, 1114, Q115, A118, E119, and K125, as described elsewhere herein. In various embodiments, the first and/or second IFN-γ chains comprise one or more substitutions selected from V5E, S20E, V22A, A23G, A23F, D24G, G26Q, H111A, H111D, 1114A, Q115A, and A118G. In various embodiments, the first and/or second IFN-γ chains comprise one or more substitutions selected from V22A, A23G, D24G, H111A, H111D, 1114A, Q115A, and A118G. In various embodiments, the first and/or second IFN-γ chains comprise the A23G and the D24G substitution. In various embodiments, the first and/or second IFN-γ chains comprise the 1114A and the A118G substitution. In another embodiment, the mutations are V5E, S20E, A23F, and G26Q.

In various embodiments, a first and/or second IFN-γ chain comprises one or more substitutions as disclosed herein and the first and/or second IFN-γ chain comprises a C-terminal truncation as disclosed herein.

In various embodiments, a first and/or second IFN-γ chain comprises one or more substitutions as disclosed herein and a C-terminal truncation as disclosed herein.

The crystal structure of human IFN-γ is known and is described in, for example, Ealick et al., (1991) Science, 252:698-702. Specifically, the structure of human IFN-γ has been shown to include a core of six α-helices and an extended unfolded sequence in the C-terminal region. In various embodiments, the modified IFN-γ has one or more mutations in the one or more helices which reduce its binding affinity and/or biological activity at a therapeutic receptor (e.g., IFN-γ receptor).

In various embodiments, the modified IFN-γ has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, about 80%-100% of the affinity and/or biological activity for the therapeutic receptor (e.g., IFN-γ receptor or any one of its IFN-γ receptor 1 and IFN-γ receptor 2 subunits) relative to the wild type IFN-γ. In some embodiments, the binding affinity and/or biological activity is at least about 2-fold lower, about 3-fold lower, about 4-fold lower, about 5-fold lower, about 6-fold lower, about 7-fold lower, about 8-fold lower, about 9-fold lower, at least about 10-fold lower, at least about 15-fold lower, at least about 20-fold lower, at least about 25-fold lower, at least about 30-fold lower, at least about 35-fold lower, at least about 40-fold lower, at least about 45-fold lower, at least about 50-fold lower, at least about 100-fold lower, at least about 150-fold lower, or about 10-50-fold lower, about 50-100-fold lower, about 100-150-fold lower, about 150-200-fold lower, or more than 200-fold lower relative to the wild type IFN-γ.

In various embodiments, the modified IFN-γ comprises one or more mutations that reduce the endogenous activity of the IFN-γ to about 75%, or about 70%, or about 60%, or about 50%, or about 40%, or about 30%, or about 25%, or about 20%, or about 10%, or about 5%, or about 3%, or about 1%, e.g., relative to the wild type IFN-γ.

In some embodiments, the modified IFN-γ comprises one or more mutations that cause the modified IFN-γ to have reduced affinity and/or biological activity for a receptor. In some embodiments, the modified IFN-γ's binding affinity and/or biological activity for a receptor is lower than the binding affinity and/or biological activity of the targeting moiety for its receptor. In some embodiments, this binding affinity and/or biological activity differential is between the modified IFN-γ/receptor and targeting moiety/receptor on the same cell. In some embodiments, this binding affinity and/or biological activity, differential allows for the modified IFN-γ to have localized, on-target effects and to minimize off-target effects that underlie side effects that are observed with wild type IFN-γ. In some embodiments, this binding affinity and/or biological activity is at least about 2-fold, or at least about 5-fold, or at least about 10-fold, or at least about 15-fold lower, or at least about 25-fold, or at least about 50-fold lower, or at least about 100-fold, or at least about 150-fold less.

Receptor binding activity may be measured using methods known in the art. For example, affinity and/or binding activity may be assessed by Scatchard plot analysis and computer-fitting of binding data (e.g., Scatchard, 1949) or by reflectometric interference spectroscopy under flow through conditions, as described by Brecht et al. (1993), the entire contents of all of which are hereby incorporated by reference.

In various embodiments, the attenuated activity at the therapeutic receptor, the weakened affinity and/or biological activity at the therapeutic receptor is restorable by attachment with a targeting moiety, having high affinity for an antigen at the site of therapeutic activity (e.g., an antibody or antibody format described herein). The targeting is realized by linking the modified IFN-γ to a targeting moiety. In an embodiment, the modified IFN-γ is linked to a targeting moiety through its amino-terminus. In another embodiment, the modified IFN-γ is linked to a targeting moiety through its carboxy-terminus. In this way, the present chimeric proteins or chimeric protein complexes provide, in some embodiments, localized, on-target, and controlled therapeutic action at the therapeutic receptor In various embodiments, a chimeric protein or chimeric protein complex of the present invention comprises an IFN-γ comprising one or more substitutions as disclosed herein and/or a C-terminal truncation as disclosed herein.

In some embodiments, the modified IFN signaling agent is a consensus interferon. The consensus interferon is generated by scanning the sequences of several human non-allelic IFN-α subtypes and assigning the most frequently observed amino acid in each corresponding position. The consensus interferon differs from IFN-α2b at 20 out of 166 amino acids (88% homology), and comparison with IFN-β shows identity at over 30% of the amino acid positions. In various embodiments, the consensus interferon comprises the following amino acid sequence:

(SEQ ID NO: 372)
MCDLPQTHSLGNRRALILLAQMRRISPFSCLKDRHDFGFPQEEFDGNQF
QKAQAISVLHEMIQQTFNLFSTKDSSAAWDESLLEKFYTELYQQLNDLE
ACVIQEVGVEETPLMNVDSILAVKKYFQRITLYLTEKKYSPCAWEVVRA
EIMRSFSLSTNLQERLRRKE.

In some embodiments, the consensus interferon comprises the amino acid sequence of SEQ ID NO: 373, which differs from the amino acid sequence of SEQ ID NO: 372 by one amino acid, i.e., SEQ ID NO: 373 lacks the initial methionine residue of SEQ ID NO: 372:

(SEQ ID NO: 373)
CDLPQTHSLGNRRALILLAQMRRISPFSCLKDRHDFGFPQEEFDGNQFQ
KAQAISVLHEMIQQTFNLFSTKDSSAAWDESLLEKFYTELYQQLNDLEA
CVIQEVGVEETPLMNVDSILAVKKYFQRITLYLTEKKYSPCAWEVVRAE
IMRSFSLSTNLQERLRRKE.

In various embodiments, the consensus interferon comprises a modified version of the consensus interferon, i.e., a consensus interferon variant, as a signaling agent. In various embodiments, the consensus interferon variant encompasses functional derivatives, analogs, precursors, isoforms, splice variants, or fragments of the consensus interferon.

In an embodiment, the consensus interferon variants are selected form the consensus interferon variants disclosed in U.S. Pat. Nos. 4,695,623, 4,897,471, 5,541,293, and 8,496,921, the entire contents of all of which are hereby incorporated by reference. For example, the consensus interferon variant may comprise the amino acid sequence of IFN-CON₂ or IFN-CON₃ as disclosed in U.S. Pat. Nos. 4,695,623, 4,897,471, and 5,541,293. In an embodiment, the consensus interferon variant comprises the amino acid sequence of IFN-CON₂:

(SEQ ID NO: 374)
CDLPQTHSLGNRRTLMLLAQMRRISPFSCLKDRHDFGFPQEEFDGNQFQ
KAQAISVLHEMIQQTFNLFSTKDSSAAWDESLLEKFYTELYQQLNDLEA
CVIQEVGVEETPLMNVDSILAVKKYFQRITLYLTEKKYSPCAWEVVRAE
IMRSFSLSTNLQERLRRKE.

In an embodiment, the consensus interferon variant comprises the amino acid sequence of IFN-CON₃:

(SEQ ID NO: 375)
CDLPQTHSLGNRRALILLAQMRRISPFSCLKDRHDFGFPQEEFDGNQFQ
KAQAISVLHEMIQQTFNLFSTKDSSAAWDESLLEKFYTELYQQLNDLEA
CVIQEVGVEETPLMNEDSILAVRKYFQRITLYLTEKKYSPCAWEVVRAE
IMRSFSLSTNLQERLRRKE.

In an embodiment, the consensus interferon variant comprises the amino acid sequence of any one of the variants disclosed in U.S. Pat. No. 8,496,921. For example, the consensus variant may comprise the amino acid sequence of:

(SEQ ID NO: 376)
MCDLPQTHSLGNRRALILLAQMRRISPFSCLKDRHDFGFPQEEFDGNQF
QKAQAISVLHEMIQQTFNLFSTKDSSAAWDESLLEKFYTELYQQLNDLE
ACVIQEVGVEETPLMNEDSILAVRKYFQRITLYLTEKKYSPCAWEVVRA
EIMRSFSLSTNLQERLRRKE.

In another embodiment, the consensus interferon variant may comprise the amino acid sequence of:

(SEQ ID NO: 377)
MCDLPQTHSLGNRRALILLAQMRRISPFSCLKDRHDFGFPQEEFDGNQF
QKAQAISVLHEMIQQTFNLFSTKDSSAAWDESLLEKFYTELYQQLNDLE
ACVIQEVGVEETPLMNEDSILAVRKYFQRITLYLTEKKYSPCAWEVVRA
EIMRSFSLCTNLQERLRRKE.

In some embodiments, the consensus interferon variant may be PEGylated, i.e., comprises a PEG moiety. In an embodiment, the consensus interferon variant may comprise a PEG moiety attached at the S156C position of SEQ ID NO: 377.

In some embodiments, the engineered interferon is a variant of human IFN-2a, with an insertion of Asp at approximately position 41 in the sequence Glu-Glu-Phe-Gly-Asn-Gln (SEQ ID NO: 378) to yield Glu-Glu-Phe-Asp-Gly-Asn-Gln (SEQ ID NO: 379) (which resulted in a renumbering of the sequence relative to IFN-α2a sequence) and the following mutations of Arg23Lys, Leu26Pro, Glu53GIn, Thr54Ala, Pro56Ser, Asp86Glu, Ile104Thr, Glyl06Glu, Thr110Glu, Lys117Asn, Arg125Lys, and Lys136Thr. All embodiments herein that describe consensus interferons apply equally to this engineered interferon.

In various embodiments, the consensus interferon variant comprises an amino acid sequence having one or more amino acid mutations. In some embodiments, the one or more amino acid mutations may be independently selected from substitutions, insertions, deletions, and truncations.

In some embodiments, the amino acid mutations are amino acid substitutions, and may include conservative and/or non-conservative substitutions.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as B methyl amino acids, C α-methyl amino acids, N α-methyl amino acids, and amino acid analogs in general).

In various embodiments, the consensus interferon is modified to have one or more mutations. In some embodiments, the mutations allow for the consensus interferon variant to have one or more of attenuated activity such as one or more of reduced binding affinity, reduced endogenous activity, and reduced specific bioactivity relative to unmutated, e.g., the wild type form of the consensus interferon (e.g., the consensus interferon having an amino acid sequence of SEQ ID NO: 372 or 373). For instance, the one or more of attenuated activity such as reduced binding affinity, reduced endogenous activity, and reduced specific bioactivity relative to unmutated, e.g. the wild type form of the consensus interferon, may be at a therapeutic receptor such as IFNAR. Consequentially, in various embodiments, the mutations allow for the consensus interferon variant to have reduced systemic toxicity, reduced side effects, and reduced off-target effects relative to unmutated, e.g. the wild type form of the consensus interferon.

In various embodiments, the consensus interferon is modified to have a mutation that reduces its binding affinity or activity at a therapeutic receptor such as IFNAR. In some embodiments, the activity provided by the consensus interferon is agonism at the therapeutic receptor (e.g. activation of a cellular effect at a site of therapy). For example, the consensus interferon may activate the therapeutic receptor. In such embodiments, the mutation results in the consensus interferon variant to have reduced activating activity at the therapeutic receptor.

In some embodiments, the reduced affinity or activity at the therapeutic receptor is restorable by attachment with a targeting moiety. In other embodiments, the reduced affinity or activity at the therapeutic receptor is not substantially restorable by attachment with the targeting moiety. In various embodiments, the therapeutic chimeric proteins or chimeric protein complexes of the present invention reduce off-target effects because the consensus interferon variant has mutations that weaken binding affinity or activity at a therapeutic receptor. In various embodiments, this reduces side effects observed with, for example, the wild type consensus interferon. In various embodiments, the consensus interferon variant is substantially inactive en route to the site of therapeutic activity and has its effect substantially on specifically targeted cell types, which greatly reduces undesired side effects.

In various embodiments, the consensus interferon variant has one or more mutations that cause the consensus interferon variant to have attenuated or reduced affinity, e.g. binding (e.g. K_{D}) and/or activation (measurable as, for example, K_{A} and/or EC₅₀) for one or more therapeutic receptors. In various embodiments, the reduced affinity at the therapeutic receptor allows for attenuation of activity and/or signaling from the therapeutic receptor.

In various embodiments, the consensus interferon variant has one or more mutations that reduce its binding to or its affinity for the IFNAR1 subunit of IFNAR. In one embodiment, the consensus interferon variant has reduced affinity and/or activity at IFNAR1. In some embodiments, the consensus interferon variant has one or more mutations that reduce its binding to or its affinity for the IFNAR2 subunit of IFNAR. In some embodiments, the consensus interferon variant has one or more mutations that reduce its binding to or its affinity for both IFNAR1 and IFNAR2 subunits.

In some embodiments, the consensus interferon variant has one or more mutations that reduce its binding to or its affinity for IFNAR1 and one or more mutations that substantially reduce or ablate binding to or its affinity for IFNAR2. In some embodiments, chimeric proteins or chimeric protein complexes with such consensus interferon variant can provide target-selective IFNAR1 activity (e.g. IFNAR1 activity is restorable via targeting through the CD8 targeting moiety).

In some embodiments, the consensus interferon variant has one or more mutations that reduce its binding to or its affinity for IFNAR2 and one or more mutations that substantially reduce or ablate binding to or its affinity for IFNAR1. In some embodiments, chimeric proteins or chimeric protein complexes with such consensus interferon variant can provide target-selective IFNAR2 activity (e.g. IFNAR2 activity is restorable via targeting through the CD8 targeting moiety).

In some embodiments, the consensus interferon variant has one or more mutations that reduce its binding to or its affinity for IFNAR1 and one or more mutations that reduce its binding to or its affinity for IFNAR2. In some embodiments, chimeric proteins or chimeric protein complexes with such consensus interferon variant can provide target-selective IFNAR1 and/or IFNAR2 activity (e.g. IFNAR1 and/IFNAR2 activity is restorable via targeting through the CD8 targeting moiety).

In some embodiments, the consensus interferon is modified to have a mutation at one or more amino acids at positions 145-155, such as amino acid positions 149, 150 and/or 154, with reference to SEQ ID NO: 373, the substitutions optionally being hydrophobic and selected from alanine, valine, leucine, and isoleucine. In some embodiments, the consensus interferon mutant comprises one or more mutations selected from M149A, R150A, and L154A, and, with reference to SEQ ID NO: 373.

In an embodiment, the consensus interferon is modified to have a mutation at amino acid position 121 (i.e., K121), with reference to SEQ ID NO: 373. In an embodiment, the consensus interferon comprises a K121E mutation, with reference to SEQ ID 373.

### Linkers in CD8-Targeted Chimeric Proteins

In some embodiments, the CD8-targeted chimeric protein or chimeric protein complex optionally comprises one or more linkers. In some embodiments, the present CD8-targeted chimeric protein or chimeric protein complex comprises a linker connecting the CD8 targeting moiety and the IFN signaling agent (e.g., modified IFN signaling agent). In some embodiments, the CD8-targeted chimeric protein or chimeric protein complex comprises a linker within the IFN signaling agent (e.g., modified IFN signaling agent). In some embodiments, the linker may be utilized to link various functional groups, residues, or moieties as described herein to the chimeric protein or chimeric protein complex. In some embodiments, the linker is a single amino acid or a plurality of amino acids that does not affect or reduce the stability, orientation, binding, neutralization, and/or clearance characteristics of the binding regions and the binding protein. In various embodiments, the linker is selected from a peptide, a protein, a sugar, or a nucleic acid.

In some embodiments, vectors encoding the CD8-targeted chimeric proteins or chimeric protein complexes linked as a single nucleotide sequence to any of the linkers described herein are provided and may be used to prepare such chimeric proteins or chimeric protein complexes.

In some embodiments, the linker length allows for efficient binding of a CD8 targeting moiety and the IFN signaling agent (e.g., modified IFN signaling agent) to their receptors. For instance, in some embodiments, the linker length allows for efficient binding of one of the CD8 targeting moieties and the IFN signaling agent to receptors on the same cell.

In some embodiments, the linker length is at least equal to the minimum distance between the binding sites of one of the CD8 targeting moieties and the IFN signaling agent to receptors on the same cell. In some embodiments the linker length is at least twice, or three times, or four times, or five times, or ten times, or twenty times, or 25 times, or 50 times, or one hundred times, or more the minimum distance between the binding sites of one of the CD8 targeting moieties and the IFN signaling agent to receptors on the same cell.

As described herein, the linker length allows for efficient binding of one of the CD8 targeting moieties and the IFN signaling agent to receptors on the same cell, the binding being sequential, e.g. CD8 targeting moiety/receptor binding preceding IFN signaling agent/receptor binding.

In some embodiments, there are two linkers in a single chimera, each connecting the IFN signaling agent to a CD8 targeting moiety. In various embodiments, the linkers have lengths that allow for the formation of a site that has a disease cell and an effector cell without steric hindrance that would prevent modulation of the either cell.

The use of a variety of linker sequences may be used to link the CD8 targeting moieties and the IFN signaling agent. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et al., (2000), Protein Eng. 13(5):309-312, the entire contents of which are hereby incorporated by reference. In various embodiments, the linker may be functional. For example, without limitation, the linker may function to improve the folding and/or stability, improve the expression, improve the pharmacokinetics, and/or improve the bioactivity of the CD8-targeted chimeric protein or chimeric protein complex.

In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is a polypeptide. In some embodiments, the linker is greater than about 100 amino acids long. For example, the linker may be greater than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid.

In embodiments directed to CD8 targeted chimeric proteins or chimeric protein complexes having two or more CD8 targeting moieties, a linker connects the two CD8 targeting moieties to each other and this linker has a short length and a linker connects a CD8 targeting moiety and an IFN signaling agent this linker is longer than the linker connecting the two CD8 targeting moieties. For example, the difference in amino acid length between the linker connecting the two targeting moieties and the linker connecting a targeting moiety and a signaling agent may be about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids.

In various embodiments, the linker is substantially comprised of glycine and serine residues (e.g. about 30%, or about 40%, or about 50%, or about 60%, or about 70%, or about 80%, or about 90%, or about 95%, or about 97% glycines and serines). For example, in some embodiments, the linker is (Gly₄Ser)ₙ, where n is from about 1 to about 8, e.g. 1, 2, 3, 4, 5, 6, 7, or 8 (SEQ ID NOs: 5-12, respectively). In an embodiment, the linker sequence is GGSGGSGGGGSGGGGS (SEQ ID NO: 13). Additional illustrative linkers include, but are not limited to, linkers having the sequence LE, GGGGS (SEQ ID NO: 5), (GGGGS)ₙ (n=1-4) (SEQ ID NOs: 5-8), (Gly)₈ (SEQ ID NO: 14), (Gly)₆ (SEQ ID NO: 15), (EAAAK)ₙ (n=1-3) (SEQ ID NOs: 16-18), A(EAAAK)₄A (n=2-5) (SEQ ID NOs: 19-22), AEAAAKEAAAKA (SEQ ID NO: 23), A(EAAAK)₄ALEA(EAAAK)₄A (SEQ ID NO: 24), PAPAP (SEQ ID NO: 25), KESGSVSSEQLAQFRSLD (SEQ ID NO: 26), EGKSSGSGSESKST (SEQ ID NO: 27), GSAGSAAGSGEF (SEQ ID NO: 28), and (XP)ₙ, with X designating any amino acid, e.g., Ala, Lys, or Glu. In various embodiments, the linker is (GGS)ₙ (n=1-20) (SEQ ID NOs: 29-48). In some embodiments, the linker is G. In some embodiments, the linker is AAA. In some embodiments, the linker is (GGGGS) n (n=5-20) (SEQ ID NOs: 9-12 and 49-60).

In some embodiments, the linker is one or more of GGGSE (SEQ ID NO: 61), GSESG (SEQ ID NO: 62), GSEGS (SEQ ID NO: 63), GEGGSGEGSSGEGSSSEGGGSEGGGSEGGGSEGGS (SEQ ID NO: 64), and a linker of randomly placed G, S, and E every 4 amino acid intervals.

In some embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). In various embodiments, the linker is a hinge region of an antibody (e.g., of IgG, IgA, IgD, and IgE, inclusive of subclasses (e.g. IgG1, IgG2, IgG3, and IgG4, and IgA1 and IgA2). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1> IgG4>IgG2.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the CH₂ domain and includes residues in CH₂. Id. The core hinge region of wild-type human IgG1 contains the sequence Cys-Pro-Pro-Cys (SEQ ID NO: 65), which when dimerized by disulfide bond formation, results in a cyclic octapeptide believed to act as a pivot, thus conferring flexibility. In various embodiments, the present linker comprises, one, or two, or three of the upper hinge region, the core region, and the lower hinge region of any antibody (e.g., of IgG, IgA, IgD, and IgE, inclusive of subclasses (e.g. IgG1, IgG2, IgG3, and IgG4, and IgA1 and IgA2). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In various embodiments, the linker of the present invention comprises one or more glycosylation sites. In various embodiments, the linker is a hinge-CH2-CH3 domain of a human IgG4 antibody.

In some embodiments, the linker is a synthetic linker such as PEG.

In various embodiments, the linker may be functional. For example, without limitation, the linker may function to improve the folding and/or stability, improve the expression, improve the pharmacokinetics, and/or improve the bioactivity of the present chimeric protein or chimeric protein complex. In another example, the linker may function to target the CD8-targeted chimeric protein or chimeric protein complex to a particular cell type or location.

### Other Therapeutic Agents

In some embodiments, the additional therapeutic agent is one or more agents selected from a phosphoinositide-3-kinase 9 (PI3K) inhibitor, anthracycline, and SMAC mimetic.

By way of example, in some embodiments the PI3K inhibitor is selected from: Wortmannin, PX-866, demethoxyviridin, LY294002, idelalisib, umbralisib, duvelisib, copanlisib, buparlisib, pilaralisib, pictilisib, alpelisib, taselisib, NCP-BEZ235, LY3023414, GSK2126458, perifosine, dactolisib, CUDC-907, voxtalisib, ME-401, IPI-549, SF1126, RP6530, INK1117, XL147 (a/k/a SAR245408), palomid 529, GSK1059615, ZSTK474, PWT33597, IC87114, TG-100-115, CAL263, RP6503, PI-103, GNE-477, and AEZS-136.

By way of example, in some embodiments the anthracycline is selected from: doxorubicin, daunorubicin, epirubicin, mitoxantrone, idarubicin, aldoxorubicin, annamycin, plicamycin, pirarubicin, aclarubicin, zorubicin, sabarubicin, zoptarelin doxorubicin, GPX-150, SP1049C, and valrubicin. In some embodiments, the anthracycline is encapsulated by a liposome (e.g., liposomal doxorubicin). In some embodiments, the liposomal anthracycline is pegylated (e.g., pegylated liposomal doxorubicin).

By way of example, in some embodiments the SMAC mimetic is selected from: birinapant (TL32711), LCL161 (Novartis), GDC-0917 (Genentech), HGS1029 (Human Genome Sciences), TPI 1237-22, AT-406/Debio1143, and GT13402.

Chimeric Protein Complexes with Fc Domains

In some embodiments, the present invention relates to chimeric protein complexes where the complexes include one or more fragment crystallizable domain (Fc domain). In some embodiments, the Fc domain has one or more mutations that reduces or eliminates one or more effector functions of the Fc domain, promotes Fc chain pairing in the Fc domain, and/or stabilizes a hinge region in the Fc domain.

In various embodiments, the present invention includes chimeric protein complexes comprising one or more targeting agents, one or more signaling agents and one or more Fc domains. In one embodiment, the chimeric protein complex includes at least one targeting moiety that specifically binds to CD13, at least one signaling agent that is a tumor necrosis factor (TNF), and at least one Fc domain. In various embodiments, the TNF signaling agent may be modified to attenuate activity. In some embodiments, the CD13-targeted chimeric protein complex may directly or indirectly recruit an immune cell to a site of action (such as, by way of non-limiting example, the tumor microenvironment).

In some embodiments, the present invention relates to a CD13-targeted chimeric protein complex having at least one targeting moiety that specifically binds to CD13, at least one signaling agent that is an interferon (IFN) or a modified form thereof and at least one Fc domain. In various embodiments, the IFN signaling agent may be modified to attenuate activity. In one embodiment, the interferon is IFN-γ or a modified form thereof.

The fragment crystallizable domain (Fc domain) is the tail region of an antibody that interacts with Fc receptors located on the cell surface of cells that are involved in the immune system, e.g., B lymphocytes, dendritic cells, natural killer cells, macrophages, neutrophils, eosinophils, basophils, and mast cells. In IgG, IgA and IgD antibody isotypes, the Fc domain is composed of two identical protein fragments, derived from the second and third constant domains of the antibody's two heavy chains. In IgM and IgE antibody isotypes, the Fc domain contains three heavy chain constant domains (CH domains 2-4) in each polypeptide chain.

In some embodiments, the Fc-based chimeric protein of complex the present technology includes a Fc domain. In some embodiments, the Fc domains are from selected from IgG, IgA, IgD, IgM or IgE. In some embodiments, the Fc domains are from selected from IgG1, IgG2, IgG3, or IgG4.

In some embodiments, the Fc domains are from selected from human IgG, IgA, IgD, IgM or IgE. In some embodiments, the Fc domains are from selected from human IgG1, IgG2, IgG3, or IgG4.

In some embodiments, the Fc domains of the Fc-based chimeric protein complex comprise the CH₂ and CH₃ regions of IgG. In some embodiments, the IgG is human IgG. In some embodiments, the human IgG is selected from IgG1, IgG2, IgG3, or IgG4.

In some embodiments, the Fc domains comprise one or more mutations. In some embodiments, the mutation(s) to the Fc domains reduces or eliminates the effector function the Fc domains. In some embodiments, the mutated Fc domain has reduced affinity or binding to a target receptor. By way of example, in some embodiments, the mutation to the Fc domains reduces or eliminates the binding of the Fc domains to FcγR. In some embodiments, the FcγR is selected from FcγRI; FcγRIIa, 131 R/R; FcγRIIa, 131 H/H, FcγRIIb; and FcγRIII. In some embodiments, the mutation to the Fc domains reduces or eliminated binding to complement proteins, such as, e.g., C1q. In some embodiments, the mutation to the Fc domains reduces or eliminated binding to both FcγR and complement proteins, such as, e.g., C1q.

In some embodiments, the Fc domains comprise the LALA mutation to reduce or eliminate the effector function of the Fc domains. By way of example, in some embodiments, the LALA mutation comprises L234A and L235A substitutions in human IgG (e.g., IgG1) (wherein the numbering is based on the commonly used numbering of the CH2 residues for human IgG1 according to EU convention (Edelman et al., PNAS, 1969; 63 (1) 78-85).

In some embodiments, the Fc domains of human IgG comprise a mutation at 46. to reduce or eliminate the effector function of the Fc domains. By way of example, in some embodiments, the mutations are selected from L234A, L234F, L235A, L235E, L235Q, K322A, K322Q, D265A, P329G, P329A, P331G, and P331S.

In some embodiments, the Fc domains comprise the FALA mutation to reduce or eliminate the effector function of the Fc domains. By way of example, in some embodiments, the FALA mutation comprises F234A and L235A substitutions in human IgG4.

In some embodiments, the Fc domains of human IgG4 comprise a mutation at one or more of F234, L235, K322, D265, and P329 to reduce or eliminate the effector function of the Fc domains. By way of example, in some embodiments, the mutations are selected from F234A, L235A, L235E, L235Q, K322A, K322Q, D265A, P329G, and P329A.

In some embodiments, the mutation(s) to the Fc domain stabilize a hinge region in the Fc domain. By way of example, in some embodiments, the Fc domain comprises a mutation at S228 of human IgG to stabilize a hinge region. In some embodiments, the mutation is S228P.

In some embodiments, the mutation(s) to the Fc domain promote chain pairing in the Fc domain. In some embodiments, chain pairing is promoted by ionic pairing (a/k/a charged pairs, ionic bond, or charged residue pair).

In some embodiments, the Fc domain comprises a mutation at one more of the following amino acid residues of IgG to promote of ionic pairing: D356, E357, L368, K370, K392, D399, and K409.

By way of example, in some embodiments, the human IgG Fc domain comprise one of the mutation combinations in Table 1 to promote of ionic pairing.

TABLE 1

| Substitution(s) on | Substitution(s) on |
|---|---|
| one Fc Chain | other Fc Chain |
| D356K D399K | K392D K409D |
| E357R L368R | K370D K409D |
| E357R L368K | K370D K409D |
| E357R D399K | K370D K409D |
| E357R | K370D |
| L368R D399K | K392D K409D |
| L368K D399K | K392D K409D |
| L368R D399K | K409D |
| L368K D399K | K409D |
| L368R | K409D |
| L368K | K409D |
| K370D K409D | E357R D399K |
| K370D K409D | E357R L368R |
| K370D K409D | E357R L368K |
| K370D K409D | E357R D399K |
| K370D K409D | E357R L368R |
| K370D K409D | E357R L368K |
| K370D | E357R |
| K370D | E357R |
| K392D K409D | D356K D399K |
| K392D K409D | L368R D399K |
| K392D K409D | L368K D399K |
| K392D K409D | D399K |
| D399K | K392D K409D |
| D399K | K409D |
| K409D | L368R |
| K409D | L368K |
| K409D | L368R D399K |
| K409D | L368K D399K |
| K409D | L368R |
| K409D | L368K |
| K409D | L368R D399K |
| K409D | L368K D399K |
| K409D | D399K |

In some embodiments, chain pairing is promoted by a knob-in-hole mutations. In some embodiments, the Fc domain comprises one or more mutations to allow for a knob-in-hole interaction in the Fc domain. In some embodiments, a first Fc chain is engineered to express the “knob” and a second Fc chain is engineered to express the complementary “hole.” By way of example, in some embodiments, human IgG Fc domain comprises the mutations of Table 2 to allow for a knob-in-hole interaction.

TABLE 2

| Substitution(s) on | Substitution(s) on |
|---|---|
| one Fc Chain | other Fc Chain |
| T366Y | Y407T |
| T366Y/F405A | T394W/Y407T |
| T366W | Y407A |
| T366W | Y407V |
| T366Y | Y407A |
| T366Y | Y407V |
| T366Y | Y407T |

In some embodiments, the Fc domains in the Fc-based chimeric protein complexes of the present technology comprise any combination of the above-disclosed mutations. By way of example, in some embodiments, the Fc domain comprises mutations that promote ionic pairing and/or a knob-in-hole interaction. By way of example, in some embodiments, the Fc domain comprises mutations that have one or more of the following properties: promote ionic pairing, induce a knob-in-hole interaction, reduce or eliminate the effector function of the Fc domain, and cause Fc stabilization (e.g. at hinge).

By way of example, in some embodiments, a human IgG Fc domains comprise mutations disclosed in Table 3, which promote ionic pairing and/or promote a knob-in-hole interaction in the Fc domain.

TABLE 3

| Substitution(s) on | Substitution(s) on |
|---|---|
| one Fc Chain | other Fc Chain |
| T366W K370D | E357R Y407A |
| T366W K370D | E357R Y407V |
| T366W K409D | L368R Y407A |
| T366W K409D | L368R Y407V |
| T366W K409D | L368K Y407A |
| T366W K409D | L368K Y407V |
| T366W K409D | L368R D399K Y407A |
| T366W K409D | L368R D399K Y407V |
| T366W K409D | L368K D399K Y407A |
| T366W K409D | L368K D399K Y407V |
| T366W K409D | D399K Y407A |
| T366W K409D | D399K Y407V |
| T366W K392D K409D | D399K Y407A |
| T366W K392D K409D | D399K Y407V |
| T366W K392D K409D | D356K D399K Y407A |
| T366W K392D K409D | D356K D399K Y407V |
| T366W K370D K409D | E357R D399K Y407A |
| T366W K370D K409D | E357R D399K Y407V |
| T366W K370D K409D | E357R L368R Y407A |
| T366W K370D K409D | E357R L368R Y407V |
| T366W K370D K409D | E357R L368K Y407A |
| T366W K370D K409D | E357R L368K Y407V |
| T366W K392D K409D | L368R D399K Y407A |
| T366W K392D K409D | L368R D399K Y407V |
| T366W K392D K409D | L368K D399K Y407A |
| T366W K392D K409D | L368K D399K Y407V |
| E357R T366W | K370D Y407A |
| E357R T366W | K370D Y407V |
| T366W L368R | Y407A K409D |
| T366W L368R | Y407V K409D |
| T366W L368K | Y407A K409D |
| T366W L368K | Y407V K409D |
| T366W L368R D399K | Y407A K409D |
| T366W L368R D399K | Y407V K409D |
| T366W L368K D399K | Y407A K409D |
| T366W L368K D399K | Y407V K409D |
| T366W D399K | Y407A K409D |
| T366W D399K | Y407V K409D |
| 1366W D399K | K392D Y407A K409D |
| T366W D399K | K392D Y407V K409D |
| T366W D356K D399K | K392D Y407A K409D |
| T366W D356K D399K | K392D Y407V K409D |
| E357R T366W D399K | K370D Y407A K409D |
| E357R T366W D399K | K370D Y407V K409D |
| E357R T366W L368R | K370D Y407A K409D |
| E357R T366W L368R | K370D Y407V K409D |
| E357R T366W L368K | K370D Y407A K409D |
| E357R T366W L368K | K370D Y407V K409D |
| T366W L368R D399K | K392D Y407A K409D |
| T366W L368R D399K | K392D Y407V K409D |
| T366W L368K D399K | K392D Y407A K409D |

By way of example, in some embodiments, a human IgG Fc domains comprise mutations disclosed in Table 4, which promote ionic pairing, promote a knob-in-hole interaction, or a combination thereof in the Fc domain. In embodiments, the “Chain 1” and “Chain 2” of Table 4 can be interchanged (e.g. Chain 1 can have Y407T and Chain 2 can have T366Y).

TABLE 4

| Chain 1 mutation | Chain 2 mutation | Reference | IgG |
|---|---|---|---|
| T366Y | Y407T | Ridgway et al., 1996 Protein | IgG1 |
| | | Engineering, Design and Selection, | |
| | | Volume 9, Issue 7, 1 July 1996, | |
| | | Pages 617-62 | |
| T366Y/F405A | T394W/Y407T | Ridgway et al., 1996 Protein | IgG1 |
| | | Engineering, Design and Selection, | |
| | | Volume 9, Issue 7, 1 July 1996, | |
| | | Pages 617-62 | |
| T366W | Y407A | Atwell et al., 1997 JMB | IgG1 |
| | | Volume 270, Issue 1, 4 July 1997, | |
| | | Pages 26-35 | |
| T366W | T366S/L368V/Y407A | Atwell et al., 1997 JMB | IgG1 |
| | | Volume 270, Issue 1, 4 July 1997, | |
| | | Pages 26-35 | |
| T366W | L368A/Y407A | Atwell et al., 1997 JMB | IgG1 |
| | | Volume 270, Issue 1, 4 July 1997, | |
| | | Pages 26-35 | |
| T366W | T366S/L368A/Y407A | Atwell et al., 1997 JMB | IgG1 |
| | | Volume 270, Issue 1, 4 July 1997, | |
| | | Pages 26-35 | |
| T366W | T366S/L368G/Y407V | Atwell et al., 1997 JMB | IgG1 |
| | | Volume 270, Issue 1, 4 July 1997, | |
| | | Pages 26-35 | |
| T366W/D399C | T366S/L368A/K392C/Y407V | Merchant et al., 1998 Nature | IgG1 |
| | | Biotechnology volume 16, | |
| | | pages 677-681 (1998) | |
| T366W/K392C | T366S/L368A/D399C/Y407V | Merchant et al., 1998 Nature | IgG1 |
| | | Biotechnology volume 16, | |
| | | pages 677-681 (1998) | |
| S354C/T366W | Y349C/T3665/L368A/Y407V | Merchant et al., 1998 Nature | IgG1 |
| | | Biotechnology volume 16, | |
| | | pages 677-681 (1998) | |
| Y349C/T366W | S354C/T366S/L368A/Y407V | Merchant et al., 1998 Nature | IgG1 |
| | | Biotechnology volume 16, | |
| | | pages 677-681 (1998) | |
| E356C/T366W | Y349C/T366S/L368A/Y407V | Merchant et al., 1998 Nature | IgG1 |
| | | Biotechnology volume 16, | |
| | | pages 677-681 (1998) | |
| Y349C/T366W | E356C/T366S/L368A/Y407V | Merchant et al., 1998 Nature | IgG1 |
| | | Biotechnology volume 16, | |
| | | pages 677-681 (1998) | |
| E357C/T366W | Y349C/T366S/L368A/Y407V | Merchant et al., 1998 Nature | IgG1 |
| | | Biotechnology volume 16, | |
| | | pages 677-681 (1998) | |
| Y349C/T366W | E357C/T366S/L368A/Y407V | Merchant et al., 1998 Nature | IgG1 |
| | | Biotechnology volume 16, | |
| | | pages 677-681 (1998) | |
| D339R | K409E | Gunasekaran et al., 2010 The Journal of | IgG1 |
| | | Biological Chemistry 285, 19637-19646. | |
| D339K | K409E | Gunasekaran et al., 2010 The Journal of | IgG1 |
| | | Biological Chemistry 285, 19637-19646. | |
| D339R | K409D | Gunasekaran et al., 2010 The Journal of | IgG1 |
| | | Biological Chemistry 285, 19637-19646. | |
| D339K | K409D | Gunasekaran et al., 2010 The Journal of | IgG1 |
| | | Biological Chemistry 285, 19637-19646. | |
| D339K | K360D/K409E | Gunasekaran et al., 2010 The Journal of | IgG1 |
| | | Biological Chemistry 285, 19637-19646. | |
| D339K | K392D/K409E | Gunasekaran et al., 2010 The Journal of | IgG1 |
| | | Biological Chemistry 285, 19637-19646. | |
| D339K/E356K | K392D/K409E | Gunasekaran et al., 2010 The Journal of | IgG1 |
| | | Biological Chemistry 285, 19637-19646. | |
| D339K/E357K | K392D/K409E | Gunasekaran et al., 2010 The Journal of | IgG1 |
| | | Biological Chemistry 285, 19637-19646. | |
| D339K/E356K | K409E/K439D | Gunasekaran et al., 2010 The Journal of | IgG1 |
| | | Biological Chemistry 285, 19637-19646. | |
| D339K/E357K | K370D/K409E | Gunasekaran et al., 2010 The Journal of | IgG1 |
| | | Biological Chemistry 285, 19637-19646. | |
| D339K/E356K/E357K | K370D/K392D/K409E | Gunasekaran et al., 2010 The Journal of | IgG1 |
| | | Biological Chemistry 285, 19637-19646. | |
| S364H/F405A | Y349T/T394F | Moore et al., 2011 mAbs, 3:6, 546-557 | IgG1 |
| S364H/T394F | Y349T/F405A | Moore et al., 2011 mAbs, 3:6, 546-557 | IgG1 |
| D221R/P228R/K409R | D221E/P228E/L368E | Strop et al., 2012 JMB Volume 420, | IgG1 |
| | | Issue 3, 13 July 2012, Pages 204-219 | |
| C223R/E225R/P228R/K409R | C223E/P228E/L368E | Strop et al., 2012 JMB Volume 420, | IgG2 |
| | | Issue 3, 13 July 2012, Pages 204-219 | |
| F405L | K409R | Labrijn et al. 2013 PNAS Mar. 26, 2013. | IgG1 |
| | | 110 (13) 5145-5150 | |
| F405A/Y407V | T394W | Von Kreudenstein et al., 2013 mAbs | IgG1 |
| | | Volume 5, 2013-Issue 5, pp. 644-654 | |
| F405A/Y407V | T366I/T394W | Von Kreudenstein et al., 2013 mAbs | IgG1 |
| | | Volume 5, 2013-Issue 5, pp. 644-654 | |
| F405A/Y407V | T366L/T394W | Von Kreudenstein et al., 2013 mAbs | IgG1 |
| | | Volume 5, 2013-Issue 5, pp. 644-654 | |
| F405A/Y407V | T366L/K392M/T394W | Von Kreudenstein et al., 2013 mAbs | IgG1 |
| | | Volume 5, 2013-Issue 5, pp. 644-654 | |
| L351Y/F405A/Y407V | T366L/K392M/T394W | Von Kreudenstein et al., 2013 mAbs | IgG1 |
| | | Volume 5, 2013-Issue 5, pp. 644-654 | |
| T350V/L351Y/F405A/Y407V | T350V/T366L/K392M/T394W | Von Kreudenstein et al., 2013 mAbs | IgG1 |
| | | Volume 5, 2013-Issue 5, pp. 644-654 | |
| T350V/L351Y/F405A/Y407V | T350V/T366L/K392L/T394W | Von Kreudenstein et al., 2013 mAbs | IgG1 |
| | | Volume 5, 2013-Issue 5, pp. 644-654 | |
| K409W | D339V/F405T | Choi et al., 2013 PNAS Jan. 2, 2013. | IgG1 |
| | | 110 (1) 270-275 | |
| K360E | Q347R | Choi et al., 2013 PNAS Jan. 2, 2013. | IgG1 |
| | | 110 (1) 270-275 | |
| K360E/K409W | D339V/Q347R/F405T | Choi et al., 2013 PNAS Jan. 2, 2013. | IgG1 |
| | | 110 (1) 270-275 | |
| Y3490/K360E/K409W | D339V/Q347R/S3540/F405T | Choi et al., 2013 PNAS Jan. 2, 2013. | IgG1 |
| | | 110 (1) 270-275 | |
| K392A/K409D | E356K/D399K | Leaver-Fey et al., 2016 Structure | IgG1 |
| | | Volume 24, Issue 4, 5 April 2016, | |
| | | Pages 641-651 | |
| T366W | T3665/L358A/Y407A | Leaver-Fey et al., 2016 Structure | IgG1 |
| | | Volume 24, Issue 4, 5 April 2016, | |
| | | Pages 641-651 | |
| D339M/Y407A | T336V/K409V | Leaver-Fey et al., 2016 Structure | IgG1 |
| | | Volume 24, Issue 4, 5 April 2016, | |
| | | Pages 641-651 | |
| D339M/K360D/Y407A | T336V/E345R/Q347R/K409V | Leaver-Fey et al., 2016 Structure | IgG1 |
| | | Volume 24, Issue 4, 5 April 2016, | |
| | | Pages 641-651 | |
| Y3495/T366V/K370Y/K409V | E357D/S364Q/Y407A | Leaver-Fey et al., 2016 Structure | IgG1 |
| | | Volume 24, Issue 4, 5 April 2016, | |
| | | Pages 641-651 | |
| Y3495/T366M/K370Y/K409V | E356G/E357D/S364Q/Y407A | Leaver-Fey et al., 2016 Structure | IgG1 |
| | | Volume 24, Issue 4, 5 April 2016, | |
| | | Pages 641-651 | |
| Y3495/T366M/K370Y/K409V | E357D/S364R/Y407A | Leaver-Fey et al., 2016 Structure | IgG1 |
| | | Volume 24, Issue 4, 5 April 2016, | |
| | | Pages 641-651 | |

And any combination as described in Tables 1-3 of US20150284475A1

By way of example, in some embodiments, a human IgG Fc domains comprise mutations disclosed in Table 5, which reduce or eliminate FcγR and/or complement binding in the Fc domain. In embodiments, the Table 5 mutations are in both chains.

TABLE 5

| Chain 1 mutation | Reference | IgG |
|---|---|---|
| L234A/L235A | Alegre et al., 1994 Transplantation | IgG1 |
| | 57:1537-1543 | |
| F234A/L235A | Alegre et al., 1994 Transplantation | IgG4 |
| | 57:1537-1543 | |
| L235E | Morgan et al., 1995 Immunology. | IgG1 |
| | 1995 October; 86(2): 319-324. | |
| L235E | Morgan et al., 1995 Immunology. | IgG4 |
| | 1995 October; 86(2): 319-324. | |
| L235A | Morgan et al., 1995 Immunology. | IgG1 |
| | 1995 October; 86(2): 319-324. | |
| G237A | Morgan et al., 1995 Immunology. | IgG1 |
| | 1995 October; 86(2): 319-324. | |
| N297H | Tao and Morrison, | IgG1 |
| | J. Immunol. 1989; 143:2595-2601 | |
| N297Q | Tao and Morrison, | IgG1 |
| | J. Immunol. 1989; 143:2595-2601 | |
| N297K | Tao and Morrison, | IgG3 |
| | J. Immunol. 1989; 143:2595-2601 | |
| N297Q | Tao and Morrison, | IgG3 |
| | J. Immunol. 1989; 143:2595-2601 | |
| D265A | Idusogie et al., 2000 J Immunol | IgG1 |
| | Apr. 15, 2000, 164 (8) 4178-4184 | |
| D270A, V, K | Idusogie et al., 2000 J Immunol | IgG1 |
| | Apr. 15, 2000, 164 (8) 4178-4184 | |
| K322A, L, M, D, E | Idusogie et al., 2000 J Immunol | IgG1 |
| | Apr. 15, 2000, 164 (8) 4178-4184 | |
| P329A, X | Idusogie et al., 2000 J Immunol | IgG1 |
| | Apr. 15, 2000, 164 (8) 4178-4184 | |
| P331A, S, G, X | Idusogie et al., 2000 J Immunol | IgG1 |
| | Apr. 15, 2000, 164 (8) 4178-4184 | |
| D265A | Idusogie et al., 2000 J Immunol | IgG1 |
| | Apr. 15, 2000, 164 (8) 4178-4184 | |
| L234A | Hezareh et al., 2001 J. Virol. | IgG1 |
| | December 2001 vol. 75 no. 24 | |
| | 12161-12168 | |
| L234A/L235A | Hezareh et al., 2001 J. Virol. | IgG1 |
| | December 2001 vol. 75 no. 24 | |
| | 12161-12168 | |
| L234F/L235E/P331S | Oganesyan et al., 2008 Acta Cryst. | IgG1 |
| | (2008). D64, 700-704 | |
| H268Q/V309L/A330S/P331S | An et al., 2009 mAbs | IgG1 |
| | Volume 1, 2009-Issue 6, | |
| | pp. 572-579 | |
| G236R/L328R | Moore et al., 2011 mAbs | IgG1 |
| | Volume 3, 2011-Issue 6, | |
| | pp. 546-557 | |
| N297G | Couch et al., 2013 Sci. Transl. | IgG1 |
| | Med., 5 (2013) | |
| | 183ra57, 1-12 | |
| N297G/D265A | Couch et al., 2013 Sci. Transl. | IgG1 |
| | Med., 5 (2013) | |
| | 183ra57, 1-12 | |
| V234A/G237A/P328S/H268A/V309L/A330S/P331S | Vafa et al., 2014 Methods | IgG2 |
| | Volume 65, Issue 1, 1 Jan. 2014, | |
| | Pages 114-126 | |
| L234A/L235A/P329G | Lo et al., 2016 The Journal of | IgG1 |
| | Biological Chemistry | |
| | 292, 3900-3908 | |
| N297D | Schlothauer et al., 2016 Protein | IgG1 |
| | Engineering, Design and | |
| | Selection, Volume 29, Issue 10, | |
| | 1 Oct. 2016, Pages 457-466 | |
| S228P/L235E | Schlothauer et al., 2016 Protein | IgG4 |
| | Engineering, Design and | |
| | Selection, Volume 29, Issue 10, | |
| | 1 Oct. 2016, Pages 457-466 | |
| S228P/L235E/P329G | Schlothauer et al., 2016 Protein | IgG4 |
| | Engineering, Design and | |
| | Selection, Volume 29, Issue 10, | |
| | 1 Oct. 2016, Pages 457-466 | |
| L234F/L235A/K322Q | Borrok et al., 2017 J Pharm Sci | IgG1 |
| | April 2017 Volume 106, Issue 4, | |
| | Pages 1008-1017 | |
| L234F/L235Q/P331G | Borrok et al., 2017 J Pharm Sci | IgG1 |
| | April 2017 Volume 106, Issue 4, | |
| | Pages 1008-1017 | |
| L234F/L235Q/K322Q | Borrok et al., 2017 J Pharm Sci | IgG1 |
| | April 2017 Volume 106, Issue 4, | |
| | Pages 1008-1017 | |
| L234A/L235A/G237A/P3285/H268A/A330S/P331S | Tam et al., 2017 Open Access | IgG1 |
| | Antibodies 2017, 6(3), 12; | |
| | doi:10.3390/antib6030012 | |
| S228P/F234A/L235A | Tam et al., 2017 Open Access | IgG4 |
| | Antibodies 2017, 6(3), 12; | |
| | doi:10.3390/antib6030012 | |
| S228P/F234A/L235A/G237A/P238S | Tam et al., 2017 Open Access | IgG4 |
| | Antibodies 2017, 6(3), 12; | |
| | doi:10.3390/antib6030012 | |
| S228P/F234A/L235A/G236□/G237A/P238S | Tam et al., 2017 Open Access | IgG4 |
| | Antibodies 2017, 6(3), 12; | |
| | doi:10.3390/antib6030012 | |

In some embodiments, the Fc domains in the Fc-based chimeric protein complexes of the present technology are homodimeric, i.e., the Fc region in the chimeric protein complex comprises two identical protein fragments.

In some embodiments, the Fc domains in the Fc-based chimeric protein complexes of the present technology are heterodimeric, i.e., the Fc domain comprises two non-identical protein fragments.

In some embodiments, heterodimeric Fc domains are engineered using ionic pairing and/or knob-in-hole mutations described herein. In some embodiments, the heterodimeric Fc-based chimeric protein complexes have a trans orientation/configuration. In a trans orientation/configuration, the targeting moiety and signaling agent are, in embodiments, not found on the same polypeptide chain in the present Fc-based chimeric protein complexes.

In some embodiments, the Fc domains includes or starts with the core hinge region of wild-type human IgG1, which contains the sequence Cys-Pro-Pro-Cys. In some embodiments, the Fc domains also include the upper hinge, or parts thereof (e.g., DKTHTCPPC; see WO 2009053368), EPKSCDKTHTCPPC, or EPKSSDKTHTCPPC; see Lo et al., Protein Engineering vol. 11 no. 6 pp. 495-500, 1998)).

### Fc-Based Chimeric Protein Complexes

The Fc-based chimeric protein complexes of the present technology comprise at least one Fc domain disclosed herein, at least one signaling agent and at least one targeting moiety (TM) disclosed herein.

It is understood that, the present Fc-based chimeric protein complexes may encompass a complex of two fusion proteins, each comprising an Fc domain.

In some embodiments, the Fc-based chimeric protein complex is heterodimeric. In some embodiments, the heterodimeric Fc-based chimeric protein complex has a trans orientation/configuration. In some embodiments, the heterodimeric Fc-based chimeric protein complex has a cis orientation/configuration.

In some embodiments, heterodimeric Fc domains are engineered using ionic pairing and/or knob-in-hole mutations described herein. In some embodiments, the heterodimeric Fc-based chimeric protein complexes have a trans orientation.

In a trans orientation, the targeting moiety and signaling agent are, in embodiments, not found on the same polypeptide chain in the present Fc-based chimeric protein complexes. In a trans orientation, the targeting moiety and signaling agent are, in embodiments, found on separate polypeptide chains in the Fc-based chimeric protein complexes. In a cis orientation, the targeting moiety and signaling agent are, in embodiments, found on the same polypeptide chain in the Fc-based chimeric protein complexes.

In some embodiments, where more than one targeting moiety is present in the heterodimeric protein complexes described herein, one targeting moiety may be in trans orientation (relative to the signaling agent), whereas another targeting moiety may be in cis orientation (relative to the signaling agent). In some embodiments, the signaling agent and target moiety are on the same ends/sides (N-terminal or C-terminal ends) of an Fc domain. In some embodiments, the signaling agent and targeting moiety are on different sides/ends of a Fc domain (N-terminal and C-terminal ends).

In some embodiments, where more than one targeting moiety is present in the heterodimeric protein complexes described herein, the targeting moieties may be found on the same Fc chain or on two different Fc chains in the heterodimeric protein complex (in the latter case the targeting moieties would be in trans relative to each other, as they are on different Fc chains). In some embodiments, where more than one targeting moiety is present on the same Fc chain, the targeting moieties may be on the same or different sides/ends of a Fc chain (N-terminal or/and C-terminal ends).

In some embodiments, where more than one signaling agent is present in the heterodimeric protein complexes described herein, the signaling agents may be found on the same Fc chain or on two different Fc chains in the heterodimeric protein complex (in the latter case the signaling agents would be in trans relative to each other, as they are on different Fc chains). In some embodiments, where more than one signaling agent is present on the same Fc chain, the signaling agents may be on the same or different sides/ends of a Fc chain (N-terminal or/and C-terminal ends).

In some embodiments, where more than one signaling agent is present in the heterodimeric protein complexes described herein, one signaling agent may be in trans orientation (as relates to the targeting moiety), whereas another signaling agent may be in cis orientation (as relates to the targeting moiety).

In some embodiments, the heterodimeric Fc-based chimeric protein complex does not comprise the signaling agent and targeting moiety on a single polypeptide.

In some embodiments, the Fc-based chimeric protein has an improved in vivo half-life relative to a chimeric protein lacking an Fc or a chimeric protein, which is not a heterodimeric complex. In some embodiments, the Fo-based chimeric protein has an improved solubility, stability and other pharmacological properties relative to a chimeric protein lacking an Fc or a chimeric protein, which is not a heterodimeric complex.

Heterodimeric Fc-based chimeric protein complexes are composed of two different polypeptides. In embodiments described herein, the targeting domain is on a different polypeptide than the signaling agent and accordingly, proteins that contain only one targeting domain copy, and also only one signaling agent. Further, in embodiments, one targeting domain (e.g. VHH) only can avoid cross-linking of the antigen on the cell surface (which could elicit undesired effects in some cases). Further, in embodiments, one signaling agent may alleviate molecular “crowding” and potential interference with avidity mediated restoration of effector function in dependence of the targeting domain. Further, in embodiments, heterodimeric Fc-based chimeric protein complexes can have two targeting moieties and these can be placed on the two different polypeptides. For instance, in embodiments, the C-terminus of both targeting moieties (e.g. VHHs) can be masked to avoid potential autoantibodies or pre-existing antibodies (e.g. VHH autoantibodies or pre-existing antibodies). Further, in embodiments, heterodimeric Fc-based chimeric protein complexes, e.g. with the targeting domain on a different polypeptide than the signaling agent may favor “cross-linking” of two cell types (e.g. a tumor cell and an immune cell). Further, in embodiments, heterodimeric Fc-based chimeric protein complexes can have two signaling agent, each on different polypeptides to allow more complex effector responses.

Further, in embodiments, heterodimeric Fc-based chimeric protein complexes, e.g. with the targeting domain on a different polypeptide than the signaling agent combinatorial diversity of targeting moiety and signaling agent. For instance, in embodiments, polypeptides with any of the targeting moieties described herein can be combined “off the shelf” with polypeptides with any of the signaling agents described herein to allow rapid generation of various combinations of targeting moieties and signaling agents in single Fc-based chimeric protein complexes.

In some embodiments, the Fc-based chimeric protein complex comprises one or more linkers. In some embodiments, the Fc-based chimeric protein complex includes a linker that connects the Fc domain, signaling agent and targeting moiety(ies). In some embodiments, the Fc-based chimeric protein complex includes a linker that connects each signaling agent and targeting moiety (or, if more than one targeting moiety, a signaling agent). In some embodiments, the Fc-based chimeric protein complex includes a linker that connects each signaling agent to the Fc domain. In some embodiments, the Fc-based chimeric protein complex includes a linker that connects each targeting moiety to the Fc domain. In some embodiments, the Fc-based chimeric protein complex includes a linker that connects a targeting moiety to another targeting moiety. In some embodiments, the Fc-based chimeric protein complex includes a linker that connects a signaling agent to another signaling agent.

In some embodiments, a Fc-based chimeric protein complex comprises two or more targeting moieties. In such embodiments, the targeting moieties can be the same targeting moiety or they can be different targeting moieties.

In some embodiments, a Fc-based chimeric protein complex comprises two or more signaling agents. In such embodiments, the signaling agents can be the same targeting moiety or they can be different targeting moieties.

By way of example, in some embodiments, the Fc-based chimeric protein complex comprise a Fc domain, at least two signaling agents (SA), and at least two targeting moieties (TM), wherein the Fc domain, signaling agents, and targeting moieties are selected from any of the Fc domains, signaling agents, and targeting moieties disclosed herein. In some embodiments, the Fc domain is homodimeric.

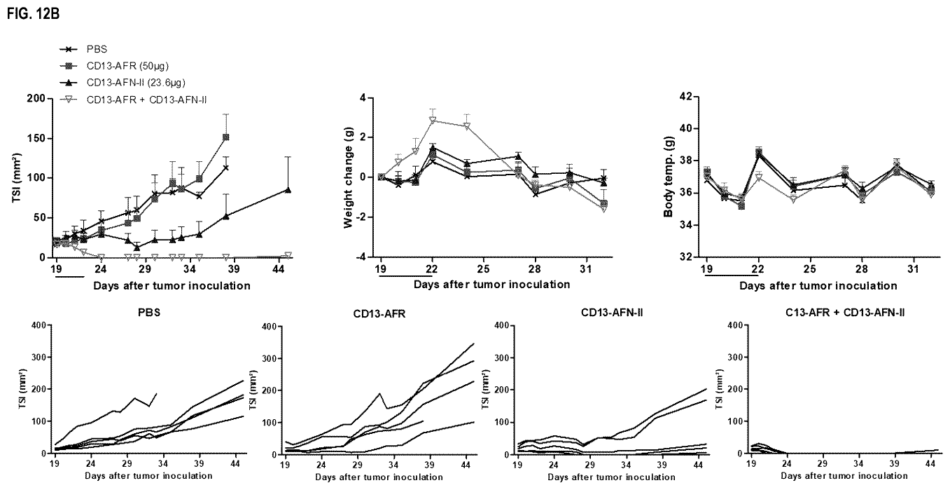

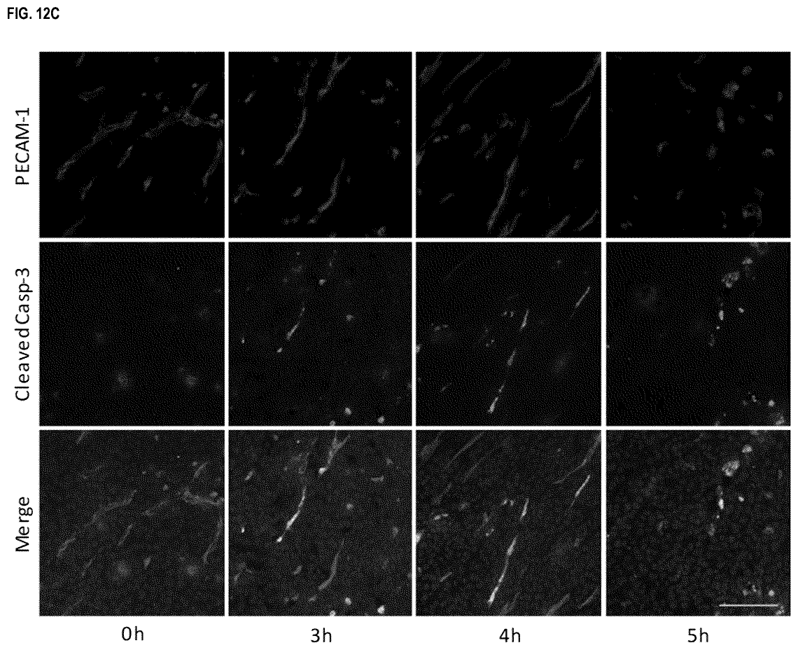

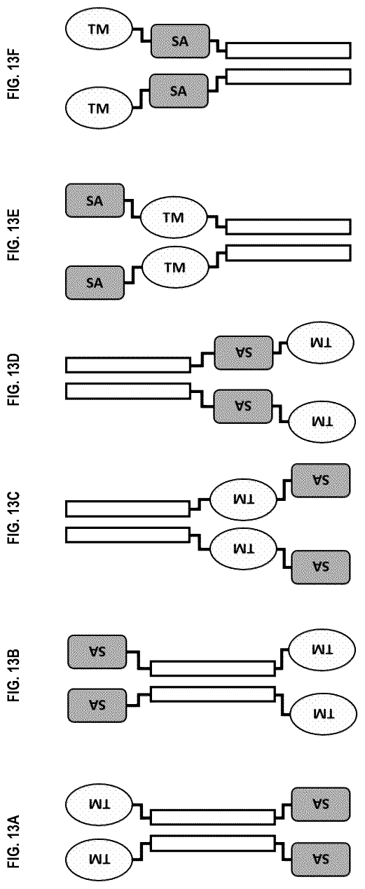

In various embodiments, the Fc-based chimeric protein complex takes the form of any of the schematics of FIGS. 13A-F.

In various embodiments, the Fc-based chimeric protein complex takes the form of any of the schematics of FIGS. 14A-H.

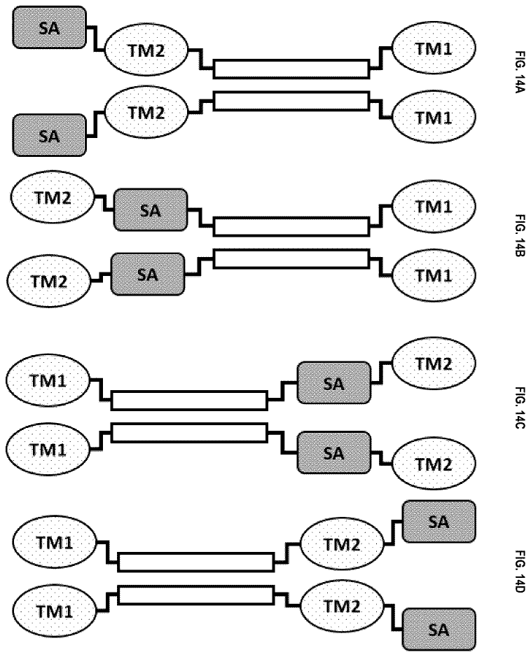

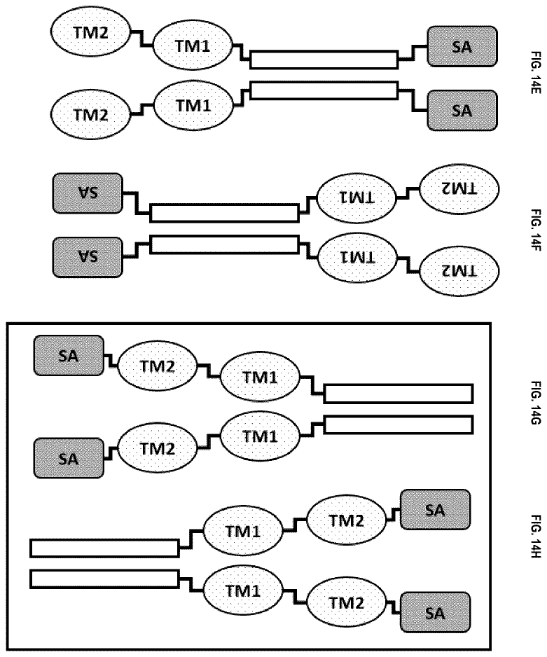

In various embodiments, the Fc-based chimeric protein complex takes the form of any of the schematics of FIGS. 15A-H.

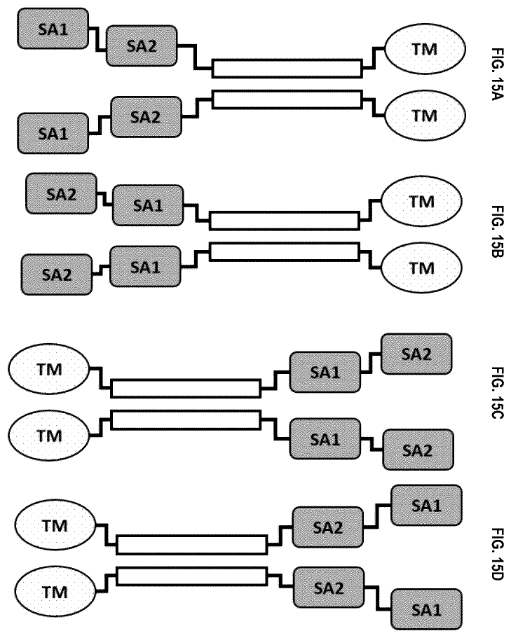

In various embodiments, the Fc-based chimeric protein complex takes the form of any of the schematics of FIGS. 16A-D.

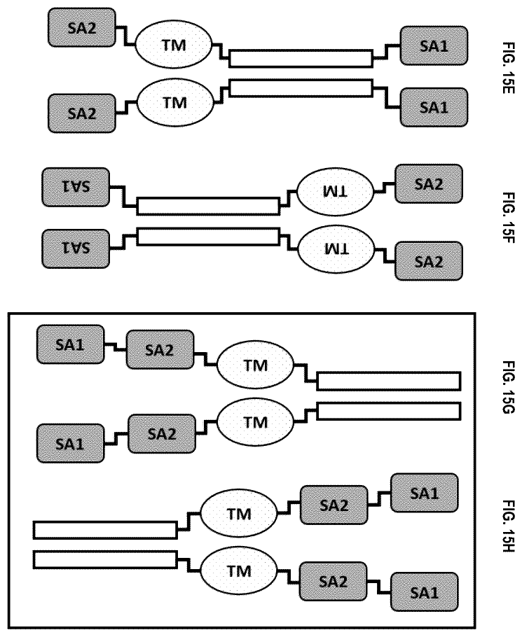

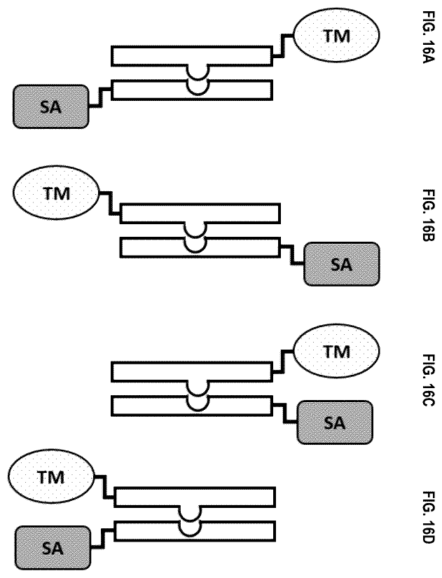

In various embodiments, the Fc-based chimeric protein complex takes the form of any of the schematics of FIGS. 17A-F.

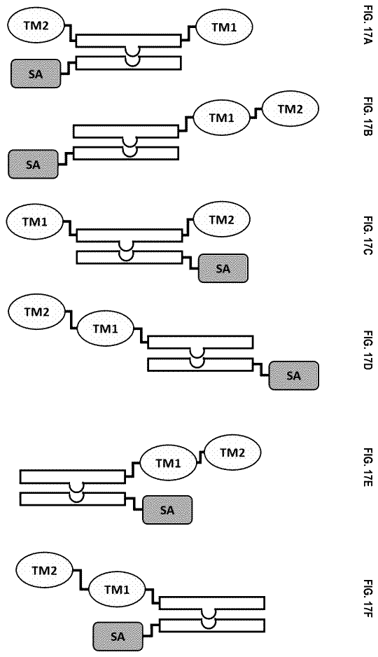

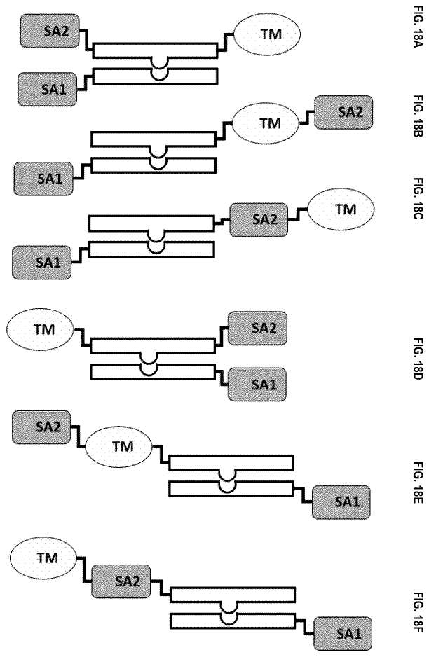

In various embodiments, the Fc-based chimeric protein complex takes the form of any of the schematics of FIGS. 18A-J.

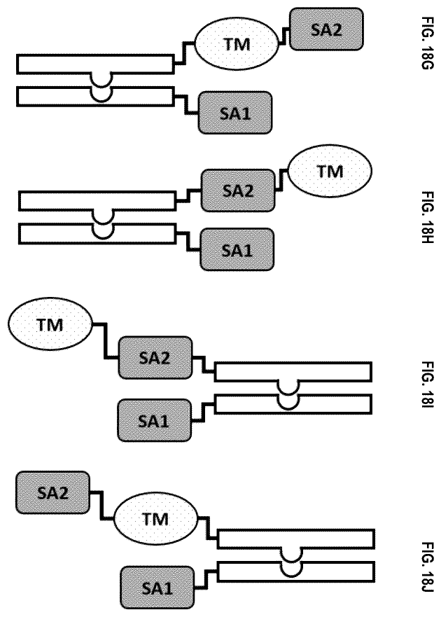

In various embodiments, the Fc-based chimeric protein complex takes the form of any of the schematics of FIGS. 19A-D.

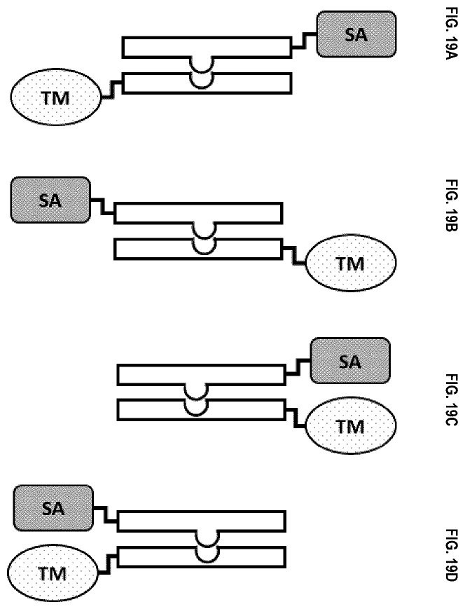

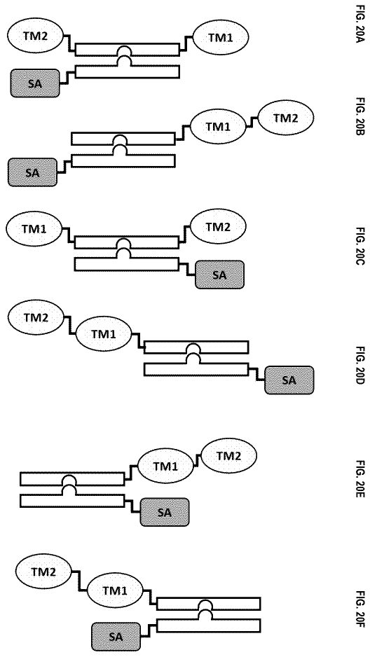

In various embodiments, the Fc-based chimeric protein complex takes the form of any of the schematics of FIGS. 20A-F.

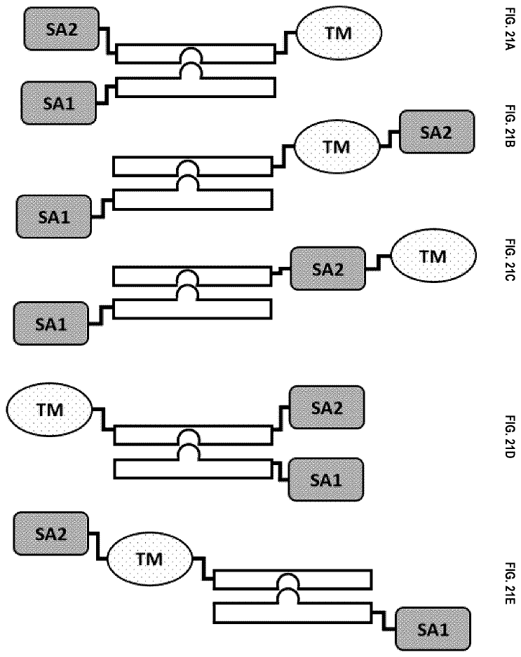

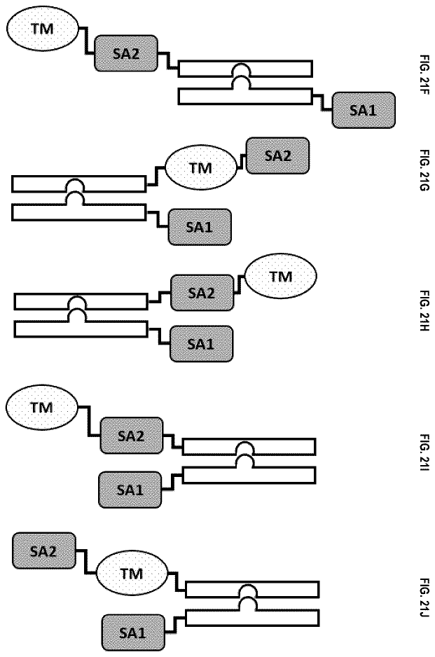

In various embodiments, the Fc-based chimeric protein complex takes the form of any of the schematics of FIGS. 21A-J.

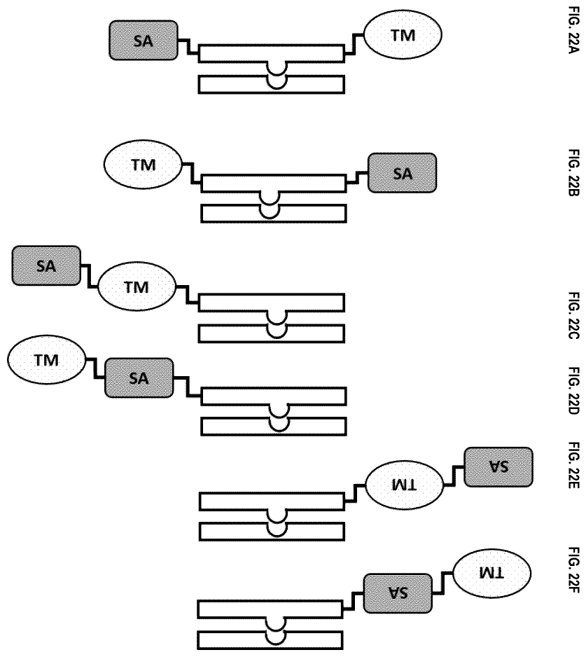

In various embodiments, the Fc-based chimeric protein complex takes the form of any of the schematics of FIGS. 22A-F.

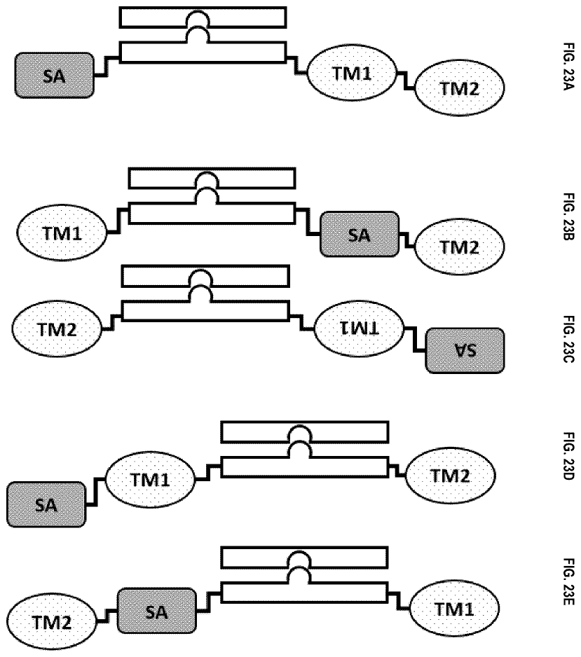

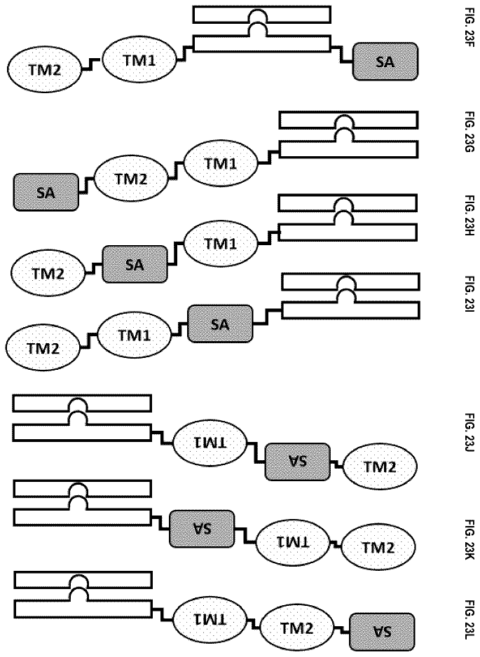

In various embodiments, the Fc-based chimeric protein complex takes the form of any of the schematics of FIGS. 23A-L.

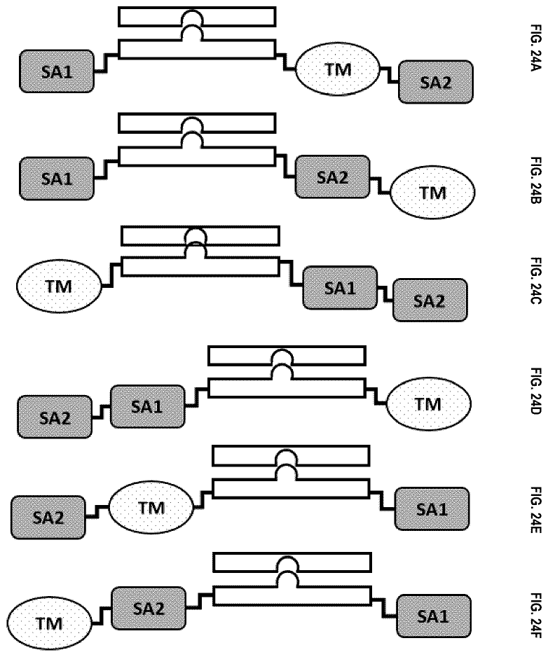

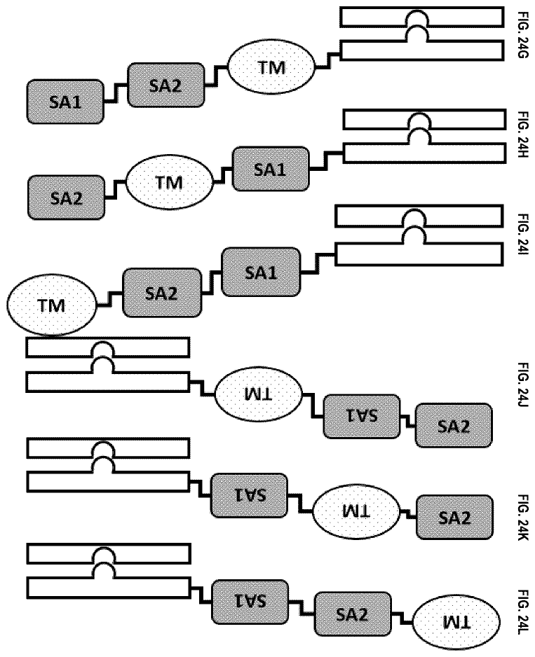

In various embodiments, the Fc-based chimeric protein complex takes the form of any of the schematics of FIGS. 24A-L.

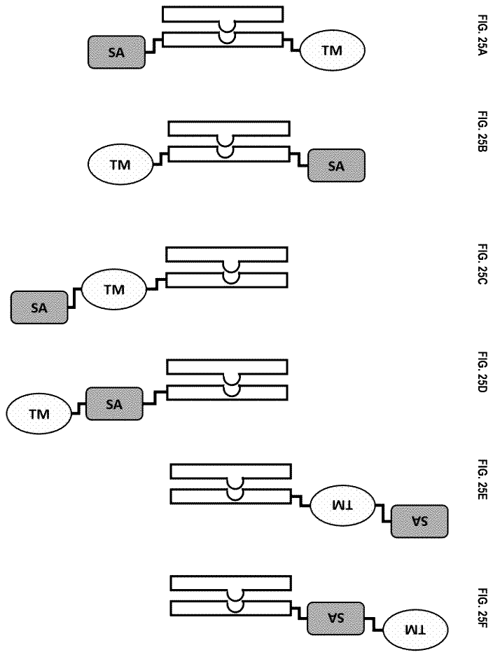

In various embodiments, the Fc-based chimeric protein complex takes the form of any of the schematics of FIGS. 25A-F.

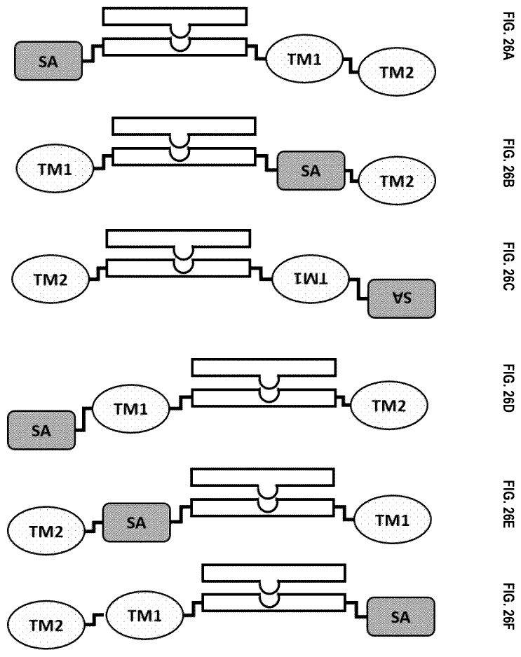

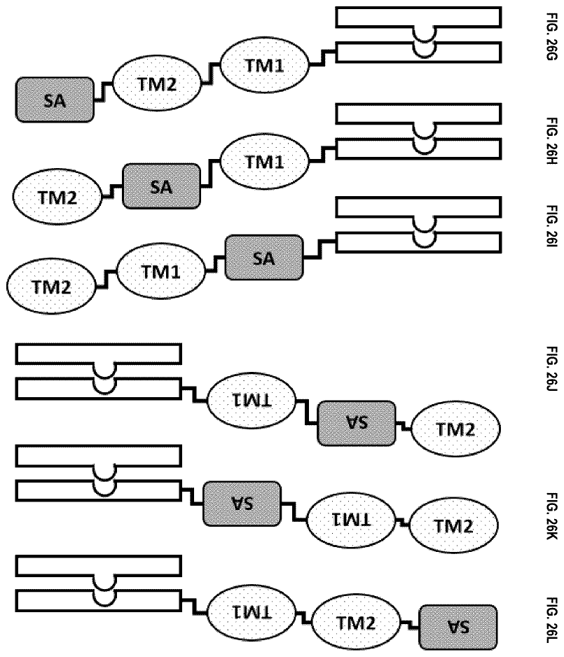

In various embodiments, the Fc-based chimeric protein complex takes the form of any of the schematics of FIGS. 26A-L.

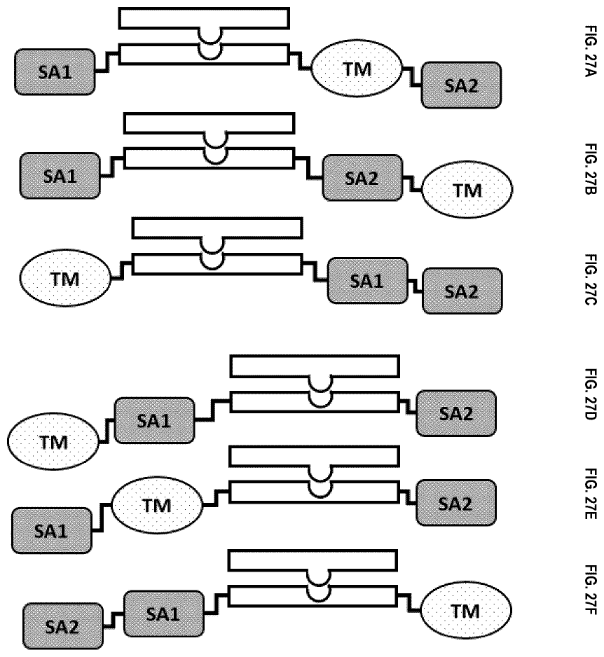

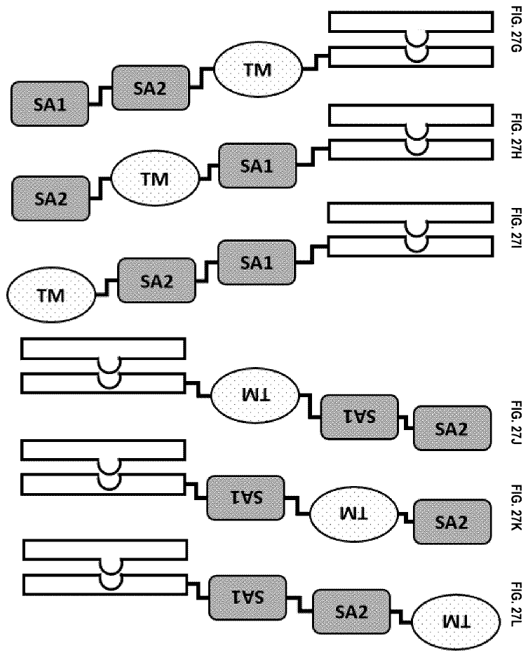

In various embodiments, the Fc-based chimeric protein complex takes the form of any of the schematics of FIGS. 27A-L.

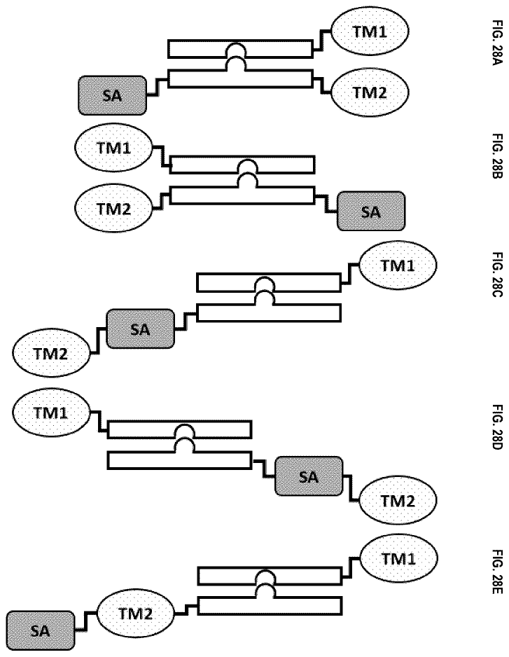

In various embodiments, the Fc-based chimeric protein complex takes the form of any of the schematics of FIGS. 28A-J.

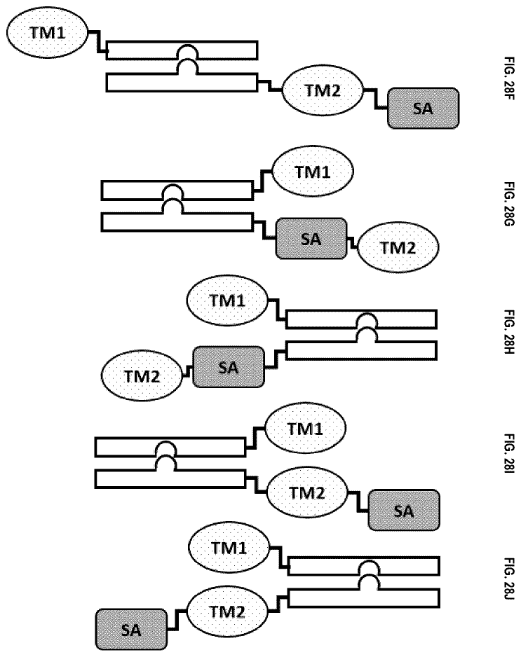

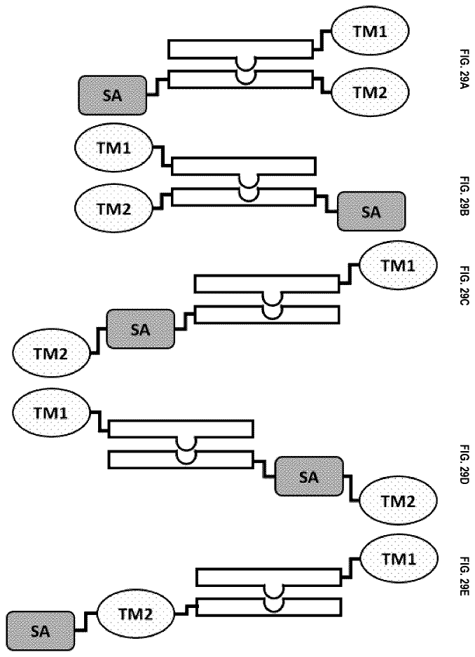

In various embodiments, the Fc-based chimeric protein complex takes the form of any of the schematics of FIGS. 29A-J.

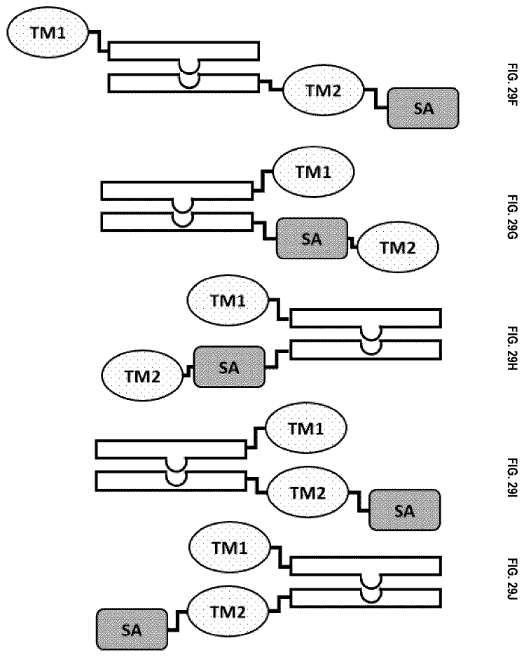

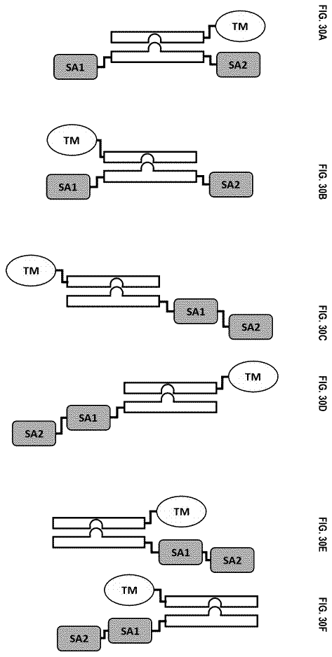

In various embodiments, the Fc-based chimeric protein complex takes the form of any of the schematics of FIGS. 30A-F.

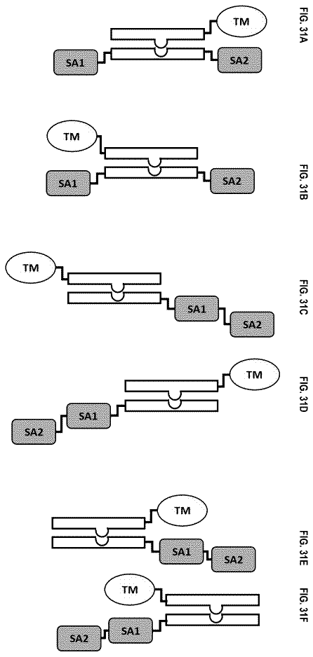

In various embodiments, the Fc-based chimeric protein complex takes the form of any of the schematics of FIGS. 31A-F.

In some embodiments, the signaling agents are linked to the targeting moieties and the targeting moieties are linked to the Fc domain on the same terminus (see FIGS. 13A-F). In some embodiments, the Fc domain is homodimeric.

In some embodiments, the signaling agents and targeting moieties are linked to the Fc domain, wherein the targeting moieties and signaling agents are linked on the same terminus (see FIGS. 13A-F). In some embodiments, the Fc domain is homodimeric.

In some embodiments, the targeting moieties are linked to signaling agents and the signaling agents are linked to the Fc domain on the same terminus (see FIGS. 13A-F). In some embodiments, the Fc domain is homodimeric.

In some embodiments, the homodimeric Fc-based chimeric protein complex has two or more targeting moieties. In some embodiments, there are four targeting moieties and two signaling agents, the targeting moieties are linked to the Fc domain and the signaling agents are linked to targeting moieties on the same terminus (see FIGS. 14A-H). In some embodiments, the Fc domain is homodimeric. In some embodiments, where there are four targeting moieties and two signaling agents, two targeting moieties are linked to the Fc domain and two targeting moieties are linked to the signaling agents, which are linked to the Fc domain on the same terminus (see FIGS. 14A-H). In some embodiments, the Fc domain is homodimeric. In some embodiments, where there are four targeting moieties and two signaling agents, two targeting moieties are linked to each other and one of the targeting moieties of from each pair is linked to the Fc domain on the same terminus and the signaling agents are linked to the Fc domain on the same terminus (see FIGS. 14A-H). In some embodiments, the Fc domain is homodimeric. In some embodiments, where there are four targeting moieties and two signaling agents, two targeting moieties are linked to each other, wherein one of the targeting moieties of from each pair is linked to a signaling agent and the other targeting moiety of the pair is linked the Fc domain, wherein the targeting moieties linked to the Fc domain are linked on the same terminus (see FIGS. 14A-H). In some embodiments, the Fc domain is homodimeric.

In some embodiments, the homodimeric Fc-based chimeric protein complex has two or more signaling agents. In some embodiments, where there are four signaling agents and two targeting moieties, two signaling agents are linked to each other and one of the signaling agents of from pair is linked to the Fc domain on the same terminus and the targeting moieties are linked to the Fc domain on the same terminus (see FIGS. 15A-H). In some embodiments, the Fc domain is homodimeric. In some embodiments, where there are four signaling agents and two targeting moieties, two signaling agents are linked to the Fc domain one the same terminus and two of the signaling agents are each linked to a targeting moiety, wherein the targeting moieties are linked to the Fc domain at the same terminus (see FIGS. 15A-H). In some embodiments, the Fc domain is homodimeric. In some embodiments, where there are four signaling agents and two targeting moieties, two signaling agents are linked to each other and one of the signaling agents of from pair is linked to a targeting moiety and the targeting moieties are linked to the Fc domain on the same terminus (see FIGS. 15A-H). In some embodiments, the Fc domain is homodimeric.

By way of example, in some embodiments, the Fc-based chimeric protein complex comprise a Fc domain, wherein the Fc domain comprises ionic pairing mutation(s) and/or knob-in-hole mutation(s), at least one signaling agent and at least one targeting moiety, wherein the ionic pairing motif and/or a knob-in-hole motif, signaling agent and targeting moiety are selected from any of the ionic pairing motif and/or a knob-in-hole motif, signaling agents, and targeting moieties disclosed herein. In some embodiments, the Fc domain is heterodimeric. In some embodiments, the Fc domain comprises a mutation that reduces or eliminates its effector function.

In some embodiments, the signaling agent is linked to the targeting moiety, which is linked to the Fc domain (see FIGS. 22A-F and 25A-F). In some embodiments, the targeting moiety is linked to the signaling agent, which is linked to the Fc domain (see FIGS. 22A-F and 25A-F). In some embodiments, the Fc domain is heterodimeric. In some embodiments, the Fc domain comprises a mutation that reduces or eliminates its effector function.

In some embodiments, the signaling agent and targeting moiety are linked to the Fc domain (see FIGS. 16A-D, 17A-D, 22A-F, and 25A-F). In some embodiments, the targeting moiety and the signaling agent are linked to different Fc chains on the same terminus (see FIGS. 16A-D and 19A-D). In some embodiments, the targeting moiety and the signaling agent are linked to different Fc chains on different termini (see FIGS. 16A-D and 19A-D). In some embodiments, the targeting moiety and the signaling agent are linked to the same Fc chain (see FIGS. 22A-F and 25A-F). In some embodiments, the Fc domain is heterodimeric. In some embodiments, the Fc domain comprises a mutation that reduces or eliminates its effector function.

In some embodiments, where there are one signaling agent and two targeting moieties, the signaling agent is linked to the Fc domain and two targeting moieties can be: 1) linked to each other with one of the targeting moieties linked to the Fc domain; or 2) each linked to the Fc domain (see FIGS. 17A-F, 20A-F, 23A-L, 26A-L, 28A-J, and 29A-J). In some embodiments, the targeting moieties are linked on one Fc chain and the signaling agent is on the other Fc chain (see FIGS. 17A-F and 20A-F). In some embodiments, the paired targeting moieties and the signaling agent are linked to the same Fc chain (see FIGS. 23A-L and 26A-L). In some embodiments, a targeting moiety is linked to the Fc domain and the other targeting moiety is linked to the signaling agent and the paired targeting moiety is linked to the Fc domain (see FIGS. 23A-L, 26A-L, 28A-J, and 29A-J). In some embodiments, the unpaired targeting moiety and paired targeting moiety are linked to the same Fc chain (see FIGS. 23A-L and 26A-L). In some embodiments, the unpaired targeting moiety and paired targeting moiety are linked to different Fc chains (see FIGS. 28A-J and 29A-J). In some embodiments, the unpaired targeting moiety and paired targeting moiety are linked on the same terminus (see FIGS. 28A-J and 29A-J). In some embodiments, the Fc domain is heterodimeric. In some embodiments, the Fc domain comprises a mutation that reduces or eliminates its effector function.

In some embodiments, where there are one signaling agent and two targeting moieties, a targeting moiety is linked to the signaling agent which is linked to the Fc domain, and the unpaired targeting moiety is linked the Fc domain (see FIGS. 23A-L, 26A-L, 28A-J, and 29A-J). In some embodiments, the paired signaling agent and unpaired targeting moiety are linked to the same Fc chain (see FIGS. 23A-L and 26A-L). In some embodiments, the paired signaling agent and unpaired targeting moiety are linked to different Fc chains (see FIGS. 28A-J and 29A-J). In some embodiments, the paired signaling agent and unpaired targeting moiety are linked on the same terminus (see FIGS. 28A-J and 29A-J). In some embodiments, the Fc domain is heterodimeric. In some embodiments, the Fc domain comprises a mutation that reduces or eliminates its effector function.

In some embodiments, where there are one signaling agent and two targeting moieties, the targeting moieties are linked together and the signaling agent is linked to one of the paired targeting moieties, wherein the targeting moiety not linked to the signaling agent is linked to the Fc domain (see FIGS. 23A-L and 26A-L). In some embodiments, the Fc domain is heterodimeric. In some embodiments, the Fc domain comprises a mutation that reduces or eliminates its effector function.

In some embodiments, where there are one signaling agent and two targeting moieties, the targeting moieties are linked together and the signaling agent is linked to one of the paired targeting moieties, wherein the signaling agent is linked to the Fc domain (see FIGS. 23A-L and 26A-L). In some embodiments, the Fc domain is heterodimeric. In some embodiments, the Fc domain comprises a mutation that reduces or eliminates its effector function.

In some embodiments, where there are one signaling agent and two targeting moieties, the targeting moieties are both linked to the signaling agent wherein one of the targeting moieties is linked to the Fc domain (see FIGS. 23A-L and 26A-L). In some embodiments, the Fc domain is heterodimeric. In some embodiments, the Fc domain comprises a mutation that reduces or eliminates its effector function.

In some embodiments, where there are one signaling agent and two targeting moieties, the targeting moieties and the signaling agent are linked to the Fc domain (see FIGS. 28A-J and 29A-J). In some embodiments, the targeting moieties are linked on the terminus (see FIGS. 28A-J and 29A-J). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked to the Fc domain on the same terminus and the targeting moiety is linked to the Fc domain (see FIGS. 18A-J and 21A-J). In some embodiments, the signaling agents are linked to the Fc domain on the same Fc chain and the targeting moiety is linked on the other Fc chain (see FIGS. 30A-F and 31A-F). In some embodiments, the Fc domain is heterodimeric. In some embodiments, the Fc domain comprises a mutation that reduces or eliminates its effector function.

In some embodiments, where there are two signaling agents and one targeting moiety, a signaling agent is linked to the targeting moiety, which is linked to the Fc domain and the other signaling agent is linked to the Fc domain (see FIGS. 18A-J, 19A-J, 24A-L, and 27A-L). In some embodiments, the targeting moiety and the unpaired signaling agent are linked to different Fc chains (see FIGS. 18A-J and 21A-J). In some embodiments, the targeting moiety and the unpaired signaling agent are linked to different Fc chains on the same terminus (see FIGS. 18A-J and 21A-J). In some embodiments, the targeting moiety and the unpaired signaling agent are linked to different Fc chains on different termini (see FIGS. 18A-J and 21A-J). In some embodiments, the targeting moiety and the unpaired signaling agent are linked to the same Fc chains (see FIGS. 24A-L and 27A-L). In some embodiments, the Fc domain is heterodimeric. In some embodiments, the Fc domain comprises a mutation that reduces or eliminates its effector function.

In some embodiments, where there are two signaling agents and one targeting moiety, the targeting moiety is linked to a signaling agent which is linked to the Fc domain and the other signaling agent is linked to the Fc domain (see FIGS. 18A-J and 21A-J). In some embodiments, the paired signaling agent and the unpaired signaling agent are linked to different Fc chains (see FIGS. 18A-J and 21A-J). In some embodiments, the paired signaling agent and the unpaired signaling agent are linked to different Fc chains on the same terminus (see FIGS. 18A-J and 21A-J). In some embodiments, the paired signaling agent and the unpaired signaling agent are linked to different Fc chains on different termini (see FIGS. 18A-J and 21A-J). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and the targeting moiety is linked to one of the paired signaling agents, wherein the targeting moiety is linked to the Fc domain (see FIGS. 24A-L and 27A-L). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and one of the signaling agents is linked to the Fc domain and the targeting moiety is linked to the Fc domain (see FIGS. 24A-L, 27A-L, 30A-F, and 31A-F). In some embodiments, the paired signaling agents and targeting moiety are linked to the same Fc chain (see FIGS. 24A-L and 27A-L). In some embodiments, the paired signaling agents and targeting moiety are linked to different Fc chains (see FIGS. 30A-F and 31A-F). In some embodiments, the paired signaling agents and targeting moiety are linked to different Fc chains on the same terminus (see FIGS. 30A-F and 31A-F). In some embodiments, the Fc domain is heterodimeric. In some embodiments, the Fc domain comprises a mutation that reduces or eliminates its effector function.

In some embodiments, where there are two signaling agents and one targeting moiety, the signaling agents are both linked to the targeting moiety, wherein one of the signaling agents is linked to the Fc domain (see FIGS. 24A-L and 27A-L). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and one of the signaling agents is linked to the targeting moiety and the other signaling agent is linked to the Fc domain (see FIGS. 24A-L and 27A-L).

In some embodiments, where there are two signaling agents and one targeting moiety, each signaling agent is linked to the Fc domain and the targeting moiety is linked to one of the signaling agents (see FIGS. 24A-L and 27A-L). In some embodiments, the signaling agents are linked to the same Fc chain (see FIGS. 24A-L and 27A-L).

In some embodiments, a targeting moiety or signaling agent is linked to the Fc domain, comprising one or both of C_{H}2 and C_{H}3 domains, and optionally a hinge region. For example, vectors encoding the targeting moiety, signaling agent, or combination thereof, linked as a single nucleotide sequence to an Fc domain can be used to prepare such polypeptides.

### Modifications and Production of Chimeric Proteins

In various embodiments, the CD13-targeted chimeric proteins or chimeric protein complexes and CD8-targeted chimeric proteins or chimeric protein complexes disclosed herein comprise a targeting moiety (e.g., CD13 and CD8) that is a VHH. In various embodiments, the VHH is not limited to a specific biological source or to a specific method of preparation. For example, the VHH can generally be obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring V_{H} domain from any animal species, such as from a mammalian species, such as from a human being, or by expression of a nucleic acid encoding such a camelized V_{H} domain; (5) by “camelization” of a “domain antibody” or “Dab” as described in the art, or by expression of a nucleic acid encoding such a camelized V_{H} domain; (6) by using synthetic or semi-synthetic techniques for preparing proteins, polypeptides or other amino acid sequences known in the art; (7) by preparing a nucleic acid encoding a VHH using techniques for nucleic acid synthesis known in the art, followed by expression of the nucleic acid thus obtained; and/or (8) by any combination of one or more of the foregoing.

In an embodiment, the CD13-targeted chimeric protein or chimeric protein complex and CD8-targeted chimeric protein or chimeric protein complex comprise a VHH that corresponds to the VHH domains of naturally occurring heavy chain antibodies directed against a target of interest. In some embodiments, such VHH sequences can generally be generated or obtained by suitably immunizing a species of Camelid with a molecule of based on the target of interest (e.g., CD13 and CD8) (i.e., so as to raise an immune response and/or heavy chain antibodies directed against the target of interest), by obtaining a suitable biological sample from the Camelid (such as a blood sample, or any sample of B-cells), and by generating VHH sequences directed against the target of interest, starting from the sample, using any suitable known techniques. In some embodiments, naturally occurring VHH domains against the target of interest can be obtained from naive libraries of Camelid VHH sequences, for example, by screening such a library using the target of interest or at least one part, fragment, antigenic determinant or epitope thereof using one or more screening techniques known in the art. Such libraries and techniques are, for example, described in WO 9937681, WO 0190190, WO 03025020 and WO 03035694, the entire contents of which are hereby incorporated by reference. In some embodiments, improved synthetic or semi-synthetic libraries derived from naive V_{H}H libraries may be used, such as V_{H}H libraries obtained from naive V_{H}H libraries by techniques such as random mutagenesis and/or CDR shuffling, as for example, described in WO 0043507, the entire contents of which are hereby incorporated by reference. In some embodiments, another technique for obtaining V_{H}H sequences directed against a target of interest involves suitably immunizing a transgenic mammal that is capable of expressing heavy chain antibodies (i.e., so as to raise an immune response and/or heavy chain antibodies directed against the target of interest), obtaining a suitable biological sample from the transgenic mammal (such as a blood sample, or any sample of B-cells), and then generating V_{H}H sequences directed against XCR1 starting from the sample, using any suitable known techniques. For example, for this purpose, the heavy chain antibody-expressing mice and the further methods and techniques described in WO 02085945 and in WO 04049794 (the entire contents of which are hereby incorporated by reference) can be used.

In an embodiment, the CD13-targeted chimeric protein or chimeric protein complex and CD8-targeted chimeric protein or chimeric protein complex comprise a VHH that has been “humanized” i.e., by replacing one or more amino acid residues in the amino acid sequence of the naturally occurring VHH sequence (and in particular in the framework sequences) by one or more of the amino acid residues that occur at the corresponding position(s) in a VH domain from a conventional 4-chain antibody from a human being. This can be performed using humanization techniques known in the art. In some embodiments, possible humanizing substitutions or combinations of humanizing substitutions may be determined by methods known in the art, for example, by a comparison between the sequence of a VHH and the sequence of a naturally occurring human V_{H} domain. In some embodiments, the humanizing substitutions are chosen such that the resulting humanized VHHs still retain advantageous functional properties. Generally, as a result of humanization, the VHHs of the invention may become more “human-like,” while still retaining favorable properties such as a reduced immunogenicity, compared to the corresponding naturally occurring V_{H}H domains. In various embodiments, the humanized VHHs of the invention can be obtained in any suitable manner known in the art and thus are not strictly limited to polypeptides that have been obtained using a polypeptide that comprises a naturally occurring VHH domain as a starting material.

In an embodiment, the CD13-targeted chimeric protein or chimeric protein complex and CD8-targeted chimeric protein or chimeric protein complex comprise a VHH that has been “camelized,” i.e., by replacing one or more amino acid residues in the amino acid sequence of a naturally occurring VH domain from a conventional 4-chain antibody by one or more of the amino acid residues that occur at the corresponding position(s) in a V_{H}H domain of a heavy chain antibody of a camelid. In some embodiments, such “camelizing” substitutions are inserted at amino acid positions that form and/or are present at the VH-VL interface, and/or at the so-called Camelidae hallmark residues (see, for example, WO 9404678, the entire contents of which are hereby incorporated by reference). In some embodiments, the VH sequence that is used as a starting material or starting point for generating or designing the camelized VHH is a VH sequence from a mammal, for example, the VH sequence of a human being, such as a VH3 sequence. In various embodiments, the camelized VHHs can be obtained in any suitable manner known in the art (i.e., as indicated under points (1)-(8) above) and thus are not strictly limited to polypeptides that have been obtained using a polypeptide that comprises a naturally occurring VH domain as a starting material.

In various embodiments, both “humanization” and “camelization” can be performed by providing a nucleotide sequence that encodes a naturally occurring V_{H}H domain or VH domain, respectively, and then changing, in a manner known in the art, one or more codons in the nucleotide sequence in such a way that the new nucleotide sequence encodes a “humanized” or “camelized” VHH, respectively. This nucleic acid can then be expressed in a manner known in the art, so as to provide the desired VHH of the invention. Alternatively, based on the amino acid sequence of a naturally occurring V_{H}H domain or VH domain, respectively, the amino acid sequence of the desired humanized or camelized VHH of the invention, respectively, can be designed and then synthesized de novo using techniques for peptide synthesis known in the art. Also, based on the amino acid sequence or nucleotide sequence of a naturally occurring V_{H}H domain or VH domain, respectively, a nucleotide sequence encoding the desired humanized or camelized VHH, respectively, can be designed and then synthesized de novo using techniques for nucleic acid synthesis known in the art, after which the nucleic acid thus obtained can be expressed in a manner known in the art, so as to provide the desired VHH of the invention. Other suitable methods and techniques for obtaining the VHHs of the invention and/or nucleic acids encoding the same, starting from naturally occurring VH sequences or V_{H}H sequences, are known in the art, and may, for example, comprise combining one or more parts of one or more naturally occurring VH sequences (such as one or more FR sequences and/or CDR sequences), one or more parts of one or more naturally occurring V_{H}H sequences (such as one or more FR sequences or CDR sequences), and/or one or more synthetic or semi-synthetic sequences, in a suitable manner, so as to provide a VHH of the invention or a nucleotide sequence or nucleic acid encoding the same.

Methods for producing the CD13-targeted chimeric protein or chimeric protein complex and CD8-targeted chimeric protein or chimeric protein complex are described herein. For example, DNA sequences encoding the CD13-targeted chimeric protein or chimeric protein complex and CD8-targeted chimeric protein or chimeric protein complex (e.g., DNA sequences encoding the signaling agent (e.g., TNF or IFN) and the targeting moiety (e.g., CD13 or CD8) and, optionally a linker) can be chemically synthesized using methods known in the art. Synthetic DNA sequences can be ligated to other appropriate nucleotide sequences, including, e.g., expression control sequences, to produce gene expression constructs encoding the desired chimeric proteins or chimeric protein complexes. Accordingly, in various embodiments, the present invention provides for isolated nucleic acids comprising a nucleotide sequence encoding the chimeric protein or chimeric protein complex of the invention.

Nucleic acids encoding the chimeric protein or chimeric protein complex of the invention can be incorporated (ligated) into expression vectors, which can be introduced into host cells through transfection, transformation, or transduction techniques. For example, nucleic acids encoding the chimeric protein or chimeric protein complex of the invention can be introduced into host cells by retroviral transduction. Illustrative host cells are E. coli cells, Chinese hamster ovary (CHO) cells, human embryonic kidney 293 (HEK 293) cells, Hela cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the chimeric protein or chimeric protein complex of the invention. Accordingly, in various embodiments, the present invention provides expression vectors comprising nucleic acids that encode the chimeric protein or chimeric protein complex of the invention. In various embodiments, the present invention additional provides host cells comprising such expression vectors.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. In another example, if the engineered gene is to be expressed in eukaryotic host cells, e.g., CHO cells, it is first inserted into an expression vector containing for example, a suitable eukaryotic promoter, a secretion signal, enhancers, and various introns. The gene construct can be introduced into the host cells using transfection, transformation, or transduction techniques.

The CD13-targeted chimeric protein or chimeric protein complex and CD8-targeted chimeric protein or chimeric protein complex can be produced by growing a host cell transfected with an expression vector encoding the chimeric protein or chimeric protein complex under conditions that permit expression of the protein. Following expression, the protein can be harvested and purified using techniques well known in the art, e.g., affinity tags such as glutathione-S-transferase (GST) and histidine tags or by chromatography. In an embodiment, the chimeric protein or chimeric protein complex comprises a His tag. In an embodiment, the chimeric protein or chimeric protein complex comprises a His tag and a proteolytic site to allow cleavage of the His tag.

Accordingly, in various embodiments, the present invention provides for a nucleic acid encoding a CD13-targeted chimeric protein or chimeric protein complex and CD8-targeted chimeric protein or chimeric protein complex. In various embodiments, the present invention provides for a host cell comprising a nucleic acid encoding a CD13-targeted chimeric protein or chimeric protein complex and CD8-targeted chimeric protein or chimeric protein complex.

In various embodiments, the CD13-targeted chimeric protein or chimeric protein complex or CD8-targeted chimeric protein or chimeric protein complex may be expressed in vivo, for instance, in a patient. For example, in various embodiments, the CD13-targeted chimeric protein or chimeric protein complex or CD8-targeted chimeric protein or chimeric protein complex is administered in the form of nucleic acid, which encodes the CD13-targeted chimeric protein or chimeric protein complex or CD8-targeted chimeric protein or chimeric protein complex. In various embodiments, the nucleic acid is DNA or RNA. In some embodiments, the CD13-targeted chimeric protein or chimeric protein complex or CD8-targeted chimeric protein or chimeric protein complex is encoded by a modified mRNA, i.e. an mRNA comprising one or more modified nucleotides. In some embodiments, the modified mRNA comprises one or modifications found in U.S. Pat. No. 8,278,036, the entire contents of which are hereby incorporated by reference. In some embodiments, the modified mRNA comprises one or more of m5C, m5U, m6A, s2U, ψ, and 2′-O-methyl-U. In some embodiments, the present invention relates to administering a modified mRNA encoding one or more of the present chimeric proteins or chimeric protein complexes. In some embodiments, the present invention relates to gene therapy vectors comprising the same. In some embodiments, the present invention relates to gene therapy methods comprising the same. In various embodiments, the nucleic acid is in the form of an oncolytic virus, e.g. an adenovirus, reovirus, measles, herpes simplex, Newcastle disease virus or vaccinia.

In some embodiments, the CD13-targeted chimeric protein or chimeric protein complex or CD8-targeted chimeric protein or chimeric protein complex described herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of tunicamycin, etc.

### Functional Groups

In various embodiments, the CD13-targeted chimeric protein or chimeric protein complex or CD8-targeted chimeric protein or chimeric protein complex may include one or more functional groups, residues, or moieties. In various embodiments, the one or more functional groups, residues, or moieties are attached or genetically fused to any of the signaling agents or targeting moieties described herein. In some embodiments, such functional groups, residues or moieties confer one or more desired properties or functionalities to the chimeric protein or chimeric protein complex of the invention. Examples of such functional groups and of techniques for introducing them into the chimeric protein or chimeric protein complex are known in the art, for example, see Remington's Pharmaceutical Sciences, 16th ed., Mack Publishing Co., Easton, Pa. (1980).

In various embodiments, the CD13-targeted chimeric protein or chimeric protein complex or CD8-targeted chimeric protein or chimeric protein complex may by conjugated and/or fused with another agent to extend half-life or otherwise improve pharmacodynamic and pharmacokinetic properties. In some embodiments, the chimeric proteins or chimeric protein complexes may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In some embodiments, the chimeric protein or chimeric protein complex may be fused or conjugated with an antibody or an antibody fragment such as an Fc fragment. For example, the CD13-targeted chimeric protein or chimeric protein complex or CD8-targeted chimeric protein or chimeric protein complex may be fused to either the N-terminus or the C-terminus of the Fc domain of human immunoglobulin (lg) G. In various embodiments, each of the CD13-targeted chimeric protein or chimeric protein complex or CD8-targeted chimeric protein or chimeric protein complex is fused to one or more of the agents described in BioDrugs (2015) 29:215-239, the entire contents of which are hereby incorporated by reference.

In some embodiments, the functional groups, residues, or moieties comprise a suitable pharmacologically acceptable polymer, such as poly(ethyleneglycol) (PEG) or derivatives thereof (such as methoxypoly(ethyleneglycol) or mPEG). In some embodiments, attachment of the PEG moiety increases the half-life and/or reduces the immunogenecity of the CD13-targeted chimeric protein or chimeric protein complex or CD8-targeted chimeric protein or chimeric protein complex. Generally, any suitable form of pegylation can be used, such as the pegylation used in the art for antibodies and antibody fragments (including but not limited to single domain antibodies such as VHHs); see, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04060965, the entire contents of which are hereby incorporated by reference. Various reagents for pegylation of proteins are also commercially available, for example, from Nektar Therapeutics, USA. In some embodiments, site-directed pegylation is used, in particular via a cysteine-residue (see, for example, Yang et al., Protein Engineering, 16, 10, 761-770 (2003), the entire contents of which is hereby incorporated by reference). In some embodiments, the CD13-targeted chimeric protein or chimeric protein complex or CD8-targeted chimeric protein or chimeric protein complex is modified so as to suitably introduce one or more cysteine residues for attachment of PEG, or an amino acid sequence comprising one or more cysteine residues for attachment of PEG may be fused to the amino- and/or carboxy-terminus of the chimeric proteins or chimeric protein complexes, using techniques known in the art.

In some embodiments, the functional groups, residues, or moieties comprise N-linked or O-linked glycosylation. In some embodiments, the N-linked or O-linked glycosylation is introduced as part of a co-translational and/or post-translational modification.

In some embodiments, the functional groups, residues, or moieties comprise one or more detectable labels or other signal-generating groups or moieties. Suitable labels and techniques for attaching, using and detecting them are known in the art and, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels include moieties that can be detected using NMR or ESR spectroscopy. Such labeled VHHs and polypeptides of the invention may, for example, be used for in vitro, in vivo or in situ assays (including immunoassays known per se such as ELISA, RIA, EIA and other “sandwich assays,” etc.) as well as in vivo diagnostic and imaging purposes, depending on the choice of the specific label.

In some embodiments, the functional groups, residues, or moieties comprise a tag that is attached or genetically fused to the CD13-targeted chimeric protein or chimeric protein complex or CD8-targeted chimeric protein or chimeric protein complex. In some embodiments, the CD13-targeted chimeric protein or chimeric protein complex or CD8-targeted chimeric protein or chimeric protein complex may include a single tag or multiple tags. The tag for example is a peptide, sugar, or DNA molecule that does not inhibit or prevent binding of the CD13-targeted chimeric protein or chimeric protein complex or CD8-targeted chimeric protein or chimeric protein complex to its target or any other antigen of interest such as tumor antigens. In various embodiments, the tag is at least about: three to five amino acids long, five to eight amino acids long, eight to twelve amino acids long, twelve to fifteen amino acids long, or fifteen to twenty amino acids long. Illustrative tags are described for example, in U.S. Patent Publication No. US2013/0058962. In some embodiment, the tag is an affinity tag such as glutathione-S-transferase (GST) and histidine (His) tag. In an embodiment, the CD13-targeted chimeric protein or chimeric protein complex or CD8-targeted chimeric protein or chimeric protein complex comprises a His tag.

In some embodiments, the functional groups, residues, or moieties comprise a chelating group, for example, to chelate one of the metals or metallic cations. Suitable chelating groups, for example, include, without limitation, diethyl-enetriaminepentaacetic acid (DTPA) or ethylenediaminetetraacetic acid (EDTA).

In some embodiments, the functional groups, residues, or moieties comprise a functional group that is one part of a specific binding pair, such as the biotin-(strept) avidin binding pair. Such a functional group may be used to link the chimeric protein or chimeric protein complex of the invention to another protein, polypeptide or chemical compound that is bound to the other half of the binding pair, i.e., through formation of the binding pair. For example, the CD13-targeted chimeric protein or chimeric protein complex or CD8-targeted chimeric protein or chimeric protein complex may be conjugated to biotin, and linked to another protein, polypeptide, compound or carrier conjugated to avidin or streptavidin. For example, such a conjugated CD13-targeted chimeric protein or chimeric protein complex or CD8-targeted chimeric protein or chimeric protein complex may be used as a reporter, for example, in a diagnostic system where a detectable signal-producing agent is conjugated to avidin or streptavidin. Such binding pairs may, for example, also be used to bind the CD13-targeted chimeric protein or chimeric protein complex or CD8-targeted chimeric protein or chimeric protein complex to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the CD13-targeted chimeric protein or chimeric protein complex or CD8-targeted chimeric protein or chimeric protein complex.

### Pharmaceutically Acceptable Salts and Excipients

The CD13-targeted chimeric protein or chimeric protein complex and CD8-targeted chimeric protein or chimeric protein complex described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, the compositions described herein are in the form of a pharmaceutically acceptable salt.

### Pharmaceutical Compositions and Formulations

In various embodiments, the present invention pertains to pharmaceutical compositions comprising the CD13-targeted chimeric protein or chimeric protein complex and CD8-targeted chimeric protein or chimeric protein complex described herein and a pharmaceutically acceptable carrier or excipient. In some embodiments, the present invention pertains to pharmaceutical compositions comprising the present chimeric protein or chimeric protein complex. In a further embodiment, the present invention pertains to pharmaceutical compositions comprising a combination of the present chimeric protein or chimeric protein complex and any other therapeutic agents described herein. Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present invention includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In a further embodiment, the pharmaceutical composition is formulated in the form of a gelatin capsule. In yet another embodiment, the pharmaceutical composition is formulated as a liquid.

Where necessary, the inventive pharmaceutical compositions (and/or additional agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In some embodiments, the administering is effected orally. In another embodiment, the administration is by parenteral injection. The mode of administration can be left to the discretion of the practitioner, and depends in-part upon the site of the medical condition. In most instances, administration results in the release of any agent described herein into the bloodstream.

In one embodiment, the chimeric protein or chimeric protein complex described herein is formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any chimeric proteins or chimeric protein complexes described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

The compositions provided herein, alone or in combination with other suitable components, can be made into aerosol formulations (i.e., “nebulized”) to be administered via inhalation. Aerosol formulations can be placed into pressurized acceptable propellants, such as dichlorodifluoromethane, propane, nitrogen, and the like.

Any inventive pharmaceutical compositions (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds. In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Administration and Dosage

It will be appreciated that the actual dose of the CD13-targeted chimeric protein or chimeric protein complex and additional therapeutic agent(s) (e.g., CD8-targeted chimeric protein or chimeric protein complex, PI3K inhibitor, anthracycline, or SMAC mimetic) to be administered according to the present invention will vary according to the particular dosage form, and the mode of administration. Many factors that may modify the action of the CD13-targeted chimeric protein or chimeric protein complex and additional therapeutic agent(s) (e.g., body weight, gender, diet, time of administration, route of administration, rate of excretion, condition of the subject, drug combinations, genetic disposition and reaction sensitivities) can be taken into account by those skilled in the art.

Administration can be carried out continuously or in one or more discrete doses within the maximum tolerated dose. Optimal administration rates for a given set of conditions can be ascertained by those skilled in the art using conventional dosage administration tests.

In some embodiments, a suitable dosage of the CD13-targeted chimeric protein or chimeric protein complex and additional therapeutic agents is in a range of about 0.01 μg/kg to about 100 mg/kg of body weight of the subject, about 0.01 μg/kg to about 10 mg/kg of body weight of the subject, or about 0.01 μg/kg to about 1 mg/kg of body weight of the subject for example, about 0.01 μg/kg, about 0.02 μg/kg, about 0.03 μg/kg, about 0.04 μg/kg, about 0.05 μg/kg, about 0.06 μg/kg, about 0.07 μg/kg, about 0.08 μg/kg, about 0.09 μg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, or about 100 mg/kg body weight, inclusive of all values and ranges therebetween.

Individual doses of the CD13-targeted chimeric protein or chimeric protein complex and additional therapeutic agents can be administered in unit dosage forms (e.g., tablets, capsules, or liquid formulations) containing, for example, from about 1 μg to about 100 mg, from about 1 μg to about 90 mg, from about 1 μg to about 80 mg, from about 1 μg to about 70 mg, from about 1 μg to about 60 mg, from about 1 μg to about 50 mg, from about 1 μg to about 40 mg, from about 1 μg to about 30 mg, from about 1 μg to about 20 mg, from about 1 μg to about 10 mg, from about 1 μg to about 5 mg, from about 1 μg to about 3 mg, from about 1 μg to about 1 mg per unit dosage form, or from about 1 μg to about 50 μg per unit dosage form. For example, a unit dosage form can be about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29, about 30 μg, about 35 μg, about 40 μg, about 45 μg, about 50 μg, about 60 μg, about 70 μg, about 80 μg, about 90 μg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween. In an embodiment, the chimeric protein or chimeric protein complex is administered as a unit dosage form containing about 9 μg of the chimeric protein or chimeric protein complex. In another embodiment, the chimeric protein or chimeric protein complex is administered as a unit dosage form containing about 15 μg of the chimeric protein or chimeric protein complex.

In one embodiment, the CD13-targeted chimeric protein or chimeric protein complex and additional therapeutic agents are each administered at an amount of from about 1 μg to about 100 mg daily, from about 1 μg to about 90 mg daily, from about 1 μg to about 80 mg daily, from about 1 μg to about 70 mg daily, from about 1 μg to about 60 mg daily, from about 1 μg to about 50 mg daily, from about 1 μg to about 40 mg daily, from about 1 μg to about 30 mg daily, from about 1 μg to about 20 mg daily, from about 01 μg to about 10 mg daily, from about 1 μg to about 5 mg daily, from about 1 μg to about 3 mg daily, or from about 1 μg to about 1 mg daily. In various embodiments, the chimeric protein or chimeric protein complex is administered at a daily dose of about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29, about 30 μg, about 35 μg, about 40 μg, about 45 μg, about 50 μg, about 60 μg, about 70 μg, about 80 μg, about 90 μg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween.

In accordance with certain embodiments of the invention, a pharmaceutical composition comprising the CD13-targeted chimeric protein or chimeric protein complex is co-administered with at least one additional therapeutic agent, for example, more than once daily (e.g., about two times, about three times, about four times, about five times, about six times, about seven times, about eight times, about nine times, or about ten times daily), about once per day, about every other day, about every third day, about once a week, about once every two weeks, about once every month, about once every two months, about once every three months, about once every six months, or about once every year. In an embodiment, the pharmaceutical composition comprising the chimeric protein or chimeric protein complex is administered about three times a week.

In various embodiments, the CD13-targeted chimeric protein or chimeric protein complex and at least one additional therapeutic agent may be co-administered for a prolonged period. For example, the CD13-targeted chimeric protein or chimeric protein complex and at least one additional therapeutic agent may be co-administered as described herein for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks. For example, the CD13-targeted chimeric protein or chimeric protein complex and at least one additional therapeutic agent may be co-administered for 12 weeks, 24 weeks, 36 weeks or 48 weeks. In some embodiments, the CD13-targeted chimeric protein or chimeric protein complex and at least one additional therapeutic agent may be co-administered for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In some embodiments, the CD13-targeted chimeric protein or chimeric protein complex and at least one additional therapeutic agent may be co-administered for at least about 1 year, at least about 2 years, at least about 3 years, at least about 4 years, or at least about 5 years.

In various embodiments, co-administration of the CD13-targeted chimeric protein or chimeric protein complex and at least one additional therapeutic agent can be simultaneous or sequential. For example, in one embodiment, the CD13-targeted chimeric protein or chimeric protein complex is administered alongwith the therapeutic agent(s). In another embodiment, the CD13-targeted chimeric protein or chimeric protein complex is administered before the therapeutic agent. In yet another embodiment, the CD13-targeted chimeric protein or chimeric protein complex is administered after the therapeutic agent.

In one embodiment, the additional therapeutic agent (e.g., CD13-targeted chimeric protein or chimeric protein complex, CD8-targeted chimeric protein or chimeric protein complex, PI3K inhibitor, anthracycline, or SMAC mimetic) and the CD13-targeted chimeric protein or chimeric protein complex of the present invention are administered to a subject simultaneously. The term “simultaneously” as used herein, means that the additional therapeutic agent and the CD13-targeted chimeric protein or chimeric protein complex are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the CD13-targeted chimeric protein or chimeric protein complex can be by simultaneous administration of a single formulation (e.g., a formulation comprising the additional therapeutic agent and the chimeric protein or chimeric protein complex) or of separate formulations (e.g., a first formulation including the additional therapeutic agent and a second formulation including the chimeric protein or chimeric protein complex).

Co-administration does not require the therapeutic agents to be administered simultaneously, if the timing of their administration is such that the pharmacological activities of the additional therapeutic agent and the chimeric protein or chimeric protein complex overlap in time, thereby exerting a combined therapeutic effect. For example, the additional therapeutic agent and the CD13-targeted chimeric protein or chimeric protein complex can be administered sequentially. The term “sequentially” as used herein means that the additional therapeutic agent and the CD13-targeted chimeric protein or chimeric protein complex are administered with a time separation of more than about 60 minutes. For example, the time between the sequential administration of the additional therapeutic agent and the CD13-targeted chimeric protein or chimeric protein complex can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the CD13-targeted chimeric protein or chimeric protein complex being administered. Either the additional therapeutic agent or the CD13-targeted chimeric protein or chimeric protein complex cell may be administered first.

Co-administration also does not require the therapeutic agents to be administered to the subject by the same route of administration. Rather, each therapeutic agent can be administered by any appropriate route, for example, parenterally or non-parenterally.

In some embodiments, the CD13-targeted chimeric protein or chimeric protein complex described herein acts synergistically when co-administered with another therapeutic agent (e.g., CD13-targeted chimeric protein or chimeric protein complex, CD8-targeted chimeric protein or chimeric protein complex, PI3K inhibitor, anthracycline, or SMAC mimetic). In such embodiments, the CD13-targeted chimeric protein or chimeric protein complex and the additional therapeutic agent may be administered at doses that are lower than the doses employed when the agents are used in the context of monotherapy.

### Methods of Treatment

Methods and compositions described herein have application to treating cancer. In some embodiments, the present invention relates to the treatment of, or a patient having cancer. As used herein, cancer refers to any uncontrolled growth of cells that may interfere with the normal functioning of the bodily organs and systems and includes both primary and metastatic tumors. Primary tumors or cancers that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject. For example, cancers can include benign and malignant cancers, polyps, hyperplasia, as well as dormant tumors or micrometastases.

Illustrative cancers that may be treated include, but are not limited to, carcinomas, e.g. various subtypes, including, for example, adenocarcinoma, basal cell carcinoma, squamous cell carcinoma, and transitional cell carcinoma), sarcomas (including, for example, bone and soft tissue), leukemias (including, for example, acute myeloid, acute lymphoblastic, chronic myeloid, chronic lymphocytic, and hairy cell), lymphomas and myelomas (including, for example, Hodgkin and non-Hodgkin lymphomas, light chain, non-secretory, MGUS, and plasmacytomas), and central nervous system cancers (including, for example, brain (e.g. gliomas (e.g. astrocytoma, oligodendroglioma, and ependymoma), meningioma, pituitary adenoma, and neuromas, and spinal cord tumors (e.g. meningiomas and neurofibroma).

Illustrative cancers that may be treated include, but are not limited to, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome. In an embodiment, the present invention relates to the treatment of leukemia including hairy cell leukemia. In another embodiment, the present invention relates to the treatment of melanoma including malignant melanoma. In a further embodiment, the present invention relates to the treatment of Kaposi's sarcoma including AIDS-related Kaposi's sarcoma.

### Kits

The invention also provides kits for the administration of any agent described herein (e.g. the CD13-targeted chimeric protein or chimeric protein complex with or without various additional therapeutic agents). The kit is an assemblage of materials or components, including at least one of the inventive pharmaceutical compositions described herein. Thus, in some embodiments, the kit contains at least one of the pharmaceutical compositions described herein.

The exact nature of the components configured in the kit depends on its intended purpose. In one embodiment, the kit is configured for treating human subjects.

Instructions for use may be included in the kit. Instructions for use typically include a tangible expression describing the technique to be employed in using the components of the kit to effect a desired outcome, such as to treat cancer. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials and components assembled in the kit can be provided to the practitioner stored in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label, which indicates the contents and/or purpose of the kit and/or its components.

### Definitions

As used herein, “a,” “an,” or “the” can mean one or more than one.

Unless specifically stated or obvious from context, as used herein, the term “or” is understood to be inclusive and covers both “or” and “and”.

Further, the term “about” when used in connection with a referenced numeric indication means the referenced numeric indication plus or minus up to 10% of that referenced numeric indication, e.g., within (plus or minus) 10%, 9%, 8%, 7%, 6%, 5%, 4%, 3%, 2%, 1%, 0.5%, 0.1%, 0.05%, or 0.01% of the stated value. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

### Example 1: Treatment of CD13-targeted TNF in Mouse Tumor Model

Study shows that mCD13-AFR activates endothelial cells similarly to wild type mTNF, as evidenced by induction of the endothelial cell adhesion marker ICAM-1 and by induction of intravascular coagulation.

FIG. 1 shows immunohistochemical analysis of B16BI6 tumor after p.l. treatment with 5 μg wild-type sc mTNF or 50 μg of the AcTakine mCD13 V_{H}H-sc mTNF Y114L. The upper panel was taken 4 hours after treatment and stained for mouse ICAM-1. The lower panel was taken 24 hours after treatment and shows a specific fluorescence in blood clots. Sections were made transverse and pictures were taken close to the skin, near the site of injection.

### Example 2: Co-administration of CD13-targeted TNF and CD8-targeted IFN in Mouse Tumor Model

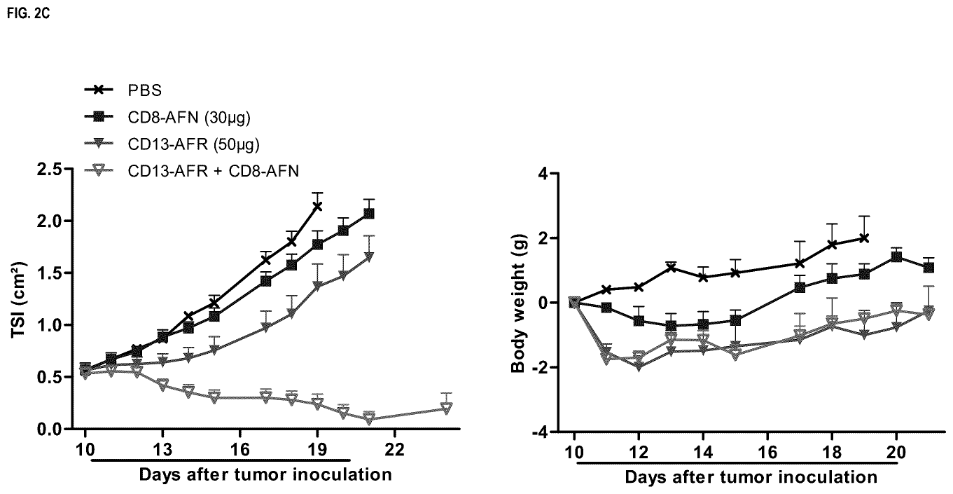

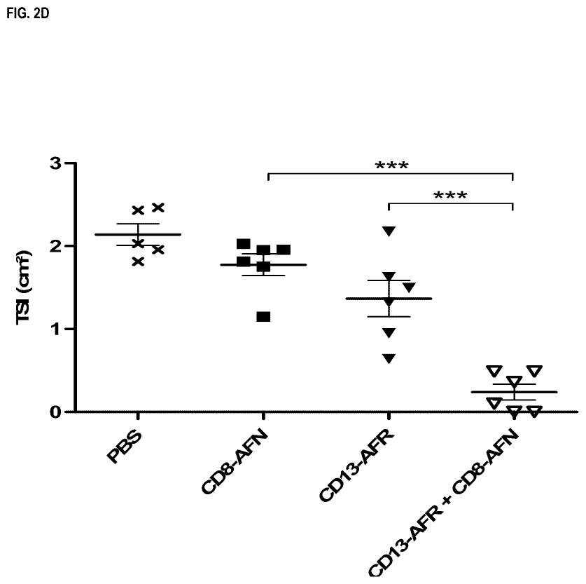

These studies demonstrate synergistic antitumor effects of mCD13-AFR with mCD8-AcTaferon, revealing that concomitant activation of tumor endothelium an activation of cytotoxic T lymphocytes generates strong antitumor effects. Importantly, antitumor activity was observed with absence of overt toxicity (e.g., no change in body weight). As shown in FIGS. 2A-B, treatment with: 1) CD8-targeted modified IFN (Q124R) (CD8-AcTaferon) alone; 2) Bcll10-targeted modified TNF (Y114L) (negative control) alone; and 3) CD13-targeted modified TNF (Y114L) alone had moderate tumor growth. Additionally, FIG. 2A shows that combination treatment with Bcll10-targeted modified TNF (Y114L) and CD8-AcTaferon had little effect on tumor growth.

FIG. 2B shows that combination treatment with CD13-targeted modified TNF (Y114L) and CD8-AcTaferon had a synergistic effect and greatly reduced tumor growth.

The combination of mCD13-AFR and mCD8α-targeted AcTaferon (mCD8-AFN) therapy was evaluated in the B16BI6 mouse melanoma model. mCD8-AFN therapy, which had no effect on itself, clearly synergized with mCD13-AFR, leading to extensive tumor regression (FIGS. 2C-D). Again, no toxic side effects were observed in all combinations tested, further underscoring the selectivity and safety of AcTakine therapy.

### Example 3: Co-administration of CD13-targeted TNF and Wortmannin in Mouse Tumor Model

Examples 3, 4 and 5 are the studies that show that mCD13-AFR synergizes with various agents to induce potent antitumor activity (tumor regression), including PI3K inhibition (Wortmannin), chemotherapy (doxorubicin, which induces immunogenic tumor cell death), and IAP inhibition (Birinapant, a SMAC mimetic, which inhibits IAP apoptosis inhibitors).

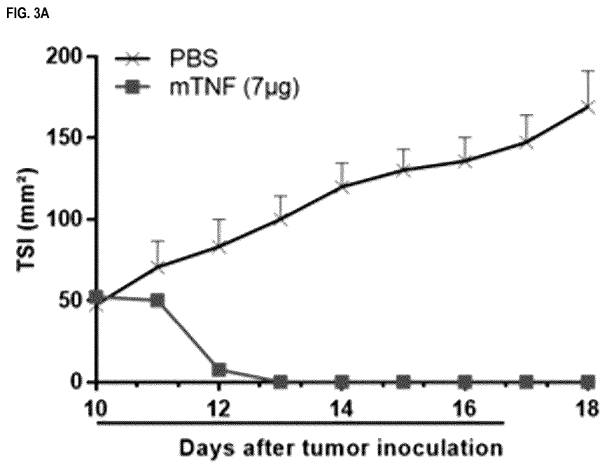

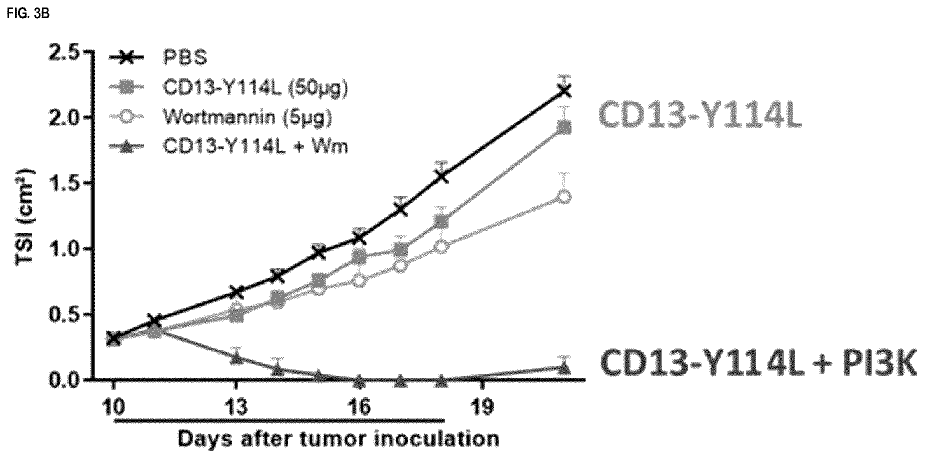

FIG. 3A shows antitumor effects of wild type mouse TNF at 7 μg. This was associated with substantial toxicity (not shown: e.g., body weight loss and lethality), consistent with the pleiotropic activity of untargeted wild type TNF. As shown in FIG. 3B, treatment with Wortmannin alone and CD13-targeted modified TNF (Y114L) alone had very little effect on tumor growth. FIG. 3B also shows that combination treatment with CD13-targeted modified TNF (Y114L) and Wortmannin had a synergistic effect and greatly reduced tumor growth.

### Example 4: Co-administration of CD13-targeted TNF and PEGylated Liposomal Doxorubicin in Mouse Tumor Model

This study shows the synergistic antitumor activity of mCD13-AFR in combination with the chemotherapy agent doxorubicin, which promotes immunogenic tumor cell death.

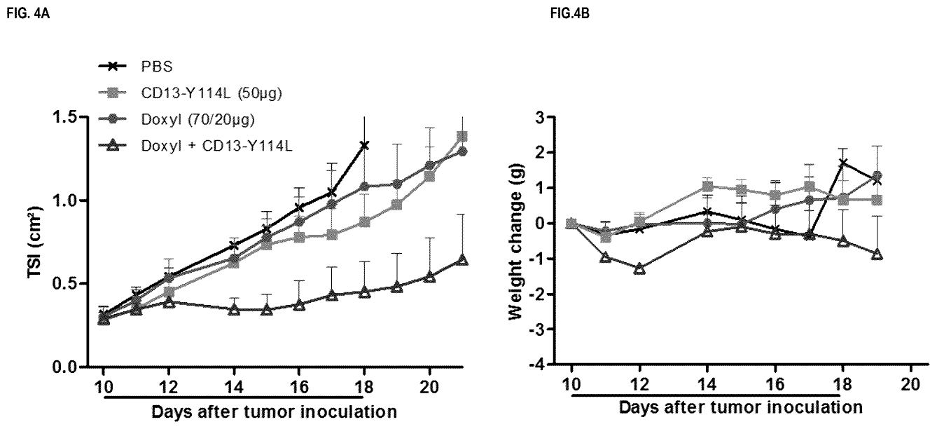

FIG. 4A shows that treatment with pegylated liposomal doxorubicin (Doxyl) alone and CD13-targeted modified TNF (Y114L) alone had very little effect on tumor growth. FIG. 4A also shows that combination treatment with CD13-targeted modified TNF (Y114L) and Doxyl had a synergistic effect and greatly reduced tumor growth.

FIG. 4B shows that the combined treatment of CD13-targeted modified TNF (Y114L) and Doxyl had minimal adverse effect on body weight.

### Example 5: Co-administration of CD13-targeted TNF and Birinapant in Mouse Tumor Model

This study shows the synergistic antitumor activity of mCD13-AFR in combination with Birinapant, an inhibitor of IAP apoptosis inhibitor proteins.

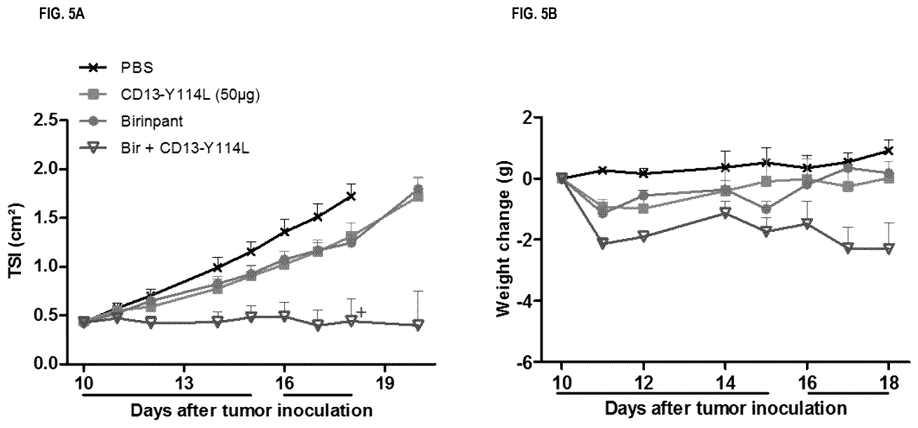

FIG. 5A shows that treatment with birinapant (Bir) alone and CD13-targeted modified TNF (Y114L) alone had very little effect on tumor growth. FIG. 5A also shows that combination treatment with CD13-targeted modified TNF (Y114L) and Bir had a synergistic effect and greatly reduced tumor growth.

FIG. 5B shows that the combined treatment of CD13-targeted modified TNF (Y114L) and Bir had minimal adverse effect on body weight.

### Example 6: AFRs Combine Safety and Activity In Vivo

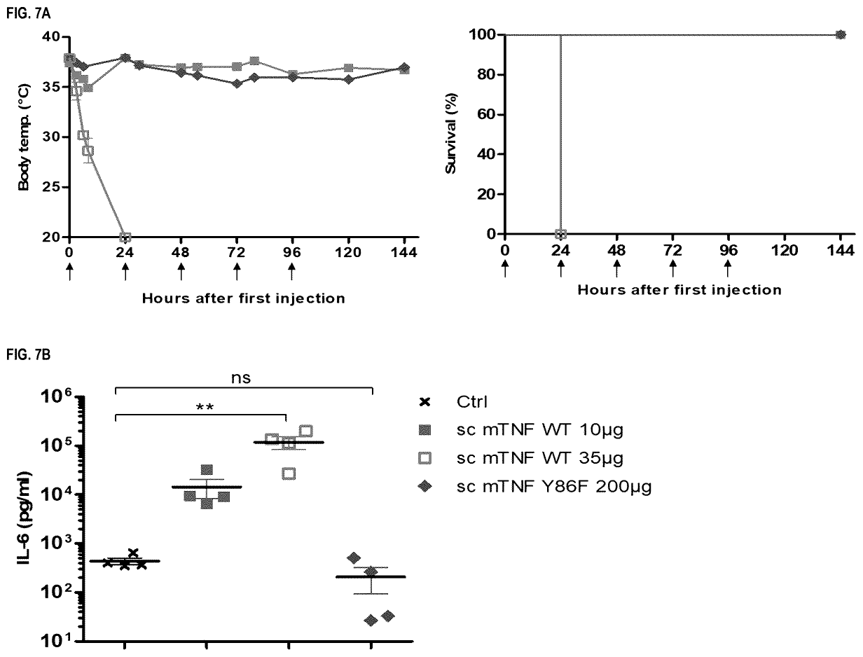

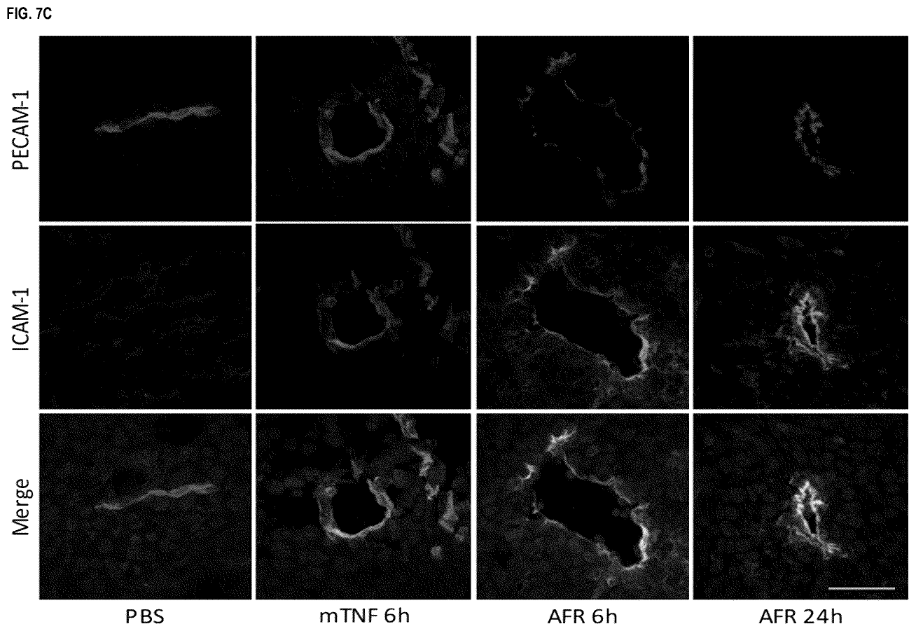

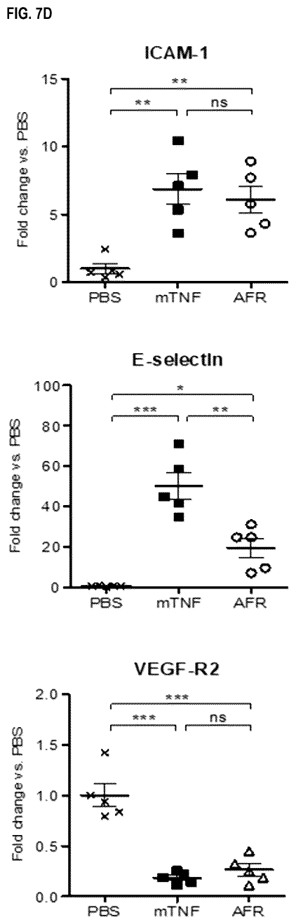

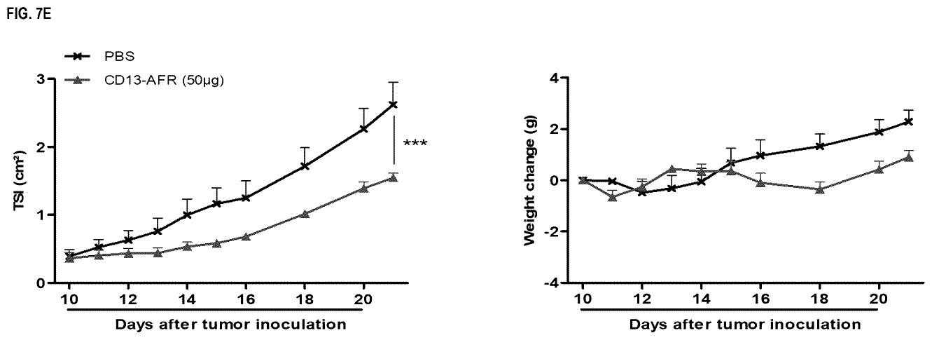

To evaluate in vivo toxicity, the i.v. shock model in naive C57BL/6 mice was used first. The Bell10-AFR was used to avoid target-specific effects. Injection of 10 μg scTNF, which is lethal when using wtTNF, only led to a moderate drop in body temperature (FIG. 7A), but induced significant levels of circulating IL-6, a sensitive marker for systemic TNF activity (FIG. 7B). 35 μg (1.75 mg/kg) of scTNF was an LD 100 in this model and was characterized by a dramatic drop in body temperature and concomitant increase in circulating IL-6. For Bcll10-AFR, 5 consecutive daily i.v. bolus injections of 200 μg (10 mg/kg) were completely non-toxic and did not induce any drop in body temperature or increase in circulating IL-6, confirming that untargeted AFRs are inactive.

To target tumor vasculature, a V_{H}H against mouse CD13 was generated (Pasqualini, R., et al., Cancer Res, 2000. 60(3): p. 722-7). This V_{H}H was fused to wt scTNF or mutant Y86F. The immunocytokine mCD13-targeted wt scTNF was slightly more efficient in the B16BI6 melanoma model than untargeted scTNF, confirming the targeting potential of this V_{H}H (data not shown). To demonstrate the in vivo activity of tumor vasculature-targeted AFRs, immunohistochemical analysis of tumor sections after injection of wtTNF or mCD13-AFR (FIGS. 1 and 7C) was performed. Six hours after injection, clear induction of ICAM-1 on tumor vessels was observed, which was even more pronounced 24 hours after injection, indicating prolonged endothelial activation. Induction of ICAM-1 expression was confirmed via qPCR analysis on whole tumor RNA (FIG. 7D). Quite similar to wtTNF, mCD13-AFR induced expression of both ICAM-1 and E-selectin and repression of VEGF-R2 at 4 hours after injection. Since tumor-bearing mice tend to be sensitized towards TNF toxicity (Cauwels, A., et al., Cancer Res, 2018. 78 (2): p. 463-474), mCD13-AFR in the B16BI6 melanoma model was tested next. Daily treatment for 10 consecutive days with 2.5 mg/kg mCD13-AFR caused no significant weight loss or other apparent toxicity, confirming its safety, while also demonstrating moderate antitumor activity (FIG. 7E).

### Example 7: IFN-γ Sensitizes Tumor Endothelial Cells for TNF Activity

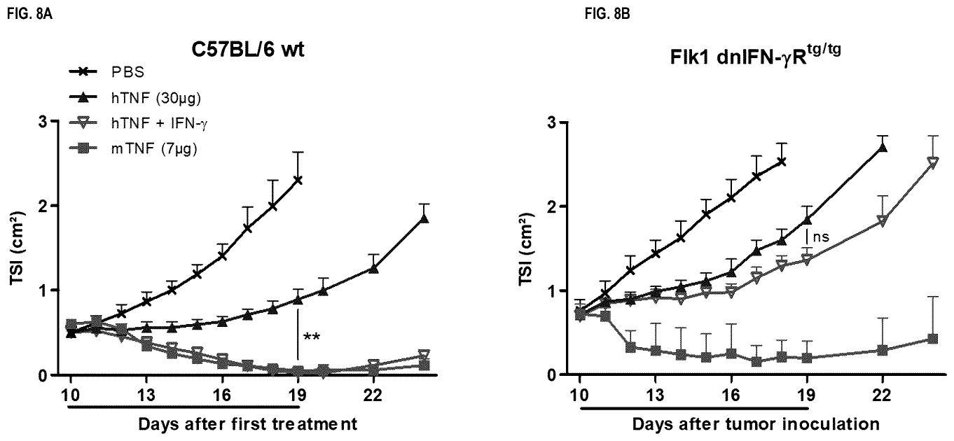

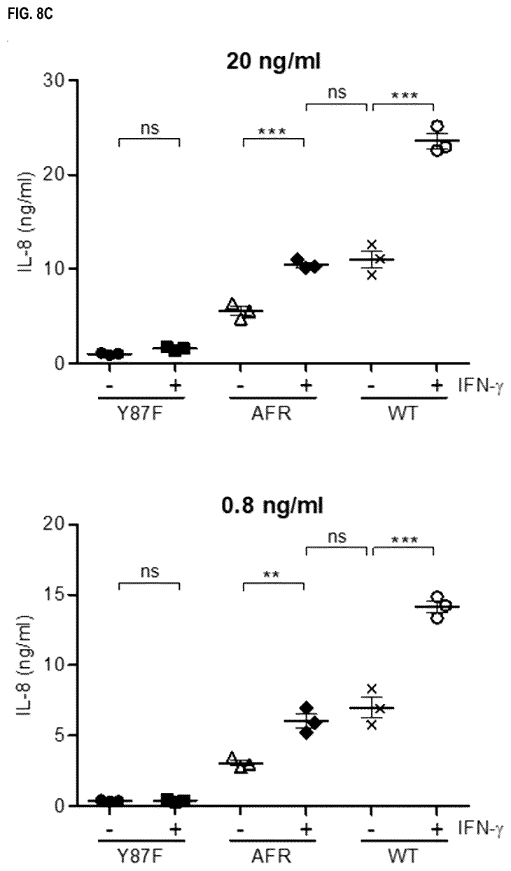

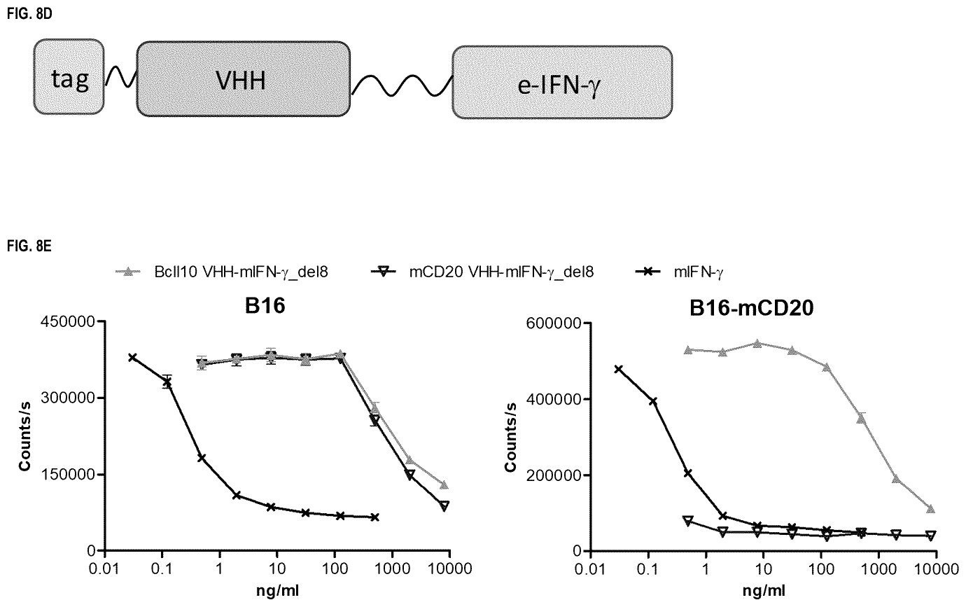

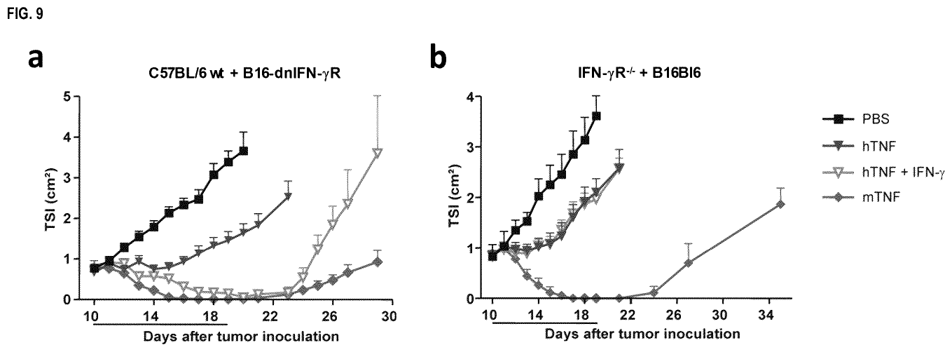

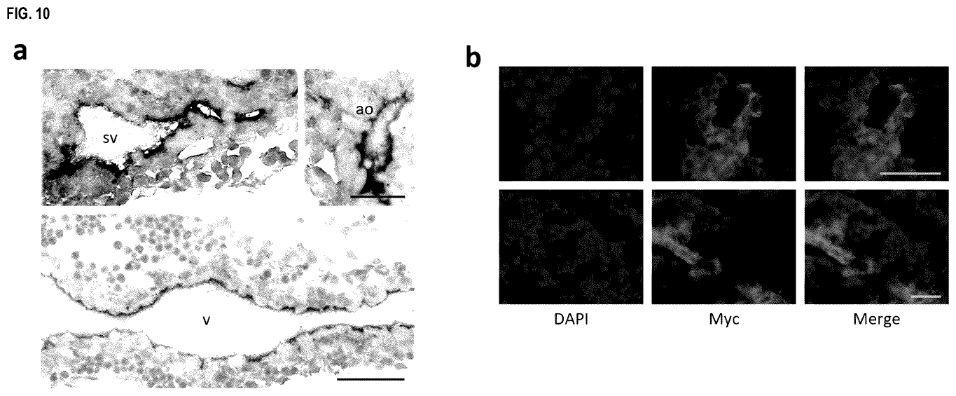

In the B16BI6 model, synergism between IFN-γ and TNF is clear when mIFN-γ is combined with hTNF. Human TNF, which can be considered as a low-toxic, fast-cleared mTNF mutant in the mouse (Ameloot, P., et al., Eur J Immunol, 2002. 32(10): p. 2759-65), is unable to induce complete tumor regression in the B16BI6 model, except when combined with mlFN-γ (FIG. 8A). To investigate whether the synergistic effect of TNF and IFN-γ depends on direct actions on tumor cells or on host cells, IFN-γ-insensitive B16BI6 tumor cells (B16-dnIFN-γR) were generated. When wt mice carrying a B16-dnIFN-γR tumor were treated with hTNF and mIFN-γ the synergistic antitumor effect was still present (FIG. 9A). By contrast, this synergy was completely absent in IFN-γR knockout mice carrying a parental B16BI6 tumor (FIG. 9B), pointing towards the important role of the host microenvironment. It was hypothesized that IFN-γ may also act on endothelial cells, sensitizing them for the effects of TNF. Therefore transgenic mice were generated in which the vasculature is selectively unresponsive to IFN-γ by endothelial-specific Flk1 promoter-driven expression of a truncated, dominant-negative IFN-γR (FIGS. 10A and 10B). As shown in FIG. 8B, treatment of these mice resulted in complete loss of the synergy between hTNF and mIFN-γ, confirming the critical role of IFN-γ signaling in endothelial cells.

It was also tested whether IFN-γ could sensitize HUVECs to TNF, using either wtTNF or hCD13-AFR. Indeed, whilst IFN-γ treatment alone had no effect on IL-8 secretion by HUVECs, it sensitized for TNF signaling leading to a 2 to 3-fold increased IL-8 secretion when compared with wtTNF or hCD13-AFR alone (FIG. 8C). Of note, IL-8 secretion levels of hCD13-AFR plus hIFN-γ stimulated cells were equal to those after stimulation with wtTNF alone.

### Example 8: Type II AcTaferon Synergizes With CD13-AFR for Tumor Destruction

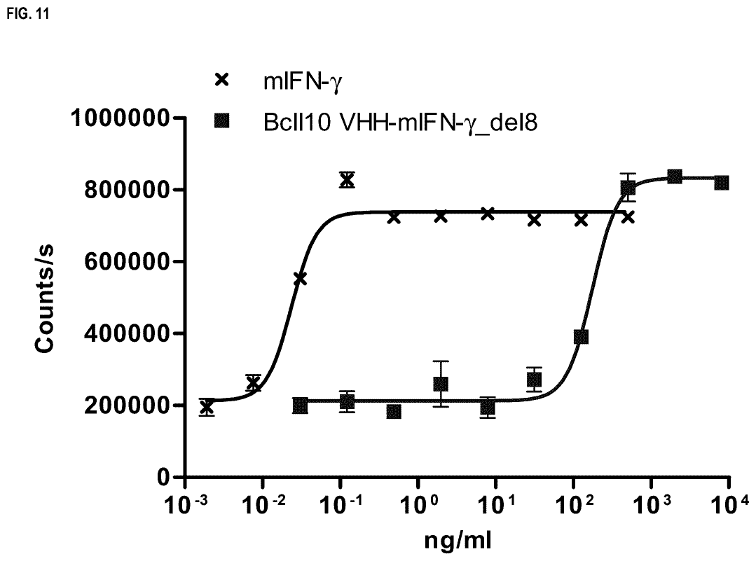

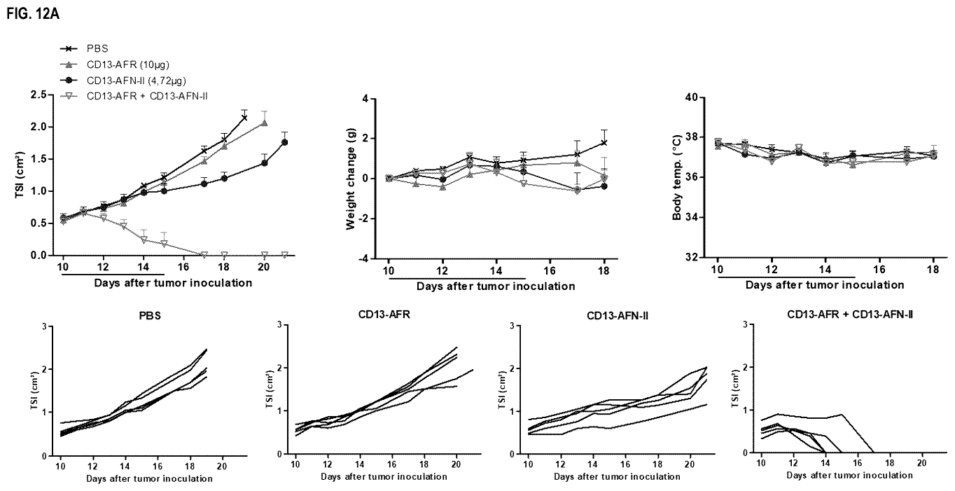

Based on the findings above, a type II AcTaferon (AFN-II) (FIG. 8D) was designed. Deletion of the eight (8)C-terminal amino acids reduced mIFN-γ activity approximately 7000-fold in an encephalomyocarditis virus (EMCV) cytopathic assay in L929 cells (FIG. 11), and more than 3000-fold in a TNF/IFN-γ cytotoxicity assay in B16BI6 cells (FIG. 8E). As seen for mCD20-AFN-II, the activity was completely recovered (FIG. 8E) and even surpassed that of wt mIFN-γ on B16BI6 cells expressing mCD20, predicting a more than 10000-fold AcTakine targeting efficiency. To target IFN-γ activity to tumor vasculature, the same mCD13 V_{H}H as for AFR was used. In both the B16BI6 model and the human RL lymphoma model in immunodeficient NSG mice, daily treatment with mCD13-AFN-II resulted in significant tumor growth suppression (FIGS. 12A and 12B). To evaluate the in vivo synergy between mCD13-AFR and mCD13-AFN-II, equimolar doses in combination treatments were used. In both tumor models, combination therapy induced very rapid tumor necrosis, resulting in complete regression within 6 days of treatment in all mice (FIGS. 12A and 12B), demonstrating the generic and direct effect on tumor vasculature, independent of an immune response. The kinetics and appearance of tumor necrosis induced by mCD13-AFN-II/mCD13-AFR combination therapy (FIGS. 12A and 12B) were similar to those observed during treatment with high-dose wtTNF alone, suggesting that mCD13-AFN-II is able to shift mCD13-AFR signaling towards cell death. This was further confirmed by the detection of selective endothelial apoptosis in B16BI6 tumors as soon as 3 to 5 hours after mCD13-AFN-II/mCD13-AFR treatment (FIG. 12C). Strikingly, even this synergistic mCD13-IFN-II/mCD13-AFR co-treatment did not evoke a detectable toxic response.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

1.-57. (canceled)

58. A method for treating cancer comprising co-administering to a subject in need thereof an effective amount of:
(a) a chimeric protein or chimeric protein complex comprising:
(i) a targeting moiety comprising a recognition domain which recognizes and binds to CD13; and
(ii) a modified tumor necrosis factor (TNF) signaling agent, the modified TNF signaling agent having one or more mutations that confer improved safety as compared to a wild type TNF signaling agent, and
(b) at least one additional therapeutic agent is selected from:
(i) a PI3K inhibitor,
(ii) an anthracycline, and
(iii) a SMAC mimetic.

59. The method of claim 58, wherein the CD13 recognition domain recognizes and/or binds an antigen or receptor on an endothelial cell of tumor neovasculature and/or tumor cell.

60. The method of claim 58, wherein the modified TNF signaling agent comprises one or more mutations conferring reduced affinity or activity at the TNF signaling agent's receptor relative to a wild type TNF signaling agent.

61. The method of claim 58, wherein the modified TNF signaling agent comprises one or more mutations conferring substantially reduced or ablated affinity or activity for a receptor relative to a wild type TNF signaling agent.

62. The method of claim 58, wherein the modified TNF signaling agent comprises both (a) one or more mutations conferring substantially reduced or ablated affinity for a receptor relative to a wild type TNF signaling agent and (b) one or more mutations conferring reduced affinity or activity for a receptor relative to a wild type TNF signaling agent; and wherein the receptors are different.

63. The method of claim 58, wherein the PI3K inhibitor is selected from Wortmannin, PX-866, demethoxyviridin, LY294002, idelalisib, umbralisib, duvelisib, copanlisib, buparlisib, pilaralisib, pictilisib, alpelisib, taselisib, NCP-BEZ235, LY3023414, GSK2126458, perifosine, dactolisib, CUDC-907, voxtalisib, ME-401, IPI-549, SF1126, RP6530, INK1117, XL147 (SAR245408), palomid 529, GSK1059615, ZSTK474, PWT33597, IC87114, TG-100-115, CAL263, RP6503, PI-103, GNE-477, and AEZS-136.

64. The method of claim 63, wherein the PI3K inhibitor is Wortmannin.

65. The method of claim 58, wherein the anthracycline is selected from doxorubicin, daunorubicin, epirubicin, mitoxantrone, idarubicin, aldoxorubicin, annamycin, plicamycin, pirarubicin, aclarubicin, zorubicin, sabarubicin, zoptarelin doxorubicin, GPX-150, SP1049C, and valrubicin.

66. The method of claim 65, wherein the anthracycline is doxorubicin.

67. The method of claim 65, wherein the anthracycline is encapsulated by a liposome.

68. The method of claim 67, wherein the liposome is pegylated.

69. The method of claim 58, wherein the SMAC mimetic is selected from birinapant, LCL161, GDC-0917, HGS1029, TPI 1237-22, AT-406/Debio1143, and GT13402.

70. The method of claim 69, wherein the SMAC mimetic is birinapant.


SEQUENCE LISTING (381 sequences)

SEQ ID NO: 1 - protein, 967 aa, synthetic construct
  REGION 1..967: note = Synthetic Sequence
MAKGFYISKSLGILGILLGVAAVCTIIALSVVYSQEKNKNANSSPVASTTPSASATTNPASATTLDQSKAWNRYRLPNTLKPDSYRVTLRPYLTPNDRGLYVFKGSSTVRFTCKEATDVIIIHSKKLNYTLSQGHRVVLRGVGGSQPPDIDKTELVEPTEYLVVHLKGSLVKDSQYEMDSEFEGELADDLAGFYRSEYMEGNVRKVVATTQMQAADARKSFPCFDEPAMKAEFNITLIHPKDLTALSNMLPKGPSTPLPEDPNWNVTEFHTTPKMSTYLLAFIVSEFDYVEKQASNGVLIRIWARPSAIAAGHGDYALNVTGPILNFFAGHYDTPYPLPKSDQIGLPDFNAGAMENWGLVTYRENSLLFDPLSSSSSNKERVVTVIAHELAHQWFGNLVTIEWWNDLWLNEGFASYVEYLGADYAEPTWNLKDLMVLNDVYRVMAVDALASSHPLSTPASEINTPAQISELFDAISYSKGASVLRMLSSFLSEDVFKQGLASYLHTFAYQNTIYLNLWDHLQEAVNNRSIQLPTTVRDIMNRWTLQMGFPVITVDTSTGTLSQEHFLLDPDSNVTRPSEFNYVWIVPITSIRDGRQQQDYWLIDVRAQNDLFSTSGNEWVLLNLNVTGYYRVNYDEENWRKIQTQLQRDHSAIPVINRAQIINDAFNLASAHKVPVTLALNNTLFLIEERQYMPWEAALSSLSYFKLMFDRSEVYGPMKNYLKKQVTPLFIHFRNNTNNWREIPENLMDQYSEVNAISTACSNGVPECEEMVSGLFKQWMENPNNNPIHPNLRSTVYCNAIAQGGEEEWDFAWEQFRNATLVNEADKLRAALACSKELWILNRYLSYTLNPDLIRKQDATSTIISITNNVIGQGLVWDFVQSNWKKLFNDYGGGSFSFSNLIQAVTRRFSTEYELQQLEQFKKDNEETGFGSGTRALEQALEKTKANIKWVKENKEVVLQWFTENSK

SEQ ID NO: 2 - protein, 157 aa, synthetic construct [sequence omitted]

SEQ ID NO: 3 - protein, 171 aa, synthetic construct
  REGION 1..171: note = Synthetic Sequence
LPGVGLTPSAAQTARQHPKMHLAHSNLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 4 - protein, 281 aa, synthetic construct [sequence omitted]

SEQ ID NO: 5 - protein, 5 aa, synthetic construct
  REGION 1..5: note = Synthetic Sequence
GGGGS

SEQ ID NO: 6 - protein, 10 aa, synthetic construct
  REGION 1..10: note = Synthetic Sequence
GGGGSGGGGS

SEQ ID NO: 7 - protein, 15 aa, synthetic construct
  REGION 1..15: note = Synthetic Sequence
GGGGSGGGGSGGGGS

SEQ ID NO: 8 - protein, 20 aa, synthetic construct
  REGION 1..20: note = Synthetic Sequence
GGGGSGGGGSGGGGSGGGGS

SEQ ID NO: 9 - protein, 25 aa, synthetic construct
  REGION 1..25: note = Synthetic Sequence
GGGGSGGGGSGGGGSGGGGSGGGGS

SEQ ID NO: 10 - protein, 30 aa, synthetic construct
  REGION 1..30: note = Synthetic Sequence
GGGGSGGGGSGGGGSGGGGSGGGGSGGGGS

SEQ ID NO: 11 - protein, 35 aa, synthetic construct
  REGION 1..35: note = Synthetic Sequence
GGGGSGGGGSGGGGSGGGGSGGGGSGGGGSGGGGS

SEQ ID NO: 12 - protein, 40 aa, synthetic construct
  REGION 1..40: note = Synthetic Sequence
GGGGSGGGGSGGGGSGGGGSGGGGSGGGGSGGGGSGGGGS

SEQ ID NO: 13 - protein, 16 aa, synthetic construct
  REGION 1..16: note = Synthetic Sequence
GGSGGSGGGGSGGGGS

SEQ ID NO: 14 - protein, 8 aa, synthetic construct
  REGION 1..8: note = Synthetic Sequence
GGGGGGGG

SEQ ID NO: 15 - protein, 6 aa, synthetic construct
  REGION 1..6: note = Synthetic Sequence
GGGGGG

SEQ ID NO: 16 - protein, 5 aa, synthetic construct
  REGION 1..5: note = Synthetic Sequence
EAAAK

SEQ ID NO: 17 - protein, 10 aa, synthetic construct
  REGION 1..10: note = Synthetic Sequence
EAAAKEAAAK

SEQ ID NO: 18 - protein, 15 aa, synthetic construct
  REGION 1..15: note = Synthetic Sequence
EAAAKEAAAKEAAAK

SEQ ID NO: 19 - protein, 12 aa, synthetic construct
  REGION 1..12: note = Synthetic Sequence
AEAAAKEAAAKA

SEQ ID NO: 20 - protein, 17 aa, synthetic construct
  REGION 1..17: note = Synthetic Sequence
AEAAAKEAAAKEAAAKA

SEQ ID NO: 21 - protein, 22 aa, synthetic construct
  REGION 1..22: note = Synthetic Sequence
AEAAAKEAAAKEAAAKEAAAKA

SEQ ID NO: 22 - protein, 27 aa, synthetic construct
  REGION 1..27: note = Synthetic Sequence
AEAAAKEAAAKEAAAKEAAAKEAAAKA

SEQ ID NO: 23 - protein, 12 aa, synthetic construct
  REGION 1..12: note = Synthetic Sequence
AEAAAKEAAAKA

SEQ ID NO: 24 - protein, 46 aa, synthetic construct
  REGION 1..46: note = Synthetic Sequence
AEAAAKEAAAKEAAAKEAAAKALEAEAAAKEAAAKEAAAKEAAAKA

SEQ ID NO: 25 - protein, 5 aa, synthetic construct
  REGION 1..5: note = Synthetic Sequence
PAPAP

SEQ ID NO: 26 - protein, 18 aa, synthetic construct
  REGION 1..18: note = Synthetic Sequence
KESGSVSSEQLAQFRSLD

SEQ ID NO: 27 - protein, 14 aa, synthetic construct
  REGION 1..14: note = Synthetic Sequence
EGKSSGSGSESKST

SEQ ID NO: 28 - protein, 12 aa, synthetic construct
  REGION 1..12: note = Synthetic Sequence
GSAGSAAGSGEF

SEQ ID NO: 29 - skipped

SEQ ID NO: 30 - protein, 6 aa, synthetic construct
  REGION 1..6: note = Synthetic Sequence
GGSGGS

SEQ ID NO: 31 - protein, 9 aa, synthetic construct
  REGION 1..9: note = Synthetic Sequence
GGSGGSGGS

SEQ ID NO: 32 - protein, 12 aa, synthetic construct
  REGION 1..12: note = Synthetic Sequence
GGSGGSGGSGGS

SEQ ID NO: 33 - protein, 15 aa, synthetic construct
  REGION 1..15: note = Synthetic Sequence
GGSGGSGGSGGSGGS

SEQ ID NO: 34 - protein, 18 aa, synthetic construct
  REGION 1..18: note = Synthetic Sequence
GGSGGSGGSGGSGGSGGS

SEQ ID NO: 35 - protein, 21 aa, synthetic construct
  REGION 1..21: note = Synthetic Sequence
GGSGGSGGSGGSGGSGGSGGS

SEQ ID NO: 36 - protein, 24 aa, synthetic construct
  REGION 1..24: note = Synthetic Sequence
GGSGGSGGSGGSGGSGGSGGSGGS

SEQ ID NO: 37 - protein, 27 aa, synthetic construct
  REGION 1..27: note = Synthetic Sequence
GGSGGSGGSGGSGGSGGSGGSGGSGGS

SEQ ID NO: 38 - protein, 30 aa, synthetic construct
  REGION 1..30: note = Synthetic Sequence
GGSGGSGGSGGSGGSGGSGGSGGSGGSGGS

SEQ ID NO: 39 - protein, 33 aa, synthetic construct
  REGION 1..33: note = Synthetic Sequence
GGSGGSGGSGGSGGSGGSGGSGGSGGSGGSGGS

SEQ ID NO: 40 - protein, 36 aa, synthetic construct
  REGION 1..36: note = Synthetic Sequence
GGSGGSGGSGGSGGSGGSGGSGGSGGSGGSGGSGGS

SEQ ID NO: 41 - protein, 39 aa, synthetic construct
  REGION 1..39: note = Synthetic Sequence
GGSGGSGGSGGSGGSGGSGGSGGSGGSGGSGGSGGSGGS

SEQ ID NO: 42 - protein, 42 aa, synthetic construct
  REGION 1..42: note = Synthetic Sequence
GGSGGSGGSGGSGGSGGSGGSGGSGGSGGSGGSGGSGGSGGS

SEQ ID NO: 43 - protein, 45 aa, synthetic construct
  REGION 1..45: note = Synthetic Sequence
GGSGGSGGSGGSGGSGGSGGSGGSGGSGGSGGSGGSGGSGGSGGS

SEQ ID NO: 44 - protein, 48 aa, synthetic construct
  REGION 1..48: note = Synthetic Sequence
GGSGGSGGSGGSGGSGGSGGSGGSGGSGGSGGSGGSGGSGGSGGSGGS

SEQ ID NO: 45 - protein, 51 aa, synthetic construct
  REGION 1..51: note = Synthetic Sequence
GGSGGSGGSGGSGGSGGSGGSGGSGGSGGSGGSGGSGGSGGSGGSGGSGGS

SEQ ID NO: 46 - protein, 54 aa, synthetic construct
  REGION 1..54: note = Synthetic Sequence
GGSGGSGGSGGSGGSGGSGGSGGSGGSGGSGGSGGSGGSGGSGGSGGSGGSGGS

SEQ ID NO: 47 - protein, 57 aa, synthetic construct
  REGION 1..57: note = Synthetic Sequence
GGSGGSGGSGGSGGSGGSGGSGGSGGSGGSGGSGGSGGSGGSGGSGGSGGSGGSGGS

SEQ ID NO: 48 - protein, 60 aa, synthetic construct
  REGION 1..60: note = Synthetic Sequence
GGSGGSGGSGGSGGSGGSGGSGGSGGSGGSGGSGGSGGSGGSGGSGGSGGSGGSGGSGGS

SEQ ID NO: 49 - protein, 45 aa, synthetic construct
  REGION 1..45: note = Synthetic Sequence
GGGGSGGGGSGGGGSGGGGSGGGGSGGGGSGGGGSGGGGSGGGGS

SEQ ID NO: 50 - protein, 50 aa, synthetic construct
  REGION 1..50: note = Synthetic Sequence
GGGGSGGGGSGGGGSGGGGSGGGGSGGGGSGGGGSGGGGSGGGGSGGGGS

SEQ ID NO: 51 - protein, 55 aa, synthetic construct
  REGION 1..55: note = Synthetic Sequence
GGGGSGGGGSGGGGSGGGGSGGGGSGGGGSGGGGSGGGGSGGGGSGGGGSGGGGS

SEQ ID NO: 52 - protein, 60 aa, synthetic construct
  REGION 1..60: note = Synthetic Sequence
GGGGSGGGGSGGGGSGGGGSGGGGSGGGGSGGGGSGGGGSGGGGSGGGGSGGGGSGGGGS

SEQ ID NO: 53 - protein, 65 aa, synthetic construct
  REGION 1..65: note = Synthetic Sequence
GGGGSGGGGSGGGGSGGGGSGGGGSGGGGSGGGGSGGGGSGGGGSGGGGSGGGGSGGGGSGGGGS

SEQ ID NO: 54 - protein, 70 aa, synthetic construct
  REGION 1..70: note = Synthetic Sequence
GGGGSGGGGSGGGGSGGGGSGGGGSGGGGSGGGGSGGGGSGGGGSGGGGSGGGGSGGGGSGGGGSGGGGS

SEQ ID NO: 55 - protein, 75 aa, synthetic construct
  REGION 1..75: note = Synthetic Sequence
GGGGSGGGGSGGGGSGGGGSGGGGSGGGGSGGGGSGGGGSGGGGSGGGGSGGGGSGGGGSGGGGSGGGGSGGGGS

SEQ ID NO: 56 - protein, 80 aa, synthetic construct
  REGION 1..80: note = Synthetic Sequence
GGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 57 - protein, 85 aa, synthetic construct
  REGION 1..85: note = Synthetic Sequence
GGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 58 - protein, 90 aa, synthetic construct
  REGION 1..90: note = Synthetic Sequence
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 59 - protein, 95 aa, synthetic construct
  REGION 1..95: note = Synthetic Sequence
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 60 - protein, 100 aa, synthetic construct
  REGION 1..100: note = Synthetic Sequence
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 61 - protein, 5 aa, synthetic construct
  REGION 1..5: note = Synthetic Sequence
GGGSE

SEQ ID NO: 62 - protein, 5 aa, synthetic construct
  REGION 1..5: note = Synthetic Sequence
GSESG

SEQ ID NO: 63 - protein, 5 aa, synthetic construct
  REGION 1..5: note = Synthetic Sequence
GSEGS

SEQ ID NO: 64 - protein, 35 aa, synthetic construct
  REGION 1..35: note = Synthetic Sequence
GEGGSGEGSSGEGSSSEGGGSEGGGSEGGGSEGGS

SEQ ID NO: 65 - protein, 4 aa, synthetic construct
  REGION 1..4: note = Synthetic Sequence
CPPC

SEQ ID NO: 66 - protein, 235 aa, synthetic construct
  REGION 1..235: note = Synthetic Sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 67 - protein, 198 aa, synthetic construct
  REGION 1..198: note = Synthetic Sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAGNRRRVCKCPRPVVKSGDKPSLSARYV

SEQ ID NO: 68 - protein, 276 aa, synthetic construct
  REGION 1..276: note = Synthetic Sequence
MRNQAPGRPKGATFPPRRPTGSRAPPLAPELRAKQRPGERVMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 69 - protein, 210 aa, synthetic construct
  REGION 1..210: note = Synthetic Sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 70 - protein, 221 aa, synthetic construct
  REGION 1..221: note = Synthetic Sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 71 - protein, 213 aa, synthetic construct
  REGION 1..213: note = Synthetic Sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGRRRRARLRFMKQPQGEGISGTFVPQCLHGYYSNTTTSQKLLNPWILKT

SEQ ID NO: 72 - protein, 246 aa, synthetic construct
  REGION 1..246: note = Synthetic Sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQ

SEQ ID NO: 73 - protein, 243 aa, synthetic construct
  REGION 1..243: note = Synthetic Sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 74 - protein, 180 aa, synthetic construct
  REGION 1..180: note = Synthetic Sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGRRRRARLRFMKQFYK

SEQ ID NO: 75 - protein, 206 aa, synthetic construct
  REGION 1..206: note = Synthetic Sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKDFTNKQRIGFWCPATKRHRSVMSTMWKNERRDTFNPGEFNGC

SEQ ID NO: 76 - protein, 198 aa, synthetic construct
  REGION 1..198: note = Synthetic Sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 77 - protein, 10 aa, synthetic construct
  REGION 1..10: note = Synthetic Sequence
GFTFDDYAMS

SEQ ID NO: 78 - protein, 10 aa, synthetic construct
  REGION 1..10: note = Synthetic Sequence
GFTFDDYAIG

SEQ ID NO: 79 - protein, 10 aa, synthetic construct
  REGION 1..10: note = Synthetic Sequence
GRSFSSYTLA

SEQ ID NO: 80 - protein, 10 aa, synthetic construct
  REGION 1..10: note = Synthetic Sequence
GRTFSSYTMG

SEQ ID NO: 81 - protein, 10 aa, synthetic construct
  REGION 1..10: note = Synthetic Sequence
GRTFSSYIMG

SEQ ID NO: 82 - protein, 10 aa, synthetic construct
  REGION 1..10: note = Synthetic Sequence
GRTFSSYTMG

SEQ ID NO: 83 - protein, 14 aa, synthetic construct
  REGION 1..14: note = Synthetic Sequence
GRTSGRTFSSYTMG

SEQ ID NO: 84 - protein, 10 aa, synthetic construct
  REGION 1..10: note = Synthetic Sequence
GRTFSSYAMG

SEQ ID NO: 85 - protein, 10 aa, synthetic construct
  REGION 1..10: note = Synthetic Sequence
GLTFSNYIMG

SEQ ID NO: 86 - protein, 10 aa, synthetic construct
  REGION 1..10: note = Synthetic Sequence
GRTFSSYTMG

SEQ ID NO: 87 - protein, 10 aa, synthetic construct
  REGION 1..10: note = Synthetic Sequence
GRTFSSDTMG

SEQ ID NO: 88 - protein, 10 aa, synthetic construct
  REGION 1..10: note = Synthetic Sequence
GLTFSNYIMG

SEQ ID NO: 89 - protein, 10 aa, synthetic construct
  REGION 1..10: note = Synthetic Sequence
GFTLDYYGIG

SEQ ID NO: 90 - protein, 10 aa, synthetic construct
  REGION 1..10: note = Synthetic Sequence
GHTFSSYTMG

SEQ ID NO: 91 - protein, 10 aa, synthetic construct
  REGION 1..10: note = Synthetic Sequence
GRTFSSYVIG

SEQ ID NO: 92 - protein, 10 aa, synthetic construct
  REGION 1..10: note = Synthetic Sequence
GFAFDGYAIG

SEQ ID NO: 93 - protein, 10 aa, synthetic construct
  REGION 1..10: note = Synthetic Sequence
GFAFGFFDMT

SEQ ID NO: 94 - protein, 10 aa, synthetic construct
  REGION 1..10: note = Synthetic Sequence
GRTFSNYVIG

SEQ ID NO: 95 - protein, 10 aa, synthetic construct
  REGION 1..10: note = Synthetic Sequence
GSIFSINVMG

SEQ ID NO: 96 - protein, 10 aa, synthetic construct
  REGION 1..10: note = Synthetic Sequence
GRTFSNYNVG

SEQ ID NO: 97 - protein, 10 aa, synthetic construct
  REGION 1..10: note = Synthetic Sequence
GHTFSSYTMG

SEQ ID NO: 98 - protein, 10 aa, synthetic construct
  REGION 1..10: note = Synthetic Sequence
GRTFSTYPVG

SEQ ID NO: 99 - protein, 10 aa, synthetic construct
  REGION 1..10: note = Synthetic Sequence
GRTFSNYAMG

SEQ ID NO: 100 - protein, 10 aa, synthetic construct
  REGION 1..10: note = Synthetic Sequence
GRTFSDYRMG

SEQ ID NO: 101 - protein, 10 aa, synthetic construct
  REGION 1..10: note = Synthetic Sequence
GLTFSNYIMA

SEQ ID NO: 102 - protein, 10 aa, synthetic construct
  REGION 1..10: note = Synthetic Sequence
GRTFSNSVMG

SEQ ID NO: 103 - protein, 10 aa, synthetic construct
  REGION 1..10: note = Synthetic Sequence
GRTFSSYIIG

SEQ ID NO: 104 - protein, 10 aa, synthetic construct
  REGION 1..10: note = Synthetic Sequence
GRTFSSYVMG

SEQ ID NO: 105 - protein, 10 aa, synthetic construct
  REGION 1..10: note = Synthetic Sequence
GGTFSNYVMG

SEQ ID NO: 106 - protein, 10 aa, synthetic construct
  REGION 1..10: note = Synthetic Sequence
GRTFSNYGIG

SEQ ID NO: 107 - protein, 10 aa, synthetic construct
  REGION 1..10: note = Synthetic Sequence
GFTFDDYAIA

SEQ ID NO: 108 - protein, 10 aa, synthetic construct
  REGION 1..10: note = Synthetic Sequence
GRTFSSYTVA

SEQ ID NO: 109 - protein, 10 aa, synthetic construct
  REGION 1..10: note = Synthetic Sequence
GFPFDDYAIA

SEQ ID NO: 110 - protein, 10 aa, synthetic construct
  REGION 1..10: note = Synthetic Sequence
GRTFSSYVMG

SEQ ID NO: 111 - protein, 10 aa, synthetic construct
  REGION 1..10: note = Synthetic Sequence
GRTLSSNPMA

SEQ ID NO: 112 - protein, 10 aa, synthetic construct
  REGION 1..10: note = Synthetic Sequence
GFTFDNYAIG

SEQ ID NO: 113 - protein, 10 aa, synthetic construct
  REGION 1..10: note = Synthetic Sequence
GRAFSSYFMG

SEQ ID NO: 114 - protein, 10 aa, synthetic construct
  REGION 1..10: note = Synthetic Sequence
TPTFSSYNMG

SEQ ID NO: 115 - protein, 10 aa, synthetic construct
  REGION 1..10: note = Synthetic Sequence
GFTFDDYAIA

SEQ ID NO: 116 - protein, 10 aa, synthetic construct
  REGION 1..10: note = Synthetic Sequence
GGTFSGYIMG

SEQ ID NO: 117 - protein, 10 aa, synthetic construct
  REGION 1..10: note = Synthetic Sequence
GRSFSSYTIA

SEQ ID NO: 118 - protein, 10 aa, synthetic construct
  REGION 1..10: note = Synthetic Sequence
GFSSDDYTIG

SEQ ID NO: 119 - protein, 10 aa, synthetic construct
  REGION 1..10: note = Synthetic Sequence
GFTFDDYTIG

SEQ ID NO: 120 - protein, 10 aa, synthetic construct
  REGION 1..10: note = Synthetic Sequence
GFSSDDYTIG

SEQ ID NO: 121 - protein, 10 aa, synthetic construct
  REGION 1..10: note = Synthetic Sequence
GFTFDQYTIA

SEQ ID NO: 122 - protein, 10 aa, synthetic construct
  REGION 1..10: note = Synthetic Sequence
GRTFSSYAMA

SEQ ID NO: 123 - protein, 10 aa, synthetic construct
  REGION 1..10: note = Synthetic Sequence
GFAFDGYAIG

SEQ ID NO: 124 - protein, 10 aa, synthetic construct
  REGION 1..10: note = Synthetic Sequence
GFSSDDYTIA

SEQ ID NO: 125 - protein, 10 aa, synthetic construct
  REGION 1..10: note = Synthetic Sequence
GFSSDDYTIG

SEQ ID NO: 126 - protein, 10 aa, synthetic construct
  REGION 1..10: note = Synthetic Sequence
GFTFDDYTIG

SEQ ID NO: 127 - protein, 10 aa, synthetic construct
  REGION 1..10: note = Synthetic Sequence
GFSSDDYTIG

SEQ ID NO: 128 - protein, 10 aa, synthetic construct
  REGION 1..10: note = Synthetic Sequence
GFSSDDYTIG

SEQ ID NO: 129 - protein, 10 aa, synthetic construct
  REGION 1..10: note = Synthetic Sequence
GFSFDDYAIA

SEQ ID NO: 130 - protein, 10 aa, synthetic construct
  REGION 1..10: note = Synthetic Sequence
GFSSDDYTIG

SEQ ID NO: 131 - protein, 10 aa, synthetic construct
  REGION 1..10: note = Synthetic Sequence
GFTGNDLAIG

SEQ ID NO: 132 - protein, 10 aa, synthetic construct
  REGION 1..10: note = Synthetic Sequence
GFSSDDYTIA

SEQ ID NO: 133 - protein, 10 aa, synthetic construct
  REGION 1..10: note = Synthetic Sequence
EGTLSSYGIG

SEQ ID NO: 134 - protein, 10 aa, synthetic construct
  REGION 1..10: note = Synthetic Sequence
GFSSDDYTIA

SEQ ID NO: 135 - protein, 10 aa, synthetic construct
  REGION 1..10: note = Synthetic Sequence
GFTFDDYAIA

SEQ ID NO: 136 - protein, 10 aa, synthetic construct
  REGION 1..10: note = Synthetic Sequence
GLSSDDYTIG

SEQ ID NO: 137 - protein, 10 aa, synthetic construct
  REGION 1..10: note = Synthetic Sequence
GLSSDDYTIG

SEQ ID NO: 138 - protein, 10 aa, synthetic construct
  REGION 1..10: note = Synthetic Sequence
GFSSDDYTIG

SEQ ID NO: 139 - protein, 10 aa, synthetic construct
  REGION 1..10: note = Synthetic Sequence
GFSFDDYTIG

SEQ ID NO: 140 - protein, 10 aa, synthetic construct
  REGION 1..10: note = Synthetic Sequence
GFTFDDYAIA

SEQ ID NO: 141 - protein, 10 aa, synthetic construct
  REGION 1..10: note = Synthetic Sequence
GFTFDDYAIG

SEQ ID NO: 142 - protein, 10 aa, synthetic construct
  REGION 1..10: note = Synthetic Sequence
GFTFGDYTIG

SEQ ID NO: 143 - protein, 10 aa, synthetic construct
  REGION 1..10: note = Synthetic Sequence
EGTFSSYGIG

SEQ ID NO: 144 - protein, 10 aa, synthetic construct
  REGION 1..10: note = Synthetic Sequence
GFSSDDYTIG

SEQ ID NO: 145 - protein, 10 aa, synthetic construct
  REGION 1..10: note = Synthetic Sequence
GVSIGDYNIG

SEQ ID NO: 146 - protein, 10 aa, synthetic construct
  REGION 1..10: note = Synthetic Sequence
GFTFDDYTIA

SEQ ID NO: 147 - protein, 10 aa, synthetic construct
  REGION 1..10: note = Synthetic Sequence
GFTFDDYTIA

SEQ ID NO: 148 - protein, 17 aa, synthetic construct
  REGION 1..17: note = Synthetic Sequence
TINWNGGSAEYAEPVKG

SEQ ID NO: 149 - protein, 17 aa, synthetic construct
  REGION 1..17: note = Synthetic Sequence
CIRVSDGSTYYADPVKG

SEQ ID NO: 150 - protein, 11 aa, synthetic construct
  REGION 1..11: note = Synthetic Sequence
ASITWGGGNTY

SEQ ID NO: 151 - protein, 11 aa, synthetic construct
  REGION 1..11: note = Synthetic Sequence
AATVWTGAGTV

SEQ ID NO: 152 - protein, 11 aa, synthetic construct
  REGION 1..11: note = Synthetic Sequence
AAIGWSADITV

SEQ ID NO: 153 - protein, 11 aa, synthetic construct
  REGION 1..11: note = Synthetic Sequence
AFIDWSGGGTY

SEQ ID NO: 154 - protein, 11 aa, synthetic construct
  REGION 1..11: note = Synthetic Sequence
ATITWGGGSTY

SEQ ID NO: 155 - protein, 11 aa, synthetic construct
  REGION 1..11: note = Synthetic Sequence
AAISWSGGPTV

SEQ ID NO: 156 - protein, 11 aa, synthetic construct
  REGION 1..11: note = Synthetic Sequence
AAITWGGGSTV

SEQ ID NO: 157 - protein, 11 aa, synthetic construct
  REGION 1..11: note = Synthetic Sequence
AAITWSGVSTV

SEQ ID NO: 158 - protein, 11 aa, synthetic construct
  REGION 1..11: note = Synthetic Sequence
GAIMWSGAFTH

SEQ ID NO: 159 - protein, 11 aa, synthetic construct
  REGION 1..11: note = Synthetic Sequence
AAITWGGGSTV

SEQ ID NO: 160 - protein, 11 aa, synthetic construct
  REGION 1..11: note = Synthetic Sequence
SCISSSDRNTY

SEQ ID NO: 161 - protein, 11 aa, synthetic construct
  REGION 1..11: note = Synthetic Sequence
AFIDWSGGGTY

SEQ ID NO: 162 - protein, 11 aa, synthetic construct
  REGION 1..11: note = Synthetic Sequence
AVITWSGDSTY

SEQ ID NO: 163 - protein, 11 aa, synthetic construct
  REGION 1..11: note = Synthetic Sequence
ACISSKDGSTY

SEQ ID NO: 164 - protein, 11 aa, synthetic construct
  REGION 1..11: note = Synthetic Sequence
SGINSIGGSTT

SEQ ID NO: 165 - protein, 11 aa, synthetic construct
  REGION 1..11: note = Synthetic Sequence
AVVTWSGDSTY

SEQ ID NO: 166 - protein, 10 aa, synthetic construct
  REGION 1..10: note = Synthetic Sequence
AKITNFGITS

SEQ ID NO: 167 - protein, 11 aa, synthetic construct
  REGION 1..11: note = Synthetic Sequence
SFISWISDITY

SEQ ID NO: 168 - protein, 11 aa, synthetic construct
  REGION 1..11: note = Synthetic Sequence
AFIDWSGGGTY

SEQ ID NO: 169 - protein, 11 aa, synthetic construct
  REGION 1..11: note = Synthetic Sequence
AVILWSGVSTY

SEQ ID NO: 170 - protein, 11 aa, synthetic construct
  REGION 1..11: note = Synthetic Sequence
AAIVWSGGSTY

SEQ ID NO: 171 - protein, 11 aa, synthetic construct
  REGION 1..11: note = Synthetic Sequence
AAISSSGYHTY

SEQ ID NO: 172 - protein, 11 aa, synthetic construct
  REGION 1..11: note = Synthetic Sequence
SCISSPDGSTY

SEQ ID NO: 173 - protein, 11 aa, synthetic construct
  REGION 1..11: note = Synthetic Sequence
AAVLWSGVSTA

SEQ ID NO: 174 - protein, 11 aa, synthetic construct
  REGION 1..11: note = Synthetic Sequence
VAITWDGSATT

SEQ ID NO: 175 - protein, 11 aa, synthetic construct
  REGION 1..11: note = Synthetic Sequence
AAIGWNGGITY

SEQ ID NO: 176 - protein, 11 aa, synthetic construct
  REGION 1..11: note = Synthetic Sequence
GFITWSGASTY

SEQ ID NO: 177 - protein, 11 aa, synthetic construct
  REGION 1..11: note = Synthetic Sequence
AGINWSGESAD

SEQ ID NO: 178 - protein, 11 aa, synthetic construct
  REGION 1..11: note = Synthetic Sequence
SCIERSDGSTY

SEQ ID NO: 179 - protein, 11 aa, synthetic construct
  REGION 1..11: note = Synthetic Sequence
SCISNTDSSTY

SEQ ID NO: 180 - protein, 11 aa, synthetic construct
  REGION 1..11: note = Synthetic Sequence
SCISNTDSSTY

SEQ ID NO: 181 - protein, 11 aa, synthetic construct
  REGION 1..11: note = Synthetic Sequence
AQISWSAGSIY

SEQ ID NO: 182 - protein, 11 aa, synthetic construct
  REGION 1..11: note = Synthetic Sequence
AGMSWNPGPAV

SEQ ID NO: 183 - protein, 11 aa, synthetic construct
  REGION 1..11: note = Synthetic Sequence
SCISRSDGSTY

SEQ ID NO: 184 - protein, 11 aa, synthetic construct
  REGION 1..11: note = Synthetic Sequence
ANIGWTGDMTY

SEQ ID NO: 185 - protein, 11 aa, synthetic construct
  REGION 1..11: note = Synthetic Sequence
AAIIWSGSMTY

SEQ ID NO: 186 - protein, 11 aa, synthetic construct
  REGION 1..11: note = Synthetic Sequence
SCISNTDSSTY

SEQ ID NO: 187 - protein, 11 aa, synthetic construct
  REGION 1..11: note = Synthetic Sequence
AANTWSGGPTY

SEQ ID NO: 188 - protein, 10 aa, synthetic construct
  REGION 1..10: note = Synthetic Sequence
SCISSDGSTG

SEQ ID NO: 189 - protein, 11 aa, synthetic construct
  REGION 1..11: note = Synthetic Sequence
SCYSSSDGSTG

SEQ ID NO: 190 - protein, 10 aa, synthetic construct
  REGION 1..10: note = Synthetic Sequence
SCISSDGSTG

SEQ ID NO: 191 - protein, 11 aa, synthetic construct
  REGION 1..11: note = Synthetic Sequence
GCIKSSDGTTG

SEQ ID NO: 192 - protein, 11 aa, synthetic construct
  REGION 1..11: note = Synthetic Sequence
SCISNTDSSTY

SEQ ID NO: 193 - protein, 11 aa, synthetic construct
  REGION 1..11: note = Synthetic Sequence
AAIAWSAGSTY

SEQ ID NO: 194 - protein, 11 aa, synthetic construct
  REGION 1..11: note = Synthetic Sequence
SCISSKEGSTY

SEQ ID NO: 195 - protein, 11 aa, synthetic construct
  REGION 1..11: note = Synthetic Sequence
SCISSSDGSTG

SEQ ID NO: 196 - protein, 11 aa, synthetic construct
  REGION 1..11: note = Synthetic Sequence
SCYSSRDGTTG

SEQ ID NO: 197 - protein, 10 aa, synthetic construct
  REGION 1..10: note = Synthetic Sequence
SCISSDGSTG

SEQ ID NO: 198 - protein, 11 aa, synthetic construct
  REGION 1..11: note = Synthetic Sequence
SCYSSSDGSTG

SEQ ID NO: 199 - protein, 11 aa, synthetic construct
  REGION 1..11: note = Synthetic Sequence
SCFSSSDGSTG

SEQ ID NO: 200 - protein, 11 aa, synthetic construct
  REGION 1..11: note = Synthetic Sequence
SCISNTDSSTF

SEQ ID NO: 201 - protein, 11 aa, synthetic construct
  REGION 1..11: note = Synthetic Sequence
SCYSSSDGSTG

SEQ ID NO: 202 - protein, 11 aa, synthetic construct
  REGION 1..11: note = Synthetic Sequence
SCISNTDSSTY

SEQ ID NO: 203 - protein, 11 aa, synthetic construct
  REGION 1..11: note = Synthetic Sequence
SCISSSDGSTG

SEQ ID NO: 204 - protein, 11 aa, synthetic construct
  REGION 1..11: note = Synthetic Sequence
GGINWSGDSTD

SEQ ID NO: 205 - protein, 11 aa, synthetic construct
  REGION 1..11: note = Synthetic Sequence
SCFSSSDGSAG

SEQ ID NO: 206 - protein, 11 aa, synthetic construct
  REGION 1..11: note = Synthetic Sequence
SCISNTDSSTY

SEQ ID NO: 207 - protein, 11 aa, synthetic construct
  REGION 1..11: note = Synthetic Sequence
SCFSTRDGNAG

SEQ ID NO: 208 - protein, 11 aa, synthetic construct
  REGION 1..11: note = Synthetic Sequence
SCFSSRDGSTG

SEQ ID NO: 209 - protein, 11 aa, synthetic construct
  REGION 1..11: note = Synthetic Sequence
SCFSSRDGSTG

SEQ ID NO: 210 - protein, 10 aa, synthetic construct
  REGION 1..10: note = Synthetic Sequence
SCISSDGSTG

SEQ ID NO: 211 - protein, 11 aa, synthetic construct
  REGION 1..11: note = Synthetic Sequence
SCISNTDSSTY

SEQ ID NO: 212 - protein, 11 aa, synthetic construct
  REGION 1..11: note = Synthetic Sequence
SCISSPDGSTY

SEQ ID NO: 213 - protein, 11 aa, synthetic construct
  REGION 1..11: note = Synthetic Sequence
SCYSSSDGNTG

SEQ ID NO: 214 - protein, 11 aa, synthetic construct
  REGION 1..11: note = Synthetic Sequence
GGINWSGDSTD

SEQ ID NO: 215 - protein, 11 aa, synthetic construct
  REGION 1..11: note = Synthetic Sequence
SCFSSSDGSTG

SEQ ID NO: 216 - protein, 11 aa, synthetic construct
  REGION 1..11: note = Synthetic Sequence
SCISSGDGTTY

SEQ ID NO: 217 - protein, 10 aa, synthetic construct
  REGION 1..10: note = Synthetic Sequence
SCISSDGSTG

SEQ ID NO: 218 - protein, 10 aa, synthetic construct
  REGION 1..10: note = Synthetic Sequence
SCISSDGSTG

SEQ ID NO: 219 - protein, 11 aa, synthetic construct
  REGION 1..11: note = Synthetic Sequence
KDADLVWYNLS

SEQ ID NO: 220 - protein, 11 aa, synthetic construct
  REGION 1..11: note = Synthetic Sequence
KDADLVWYNLR

SEQ ID NO: 221 - protein, 24 aa, synthetic construct
  REGION 1..24: note = Synthetic Sequence
AGSLYTCVQSIVVVPARPYYDMDY

SEQ ID NO: 222 - protein, 19 aa, synthetic construct
  REGION 1..19: note = Synthetic Sequence
AKGLRNSDWDLRRGYEYDY

SEQ ID NO: 223 - protein, 25 aa, synthetic construct
  REGION 1..25: note = Synthetic Sequence
ADQASVPPPYGSERYDIASPSEYDY

SEQ ID NO: 224 - protein, 21 aa, synthetic construct
  REGION 1..21: note = Synthetic Sequence
ANSRAYYSSSYDLGRLASYDY

SEQ ID NO: 225 - protein, 16 aa, synthetic construct
  REGION 1..16: note = Synthetic Sequence
AAQRLGSVTDYTKYDY

SEQ ID NO: 226 - protein, 21 aa, synthetic construct
  REGION 1..21: note = Synthetic Sequence
ASVKVVAGSGIDISGSRNYDY

SEQ ID NO: 227 - protein, 21 aa, synthetic construct
  REGION 1..21: note = Synthetic Sequence
AKRLDYSATDKGVDLSDEYDY

SEQ ID NO: 228 - protein, 19 aa, synthetic construct
  REGION 1..19: note = Synthetic Sequence
AAGGSGRLRDLKVGQNYDY

SEQ ID NO: 229 - protein, 23 aa, synthetic construct
  REGION 1..23: note = Synthetic Sequence
ADSPPRTYSSGSVNLEDGSEYDY

SEQ ID NO: 230 - protein, 21 aa, synthetic construct
  REGION 1..21: note = Synthetic Sequence
VIPGRGSALPIDVGKSDEYEY

SEQ ID NO: 231 - protein, 19 aa, synthetic construct
  REGION 1..19: note = Synthetic Sequence
AAGASGRLRDLKVGQNYDY

SEQ ID NO: 232 - protein, 23 aa, synthetic construct
  REGION 1..23: note = Synthetic Sequence
ADGNVWSPPICGSAGPPPGGMDY

SEQ ID NO: 233 - protein, 16 aa, synthetic construct
  REGION 1..16: note = Synthetic Sequence
AAQRLGSVTDYTKYDY

SEQ ID NO: 234 - protein, 22 aa, synthetic construct
  REGION 1..22: note = Synthetic Sequence
AIPPRAYSGGSYSLKDQSKYEY

SEQ ID NO: 235 - protein, 23 aa, synthetic construct
  REGION 1..23: note = Synthetic Sequence
ADGNVWSPPICSSAGPPPGGMDY

SEQ ID NO: 236 - protein, 9 aa, synthetic construct
  REGION 1..9: note = Synthetic Sequence
KSRSSYSNN

SEQ ID NO: 237 - protein, 22 aa, synthetic construct
  REGION 1..22: note = Synthetic Sequence
AMPPRAYTGRSVSLKDQSKYEY

SEQ ID NO: 238 - protein, 13 aa, synthetic construct
  REGION 1..13: note = Synthetic Sequence
LDTTGWGPPPYQY

SEQ ID NO: 239 - protein, 22 aa, synthetic construct
  REGION 1..22: note = Synthetic Sequence
AHPPDPSRGGEWRLQTPSEYDY

SEQ ID NO: 240 - protein, 16 aa, synthetic construct
  REGION 1..16: note = Synthetic Sequence
AAQRLGSVTDYTKYDY

SEQ ID NO: 241 - protein, 24 aa, synthetic construct
  REGION 1..24: note = Synthetic Sequence
VPRSHFTTAQDMGQDMGAPSWYEY

SEQ ID NO: 242 - protein, 22 aa, synthetic construct
  REGION 1..22: note = Synthetic Sequence
AVLIRYYSGGYQGLSDANEYDY

SEQ ID NO: 243 - protein, 17 aa, synthetic construct
  REGION 1..17: note = Synthetic Sequence
VVKYLSGSYSYAGQYNF

SEQ ID NO: 244 - protein, 23 aa, synthetic construct
  REGION 1..23: note = Synthetic Sequence
ADFNVWSPPICGSVGPPPGGMDY

SEQ ID NO: 245 - protein, 21 aa, synthetic construct
  REGION 1..21: note = Synthetic Sequence
AHESTYYSGTYYLTDPRRYVY

SEQ ID NO: 246 - protein, 19 aa, synthetic construct
  REGION 1..19: note = Synthetic Sequence
AVPARGLTMDLENSDIYDH

SEQ ID NO: 247 - protein, 22 aa, synthetic construct
  REGION 1..22: note = Synthetic Sequence
AATLQVTGSYYLDLSTVDIYDN

SEQ ID NO: 248 - protein, 20 aa, synthetic construct
  REGION 1..20: note = Synthetic Sequence
ATLFRSNGPKDLSSGYEYDY

SEQ ID NO: 249 - protein, 13 aa, synthetic construct
  REGION 1..13: note = Synthetic Sequence
AGESGVWVGGLDY

SEQ ID NO: 250 - protein, 22 aa, synthetic construct
  REGION 1..22: note = Synthetic Sequence
VGSANSGEFRFGWVLKPDLYNY

SEQ ID NO: 251 - protein, 23 aa, synthetic construct
  REGION 1..23: note = Synthetic Sequence
ADGNVWSPPICGSAGPPPGGMDY

SEQ ID NO: 252 - protein, 23 aa, synthetic construct
  REGION 1..23: note = Synthetic Sequence
ADGNVWSPPICGSAGPPPGGMDY

SEQ ID NO: 253 - protein, 20 aa, synthetic construct
  REGION 1..20: note = Synthetic Sequence
ERGYAYCSDDGCQRTQDYDY

SEQ ID NO: 254 - protein, 21 aa, synthetic construct
  REGION 1..21: note = Synthetic Sequence
GAARAWWSGSYDYTRMNNYDY

SEQ ID NO: 255 - protein, 23 aa, synthetic construct
  REGION 1..23: note = Synthetic Sequence
AETSADSGEFRFGWVLKPSLYDY

SEQ ID NO: 256 - protein, 21 aa, synthetic construct
  REGION 1..21: note = Synthetic Sequence
AAGSAYSGSYWNITMAANYDY

SEQ ID NO: 257 - protein, 18 aa, synthetic construct
  REGION 1..18: note = Synthetic Sequence
AQRIFGAQPMDLSGDYEY

SEQ ID NO: 258 - protein, 23 aa, synthetic construct
  REGION 1..23: note = Synthetic Sequence
ADGNVWSPPICGSAGPPPGGMDY

SEQ ID NO: 259 - protein, 18 aa, synthetic construct
  REGION 1..18: note = Synthetic Sequence
ARDYRGIKDLDLKGDYDY

SEQ ID NO: 260 - protein, 25 aa, synthetic construct
  REGION 1..25: note = Synthetic Sequence
ADFNVWSPPICGSIWYGPPPRGMDY

SEQ ID NO: 261 - protein, 25 aa, synthetic construct
  REGION 1..25: note = Synthetic Sequence
ADSNVWSPPICGSRWYGPPPGGMAY

SEQ ID NO: 262 - protein, 25 aa, synthetic construct
  REGION 1..25: note = Synthetic Sequence
ADFNVWSPPICGSNWYGPPPGGMDY

SEQ ID NO: 263 - protein, 25 aa, synthetic construct
  REGION 1..25: note = Synthetic Sequence
ADFNVWSPPICGSIWYGPPPGGMDY

SEQ ID NO: 264 - protein, 23 aa, synthetic construct
  REGION 1..23: note = Synthetic Sequence
ADGNVWSPPICGSAGPPPGGMDY

SEQ ID NO: 265 - protein, 17 aa, synthetic construct
  REGION 1..17: note = Synthetic Sequence
ARIITVATMRLDSDYDY

SEQ ID NO: 266 - protein, 23 aa, synthetic construct
  REGION 1..23: note = Synthetic Sequence
ADGNVWSPPICGSAGPPPGGMDY

SEQ ID NO: 267 - protein, 25 aa, synthetic construct
  REGION 1..25: note = Synthetic Sequence
ADSNVWSPPICGRTWYGPPPGGMDY

SEQ ID NO: 268 - protein, 25 aa, synthetic construct
  REGION 1..25: note = Synthetic Sequence
ADFNVWSPPICGSIWYGPPPGGMAY

SEQ ID NO: 269 - protein, 25 aa, synthetic construct
  REGION 1..25: note = Synthetic Sequence
ADFNVWSPPICGSNWYGPPPGGMDY

SEQ ID NO: 270 - protein, 25 aa, synthetic construct
  REGION 1..25: note = Synthetic Sequence
ADFNVWSPPICGSSWYGPPPGGMDY

SEQ ID NO: 271 - protein, 25 aa, synthetic construct
  REGION 1..25: note = Synthetic Sequence
ADFNVWSPPICGSRWYGPPPGGMEY

SEQ ID NO: 272 - protein, 23 aa, synthetic construct
  REGION 1..23: note = Synthetic Sequence
ADGNVWSPPICGSAGPPPGGMDY

SEQ ID NO: 273 - protein, 25 aa, synthetic construct
  REGION 1..25: note = Synthetic Sequence
ADFNVWSPPICGSRWYGPPPGGMAY

SEQ ID NO: 274 - protein, 23 aa, synthetic construct
  REGION 1..23: note = Synthetic Sequence
ADGNVWSPPICGSAGPPPGGMDY

SEQ ID NO: 275 - protein, 25 aa, synthetic construct
  REGION 1..25: note = Synthetic Sequence
ADSNVWSPPICGKTWYGPPPGGMDY

SEQ ID NO: 276 - protein, 13 aa, synthetic construct
  REGION 1..13: note = Synthetic Sequence
AGESGVWVGGLDY

SEQ ID NO: 277 - protein, 25 aa, synthetic construct
  REGION 1..25: note = Synthetic Sequence
ADSNVWSPPICGSTWYGPPPGGMAY

SEQ ID NO: 278 - protein, 23 aa, synthetic construct
  REGION 1..23: note = Synthetic Sequence
ADGNVWSPPICGSAGPPPGGMDY

SEQ ID NO: 279 - protein, 25 aa, synthetic construct
  REGION 1..25: note = Synthetic Sequence
ADFNVWSPPICGSRWYGPPPGGMDY

SEQ ID NO: 280 - protein, 25 aa, synthetic construct
  REGION 1..25: note = Synthetic Sequence
ADFNVWSPPICGSRWYGPPPGGMDY

SEQ ID NO: 281 - protein, 25 aa, synthetic construct
  REGION 1..25: note = Synthetic Sequence
ADFNVWSPPICGSRWYGPPPGGMDY

SEQ ID NO: 282 - protein, 25 aa, synthetic construct
  REGION 1..25: note = Synthetic Sequence
ADFNVWSPPICGSIWYGPPPGGMDY

SEQ ID NO: 283 - protein, 23 aa, synthetic construct
  REGION 1..23: note = Synthetic Sequence
ADGNVWSPPICGSAGPPPGGMDY

SEQ ID NO: 284 - protein, 23 aa, synthetic construct
  REGION 1..23: note = Synthetic Sequence
ADFNVWSPPICGSVGPPPGGMDY

SEQ ID NO: 285 - protein, 25 aa, synthetic construct
  REGION 1..25: note = Synthetic Sequence
ADFNVWSPPICGSSWYGPPPGGMAY

SEQ ID NO: 286 - protein, 13 aa, synthetic construct
  REGION 1..13: note = Synthetic Sequence
AGESGVWVGGLDY

SEQ ID NO: 287 - protein, 25 aa, synthetic construct
  REGION 1..25: note = Synthetic Sequence
ADFNVWSPPICGSSWYGPPPGGMEY

SEQ ID NO: 288 - protein, 23 aa, synthetic construct
  REGION 1..23: note = Synthetic Sequence
ADGNVWSPPICGSAGPPPGGMDY

SEQ ID NO: 289 - protein, 25 aa, synthetic construct
  REGION 1..25: note = Synthetic Sequence
ADFNVWSPPICSSNWYGPPPRGMDY

SEQ ID NO: 290 - protein, 25 aa, synthetic construct
  REGION 1..25: note = Synthetic Sequence
ADFNVWSPPICGSIWYGPPPRGMDY

SEQ ID NO: 291 - protein, 132 aa, synthetic construct
  REGION 1..132: note = Synthetic Sequence
QVQLQESGGGSVQPGGSLRLSCAASGFTFDDYAMSWVRQVPGKGLEWVSTINWNGGSAEYAEPVKGRFTISRDNAKNTVYLQMNSLKLEDTAVYYCAKDADLVWYNLSTGQGTQVTVSSAAAYPYDVPDYGS

SEQ ID NO: 292 - protein, 132 aa, synthetic construct
  REGION 1..132: note = Synthetic Sequence
QVQLQESGGGLVQPGGSLRLSCAASGFTFDDYAMSWVRQVPGKGLEWVSTINWNGGSAEYAEPVKGRFTISRDNAKNTVYLQMNSLKLEDTAVYYCAKDADLVWYNLRTGQGTQVTVSSAAAYPYDVPDYGS

SEQ ID NO: 293 - protein, 145 aa, synthetic construct
  REGION 1..145: note = Synthetic Sequence
QVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVVVPARPYYDMDYWGKGTQVTVSSAAAYPYDVPDYGS

SEQ ID NO: 294 - protein, 146 aa, synthetic construct
  REGION 1..146: note = Synthetic Sequence
QVQLQESGGGLVQAGGSLRLSCAASGRSFSSYTLAWFRQAPGKEREFVASITWGGGNTYYPDSVKGRFTISRDDAKNTVYLQMNSLKPEDTAVYYCAAKGLRNSDWDLRRGYEYDYWGQGTQVTVSSAAAYPYDVPDYGSHHHHHH

SEQ ID NO: 295 - protein, 152 aa, synthetic construct
  REGION 1..152: note = Synthetic Sequence
QVQLQESGGGLVQDGGSLRLSCAFSGRTFSSYTMGWFRQGPGKEREFVAATVWTGAGTVYADSVKGRFTISRDNAKNTVYLQMNSLRPEDTAVYYCAADQASVPPPYGSERYDIASPSEYDYWGQGTQVTVSSAAAYPYDVPDYGSHHHHHH

SEQ ID NO: 296 - protein, 148 aa, synthetic construct
  REGION 1..148: note = Synthetic Sequence
QVQLQESGGGLVQAGASLRLSCAASGRTFSSYIMGWFRQAPGKEREFVAAIGWSADITVYADSVKGRFTISRDNAENMVYLQMNSLNPEDTAVYYCAANSRAYYSSSYDLGRLASYDYWGQGTQVTVSSAAAYPYDVPDYGSHHHHHH

SEQ ID NO: 297 - protein, 143 aa, synthetic construct
  REGION 1..143: note = Synthetic Sequence
QVQLQESGGGLVQAGGSLRLSCAASGRTFSSYTMGWFRQAPGKEREFVAFIDWSGGGTYYDDSVKGRFTISRDNAENTVYLQMNNLEPEDTAVYYCAAAQRLGSVTDYTKYDYWGQGTQVTVSSAAAYPYDVPDYGSHHHHHH

SEQ ID NO: 298 - protein, 152 aa, synthetic construct
  REGION 1..152: note = Synthetic Sequence
QVQLQESGGGLVQAGGSLRLSCAASGRTSGRTFSSYTMGWFRQAPGKEREFVATITWGGGSTYYADSVKGRFTISRDNANNTVYLQMNSLKPEDTAVYYCAASVKVVAGSGIDISGSRNYDYWGQGTQVTVSSAAAYPYDVPDYGSHHHHHH

SEQ ID NO: 299 - protein, 148 aa, synthetic construct
  REGION 1..148: note = Synthetic Sequence
QVQLQESGGGLVQPGGSLRLSCLASGRTFSSYAMGWFRQAPGKEREFVAAISWSGGPTVYADHVKGRFTISRDNAKNTVYLQVNSLKPEDTADYYCAAKRLDYSATDKGVDLSDEYDYWGQGTQVTVSSAAAYPYDVPDYGSHHHHHH

SEQ ID NO: 300 - protein, 146 aa, synthetic construct
  REGION 1..146: note = Synthetic Sequence
QVQLQESGGGLVQAGDSLRLSCAASGLTFSNYIMGWFRQAPGKEREFVAAITWGGGSTVYADSVEGRFTISRDGTKNTVSLQMNSLLPEDTAVYYCAAAGGSGRLRDLKVGQNYDYWGQGTQVTVSSAAAYPYDVPDYGSHHHHHH

SEQ ID NO: 301 - protein, 150 aa, synthetic construct
  REGION 1..150: note = Synthetic Sequence
QVQLQESGGGLVQAGGSLRLSCAASGRTFSSYTMGWFRQAPGREREFVAAITWSGVSTVYTDSVKGRFTVSRDNAKNTVYLQMNSLKPEDTAVYYCAADSPPRTYSSGSVNLEDGSEYDYWGQGTQVTVSSAAAYPYDVPDYGSHHHHHH

SEQ ID NO: 302 - protein, 148 aa, synthetic construct
  REGION 1..148: note = Synthetic Sequence
QVQLQESGGGLVQAGGSLRLSCAASGRTFSSDTMGWFRQAPGKEREFVGAIMWSGAFTHYADSVKGRFTISRDNAKNTVYLQMNALKPEDTAVYYCAVIPGRGSALPIDVGKSDEYEYWGQGTQVTVSSAAAYPYDVPDYGSHHHHHH

SEQ ID NO: 303 - protein, 146 aa, synthetic construct
  REGION 1..146: note = Synthetic Sequence
QVQLQESGGGLVQAGDSLRLSCAASGLTFSNYIMGWFRQAPGKEREFVAAITWGGGSTVYADSVEGRFTISRDGTKNTVSLQMNSLQPEDTAVYYCAAAGASGRLRDLKVGQNYDYWGQGTQVTVSSAAAYPYDVPDYGSHHHHHH

SEQ ID NO: 304 - protein, 150 aa, synthetic construct
  REGION 1..150: note = Synthetic Sequence
QVQLQESGGGLVQAGGSLRLSCAGSGFTLDYYGIGWFRQAPGKEREGVSCISSSDRNTYYADSVKGRFTISGDNAKNTVYLQMNNLKPEDTAVYYCAADGNVWSPPICGSAGPPPGGMDYWGKGTQVTVSSAAAYPYDVPDYGSHHHHHH

SEQ ID NO: 305 - protein, 143 aa, synthetic construct
  REGION 1..143: note = Synthetic Sequence
QVQLQESGGGLVQAGGSLRLSCVASGHTFSSYTMGWFRQAPGKEREFVAFIDWSGGGTYYANSVKGRFTISRDNAENTVYLQMNNLKPEDTAVYYCAAAQRLGSVTDYTKYDYWGQGTQVTVSSAAAYPYDVPDYGSHHHHHH

SEQ ID NO: 306 - protein, 149 aa, synthetic construct
  REGION 1..149: note = Synthetic Sequence
QVQLQESGGGLVQAGGSLRLSCAASGRTFSSYVIGWFRQAPGKEREFVAVITWSGDSTYSSDSLKGRFTISRDNAKNTVYLQMNALNPEDTAVYYCAAIPPRAYSGGSYSLKDQSKYEYWGQGTQVTVSSAAAYPYDVPDYGSHHHHHH

SEQ ID NO: 307 - protein, 149 aa, synthetic construct
  REGION 1..149: note = Synthetic Sequence
QVQLQESGGGLVQAEGSLKLSCISGFAFDGYAIGWFRQAPGKEREGVACISSKDGSTYYADSVKGRFTMSVDKTKNTVYLQMSSLKPEDTAVYYCAADGNVWSPPICSSAGPPPGGMDYWGKGTQVTVSSAAAYPYDVPDYGSHHHHHH

SEQ ID NO: 308 - protein, 139 aa, synthetic construct
  REGION 1..139: note = Synthetic Sequence
QVQLQESGGGLVQPGGSLTLSCAASGFAFGFFDMTWVRQAPGKGLEWVSGINSIGGSTTYADSVKGRFTISRDNAKNELYLQMNSLKPDDTAVYYCAKSRSSYSNNWRPPGQGTQVTVSSAAAYPYDVPDYGSHHHHHH

SEQ ID NO: 309 - protein, 149 aa, synthetic construct
  REGION 1..149: note = Synthetic Sequence
QVQLQESGGGLVQARGSLTLSCAASGRTFSNYVIGWFRQAPGEEREFVAVVTWSGDSTYSSDSLKGRFTISRDNAKNTVYLQMNNLNPEDTAVYYCAAMPPRAYTGRSVSLKDQSKYEYWGQGTQVTVSSAAAYPYDVPDYGSHHHHHH

SEQ ID NO: 310 - protein, 139 aa, synthetic construct
  REGION 1..139: note = Synthetic Sequence
QVQLQESGGGLVQPGGSLRLSCAASGSIFSINVMGWYRQTPGKERELVAKITNFGITSYADSAQGRFTISRGNAKNTVYLQMNSLKPEDTAVYYCNLDTTGWGPPPYQYWGQGTQVTVSSAAAYPYDVPDYGSHHHHHH

SEQ ID NO: 311 - protein, 149 aa, synthetic construct
  REGION 1..149: note = Synthetic Sequence
QVQLQESGGGLVQAGASLRLSCAASGRTFSNYNVGWFRQAPGKEREFVSFISWISDITYYSDSVKGRFIISRDNAKNMVYLQMNSLKPEDTAVYYCAAHPPDPSRGGEWRLQTPSEYDYWGQGTQVTVSSAAAYPYDVPDYGSHHHHHH

SEQ ID NO: 312 - protein, 143 aa, synthetic construct
  REGION 1..143: note = Synthetic Sequence
QVQLQESGGGLVQAGGSLRLSCAASGHTFSSYTMGWFRQAPGKEREFVAFIDWSGGGTYYADSVKGRFTISRDNAENTVYLQMNNLKPEDTAVYYCAAAQRLGSVTDYTKYDYWGQGTQVTVSSAAAYPYDVPDYGSHHHHHH

SEQ ID NO: 313 - protein, 151 aa, synthetic construct
  REGION 1..151: note = Synthetic Sequence
QVQLQESGGGLVQAGGSLRLSCAASGRTFSTYPVGWFRQAPGKEREFVAVILWSGVSTYYADSVKGRFTISRDNAQNTVYLQMDSLKPEDTAVYYCAVPRSHFTTAQDMGQDMGAPSWYEYWGQGTQVTVSSAAAYPYDVPDYGSHHHHHH

SEQ ID NO: 314 - protein, 149 aa, synthetic construct
  REGION 1..149: note = Synthetic Sequence
QVQLQESGGGLVQAGGSLRLSCAASGRTFSNYAMGWFRQAPGKEREFVAAIVWSGGSTYYADSVKGRFTISRDNAKNTVYLQMNSLKPEDTAVYYCAAVLIRYYSGGYQGLSDANEYDYWGQGTQVTVSSAAAYPYDVPDYGSHHHHHH

SEQ ID NO: 315 - protein, 144 aa, synthetic construct
  REGION 1..144: note = Synthetic Sequence
QVQLQESGGGLVQAGASLRLSCSASGRTFSDYRMGWFRQAPGKEREWVAAISSSGYHTYYADSVKGRFTISRDNAKNTGYLQMSSLKPEDTAVYYCAVVKYLSGSYSYAGQYNFWGQGTQVTVSSAAAYPYDVPDYGSHHHHHH

SEQ ID NO: 316 - protein, 150 aa, synthetic construct
  REGION 1..150: note = Synthetic Sequence
QVQLQESGGGLVQAGDSLKLSCAASGLTFSNYIMAWFRQAPGKEREGVSCISSPDGSTYYADSVKGRFTISSDNAKNTVYLQMNSLKPEDTAVYYCAADFNVWSPPICGSVGPPPGGMDYWGKGTQVTVSSAAAYPYDVPDYGSHHHHHH

SEQ ID NO: 317 - protein, 148 aa, synthetic construct
  REGION 1..148: note = Synthetic Sequence
QVQLQESGGGLVQAGGSLRLSCAASGRTFSNSVMGWFRQPPGKEREFVAAVLWSGVSTAYADSVKGRFTISRDNAKNTVYLQMNNLKPDDTAVYYCAAHESTYYSGTYYLTDPRRYVYWGQGTQVTVSSAAAYPYDVPDYGSHHHHHH

SEQ ID NO: 318 - protein, 146 aa, synthetic construct
  REGION 1..146: note = Synthetic Sequence
QVQLQESGGGLVQAGGSLRLSCVGDGRTFSSYIIGWFRQAPGNEREFVVAITWDGSATTYADSVKGRFTVSRDSAKNTAYLQMNSLKPEDTAVYYCAAVPARGLTMDLENSDIYDHWGRGTQVTVSSAAAYPYDVPDYGSHHHHHH

SEQ ID NO: 319 - protein, 149 aa, synthetic construct
  REGION 1..149: note = Synthetic Sequence
QVQLQESGGGLVQAGGSLRLSCAASGRTFSSYVMGWFRQALGKEREFVAAIGWNGGITYYADSVKGRFAISRDNAKNTVYLQMNSLKPEDTAVYYCAAATLQVTGSYYLDLSTVDIYDNWGQGTQVTVSSAAAYPYDVPDYGSHHHHHH

SEQ ID NO: 320 - protein, 147 aa, synthetic construct
  REGION 1..147: note = Synthetic Sequence
QVQLQESGGGLVQAGGSLRLSCAASGGTFSNYVMGWFRQAPGKEREFVGFITWSGASTYYADSVKGRFTISRDNAENTVYLQMNSLKPEDTAVYYCAATLFRSNGPKDLSSGYEYDYWGQGTQVTVSSAAAYPYDVPDYGSHHHHHH

SEQ ID NO: 321 - protein, 140 aa, synthetic construct
  REGION 1..140: note = Synthetic Sequence
  VAR_SEQ 112: note = Xaa can be any naturally occurring amino acid
QVQLQESGGGLVQAGDSLRLTCTASGRTFSNYGIGWFRQAPGKEREFVAGINWSGESADYADSVKGRFTISRDNAKNTVYLQMNSLKPEDTAVYYCAAGESGVWVGGLDYWXQGTQVTVSSAAAYPYDVPDYGSHHHHHH

SEQ ID NO: 322 - protein, 149 aa, synthetic construct
  REGION 1..149: note = Synthetic Sequence
QVQLQESGGGLVQAGGSLRLSCAASGFTFDDYAIAWFRQAPGKEREGVSCIERSDGSTYYADSVKGRFTISSDNAKNTVYLQMNSLKPEDTAVYYCAVGSANSGEFRFGWVLKPDLYNYWGQGTQVTVSSAAAYPYDVPDYGSHHHHHH

SEQ ID NO: 323 - protein, 150 aa, synthetic construct
  REGION 1..150: note = Synthetic Sequence
QVQLQESGGGLVQAGGSLRLSCTASGRTFSSYTVAWFRQSPGKEREGISCISNTDSSTYYADSVKGRFTISSDNAKSTVHLQMSSLKPEDTAVYYCAADGNVWSPPICGSAGPPPGGMDYWGKGTQVTVSSAAAYPYDVPDYGSHHHHHH

SEQ ID NO: 324 - protein, 150 aa, synthetic construct
  REGION 1..150: note = Synthetic Sequence
QVQLQESGGGLVQPGGSLRLSCATFGFPFDDYAIAWFRQAPGKEREGVSCISNTDSSTYYADSVKGRFTISSDNAKNTVHLQMSSLKPEDTAVYYCAADGNVWSPPICGSAGPPPGGMDYWGKGTQVTVSSAAAYPYDVPDYGSHHHHHH

SEQ ID NO: 325 - protein, 147 aa, synthetic construct
  REGION 1..147: note = Synthetic Sequence
QVQLQESGGGLVQAGGSLRLSCAASGRTFSSYVMGWFRQAPGKEREFVAQISWSAGSIYYADSVKGRFTISNDNAKRTVYLQMNSLKPEDTAVYYCAERGYAYCSDDGCQRTQDYDYWGQGTQVTVSSAAAYPYDVPDYGSHHHHHH

SEQ ID NO: 326 - protein, 148 aa, synthetic construct
  REGION 1..148: note = Synthetic Sequence
QVQLQESGGGLVQAGGSLRLSCAASGRTLSSNPMAWFRQAAGKEREFVAGMSWNPGPAVYADSVKGRFTISRDSAENTVYLQMNSLKPEDTAVYYCAGAARAWWSGSYDYTRMNNYDYWGPGTQVTVSSAAAYPYDVPDYGSHHHHHH

SEQ ID NO: 327 - protein, 150 aa, synthetic construct
  REGION 1..150: note = Synthetic Sequence
QVQLQESGGGLVQAGGSLRLSCAVSGFTFDNYAIGWFRQAPGKEREGVSCISRSDGSTYYADSVRGRFTISSDNAKNTVYLQMNSLKPEDTAVYYCAAETSADSGEFRFGWVLKPSLYDYWGQGTQVTVSSAAAYPYDVPDYGSHHHHHH

SEQ ID NO: 328 - protein, 148 aa, synthetic construct
  REGION 1..148: note = Synthetic Sequence
QVQLQESGGGLVQAGGSLRLSCAASGRAFSSYFMGWFRQTPGKEREFVANIGWTGDMTYYADSVKGRFTISRDNAKNTVYLQMNSLKPEDTAVYYCAAAGSAYSGSYWNITMAANYDYWGQGTQVTVSSAAAYPYDVPDYGSHHHHHH

SEQ ID NO: 329 - protein, 145 aa, synthetic construct
  REGION 1..145: note = Synthetic Sequence
QVQLQESGGGLVQAGGSLRLSCAASTPTFSSYNMGWFRQAPGKEREFVAAIIWSGSMTYYADSMKGRFTVSIDNAKNTVYLQMNSLKPEDTAVYYCAAQRIFGAQPMDLSGDYEYWGQGTQVTVSSAAAYPYDVPDYGSHHHHHH

SEQ ID NO: 330 - protein, 150 aa, synthetic construct
  REGION 1..150: note = Synthetic Sequence
QVQLQESGGGLVQAGGSLRLSCATFGFTFDDYAIAWFRQAPGKEREGISCISNTDSSTYYADSVKGRFTISSDSAKNTVHLQMSSLKPEDTAVYYCAADGNVWSPPICGSAGPPPGGMDYWGKGTQVTVSSAAAYPYDVPDYGSHHHHHH

SEQ ID NO: 331 - protein, 145 aa, synthetic construct
  REGION 1..145: note = Synthetic Sequence
QVQLQESGGGLVQAGGSLRLSCKASGGTFSGYIMGWFRQAPGKEREFVAANTWSGGPTYYSDSVKGRFTISRDNAKNTVYLQMNTLKPEDTAVYQCAARDYRGIKDLDLKGDYDYWGQGTQVTVSSAAAYPYDVPDYGSHHHHHH

SEQ ID NO: 332 - protein, 151 aa, synthetic construct
  REGION 1..151: note = Synthetic Sequence
QVQLQESGGGLVQAGDSLKLSCATSGRSFSSYTIAWFRQAPGKEREGISCISSDGSTGYADSVRGRFTISSDNAKNTVYLQMNSLKPEDTAVYYCAADFNVWSPPICGSIWYGPPPRGMDYWGKGTQVTVSSAAAYPYDVPDYGSHHHHHH

SEQ ID NO: 333 - protein, 152 aa, synthetic construct
  REGION 1..152: note = Synthetic Sequence
QVQLQESGGGLVQAGGYLRLSCAASGFSSDDYTIGWFRQAPGKEREGISCYSSSDGSTGFADSVKGRFTISSDNAKNTVYLQMNNLRPEDTAVYYCAADSNVWSPPICGSRWYGPPPGGMAYWGKGTQVTVSSAAAYPYDVPDYGSHHHHHH

SEQ ID NO: 334 - protein, 151 aa, synthetic construct
  REGION 1..151: note = Synthetic Sequence
QVQLQESGGGLAQVGGSLRLSCTASGFTFDDYTIGWFRQAPGKEREGISCISSDGSTGYADSVKGRFTISSDNAKNTVYLQMNSLKPEDTAVYYCAADFNVWSPPICGSNWYGPPPGGMDYWGKGTQVTVSSAAAYPYDVPDYGSHHHHHH

SEQ ID NO: 335 - protein, 152 aa, synthetic construct
  REGION 1..152: note = Synthetic Sequence
QVQLQESGGGLVQAGGSLRLSCAASGFSSDDYTIGWFRQAPGKEREGIGCIKSSDGTTGYADSVKGRFTISSDNAKNTVYLQMNSLKPEDTAVYYCAADFNVWSPPICGSIWYGPPPGGMDYWGKGTQVTVSSAAAYPYDVPDYGSHHHHHH

SEQ ID NO: 336 - protein, 150 aa, synthetic construct
  REGION 1..150: note = Synthetic Sequence
QVQLQESGGGLAQAGGSLRLSCAASGFTFDQYTIAWFRQAPGKEREGVSCISNTDSSTYYADSVKGRFTISSDNAKNTVYLQMSSLKPEDTAVYYCAADGNVWSPPICGSAGPPPGGMDYWGKGTQVTVSSAAAYPYDVPDYGSHHHHHH

SEQ ID NO: 337 - protein, 144 aa, synthetic construct
  REGION 1..144: note = Synthetic Sequence
QVQLQESGGGLVQAGGSLRLSCAASGRTFSSYAMAWFRQAPGKEREFVAAIAWSAGSTYYADSVKGRFAISRDNAENTVYLQMNSLKPEDTAVYYCAARIITVATMRLDSDYDYWGQGTQVTVSSAAAYPYDVPDYGSHHHHHH

SEQ ID NO: 338 - protein, 150 aa, synthetic construct
  REGION 1..150: note = Synthetic Sequence
QVQLQESGGGLVQAGGSLRLSCAASGFAFDGYAIGWFRQAPGKEREGVSCISSKEGSTYYADSVKGRFTISSDNAKNTVYLQMSSLKPEDTAVYYCAADGNVWSPPICGSAGPPPGGMDYWGKGTQVTVSSAAAYPYDVPDYGSHHHHHH

SEQ ID NO: 339 - protein, 152 aa, synthetic construct
  REGION 1..152: note = Synthetic Sequence
QVQLQESGGGLVQAGGSLRLSCAASGFSSDDYTIAWFRRAPGKEREGISCISSSDGSTGYADSVKGRFTITSDSAKNTVYLQMNSLKPEDTAVYYCAADSNVWSPPICGRTWYGPPPGGMDYWGKGTQVTVSSAAAYPYDVPDYGSHHHHHH

SEQ ID NO: 340 - protein, 152 aa, synthetic construct
  REGION 1..152: note = Synthetic Sequence
QVQLQESGGGLVQPGGSLRLSCAASGFSSDDYTIGWFRQAPGKEREGISCYSSRDGTTGYADSVKGRFTISSDNAKNTVYLQMNSLKPEDTAVYYCAADFNVWSPPICGSIWYGPPPGGMAYWGQGTQVTVSSAAAYPYDVPDYGSHHHHHH

SEQ ID NO: 341 - protein, 151 aa, synthetic construct
  REGION 1..151: note = Synthetic Sequence
QVQLQESGGGLVQAGGSLRLSCAASGFTFDDYTIGWFRQAPGKEREGISCISSDGSTGYADSVKGRFTISSDNAKNTVYLQMNSLKPEDTAVYYCAADFNVWSPPICGSNWYGPPPGGMDYWGKGTQVTVSSAAAYPYDVPDYGSHHHHHH

SEQ ID NO: 342 - protein, 152 aa, synthetic construct
  REGION 1..152: note = Synthetic Sequence
QVQLQESGGGLVQAGGSLRLSCAASGFSSDDYTIGWFRQAPGKEREGISCYSSSDGSTGYADSVKGRFTISSDNAKNTVYLQMNSLKPEDTAVYYCAADFNVWSPPICGSSWYGPPPGGMDYWGKGTQVTVSSAAAYPYDVPDYGSHHHHHH

SEQ ID NO: 343 - protein, 152 aa, synthetic construct
  REGION 1..152: note = Synthetic Sequence
QVQLQESGGGLVQAGGSLRLSCAASGFSSDDYTIGWFRQAPGKEREGISCFSSSDGSTGFADSVKGRFTISSDNATNTVYLEMNSLKPEDTAVYYCAADFNVWSPPICGSRWYGPPPGGMEYWGKGTQVTVSSAAAYPYDVPDYGSHHHHHH

SEQ ID NO: 344 - protein, 150 aa, synthetic construct
  REGION 1..150: note = Synthetic Sequence
QVQLQESGGGLVQAGGSLRLSCATFGFSFDDYAIAWFRQAPGKEREGISCISNTDSSTFYADSVKGRFTISSDNAKNTVHLQMSSLKPEDTAVYYCAADGNVWSPPICGSAGPPPGGMDYWGKGTQVTVSSAAAYPYDVPDYGSHHHHHH

SEQ ID NO: 345 - protein, 152 aa, synthetic construct
  REGION 1..152: note = Synthetic Sequence
QVQLQESGGGLVQAGGSLRLSCAASGFSSDDYTIGWFRQAPGKEREGISCYSSSDGSTGFADSVKGRFTISSDNAKNTVYLQMNSLRPEDTAVYYCAADFNVWSPPICGSRWYGPPPGGMAYWGKGTQVTVSSAAAYPYDVPDYGSHHHHHH

SEQ ID NO: 346 - protein, 151 aa, synthetic construct
  REGION 1..151: note = Synthetic Sequence
QVQLQESGGGLVQVGGSLRLSCTISGFTGNDLAIGWFRQAPGKDQREGISCISNTDSSTYYADSVKGRFTISSDNAKNTVHLQMSSLKPEDTAVYYCAADGNVWSPPICGSAGPPPGGMDYWGKGTQVTVSSAAAYPYDVPDYGSHHHHHH

SEQ ID NO: 347 - protein, 152 aa, synthetic construct
  REGION 1..152: note = Synthetic Sequence
QVQLQESGGGLVQAGGSLRLSCAASGFSSDDYTIAWFRRAPGKEREGISCISSSDGSTGYADSVKGRFTISSDNAKNTVYLQMTSLKPEDTAVYYCAADSNVWSPPICGKTWYGPPPGGMDYWGKGTQVTVSSAAAYPYDVPDYGSHHHHHH

SEQ ID NO: 348 - protein, 140 aa, synthetic construct
  REGION 1..140: note = Synthetic Sequence
QVQLQESGGGLVQAGDSLRLSCAGSEGTLSSYGIGWFRQAPGKEREFVGGINWSGDSTDYADSVKGRFTISRDSAKNTVYLQMNSLKPEDTAVYYCAAGESGVWVGGLDYWGQGTQVTVSSAAAYPYDVPDYGSHHHHHH

SEQ ID NO: 349 - protein, 152 aa, synthetic construct
  REGION 1..152: note = Synthetic Sequence
QVQLQESGGGLVQAGGSLRLSCAASGFSSDDYTIAWFRRAPGKEREGISCFSSSDGSAGYADSVKGRFTVSSDNAKNTVYLQMNSLKPEDTAVYYCAADSNVWSPPICGSTWYGPPPGGMAYWGKGTQVTVSSAAAYPYDVPDYGSHHHHHH

SEQ ID NO: 350 - protein, 150 aa, synthetic construct
  REGION 1..150: note = Synthetic Sequence
QVQLQESGGGLVQAGGSLRLSCATSGFTFDDYAIAWFRQAPGKEREGVSCISNTDSSTYYADSVKGRFTISSDNAKNTVYLQMSSLKPEDTAVYYCAADGNVWSPPICGSAGPPPGGMDYWGKGTQVTVSSAAAYPYDVPDYGSHHHHHH

SEQ ID NO: 351 - protein, 152 aa, synthetic construct
  REGION 1..152: note = Synthetic Sequence
QVQLQESGGGLVQAGGSLRLSCEVSGLSSDDYTIGWFRQAPGKEREGFSCFSTRDGNAGYADSVKGRFTISSDNAKNTVYLQMNNLKPEDTAVYYCAADFNVWSPPICGSRWYGPPPGGMDYWGKGTQVTVSSAAAYPYDVPDYGSHHHHHH

SEQ ID NO: 352 - protein, 152 aa, synthetic construct
  REGION 1..152: note = Synthetic Sequence
QVQLQESGGGLVQAGGSLRLSCEVSGLSSDDYTIGWFRQAPGKKREGFSCFSSRDGSTGYADSVKGRFTISSDNAKNTVYLQMNSLKPEDTAVYYCAADFNVWSPPICGSRWYGPPPGGMDYWGKGTQVTVSSAAAYPYDVPDYGSHHHHHH

SEQ ID NO: 353 - protein, 152 aa, synthetic construct
  REGION 1..152: note = Synthetic Sequence
QVQLQESGGGLVQAGGSLRLSCAASGFSSDDYTIGWFRQAPGKEREGFSCFSSRDGSTGYADSVKGRFTISSDNAKNTVYLQMNSLKPEDTAVYYCAADFNVWSPPICGSRWYGPPPGGMDYWGKGTQVTVSSAAAYPYDVPDYGSHHHHHH

SEQ ID NO: 354 - protein, 151 aa, synthetic construct
  REGION 1..151: note = Synthetic Sequence
QVQLQESGGGLVQAGGSLRLSCAASGFSFDDYTIGWFRQVPGKEREGISCISSDGSTGYADSVKGRFTISSDNAKNTVYLQINSLKPEDTAVYYCAADFNVWSPPICGSIWYGPPPGGMDYWGKGTQVTVSSAAAYPYDVPDYGSHHHHHH

SEQ ID NO: 355 - protein, 150 aa, synthetic construct
  REGION 1..150: note = Synthetic Sequence
QVQLQESGGGLVQAGGSLRLSCATFGFTFDDYAIAWFRQAPGKEREGISCISNTDSSTYYADSVKGRFTISSDNAKNTVHLQMSSLKPEDTAVYYCAADGNVWSPPICGSAGPPPGGMDYWGKGTQVTVSSAAAYPYDVPDYGSHHHHHH

SEQ ID NO: 356 - protein, 150 aa, synthetic construct
  REGION 1..150: note = Synthetic Sequence
  VAR_SEQ 19: note = Xaa can be any naturally occurring amino acid
QVQLQESGGGLVQAGGSLXLSCAASGFTFDDYAIGWFRQAPGKEREGVSCISSPDGSTYYADSVKGRFTISSDNAKNTVYLQMNSLKPEDTAVYYCAADFNVWSPPICGSVGPPPGGMDYWGKGTQVTVSSAAAYPYDVPDYGSHHHHHH

SEQ ID NO: 357 - protein, 152 aa, synthetic construct
  REGION 1..152: note = Synthetic Sequence
QVQLQESGGGLVQAGASLRLSCKASGFTFGDYTIGWFRQAPGKEREGISCYSSSDGNTGYADSVKGRFTISSDNAKNTVYLQMNSLRPEDTAVYYCAADFNVWSPPICGSSWYGPPPGGMAYWGKGTQVTVSSAAAYPYDVPDYGSHHHHHH

SEQ ID NO: 358 - protein, 140 aa, synthetic construct
  REGION 1..140: note = Synthetic Sequence
QVQLQESGGGLVQAGDSLRLSCAGSEGTFSSYGIGWFRQAPGKEREFVGGINWSGDSTDYADSVKGRFTISRDNAKNTVYLQMNSLKPEDTAVYYCAAGESGVWVGGLDYWGQGTQVTVSSAAAYPYDVPDYGSHHHHHH

SEQ ID NO: 359 - protein, 152 aa, synthetic construct
  REGION 1..152: note = Synthetic Sequence
QVQLQESGGGLVQAGGSLRLSCAASGFSSDDYTIGWFRQAPGKEREGISCFSSSDGSTGFADSVKGRFTISSDNATNTVYLQMNSLKPEDTAVYYCAADFNVWSPPICGSSWYGPPPGGMEYWGKGTQVTVSSAAAYPYDVPDYGSHHHHHH

SEQ ID NO: 360 - protein, 150 aa, synthetic construct
  REGION 1..150: note = Synthetic Sequence
QVQLQESGGGLVQAGDSLRLSCTASGVSIGDYNIGWFRQAPGKEREGVSCISSGDGTTYYTDSVKGRFTISTDNAKNTVYLQMNSLKPEDTAVYYCAADGNVWSPPICGSAGPPPGGMDYWGKGTQVTVSSAAAYPYDVPDYGSHHHHHH

SEQ ID NO: 361 - protein, 152 aa, synthetic construct
  REGION 1..152: note = Synthetic Sequence
QVQLQESGGGLVQAGGSLRLSCAASGFTFDDYTIAWFRQAPGGKEREGISCISSDGSTGYADSVKGRFTISSDNAKNMVYLQMNSLKPEDTALYYCAADFNVWSPPICSSNWYGPPPRGMDYWGKGTQVTVSSAAAYPYDVPDYGSHHHHHH

SEQ ID NO: 362 - protein, 151 aa, synthetic construct
  REGION 1..151: note = Synthetic Sequence
QVQLQESGGGLVQAGGSLRLSCAASGFTFDDYTIAWFRQAPGKEREGISCISSDGSTGYADSVRGRFTISSDNAKNTVYLQMNSLKPEDTAVYYCAADFNVWSPPICGSIWYGPPPRGMDYWGKGTQVTVSSAAAYPYDVPDYGSHHHHHH

SEQ ID NO: 363 - protein, 6 aa, synthetic construct
  REGION 1..6: note = Synthetic Sequence
HHHHHH

SEQ ID NO: 364 - protein, 10 aa, synthetic construct
  REGION 1..10: note = Synthetic Sequence
YPYDVPDYGS

SEQ ID NO: 365 - protein, 19 aa, synthetic construct
  REGION 1..19: note = Synthetic Sequence
AAAYPYDVPDYGSHHHHHH

SEQ ID NO: 366 - protein, 118 aa, synthetic construct
  REGION 1..118: note = Synthetic Sequence
EVQLVESGGGLVQPGGSLRLSCAASGFNIKDTYIHWVRQAPGKGLEWVARIDPANDNTLYASKFQGRATISADTSKNTAYLQMNSLRAEDTAVYYCGRGYGYYVFDHWGQGTLVTVSS

SEQ ID NO: 367 - protein, 118 aa, synthetic construct
  REGION 1..118: note = Synthetic Sequence
QVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSS

SEQ ID NO: 368 - protein, 107 aa, synthetic construct
  REGION 1..107: note = Synthetic Sequence
DVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIK

SEQ ID NO: 369 - protein, 165 aa, synthetic construct
  REGION 1..165: note = Synthetic Sequence
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 370 - protein, 165 aa, synthetic construct [sequence omitted]

SEQ ID NO: 371 - protein, 166 aa, synthetic construct
  REGION 1..166: note = Synthetic Sequence
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 372 - protein, 167 aa, synthetic construct
  REGION 1..167: note = Synthetic Sequence
MCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE

SEQ ID NO: 373 - protein, 166 aa, synthetic construct
  REGION 1..166: note = Synthetic Sequence
CDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE

SEQ ID NO: 374 - protein, 166 aa, synthetic construct
  REGION 1..166: note = Synthetic Sequence
CDLPQTHSLGNRRTLMLLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE

SEQ ID NO: 375 - protein, 166 aa, synthetic construct
  REGION 1..166: note = Synthetic Sequence
CDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNEDSILAVRKYFQRITLYLTEKKYSPCAWEVVRAEIMRSFSLSTNLQERLRRKE

SEQ ID NO: 376 - protein, 167 aa, synthetic construct
  REGION 1..167: note = Synthetic Sequence
MCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNEDSILAVRKYFQRITLYLTEKKYSPCAWEVVRAEIMRSFSLSTNLQERLRRKE

SEQ ID NO: 377 - protein, 167 aa, synthetic construct
  REGION 1..167: note = Synthetic Sequence
MCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNEDSILAVRKYFQRITLYLTEKKYSPCAWEVVRAEIMRSFSLCTNLQERLRRKE

SEQ ID NO: 378 - protein, 6 aa, synthetic construct
  REGION 1..6: note = Synthetic Sequence
EEFGNQ

SEQ ID NO: 379 - protein, 7 aa, synthetic construct
  REGION 1..7: note = Synthetic Sequence
EEFDGNQ

SEQ ID NO: 380 - protein, 166 aa, synthetic construct
  REGION 1..166: note = Synthetic Sequence
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 381 - protein, 143 aa, synthetic construct
  REGION 1..143: note = Synthetic Sequence
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ